(19) United States
(12) Patent Application Publication
(10) Pub. No.: US 2021/0369661 A1
(43) Pub. Date: Dec. 2, 2021

(54) METHODS AND COMPOSITIONS FOR TREATING AND RECOVERING FROM VIRAL INFECTIONS

(71) Applicant: IMMUNOFLEX THERAPEUTICS INC., Vancouver (CA)

(72) Inventors: VERNON TOSHIAKI OI, Kamuela, HI (US); CHRISTOPHER WILLIAM WAGNER, West Vancouver (CA); TIMOTHY ROBERT COTTRELL, Honolulu, HI (US); IAN DAVID MACDONALD, Surrey (CA)

(73) Assignee: IMMUNOFLEX THERAPEUTICS INC., Vancouver (CA)

(21) Appl. No.: 17/332,857

(22) Filed: May 27, 2021

Related U.S. Application Data
(60) Provisional application No. 63/030,832, filed on May 27, 2020.
(60) Provisional application No. 63/155,656, filed on Mar. 2, 2021.

(51) Int. Cl. A61K 31/198; A61K 31/352; A61K 31/12; A61K 31/05; A61K 31/27; A61P 37/04; A61K 36/28

(52) U.S. Cl. CPC A61K 31/198; A61K 31/352; A61K 31/12; A61K 36/28; A61K 31/27; A61P 37/04; A61K 31/05

(57) ABSTRACT

The described invention provides compositions and methods for improving or restoring immune system health in a susceptible subject and/or a subject infected with a respiratory virus that impacts the immune system by reducing functional diversity of T cells and promoting T cell exhaustion, compared to a control.

### CROSS-REFERENCE TO RELATED APPLICATIONS

This application claims the benefit of priority to U.S. provisional application 63/155,656 (filed Mar. 2, 2021) and to U.S. provisional patent application 63/030,832 (filed May 27, 2020). The content of each of these applications is incorporated by reference in its entirety.

### FIELD OF THE INVENTION

The described invention relates to compositions and methods that improve immune system vigor in susceptible subjects and in subjects infected with a respiratory virus.

### BACKGROUND OF THE INVENTION

Generally speaking, immune responses are initiated by an encounter between an individual and a foreign substance, e.g., an infectious microorganism. The infected individual rapidly responds with both a humoral immune response with the production of antibody molecules specific for the antigenic determinants/epitopes of the immunogen, and a cell mediated immune response with the expansion and differentiation of antigen-specific regulatory and effector T-lymphocytes, including cells that produce cytokines and killer T cells, capable of lysing infected cells. Primary immunization with a given microorganism evokes antibodies and T cells that are specific for the antigenic determinants/epitopes found on that microorganism; these usually fail to recognize or recognize only poorly antigenic determinants expressed by unrelated microbes [Paul, W. E., “Chapter 1: The immune system: an introduction,” Fundamental Immunology, 4th Edition, Ed. Paul, W. E., Lippincott-Raven Publishers, Philadelphia, (1999), at p. 102].

As a consequence of this initial response, the immunized individual develops a state of immunologic memory. If the same or a closely related microorganism is encountered again, a secondary response ensues. This secondary response generally consists of an antibody response that is more rapid, greater in magnitude and composed of antibodies that bind to the antigen with greater affinity and that are more effective in clearing the microbe from the body, and a similarly enhanced and often more effective T-cell response. However, immune responses against infectious agents do not always lead to elimination of the pathogen [Paul, W. E., “Chapter 1: The immune system: an introduction,” Fundamental Immunology, 4th Edition, Ed. Paul, W. E., Lippincott-Raven Publishers, Philadelphia, (1999), at p. 102].

The human immune system is a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other, but rather work together to elicit effective immune responses.

The innate arm of the immune system is a nonspecific fast response to pathogens that is predominantly responsible for an initial inflammatory response via a number of soluble factors, including the complement system and the chemokine/cytokine system; and a number of specialized cell types, including mast cells, macrophages, dendritic cells (DCs), and natural killer cells (NKs).

The adaptive immune arm involves a specific, delayed and longer-lasting response by various types of cells that create long-term immunological memory against a specific antigen. It can be further subdivided into cellular and humoral branches, the former largely mediated by T cells and the latter by B cells. This arm further encompasses cell lineage members of the adaptive arm that have effector functions in the innate arm, thereby bridging the gap between the innate and adaptive immune response.

### Human Immunodeficiency Virus

Human Immunodeficiency Virus (HIV) is a complex retrovirus which may be transmitted to humans from primates and between humans through the exchange of fluid, such as semen, vaginal and anal mucus, blood, and breast milk, through cuts, openings or mucous membranes of the human body. HIV is a rapidly mutating and recombining RNA virus that exhibits considerable genetic diversity with nine subtypes within just the major group of HIV Type I (HIV-1), rapid turnover rates, and persistency. HIV-1 can further be classified into 4 viral groups, or isolates: M, N, O, and P. The other major group of HIV is HIV Type 2 (HIV-2) (Moss, J. (2013) “HIV/AIDS Review” Radiologic Technology, Vol. 84, No. 3, 247-267). The majority of HIV/AIDS related deaths are concentrated in South Africa, however, the HIV infection is seen globally, including in sub-Saharan Africa, the United States, Europe, and Asia (Id.).

### 1.1 Structure-Based Functional Analyses of HIV

The mature HIV particle is round, measuring approximately 100 nm in diameter, with an outer lipid membrane as its envelope. The envelope contains 72 knobs, composed of trimers of the Env proteins. The trimers of gp120 surface protein (SU) are anchored to the membrane by the trimers of the transmembrane protein gp41 (TM). Conformation-dependent neutralizing epitopes are found on the gp120 protein. These are present on the native protein but are only partially expressed on the unfolded denatured protein. The viral envelope is composed of a lipid bi-layer and, in mature virus particles, the envelope proteins SU and TM. It covers the symmetrical outer capsid membrane, which is formed by the matrix protein (MA). The conical capsid is assembled from the inner capsid protein (CA). Depending on the section plane, the capsid appears as a cone, a ring or an ellipse. The tapered pole of the capsid is attached to the outer capsid membrane. Two identical molecules of viral genomic RNA are located inside the capsid and several molecules of the viral enzymes RT/RNase H and IN bound to the nucleic acid. Also present in virus particles are oligopeptides that are generated after release from the cell during the maturation of virions by proteolytic processing of the precursor proteins (p55, p160). [German Advisory Committee Blood (Arbeitskreis Blut), Subgroup ‘Assessment of Pathogens Transmissible by Blood’. (2016) “Human Immunodeficiency Virus (HIV).” Transfusion medicine and Chemotherapy: offizielles Organ der Deutschen Gesellschaft fur Transfusionsmedizin und Immunhamatologie, Vol. 43, 3: 203-22. doi:10.1159/000445852].

### 1.2 Immune Response to Infection with HIV

During the first stage of infection, known as the primary infection stage, the immune system of the infected person begins responding to the virus by generating HIV antibodies (in response to HIV antigens, a process known as seroconversion) and cytotoxic lymphocytes. Following seroconversion, a clinically asymptomatic period normally follows initial HIV infection, where levels of HIV in the peripheral blood decrease but function highly in the lymph nodes to destroy CD4 lymphocytes. The immune system becomes progressively damaged as the patient's immune system deteriorates from excessive damage to tissues and lymph nodes, viral mutation and increased destruction and reduced replacement of T cells [see Moss, J. (2013) “HIV/AIDS Review” Radiologic Technology, Vol. 84, No. 3, 247-267]. In the second stage, laboratory results indicate 14% to 29% CD4+ T cells per μL of blood and mild symptoms are perceived. In the third stage, CD4+ T cell count is below 14% and advanced symptoms are seen. By the fourth stage, acquired immunodeficiency syndrome has developed and severe symptoms are seen. [Id.].

A substantial reduction in the number of T cells seriously weakens the immune system. As CD4 lymphocyte counts decrease to fewer than 200 cells/μL of blood, symptomatic HIV infection can be triggered by the emergence of certain opportunistic infections that the immune system would normally prevent. Examples include pneumonia, diarrhea, eye infections, and meningitis. HIV patients are also susceptible to cancers and illnesses for example Kaposi sarcoma, non-Hodgkin lymphoma, central nervous system lymphoma, HIV encephalopathy, progressive multifocal leukoencephalopathy, lymphoid interstitial pneumonia, and HIV wasting syndrome. (Id.).

HIV initially infects CD4+CCR5+ T cells. The virus then spreads via the blood from the mucosal-associated lymphoid tissue to other lymphoid tissue, especially in the gut associated lymphoid tissue where it can replicate liberally. See Id. In acute HIV-1 infection, memory CD4+ T cells are massively depleted from the lymphoid system, particularly in the gut involving both direct targeting by the virus and bystander activation-induced cell death [See Mattapallil J J, et al. Nature 434: 1093-1097; see also Douek D C, et al. (2002) Nature 417: 95-98]. This applies to all memory CD4+ T-cell populations but those specific for HIV may be preferentially infected and destroyed [See Douek D C, et al. (2009) Annu Rev Med 60: 471-484]. However, the percentage of HIV-specific CD4+ T cells that are infected, even in the presence of high level viremia, is typically only a few percent or less, suggesting that the majority of these cells somehow escape infection despite being activated at a time of very high viremia.

Despite initial and persistent damage to CD4+ T cells, and a lack of detectable HIV-specific CD4+ T helper cells, the magnitude and breadth of CD8+ T-cell responses to HIV in infected humans were found to be robust, with direct effector function of such a magnitude that it could be readily detected in freshly isolated lymphocytes from peripheral blood and bronchoalveolar lavage in persons with AIDS. [Walker, B and McMichael, A. (2012 November) “Cold Spring Harb Perspect Med. 2(11): a007054; citing Murray, H W et al. (1984) N. Engl. J. Med. 310: 883-889; Lane, H C et al. (1985) “N. Engl. J. Med. 313: 79-84]. Acute phase CD8+ T-cell responses occur in the setting of acute phase proteins and pro-inflammatory cytokines. The initial response is narrowly directed, predominantly at epitopes in Env and Nef, regions that are among the most variable in the virus. The breadth of responses increases over time, as do the number of HLA alleles that are involved in recognition of infected cells. Immunization studies in animal models indicate that the CD8+ T-cell compartment has enormous expansion capacity, without affecting the size of the naïve CD4+, CD8+, or B-cell populations, and while preserving memory CD8+ T-cell populations to other pathogens. HIV-specific CD8+ T-cell responses remain detectable throughout the course of disease, and are actually broader and higher in persons with progressive infection than in those with controlled infection. [Id., citing Vezys, V. et al. (2009) Nature 457: 196-199; Pereyra F. et al. (2008) J. Infect. Dis. 197: 563-571].

Specificity of responses during the chronic phase of infection repeatedly suggests that Gag targeting is associated with lower viral load. [Id., citing Edwards, B H et al. (2002) J Virol 76: 2298-2305; Zuniga, R. et al. (2006) J Virol 80: 3122-3125; Kiepiela et al. (2007) Nat Med 13: 46-53]. In a large study of persons with clade C virus infection, the broader the Gag-specific response, the lower the viral load, and somewhat paradoxically, the broader the Env-specific response, the higher the viral load. [Id.; citing Kiepiela et al. (2007) Nat Med 13: 46-53; Ngumbela, K C et al. (2008) AIDS Res Hum Retroviruses 24: 72-82].

Generally, in those infected with HIV-1, the T-cell responses are dominated by CD8+ T cells. These are much stronger than CD4+ T-cell responses which are damaged by the virus. [Id., citing Ramduth, D et al. (2005) J Infect Dis 192: 1588-1596]. In murine models in which CD4+ T cells are depleted either with antibody infusion or genetically, CD8+ T-cell responses are greatly impaired. [Id., citing Janssen, E M et al. (2003) Nature 421: 852-856; Shedlock, D J and Shen, H (2003) Science 300: 337-339; Sun, J C and Bevan, MJ (2003) Science 300: 339-342]. On antigen stimulation, they expand rapidly to exhaustion and their IL-2-dependent progression to long term memory populations is abrogated. [Id., citing Kamimura, D and Bevan, MJ (2007) J Exp Med 204: 1803-1812]. In HIV-1 infection CD4+ T cells, though greatly depleted, are not entirely absent, but abnormalities in the development of CD8+ T-cell responses could be consistent with partial loss of CD4+ T-cell help, or impaired function of what cells remain [Id., citing Pitcher, C J et al. (1999) Nat Med 5: 518-525].

Cross-sectional data in chronically infected persons indicate a link between strong CD4+ T-cell responses and effective CD8+ T-cell responses. [Id., citing Kalams, S A et al. (1999) J Virol 73: 6715-6720]. Recent data implicated CD4+ T cells that make IL-21 as particularly important in maintaining CD8+ responses. [Id., citing Chevalier M F, et al. (2011) J Virol 85: 733-741; Williams L D, et al. J Virol (2011)85: 2316-2324)). While early studies showed a lack of CD4+ T-cell responses, it has been reported that, when patients were treated very early with antiretroviral drugs, strong CD4+ T-cell responses to HIV antigens could be rescued.

Antiviral CD8+ T cells were first identified as T cells that mediate lysis of virus-infected cells and are often referred to as cytotoxic T lymphocytes [Id., citing Plata F., et al. (1975) Eur J Immunol 5: 227-233]. Although most antigen-specific CD8+ T cells have this activity, they can use other effector mechanisms in addition. These include production of interferon-γ, IL-2, TNF-α, MIP-1α (renamed CCL3), MIP-10 (CCL4), and RANTES (CCL5). However, this effector function may not always be present and may take several days to appear. In contrast memory CD8+ T cells respond rapidly producing interferon-γ within a few hours. [Id., citing Lalvani, A. et al. (1997) J Exp Med 186: 859-865]. Production of lytic granules requires a bit longer but once activated, effector memory CD8+ T cells can release perforin and granzymes within minutes. [Id., citing Barber, D L et al. (2003) J Immunol 171: 27-31]. The delay in activating lytic functions in memory T cells probably protects the body from autoimmune attack when the TCR encounters weakly binding self antigens.

In patients who control virus well, the T cells are more quiescent than in acute infection. Many studies have shown that T cells in those who control HIV-1 well are polyfunctional, showing not only cytolytic potential but also have the capacity to produce cytokines and chemokines, although it is not clear whether this is cause or effect. [Id., citing Betts, M R and Harari, A (2008) Curr Opin HIV AIDS 3: 349-355]. Prolonged antigen stimulation in the absence of excessive activation and exhaustion, as occurs in slow progressors, could favor expression of multiple functions. Production of IL-2 may be important in the long term persistence of CD8+ T cells and can be provided by the CD8+ T cell itself or by CD4+ T cells, which survive much better in those whose disease progresses slowly. [Id.; citing Rosenberg, E S et al. (1997) Science 278: 1447-1450; Zimmerli, S C et al. (2005) Proc Natl Acad Sci 102: 7239-7244]. Similar observations have been made in HIV-2 infection in which elite controllers are relatively common. [Id.; citing Duvall, M G et al. (2008) Polyfunctional T cell responses are a hallmark of HIV-2 infection. Eur J Immunol 38: 350-363]. These findings are entirely consistent with data in CD4+ T-cell-depleted mice that show the importance of IL-2 in the maintenance of long-term CD8+ T-cell memory. [Id.; citing Williams M A, et al. (2006) Nature 441: 890-893)).

Evidence shows that CD8+ T cells are vital in controlling early HIV infection. Studies of human tissue samples have revealed that tissue related memory cells (T_{RM}) are generated in response to HIV infection in multiple locations, including the gastrointestinal tract and the female reproductive tract. Furthermore, individuals who appeared to naturally control infection had T_{RM} that were capable of producing the highest polyfunctional immune responses when compared to individuals who did not. [Muruganandah, V., et al. (2018). Frontiers in Immunology, 9, 1574. doi:10.3389/fimmu.2018.01574]. However, the TRM population within the HIV-specific CD8+ T cell compartment in individuals who controlled infection was under-represented when compared to individuals who were viremic. (Id.)

Similar to other infections in various sites, CD8+ T_{RM} in the context of HIV can be sub-divided into two subsets based on the expression of CD103 (also called human mucosal lymphocyte antigen 1, alpha E beta 7 integrin). Analysis of the ectocervical epithelium and menstrual blood revealed that HIV-infected women were more likely to have CD103-TRM when compared to healthy individuals. This reduced expression of CD103 may be explained by the HIV-induced depletion of CD4+ T cells which appear to be vital in providing help to CD8+ T cells for up-regulating CD103. The CD103− populations of the ectocervix resided closer to the basement membrane of the epithelium when compared to their CD103+ counterparts. The CD103+ population from infected individuals appears to express higher levels of PD-1. In a separate study, adipose PD-1+CD4+ TRM, appeared to remain relatively inactive during HIV infection and may serve as a reservoir for HIV. As such chronically activated T_{RM} and T_{RM} exposed to immunomodulated environments (such as the adipose tissue) may be unable to elicit a full effector response, favoring the progression of HIV infection. [Muruganandah, V., et al. (2018). Frontiers in Immunology, 9, 1574. doi:10.3389/fimmu.2018.01574].

It also appears that HIV has the ability to disrupt CCR5-mediated CD8+ T cell migration into the cervical mucosa, thereby impairing the development of T_{M} populations. Human studies suggest that T_{RM}, especially CD8+ TRM, play an important role in combating HIV infection In a Simian Immunodeficiency Virus model of rhesus macaques, intravenous administration of SIVmac239Anef generated a population of CD8+ T_{RM} in vaginal tissue and the gut that participated in protection. In a murine model, a mucosal vaccination strategy in which intranasal administration of an influenza-vector expressing the HIV-1 Gag protein p24 followed by an intravaginal booster induced CD8+ T_{RM} in the vagina. Antigen stimulation of these CD8+ T_{RM} resulted in the recruitment of B cells, natural killer cells, and CD4+ T cells. While the recruitment of innate and adaptive immune cells may be beneficial in early viral clearance, the recruitment of CD4+ T cells may be detrimental in the context of HIV as they are the target for HIV. Hence, incidental recruitment of CD4+ T cells to sites of HIV entry (female reproductive tract and rectum) by prime and pull vaccination strategies may unintentionally increase susceptibility to infection. [Muruganandah, V., et al. (2018). Frontiers in Immunology, 9, 1574. doi:10.3389/fimmu.2018.01574].

### 2. Influenza Virus

Influenza viruses are ubiquitous, causing acute respiratory disease and substantial morbidity and mortality each year [Bakre, A. et al., PLoS One (2013) 6: e66796, citing Thompson W W, et al. (2004) Influenza-associated hospitalizations in the United States. JAMA 292: 1333-1340; Thompson W W, et al. (2003) JAMA 289: 179-186; MMWR (2010) Estimates of Deaths Associated with Seasonal Influenza—United States, 1976-2007. Atlanta, Ga.: Centers for Disease Control and Prevention. pp. 1057-1062]. Influenza viruses belong to the family Orthomyxoviridae, are enveloped, and have an eight segmented, negative-sense, single-stranded RNA genome that encodes up to 11 proteins [Id., citing Palese P SM (2007) Fields Virology; Knipe D M H P, Ed. Philadelphia: Raven]. The viral envelope contains the surface glycoproteins and antigenic determinants, hemagglutinin (HA) and neuraminidase (NA), as well as the membrane ion channel protein, M2. Within the virion, the matrix protein (M1) provides structure and secures the viral ribonucleoprotein (vRNP) complexes consisting of viral RNA coupled to nucleoprotein (NP) and the three polymerase proteins (PB1, PB2 and PA). The remaining viral proteins include the nonstructural proteins, NS1 and NS2, and the PB1-F2 protein found in some virus species. The virus must infect a host cell to co-opt host proteins and pathways for the successful generation of progeny virus. Overlap was identified in pathways used for virus entry [Id., citing Shapira S D, et al. (2009) Cell 139: 1255-1267; Karlas, A. et al., Nature (2010) 463 (7282): 818-22; Konig R, et al. Nature (2010) 463: 813-817; Brass A L, et al. (2008) Science 319: 921-926], fusion of the endosomal and viral membrane [Id., citing Hao L, et al. (2008) Nature 454: 890-893 Karlas, A. et al., Nature (2010) 463 (7282): 818-22; Konig R, et al. Nature (2010) 463: 813-817; Brass A L, et al. (2008) Science 319: 921-926], transport of the viral components to the nucleus [Id., citing Karlas, A. et al., Nature (2010) 463 (7282): 818-22; Konig R, et al. (2009) Nature 463: 813-817], as well as late events including export of the vRNP complex and RNA into the cytoplasm [Id., citing Brass A L, et al. (2009) Cell 139: 1243-1254; Hao L, et al. (2008) Nature 454: 890-893; Shapira S D, Gat-Viks I, Shum B O, Dricot A, de Grace M M, et al. (2009) Cell 139: 1255-1267; Karlas, A. et al., Nature (2010) 463 (7282): 818-22; Konig R, et al. Nature (2010) 463: 813-817].

There are three genera of influenza viruses: influenza virus A, B, and C. The influenza viruses, especially influenza virus A, are the most variable of the respiratory viruses. Influenza A viruses are subtyped based on their two surface antigens: hemagglutinin (HA; H1-H16) and neuraminidase (NA; N1-N9), which are responsible for host receptor binding/cell entry and cleavage of the HA-receptor complex to release newly formed viruses, respectively. Aquatic birds are the natural reservoir of influenza A viruses, harboring all possible subtypes. (Id.)

Both influenza virus A and B exhibit antigenic drift. This phenomenon occurs when the surface antigens of the virus gradually change, progressively and directionally, to escape immunological pressure from the host species. Yearly epidemics of influenza virus A and B are caused worldwide by these drift variants, and contribute to mortality (an estimate 250-500,000 every year) in the elderly, and in those with pre-existing conditions, such as chronic cardiopulmonary or renal disease; diabetes, immunosuppression, or severe anemia). New lineages of influenza virus A emerge every few decades through re-assortment of gene segments in animal hosts infected with two different viruses (antigenic shift), resulting in global pandemics with varying severity due to the absence of immunity in the human population (e.g., 1918 Spanish flu: H1N1, 40-100 million deaths; 1957 Asian flu: H2N2, 2 million deaths; 1968 Hong Kong flu: H3N2, 500,000 deaths; 2009 H1N1-pdm09, 15,000 deaths). Sporadic dead-end human infections of animal (especially avian) viruses are known to occur and have caused concern regarding pandemic potential. Highly pathogenic H5N1 viruses were first detected in birds in 1996 in China. In 2003, the virus re-emerged in China. Since then it has become panzootic among poultry and wild birds. The disease presents as a rapidly progressive viral pneumonia with severe leucopenia and lymphopenia, progressing to acute respiratory distress syndrome (ARDS) and multi-organ dysfunction. In 2013, another avian influenza virus (H7N9) caused zoonotic transmission events to humans in China, with no recorded sustained human-to-human transmission. The case fatality rate was around 20% and the elderly were most affected. [Id.)

### 2.1 Host Immune Response to Influenza A Virus (IAV) Infection

The innate immune response, the first line of defense against viral infection, is rapid in response, but nonspecific. During IAV infection, viral conserved components called pathogen associated molecular patterns (PAMPs) are recognized by host pathogen recognition receptors (PRRs), such as retinoic acid-inducible gene-I protein (RIG-I) and toll-like receptor (TLR), leading to activation of innate immune signaling that finally induces the production of various cytokines and antiviral molecules [Chen, X, et al., Front. Immunol. (2018) 9: 320, citing Cao X. Nat Rev Immunol (2016) 16(1):35, Ouyang J, et al. Cell Host Microbe (2014) 16(5):616-26]. These PAMPs have certain characteristic of viral RNA that are not shared by cellular RNAs, such as regions of double-stranded RNA (dsRNA) or the presence of a 5′-triphosphate group [Id., citing Rehwinkel J, et al. Cell (2010) 140(3):397-408; Baum A, et al. Proc Natl Acad Sci USA (2010) 107(37):16303].

Pathogen recognition receptors have the ability to distinguish self from non-self molecules within the infected cells. RIG-I is the main receptor to recognize the intracellular ssRNA and transcriptional intermediates of IAVs in the infected host cells. [Id., citing Nturibi E, et al. J Virol (2017) 91(19):e1179-1117]. Non-self RNA and transcriptional products of IAVs in the cytoplasm are also sensed by melanoma differentiation-associated gene 5 [Id., citing Pichlmair A, et al. Science (2006) 314(5801):997-1001]. Following the recognition of PAMPs, RIG-I is activated and its caspase activation and recruitment domains (CARDs) are exposed. Then the CARD is modulated by dephosphorylation or ubiquitination by E3 ligases, such as TRIM-containing protein 25 (TRIM25) [Id., citing Munir M. Sci Signal (2010) 3(118):jc2.10.1126/scisignal.3118jc2]. Thus, CARD-dependent association of RIG-I and MAVS trigger the downstream transduction signaling at the outer mitochondrial membrane [Id., citing Yoneyama M, et al. Curr Opin Immunol (2015) 32:48]. Subsequently, the transcription factors, including interferon regulatory factor 3 (IRF3) and IRF7, and nuclear factor kappa-light-chain-enhancer of activated B cells (NF-κB) are activated, causing the expression of a variety of IFNs and cytokines [Id., citing Hiscott J, et al. Trends Mol Med (2006) 12(2):53-6].

Analysis of human samples has revealed that influenza-specific tissue-resident memory T cells (T_{RM}) can be found in substantial numbers in lung tissue, highlighting their role in natural infection. Despite expressing low levels of granzyme B and CD107a, these CD8+ TRM had a diverse T cell receptor (TCR) repertoire, high proliferative capacities, and were polyfunctional [Muruganandah, V., et al. (2018). Frontiers in Immunology, 9, 1574. doi:10.3389/fimmu.2018.01574]. Influenza infection history suggests a greater level of protection against re-infections is likely due to the accumulation of CD8+ TRM in the lungs. Furthermore, the natural immune response to influenza A virus infection in a rhesus monkey model demonstrated that a large portion of influenza-specific CD8+ T cells generated in the lungs were phenotypically confirmed as CD69+CD103+ TRM. Unlike lung parenchymal TRM, airway CD8+ TRM are poorly cytolytic and participate in early viral replication control by producing a rapid and robust IFN-γ response. Bystander CD8+ TRM may also take part in the early immune response to infection through antigen non-specific, NKG2D-mediated immunity. The generation of functional TRM that protect against heterosubtypic influenza infection appear to be dependent on signals from CD4+ T cells. [Muruganandah, V., et al. (2018). Frontiers in Immunology, 9, 1574. doi:10.3389/fimmu.2018.01574].

Toll-like receptors are critical PRRs that sense the pathogen outside of cell membrane and internally at endosomes and lysosomes [Chen, X, et al., Front. Immunol. (2018) 9: 320, citing Takeshita F, et al. J Virol (2006) 80(13):6218]. TLRs expressed on cell membrane are TLR1, 2, 4, 5, and 6 that recognize PAMPs derived from bacteria, fungi, and protozoa, while TLR3, 7, 8, and 9 are expressed on the surface of endosomes and lysosomes and exclusively recognize nucleic acid PAMPs derived from various viruses, including IAVs [Id., citing Kawai T, Akira S. Nat Immunol (2010) 11(5):373; Takeuchi O, Akira S. Cell (2010) 140(6):805; Kumar H, et al. Int Rev Immunol (2011) 30(1):16-34]. TLR3, TLR7, and TLR8 are involved in sensing the IAV components in cytoplasmic endosomes during the virus replication. It is known that TLR3 recognizes dsRNA in endosomes (Id., citing Goubau D, et al. Nature (2014) 514(7522):). Interestingly, it was shown that TLR3 may recognize recently unidentified RNA structures that are present in phagocytosed cells infected with IAVs [Id., citing Schulz O, et al. Nature (2005) 433(7028):887]. In plasmacytoid dendritic cells (pDCs), TLR7 recognizes the ssRNA of the influenza virions that are taken up into the endosomes [Id., citing Lund J M, et al. Recognition of single-stranded RNA viruses by toll-like receptor 7. Proc Natl Acad Sci USA (2004) 101(15):5598-603]. Then downstream signaling of TLR7 is activated via the adaptor protein myeloid differentiation factor 88 in pDCs, which results in the activation of either NF-κB or IRF7 to induce the expression of pro-inflammatory cytokines and type I IFNs, respectively [Id., citing Lund J M, et al. Proc Natl Acad Sci USA (2004) 101(15):5598-603]. In macrophages and DCs, TLR3 interacts with TIR-domain-containing adapter-inducing interferon-β. Such interaction results in activation of the serine-threonine kinases IKKE (IKKE) and TBK1 that phosphorylate IRF3 to regulate the expression of IFN-β [Id., citing Le Goffic R, et al. J Immunol (2007) 178(6):3368-72]. In human monocytes and macrophages, TLR8 is stimulated by its ligand ssRNA, leading to the production of IL-12. However, the relationship between TLR8 and IAV infection has not been defined [Id., citing Ablasser A, et al. J Immunol (2009) 182(11):6824].

Moreover, some NOD-like receptors, such as NOD-like receptor family pyrin domain containing 3 (NLRP3, also known as cryopyrin) and NLR apoptosis inhibitory protein 5, have been observed to be activated upon cellular infection with IAV [Id., citing Philpott D J, et al. Nat Rev Immunol (2014) 14(1):9]. NLRP3 is expressed by number of cells, including DCs, macrophages, neutrophils, monocytes, and human pulmonary epithelial cells [Id., citing Guarda G, et al. J Immunol (2011) 186(4):2529-34, Pothlichet J, et al. PLoS Pathog (2013) 9(4):e1003256]. Three signals are required for activation of inflammosome to trigger cytokine production. First, NLRP3 is activated through pathogen detection, which induces the expression of the genes encoding pro-IL-10, pro-IL-18, and pro-caspase-1 [Id., citing Martinon F, Mayor A, Tschopp J. Annu Rev Immunol (2009) 27(27):229]. Second, the IAV-encoded M2 ion channel is required to trigger NLRP3 inflammasome activation and cleavage of pro-IL-10 and pro-IL-18 [Id., citing Ichinohe T, et al. Nat Immunol (2010) 11(5):404-10]. Accumulation of IAV PB1-F2 in the lysosome of macrophages was reported to act as the third signal leading to activation of the NLRP3 inflammasome [Id., citing McCauley J L, et al. PLoS Pathog (2013) 9(5):e1003392].

### 2.2 Antiviral Molecules Involved in Innate Immunity against IAV Infection

Activation of specific transcription factors including, NF-κB, IRF3, and IRF7 during IAV infection results in translocation of these factors into the nucleus where they initiate the transcription of genes encoding IFNs and pro-inflammatory cytokines (TNF, IL6, IL10, etc.). It is well known that type I IFNs, such as IFN-α and IFN-β, and type III IFNs also known as interferon lambdas (IFN-λ1, IFN-λ2, IFN-λ3, IFN-λ4) play important roles in antiviral response in both virus-infected and uninfected cells [Id., citing Shan N C, et al. Dev Comp Immunol (2016) 61: 208]. Infection with IAV induces robust expression of type I and type III IFN genes [Id., citing Wang J, et al. J Immunol (2009) 182(3):1296-304]. Following the expression, IFN-α and IFN-β interact with IFN-x/o receptors (IFNAR), while IFN-λs interact with IFNL receptors (IFNLR) in an autocrine or paracrine manner, which activate Janus kinase-signal transducer and activator of transcription (JAK-STAT) signaling pathway. Phosphorylated STAT1 and STAT2 bind with IRF9 to form a complex ISG factor 3 (ISGF3). ISGF3 translocates into nucleus and binds with IFN-stimulated response element, which triggers the transcription of numerous IFN-stimulated genes (ISGs) [Id., citing Schneider W M, et al. Annu Rev Immunol (2014) 32(1):513]. Previous studies suggest that type I and type III IFNs provide similar defense against IAV infection in wild-type mice [Id., citing Mordstein M, et al. PLoS Pathog (2008) 43(3):e1000151]. It was further shown that IAV infection induced expression of same type of ISGs in epithelial cells of wild type, IFNAR- or IFNLR-deficient mice [Id., citing Crotta S, et al. PLoS Pathog (2013) 9(11):e1003773]. Only when both IFN-α/β and IFN-λ receptors in mice were knocked out, the animals failed to restrict non-pathogenic influenza virus [Id., citing Mordstein M, et al. PLoS Pathog (2008) 43(3):e1000151]. In spite of similar role of IFNα/β and IFN-λs to a certain extent, some notable differences exist. For example, it has been observed that mice infected with influenza virus showed higher pulmonary inflammation and mortality after treatment with IFNα, while IFN-k remained protective [Id., citing Davidson S, et al. EMBO Mol Med (2016) 8(9):1099-112, Kim S, et al. Am J Respir Cell Mol Biol (2016) 56(2):202-12].

These ISGs target different steps of IAV life cycle. For example, viral entry into cells can be restricted by several ISGs, including Mx family, interferon-induced transmembrane protein family (IFITMs), cholesterol 25-hydroxylase (CH25H), and TRIM proteins. Mx family is comprised of MxA and MxB in human, Mx1 and Mx2 in mice. Mx proteins are produced by various cells, such as hepatocytes, DCs, endothelial cells, and immune cells [Id., citing Femindez M, et al. J Infect Dis (1999) 180(2):262]. Mx proteins were the first ISGs identified to restrict IAV infection of mice [Id., citing Staeheli P, et al. Cell (1986) 44(1):147]. One study showed that MxA could retain incoming viral genome in the cytoplasm of human cell [Id., citing Xiao H, et al. J Virol (2013) 87(23):13053]. In addition, nuclear Mx1 in mice impedes the process of early transcription of IAV activated by the polymerase in the nucleus [Id., citing Pavlovic J, et al. J Virol (1992) 66(4):2564]. It is thought that the sensitivity of IAVs to MxA depends on their nucleocapsid proteins, and usually avian strains of IAVs are more sensitive to MxA than human strains [Id., citing Manz B, et al. PLoS Pathog (2013) 9(3):e1003279, Zimmermann P, et al. J Virol (2011) 85(16):8133-40]. However, role of MxB in humans or Mx2 in mice during IAV infection is poorly understood [Id., citing Iwasaki A, Pillai P S. Nat Rev Immunol (2014) 14(5):315-28].

Interferon-induced transmembrane protein families are known as new ISGs that restrict early viral entry by altering the cellular membrane properties like cell adhesion, fluidity, and spontaneous curvatures [Id., citing Evans S S, et al. Blood (1990) 76(12):2583-93, Kelly J M, et al. Eur J Biochem (1985) 153(2):367-71]. It has been found that IFITM proteins restrict the replication of IAVs by interfering virus-host cell fusion following viral attachment and endocytosis [Id., citing Brass A L, et al. Cell (2009) 139(7):1243-54]. Another antiviral ISG, CH25H is an integral element of cellular membranes and upregulated by IFN signaling. CH25H enzymatic activity converts cholesterol to soluble 25-hydroxycholesterol (25HC), which is involved in antiviral defense against enveloped viruses, including influenza virus through blocking viral fusion. Recently, it was suggested that high concentration of 25HC causes physical changes of cellular membrane properties to prevent viral fusion [Id., citing Liu S Y, et al. Immunity (2013) 38(1):92-105]. Moreover, previous studies showed that IFN-activated STAT1 bound to the promoter proximal region of the Ch25h gene to stimulate the production of 25HC that enhanced innate immune response against IAV [Id., citing Blanc M, et al. Immunity (2013) 38(1):106-18, Gold E S, et al. Proc Natl Acad Sci USA (2014) 111(29):10666-71]. In addition, TRIM proteins play multiple roles in antiviral immunity. TRIM25, an E3 ubiquitin ligase, is considered to regulate the re-localization of RIG-I to mitochondrion and signal transduction to MAVS for innate immune response against the viral infection [Id., citing Gack M U, et al. Nature (2007) 446(7138):916-20]. TRIM22 blocks IAV genome encapsidation and degrades nucleoprotein of IAV by polyubiquitination [Id., citing Di Pietro A, et al. J Virol (2013) 87(8):4523-33]. TRIM32 binds with influenza PB1 RNA polymerase, reduces the polymerase activity, and thus restricts the viral replication [Id., citing Fu B, et al. PLoS Pathog (2015) 11(6):e1004960].

There are increasing number of ISGs that regulate viral mRNA expression and protein translation. For example, zinc finger antiviral protein (ZAP), oligoadenylate synthase and ribonuclease L (OAS-RNase L), PKR, and ISG15 are involved in the regulation of IAV mRNA levels and protein synthesis. ZAPs inhibit the expression of IAV PB2 and PA proteins by reducing the viral mRNA expression and blocking its translation [Id., citing Tang Q, et al. J Virol (2017) 91(2):e1909-16, Liu C-H, et al. Proc Natl Acad Sci USA (2015) 112(45):14048-53]. OAS-RNase L can destroy viral RNA in the cytosol of host cells and finally halt the protein synthesis process and viral replication. Mice with reduced expression of RNase L are more prone to influenza virus infection [Id., citing Silverman R H. J Virol (2007) 81(23):12720-9]. PKR is expressed by all kind of cells and upregulated by type I and type III IFNs [Id., citing Ank N, et al. J Virol (2006) 80(9):4501-9]. PKR expressed in inactive form is activated by influenza virus infection. PKR is a known anti-IAV factor that binds to viral dsRNA and suppresses viral protein synthesis. Genetically deficient PKR mice are highly susceptible to influenza virus [Id., citing Dauber B, et al. Influenza B virus ribonucleoprotein is a potent activator of the antiviral kinase PKR. PLoS Pathog (2009) 5(6):e1000473]. ISG15 is an ubiquitin-like protein and restrict viral replication by interfering with virus release and translation of viral proteins [Id., citing Yuan W, Krug R M. EMBO J (2001) 20(3):362-71].

In addition, many other ISGs are also involved in innate immunity against IAV infection. These include viperin, tetherin, and so on (Id., citing Hu S, et al. Biochem J (2017) 474(5):715-30, GnirB K, et al. J Virol (2015) 89(18):9178-88). It has been shown that overexpression of viperin (also known as RSAD2) restricts the release of influenza virus by affecting the formation of lipid rafts specific microdomains that are particular budding sites of the virus [Id., citing Wang X, et al Cell Host Microbe (2007) 2(2):96]. Tetherin is another potential host antiviral factor. In 2008, it was reported that tetherin inhibited retrovirus release [Id., citing Neil S J, Zang T, Bieniasz P D. Nature (2008) 451(7177):425]. Tetherin appeared to limit cellular export of viral progenies by internalizing and degrading them exported to the surface of infected cells [d., citing Evans D T, S et al. Trends Microbiol (2010) 18(9):388-96]. It was also found that tetherin restrained the influenza virus by tethering and degrading newly budded viral particles [Id., citing Mangeat B, et al. J Biol Chem (2012) 287(26):22015-29]. Recently, it has been known that tetherin was able to suppress the budding of several laboratory oriented and seasonal influenza strains, but unable to restrict pandemic influenza A/Hamburg/4/2009 and wild-type influenza virus particles [Id., citing Hu S, et al. Biochem J (2017) 474(5):715-30, GnirB K, et al. J Virol (2015) 89(18):9178-88, Mangeat B, et al. J Biol Chem (2012) 287(26):22015-29].

### 2.3 Cells Involved in Innate Immunity against the IAV Infection

Airway epithelial cells are the first target of IAVs. These cells produce antiviral and chemotactic molecules that initiate immune responses by rapid recruitment of innate effector cells, such as NK cells, monocytes, and neutrophils. All cell types have their own unique mechanisms to interact with virus-infected cells to limit viral replication, and also prime adaptive immune cells for antigen-specific immunity and memory. Tumor necrosis factor alpha (TNF-α) and IL-1 induce endothelial adhesion molecules, which trigger the migration of innate immune cells, such as macrophages, blood borne DCs, and natural killer (NK) cells to the site of infection.

Alveolar macrophages are critical for limiting viral spread. Activated macrophages phagocytose IAV-infected cells and thus limit viral spread and regulate the following adaptive immune response [Id., citing Tumpey™, et al. J Virol (2005) 79(23):14933-44]. Monocytes derived from bone marrow precursors circulate in bloodstream. During IAV infection, MCP-1 (CCL-2) produced by infected epithelial cells attracts alveolar macrophages and monocytes via their CCR2 receptors [Id., citing Herold S, et al. J Immunol (2006) 177(3):1817.10.4049/jimmunol.177.3.1817]. NK cells are important cytotoxic lymphocytes of innate immune system to eliminate IAV infection. It has been reported that lysis of IAV-infected cells is mediated by the binding of IAV-HA with cytotoxicity NKp44 and NKp46 receptors [Id., citing Mendelson M, et al. J Virol (2010) 84(8):3789]. Expression of IAV-HA on the surface of infected cells is recognition signal for NK cells, and thereby NK cells target and lyse the infected cells [Id., citing Guo H, et al. J Leukoc Biol (2011) 89(2):189-94, van Helden M J, et al. J Immunol (2012) 189(5):2333-7].

Dendritic cells, the specialized antigen-presenting cells, bridge up the innate and adaptive immune responses during the IAV infection. Adaptive immune response begins when naïve and memory T lymphocytes recognize viral antigens presented by DCs. In the naïve steady state, DCs are orchestrated underneath the respiratory tract, including the airway epithelial tissue, lung parenchyma, and the alveolar spaces of the lungs [Id., citing Holt P G, et al. Nat Rev Immunol (2008) 8(2):142-52, Bahadoran A, et al. Front Microbiol (2016) 7:1841], where they constantly monitor for invading pathogens by their dendrites that are extended to airway lumen through the tight junctions of epithelial cells. Upon infection with IAV, the conventional DCs (cDCs) migrate from lungs to lymph nodes through interaction between CCR7 and its ligand CCL19 and CCL21 [Id., citing Heer A K, et al. J Immunol (2008) 181(10):6984-94]. In the lymph nodes, cDCs present antigens derived from IAV to T lymphocytes [Id., citing Geurts van Kessel C H, et al. J Exp Med (2008) 205(7):1621-34, Hintzen G, et al. J Immunol (2006) 177(10):7346-54]. The self-infected DCs degrade the viral protein into immune peptides. Immune peptides (epitopes) in the cytosol are exported to the endoplasmic reticulum, where they bind with major histocompatibility complex (MHC) class I molecule. Following the binding with epitopes, MHC class I is transported to the cell membrane via the Golgi complex for recognition by virus-specific CD8+ cytotoxic T cells (CTL). However, viral proteins degraded in endosomes/lysosomes are associated with MHC class II molecule. These complexes are presented on the cell membrane for recognition by CD4+ T helper (Th) cells. It has been proposed that this process may lead to B cell proliferation and maturation to antibody producing plasma cells [Id., citing van de Sandt C E, et al. Viruses (2012) 4(9):1438-76). In addition, DCs can exert cytolytic activity and contribute to the formation of bronchus-associated lymphoid tissue (BALT) during the IAV infection (Id., citing Geurts van Kessel C H, et al. PLoS One (2009) 4(9):e7187).

### 2.4 T Cells and B Cells Play Key Roles in Adaptive Immunity Against the IAV Infection

Studies have shown that IAV-specific CD8+ T cells can last for 2 years in murine models [Id., citing Valkenburg S A, et al. PLOS Pathog (2012) 8(2):e1002544]. The cytotoxicity of the memory CD8+ T cells decreases significantly, which is related to their declined target competence and reduced cytolytic molecule expression [Id., citing Grant E J, et al. Curr Opin Virol (2016) 16:132-42]. Autophagy plays a critical role in the establishment of memory CD8+ T cells, as Atg7-deficient mice are unable to form CD8+ T cell memory against IAV infection [Id., citing Puleston D J, et al. Elife (2014) 3(3):e03706]. IAV-specific memory CD8+ T cells in the nasal epithelia prevent the spread of the virus from the URT to the lung, thus blocking the development of pulmonary disease [Id., citing Pizzolla A, et al. Sci Immunol (2017) 2(12):eaam6970]. Besides, lung-resident memory CD8+ T cells can defend against heterologous IAV infection, via restraining viral replication and facilitating viral elimination [Id., citing Van B N, Harty J T. Immunol Cell Biol (2017) 95(8):651-5]. Additionally, lung-resident monocytes support to establish lung-resident CD8+ T cell during IAV infection [Id., citing Dunbar P, et al. J Immunol (2016) 196(1 Suppl):68-7].

CD4+ T cell is another important type of immune cells that is involved in adaptive immunity against the IAV infection. CD4+ T cells can also target IAV-infected epithelial cells through MHC class II and induce MHC class II expression in epithelial cells in murine models [Id., citing Brown D M, et al. J Virol (2012) 86(12):6792-803, McKinstry K K, et al. J Clin Invest (2012) 122(8):2847-56]. Multiple co-stimulatory ligands expressed by CD4+ T cells contribute to B cell activation and antibody production, among which CD40 ligand (CD40L) is noteworthy [Id., citing Swain S L, et al. Nat Rev Immunol (2012) 12(2):136-48]. CD40L has been shown to enhance immune response against the highly mutated HA protein of IAV [Id., citing Yao Q, et al. Vaccine (2010) 28(51):8147-56]. Similar to CD8+ T cells, CD4+ T cells are activated by DCs that migrate from the lung to the draining lymph nodes during the IAV infection [Id., citing Ingulli E, et al J Exp Med (1997) 185(12):2133-41, Lukens M V, et al. J Virol (2009) 83(14):7235-43]. CD4+ T cells differentiate into Th1 cells in response to IAV infection, according to their stimulators, including antigen, co-stimulatory molecules, and cytokines secreted by DCs, epithelial cells, and inflammatory cells [Id., citing Magram J, et al. Immunity (1996) 4(5):471-81, Pape K A, et al. J Immunol (1997) 159(2):591-8]. Th1 effector CD4+ T cells express antiviral cytokine, such as IFN-γ, TNF, and IL-2 [Id., citing Szabo S J, et al. Cell (2000) 100(6):655-69], and activate alveolar macrophages [Id., citing Liu S Y, et al. Proc Natl Acad Sci USA (2012) 109(11):4239-44]. The IL-2 and IFN-γ produced by Th1 cells regulate CD8+ T-cell differentiation to clear the viral infection [Id., citing Shu U, et al. Eur J Immunol (1995) 25(4):1125-8, Stuber E, et al. J Exp Med (1996) 183(2):693-8]. CD4+ T cells are also able to differentiate into Th2, Th17, regulatory T cells (Treg cells), follicular helper T cells, and sometimes as killer cells [Id., citing Zhu J, et al. Annu Rev Immunol (2010) 28:445-89]. Th2 cells bind to virus-derived MHC class II-associated peptides by antigen-presenting cells and produce IL-4 and IL-13 to promote B cell responses predominantly [Id., citing Lamb J R, et al. J Immunol (1982) 128(1):233-8]. It has been observed that Th17 and Treg cells are involved in regulating cellular immunity against IAV infection [Id., citing Mukherjee S, et al. Am J Pathol (2011) 179(1):248-58]. Although it is known that CD4+ T cells can direct CD8+ T cell responses by secreting various cytokines, the precise roles of CD4+ T cells to facilitate and regulate CD8+ T cell responses to IAV infection remain elusive, because primary CD8+ T cell response against IAV infection could be initiated independently of CD4+ T cells in mice [Id., citing La Gruta N L, Turner S J. Trends Immunol (2014) 35(8):396-402].

### 2.5 Lymphoid Tissues and Immunoglobulins in the Respiratory Tract Involved in Immunity against the IAV Infection

The nasal openings and URT are the main entry sites for IAVs and mucosal immune system also acts as the first line to limit the IAV infection apart from innate immunity. Secretory IgA (s-IgA) and IgM are the major neutralizing antibodies present on mucosa to prevent viral entry. Nasal secretions contain IgA which can neutralize HA and NA of IAVs [Id., citing Rangel-Moreno J, et al. Nat Immunol (2011) 12(7):639-46, Mazanec M B, et al. J Virol (1995) 69(2):1339-43]. During primary infection with IAVs, all three major immunoglobulin classes (IgG, IgA, and IgM) are present in mucosal secretion to limit the infection, though IgA and IgM are higher in concentration than IgG [Id., citing Murphy B R, Clements M L. Curr Top Microbiol Immunol (1989) 146:107-16]. It is thought that IgM response is dominant during primary infection, whereas during secondary infection IgG response is dominant for immunoglobulin secretion [Id., citing Cox R J, et al. Scand J Immunol (2004) 59(1):1-15, van de Sandt C E, et al. Viruses (2012) 4(9):1438-76, Kreijtz J H, et al. Virus Res (2011) 162(1-2):19-30]. In the URT, mucosal response is induced in the nasopharyngeal-associated lymphoid tissues (NALT) [Id., citing Zuercher A W, et al. J Immunol (2002) 168(4):1796-803; Fujimura Y, et al. Virchows Arch (2004) 444(1):36-42; Asanuma H, et al. J Immunol Methods (1997) 202(2):123-31]. When antigens are pinocytosed or phagocytosed by macrophages present on the NALT, they interact with local T and B cells, resulting in development of a large number of IgA Ab-forming cell (IgA-AFC) precursors [Id., citing Harty J T, Badovinac V P. Nat Rev Immunol (2008) 8(2):107-19, Wu T, et al. J Leukoc Biol (2014) 95(2):215-24]. The primed T and B cells migrate from NALT to the lungs via general circulation, where they differentiate into specific IgA-AFC to secrete antiviral antibodies. Thus, NALT appears to be initial inductive site for secretion of s-IgA against IAV infection. In the LRT, mucosal immune responses occur in the BALT [Id., citing Bienenstock J, et al. Immunologic and Infectious Reactions in the Lung, (1976) 1:29-58]. BALT is the site for AFC development and production of mucosal s-IgA against IAV infection [Id., citing Mikhak Z, et al. J Exp Med (2013) 210(9):1855-69].

Analysis of human samples has revealed that influenza-specific tissue resident memory T cells (T_{R}m) can be found in substantial numbers in lung tissue, highlighting their role in natural infection. Despite expressing low levels of granzyme B and CD107a, these CD8+ TRM had a diverse T cell receptor (TCR) repertoire, high proliferative capacities, and were polyfunctional [Muruganandah, V., et al. (2018). Frontiers in Immunology, 9, 1574. doi:10.3389/fimmu.2018.01574]. Influenza infection history suggests a greater level of protection against re-infections is likely due to the accumulation of CD8+ T_{RM} in the lungs. Furthermore, the natural immune response to influenza A virus infection in a rhesus monkey model demonstrated that a large portion of influenza-specific CD8+ T cells generated in the lungs were phenotypically confirmed as CD69+CD103+ T_{RM}. Unlike lung parenchymal T_{RM}, airway CD8+ TRM are poorly cytolytic and participate in early viral replication control by producing a rapid and robust IFN-γ response. Bystander CD8+ T_{RM} may also take part in the early immune response to infection through antigen non-specific, NKG2D-mediated immunity. The generation of functional TRM that protect against heterosubtypic influenza infection appear to be dependent on signals from CD4+ T cells. [Muruganandah, V., et al. (2018). Frontiers in Immunology, 9, 1574. doi:10.3389/fimmu.2018.01574].

A role for CD4+ TRM has also been reported. Much like their CD8+ counterparts, CD4+ T_{RM} also produce a significant IFN-γ response during early infection. Aside from the CD8+ and CD4+ subsets of T_{RM}, a subset of NK1.1+ double negative T memory cells which reside in the lungs also play a role in influenza infection. Taken together, these studies and others demonstrate that T_{RM} are required for optimal protection. However, unlike T_{R}m in other locations, such as the skin, lung T_{RM} are not maintained for extended periods of time. The gradual loss of lung T_{RM} appears to be the reason for the loss in heterotypic immunity against influenza infection. Lung T_{RM} exhibit a transcriptional profile that renders them susceptible to apoptosis. Despite conflicting evidence, it appears that maintenance of the lung CD8+ T_{RM} populations relies on the continual seeding from circulating CD8+ T cells. However, with time, circulating CD8+ T cells adopt a transcriptional profile that reduces their capacity to differentiate into Tm. There is also conflicting evidence regarding the requirement of local antigen for the generation of T_{M} within the lung. Intranasal administration of Live Attenuated Influenza Vaccine (FluMist®) in a mouse model induced both CD4+ and CD8+ T_{RM} that provided a degree of cross-strain protection independent of TCM and antibodies. The intranasal administration of a PamCys2 or Adjuplex™ has demonstrated capacity for producing protective influenza-specific lung CD8+ T_{RM} in similar numbers and IFN-γ secreting potential when compared to the natural response to influenza infection. [Muruganandah, V., et al. (2018). Frontiers in Immunology, 9, 1574. doi:10.3389/fimmu.2018.01574].

### 2.6 Role of s-IgA Antibody in Defense Against the IAV Infection

Secretory IgA is the primary isotype detected at the mucosal surface [Chen, X. et al., Frontiers Immunol. (2018) 9: 320, Id., citing Chiu C, Ellebedy A H, Wrammert J, Ahmed R. B Cell Responses to Influenza Infection and Vaccination. Chain, Z G: Springer International Publishing; (2014). 381], which contributes to mucosal protection through its distinct ability to remove an agent before it traverses the mucosal barrier and infects the cell [Id., citing Van R E, A et al. Vaccine (2012) 30(40):5893]. By covering the viral surface, s-IgA prevents the influenza virions from adhering to the susceptible cells, and thus inhibits their invading host cells and neutralizes the viruses without complement participation. Investigations have been demonstrated that s-IgA plays vital roles both in protection against homologous IAV infection and in cross-protection against URT infection by the viral variants [Id., citing Asahi Y, et al. J Immunol (2002) 168(6):2930]. Generally, parenteral administration of IAV vaccine leads to the generation of serum IgG, but not s-IgA, while s-IgA and IgG are both induced by intranasal administration [Id., citing Van R E, et al. Vaccine (2012) 30(40):5893; Akira A, et al. Hum Vaccin Immunother (2013) 9(9):1962-70]. Further, polymeric s-IgA is involved in defending against influenza in humans. Moreover, the quaternary structure of the polymeric s-IgA seems to play a key role in protecting human URT from influenza, and have more neutralizing capacity against IAVs than dimeric s-IgA [Id., citing Suzuki T, et al. Proc Natl Acad Sci USA (2015) 112(25):7809].

### 2.7 Escape of IAVs from Host Immune Surveillance: The Antagonism of Major IAV Proteins

To establish a successful infection, IAVs have evolved multiple strategies to circumvent the host immunity. For example, it is well known that IAV infection triggers robust production of IFNs that induce the expression of numerous antiviral molecules or ISGs. Although IFNs have a strong antiviral activity, they cannot fully control IAV infection due to the virus-mediated suppression of IFNs signaling.

Hemagglutinin of IAVs has been shown to facilitate IFNAR ubiquitination and degradation, reducing the levels of IFNAR, and thus suppressing the expression of IFN-stimulated antiviral proteins [Id., citing Xia C, et al. J Virol (2015) 90(5):2403-17]. It has been described that two discrete antigenic sites, H9-A and H9-B, may provide a novel mechanism for H9N2 virus to counteract humoral immunity [Id., citing Peacock T, et al. Sci Rep (2016) 6:18745]. In addition, a study has shown that the escape of H5N1 from vaccine-mediated immunity is caused by the addition of N-glycosylation sites on the globular head of HA [Id., citing Herve P L, et al. Virology (2015) 486(8):134-45]. In contrast, antibody response against NA of IAV cannot inhibit viral infection, but restrain its diffusion, thus lowering the severity of influenza. IAVs employ NA protein to block the recognition of HA by natural cytotoxicity receptors, NKp46, and NKp44 receptors and evade the NKp46-mediated elimination, leading to minimized clearance of infected cells by NK cells [Id., citing Baron Y, et al. J Infect Dis (2014) 210(3):410].

Nonstructural protein-1 of IAVs is the most important IFNs antagonist protein, acting on multiple targets and suppressing the host IFN response. Viral RNA invading the host cell causes RIG-I ubiquitination by a RING-finger E3 ubiquitin ligase named as TRIM25, which is essential for RIG-I signaling pathway to trigger host antiviral innate immunity [Id., citing Gack M U, et al. Proc Natl Acad Sci USA (2008) 105(43):16743]. However, NS1 protein can inhibit the TRIM25-mediated RIG-I ubiquitination, thereby blocking RIG-I activation [Id., citing Gack M U, et al Cell Host Microbe (2009) 5(5):439-49]. Moreover, NS1 has an inhibitory effect on protein kinase RNA-activated (also known as protein kinase R, PKR), but the effect relies on the induced expression of vault RNAs (a kind of small non-coding RNA with approximately 100 bases). They are initially described as fornix RNP complex components [Id., citing Kedersha N L, Rome L H. J Cell Biol (1986) 103(3):699-709]. Through NS1 protein, influenza virus induces the expression of vault RNA that inhibits the activation of PKR and the production of IFNs and ultimately promotes the replication of the virus. In a recent reverse genetic investigation, it was found that after interfering with NS1, the phosphorylation level of PKR dramatically increased, which was attenuated by forced expression of vault RNAs [Id., citing Li F, et al. Nucleic Acids Res (2015) 43(21):10321-37]. These data indicate that IAV has evolved a critical mechanism by which NS1-mediated PKR inhibition is mediated by upregulation of the host factor vault RNAs that inactivates PKR and blocks the production of downstream effector molecules of IFNs.

In addition, studies have shown that through the interaction with IκB kinases (IKK) α and β, two important kinases in NF-κB pathway, NS1 protein can block the phosphorylation of these kinases and eventually destroy the NF-κB complex predominating in nucleus as well as the expression of downstream genes [Id., citing Rückle A, et al. J Virol (2012) 86(18):10211-7, Gao S, et al Cell Microbiol (2012) 14(12):1849]. Also, through the JAK-STAT pathway, NS1 protein can block IFN-mediated downstream signaling pathway and weaken the antiviral effect mediated by the downstream effector molecules induced by IFNs. Specifically, NS1 acts mainly by lowering the phosphorylation levels of STAT1, STAT2, and STAT3, preventing STAT2 from entering into the nucleus to bind to the DNA sequence of ISGs promoter region, leading to reduced expression of ISGs [Id., citing Jia D, et al PLoS One (2010) 5(11):e13927]. NS1 is not only involved in host innate immunity, but also affects adaptive immunity via modulating the maturation and the capacity of DCs to induce T cell responses [Id., citing Femandez-Sesma A, et al. J Virol (2006) 80(13):6295-304]. Evidence also indicates that influenza virus NS1 can bind to cellular double-stranded DNA (dsDNA), counteract the recruitment of RNA polymerase II (Pol II) to DNA, and finally block the transcription of IFNs and ISGs [Id., citing Anastasina M, et al. Biochim Biophys Acta (2016) 1859(11):1440-8].

### 2.8 The Antagonism of Other IAV Proteins

Studies have found that PB1-F2 protein has a mitochondrial positioning signal, via interacting with MAVS, to counteract RLR-mediated activation of IFN signaling pathway (Id., citing Varga Z T, et al. PLoS Pathog (2011) 7(6):e1002067). Investigation on the interaction between the virus and host by systematic biology analysis has revealed that PB2 protein, a member of the viral polymerase complex, also plays roles in IFN antagonism [Id., citing Iwai A, et al. J Biol Chem (2010) 285(42):32064-74]. Furthermore, PB2 interacts with the MAVS to evade from the host IFN antiviral response, which is similar to the action mode of PB1-F2 protein [Id., citing Grimm D, et al. Proc Natl Acad Sci USA (2007) 104(16):6806-11]. Recently, viral M2 protein has been found to interfere with the host autophagy [Id., citing Mtnz C. et al. Cell Host Microbe (2014) 15(2):130-1, Beale R, et al. Cell Host Microbe (2014) 15(2):239-47]. These studies have suggested that viral M2 may inhibit the activation of TLR pathway and the generation of IFNs via blocking the host autophagy

### 3. Coronavirus

Coronaviruses (CoVs), a large family of single-stranded RNA viruses, can infect a wide variety of animals, including humans, causing respiratory, enteric, hepatic and neurological diseases. [Yin, Y., Wunderink, R G, Respirology (2018) 23 (2): 130-37, citing Weiss, S R, Leibowitz, I L. Adv. Virus Res. (2011) 81: 85-164]. Human coronaviruses, which were considered to be relatively harmless respiratory pathogens in the past, have now received worldwide attention as important pathogens in respiratory tract infection. As the largest known RNA viruses, CoVs are further divided into four genera: alpha-, beta-, gamma- and delta-coronavirus. In humans, CoVs cause mainly respiratory tract infections. Until November 2019, only six human coronaviruses (HCoVs) had been identified. These included the alpha-CoVs HCoV-NL63 and HCoV-229E and the beta-CoVs HCoV-OC43, HCoV-HKU1, severe acute respiratory syndrome-CoV (SARS-CoV), [Id., citing Drosten, C. et al. N. Engl. J. Med. (2003) 348: 1967-76] and Middle East respiratory syndrome-CoV (MERS-CoV) [Id., citing Zaki, A M et al. N. Engl. J. Med. (2012) 367: 1814-20].

Human-to-human transmission of SARS-CoV and MERS-CoV occurs mainly through nosocomial transmission. From 43.5-100% of MERS patients in individual outbreaks were linked to hospitals, [Id., citing Hunter, I C et al. Emerg. Infect. Dis. (2016) 22: 647-56. Osong Public Health Res. Perspect. (2015) 6: 269-78], which was similar in SARS patients. [Anderson, R M et al. Philos. Trans. R. Soc. Lond. B. Biol. Sci. (2004) 359: 1091 105]. A study from the Republic of Korea revealed that index patients who transmitted to others had more non-isolated days in the hospital, body temperature of >38.5° C. and pulmonary infiltration of >3 lung zones. [Id., citing Kang, C K, et al. J. Korean Med. Sci. (2017) 32: 744-49]. Transmission between family members occurred in only 13-21% of MERS cases and 22-39% of SARS cases. [Id., citing Kang, C K, et al. J. Korean Med. Sci. (2017) 32: 744-49]. Another Korean study suggested that transmission of MERS from an asymptomatic patient is rare. [Id., citing Moon, S Y, Son, J S. Clin. Infect. Dis. (2017) 64: 1457-58]. In contrast to SARS-CoV and MERS-CoV, direct human-to-human transmission was not reported for the other four HCoVs. [Id., citing Woo, P C et al. Hong Kong Med. J. (2008) 15 (Suppl. 9): 46-47].

Current understanding of the pathogenesis of HCoVs infection is still limited. However, several significant differences in the pathogenesis exist among SARS-CoV, MERS-CoV and the other HCoVs.

### 3.1 Cell Entry and Receptors

The critical first step for HCoV infection is entry into the susceptible host cells by combining with a specific receptor. Spike proteins (S proteins) of HCoVs are a surface-located trimeric glycoprotein consisting of two subunits: the N-terminal S1 subunit and the C-terminal S2 subunit. The S1 subunit specializes in recognizing and binding to the host cell receptor while the S2 region is responsible for membrane fusion. [Id., citing Gallagher, T M, Buchmeier, M J. Virology (2001) 279: 371-74]. To date, a wide range of diverse cellular receptors specifically recognized by the S1 domains have been identified for all HCoVs except HCoV-HKU1.

ACE2, the receptor for SARS-CoV and HCoV-NL63 [Id., citing Weiss, S R, Leibowitz, I L, Adv. Virus Res. (2011) 81: 85-164), Li, W. et al. Angiotensin-converting enzyme 2 is a functional receptor for the SARS coronavirus. Nature (2003) 426: 450-54; Hofmann, H. et al., Proc. Natl Acad. Sci. USA (2005) 102: 7988-93] is a surface molecule localized on arterial and venous endothelial cells, arterial smooth muscle cells, epithelia of the small intestine and the respiratory tract. In the respiratory tract, ACE2 is expressed on the epithelial cells of alveoli, trachea, and bronchi, bronchial serous glands, and alveolar monocytes and macrophages. ACE2 is a homologue of the ACE protein, and both are key enzymes of the renin-angiotensin system. ACE2 plays a protective role in lung failure and its counterpart ACE promoting lung edema and impaired lung function. [Id., citing Imai, Y., et al. Cir. J. (2010) 74: 405-10]. Downregulation of ACE2, as occurs during SARS-CoV infection, is believed to contribute to pathological changes in the lung. [Id., citing Imai, Y., et al. Cir. J. (2010) 74: 405-10; Kuba, K. et al. Nat. Med. (2005) 11: 875-79]. This form of lung damage can be attenuated by blocking the renin-angiotensin pathway. [Id., citing Kuba, K. et al. Nat. Med. (2005) 11: 875-79]. HCoV-NL63 also employs the SARS receptor for cellular entry [Id., citing Hofmann, H. et al., Proc. Natl Acad. Sci. USA (2005) 102: 7988-93] despite their markedly different pathogenicity and disease courses. This finding suggests that receptor usage may not be the only factor that determines the severity of HCoV infection.

Dipeptidyl peptidase 4 (DPP4, also known as CD26), the receptor for MERS-CoV, [Id., citing Meyerholz, D K et al. Am. J. Pathol. (2016) 186: 78-86] is a multifunctional cell-surface protein widely expressed on epithelial cells in kidney, small intestine, liver and prostate and on activated leukocytes. DPP4 is expressed in the upper respiratory tract epithelium of camels. [Id., citing Widagdo, W. et al. J. Vir. (2016) 90: 4838-42]. In the human respiratory tract, DPP4 is mainly expressed in alveoli rather than the nasal cavity or conducting airways. [Id., citing Meyerholz, D K et al. Am. J. Pathol. (2016) 186: 78-86] DPP4 is a key factor in the activation of T cells and immune response costimulatory signals in T cells, which could indicate a possible manipulation of the host immune system. [Id., citing Boonacker, E., Van Noorden, C J. Eur. J. Cell Biol. (2003) 82: 53-73].

Human aminopeptidase N (CD13), a cell-surface metalloprotease on intestinal, lung and kidney epithelial cells, has been identified as the receptor for hCoV-229E. [Id., citing Yeager, C L et al. Nature (1992) 357: 420-22]. The receptor for HCoV-OC43 is 9-O-acetylated sialic acid. Currently, the receptor for HCoV-HKU1 has not been identified.

### 3.2 Interferon and Interferon-Stimulated Genes

The interferon (IFN) family of cytokines, including IFN-α, IFN-β and IFN-γ, provide the first line of defense against viral pathogens. They initiate transcription of hundreds of IFN-stimulated genes (ISGs) that have antiviral, immune modulatory and cell regulatory functions.

Delayed recognition is critical for HCoVs to survive and replicate in the host. In vitro studies showed that both SARS-CoV and MERS-CoV have evolved genetic mechanisms to delay IFN induction and dysregulate ISG effector functions in primary human airway epithelial cells or in cultured cells. [Id., citing Manachery, V D, et al. Mol. Bio. (2014) 5: e01174-14; Lau, S K et al. J. Gen. Virol. (2013) 94: 2679-90]. It was reported that SARS-CoV infection could result in IFN-α induction only after 12 h in cultured Calu3 cells, with IFN-β5 and IFN-γ1 induction even further delayed. [Id., citing Manachery, V D, et al. Mol. Bio. (2014) 5: e01174-14] Similar to SARS-CoV, MERS-CoV also fails to induce IFNs prior to 12 h, with the exception of IFN-α5. Lau et al. serially measured mRNA levels of eight cytokine genes up to 30 h post-infection in Calu-3 cells infected with MERS-CoV and SARS-CoV. [Id., citing Lau, S K et al. J. Gen. Virol. (2013) 94: 2679-90]. Calu-3 cells infected by MERS-CoV showed marked induction of the proinflammatory cytokines IL-1β, IL-6 and IL-8 at 30 h but lack of production of the innate antiviral cytokines tumor necrosis factor (TNF)-α, IFN-β and IFN-γ-induced protein-10, compared with SARS-CoV. These data suggest that MERS-CoV attenuates innate immunity and induces a delayed proinflammatory response in human lung epithelial cells, which correlates with disease severity and clinical course.

### 3.3 Demographic and Clinical Features

Both SARS and MERS present with a spectrum of disease severity ranging from flu-like symptoms to acute respiratory distress syndrome (ARDS).

Age and underlying disease are significant independent predictors of various adverse outcomes in SARS. [Id., citing Chan, K S, et al. Respirology (2003) 8-Suppl.: 536 40]. SARS cases were mainly seen in young healthy individuals; whereas half of the cases of MERS-CoV infection occurred in individuals older than 50 years. [Id., citing Assiri, A. et al. Lancet Inf. Dis. (2013) 13: 752-61]. Compared with SARS patients, pre-existing chronic illnesses, such as diabetes (31%), hypertension (33%), chronic renal failure (15%), chronic heart disease (15%) and chronic pulmonary disease (13%), were more frequent in MERS patients. Clinical symptoms on admission included fever, cough, myalgia and shortness of breath in both SARS and MERS patients, while symptoms of upper respiratory tract infection such as sore throat were also frequent. Atypical symptoms such as diarrhea and vomiting developed in both SARS and MERS patients.

### 3.4 Kidney Impairment

Acute kidney injury (AM) is a significant characteristic of both SARS and MERS patients. One study reported that 6.7% of SARS patients had acute renal impairment and 84.6% had proteinuria.[Id., citing Chu, K H et al. Kidney Int. (2005) 67: 98-705]. AKI is much more common in MERS patients, occurring in up to 43%. [Id., citing Saad, M. et al. Int. J. Infect. Dis. (2014). 29: 301-6].

The mechanism of the high AM incidence in both SARS and MERS patients is not well clarified. Pre-existing co-morbid conditions and direct viral involvement of the kidneys [Id., citing Das, K M, et al. Am. J. Roentgenol. (2015) 204: 736-42; Morgenstern, B. et al., Biochem. Biophys. Res. Commun. (2005) 326: 905-8] may contribute to development of AKI. [Id., citing Chu, K H et al. A Kidney Intl. (2005) 67: 698-705; Saad, M. et al. Int. J. Infect. Dis. (20140 29: 301-06). Since ACE2 and DPP4, the receptors for SARS-CoV and MERS-CoV, are expressed at high levels in the kidney, functional impairment of these cell receptors by viral binding may contribute to the risk of AM. Elevated creatinine kinase (CK) values (176-1466 U/L) observed in 36% of SARS patients suggests rhabdomyolysis may also contribute. [Id. citing Peiris, J S, et al. Lancet (2003) 361: 1767-72].

### 3.5 Cardiovascular Manifestations

A cardinal difference between MERS and SARS is the frequency of cardiovascular involvement. Despite the high lethality, shock was distinctly unusual in SARS until late stages when hypotension likely resulted from bacterial superinfections.[Id., citing Booth, C M, et al. JAMA (2003) 289: 2801-9; Lee, N. et al. A major outbreak of severe acute respiratory syndrome in Hong Kong. N. Engl. J. Med. (2003) 348: 1986-94; Almekhlafi, G. A., et al., Crit. Care (2016) 20: 123]. In contrast, need for vasopressor therapy was much more common in MERS, [Id., citing Chan, K S, et al. Respirology (2003): 8-(Suppl.: S36-40; Almekhlafi, G. A. et al., Crit. Care (2016) 20: 123) up to 81% in one series. (Id., citing Almekhlafi, G. A. et al., Crit. Care (2016) 20: 123). Need for vasopressors was an independent risk factor for death in the intensive care unit (ICU) (odds ratio=18.3, 95% confidence interval: 1.1-302.1, P=0.04). [Id., citing Almekhlafi, G. A. et al., Crit. Care (2016) 20: 123]. Multi-organ involvement was seldom reported with the endemic HCoV infections, despite occasional fatal pneumonia in highly immunocompromised patients.

### 3.6 Other Manifestations

Hematological abnormalities such as thrombocytopenia and lymphopenia were common in both SARS [Id., citing Peiris, J S, et al., Lancet (2003) 361: 1767-72; Booth, C M et al., JAMA (2003) 289: 2801-9] and MERS patients. [Id., citing Assiri, A. et al. Lancet Infect. Dis. (2013) 13: 752-61; Arabi, Y M, et al, Ann. Intern. Med. (2014) 160: 387-97]. Thrombocytopenia and lymphopenia may be predictive of fatal outcome in MERS-CoV patients. [Id., citing Arabi, Y M, et al, Ann. Intern. Med. (2014) 160: 387-97]. Other laboratory findings included elevated CK, lactate dehydrogenase, alanine aminotransferase and aspartate aminotransferase levels.

### 3.7 Radiological Features

Air-space opacities are the main radiographical feature in SARS patients. [Id., citing Booth, C M, et al. JAMA (2003) 289: 2801-9; Wong, K T, et al. Radiology (2003) 228: 401-6]. In one retrospective study, initial chest radiographs were abnormal in 108 of 138 (78.3%) of SARS patients and all showed air-space opacities. [Id., citing Wong, K T, et 1. Radiology (2003) 228: 401-6]. Of these 108 patients, 59 had unilateral focal involvement while 49 had either unilateral multifocal or bilateral involvement. Lower lung zone (64.8%) and right lung (75.9%) were more commonly involved. Four patterns of radiographical progression were recognized in those patients: type 1) initial radiographical deterioration to peak level followed by radiographical improvement occurred in in the majority (97 of 138 patients, 70.3%); type 2) fluctuating radiographical changes were seen in 24 patients (17.4%); type 3) static radiographical appearance in 10 patients (7.3%); and type 4) progressive radiographic deterioration in 7 patients (5.1%). In contrast, the most common radiographical features in MERS patients were ground-glass opacities and consolidation. [Id., citing Choi, W S, et al. Infect. Chemother. (2016) 48: 118-26; Das, K M, et al. Am. J. Roentgenol. (2015) 205: W267-74]. It was reported that ground-glass opacity was the most common abnormality (66%) in MERS patients, followed by consolidation (18%). [Id., citing Das, K M et al., Am. J. Roentgenol. (2015) 205: W267-74]. Meanwhile, type 2 radiographical progression (20 patients) was most common in those MERS patients, followed by type 4 (14 patients) and type 3 (7 patients). Type 1 radiographical progression was observed only in four patients. Pleural effusion (P=0.001), pneumothorax (P=0.001) and type 4 radiographical progression (P=0.001) were more frequent in MERS patients who died compared with recovered patients. Similar to the radiographical findings, computed tomography findings in MERS patients also included ground-glass opacity (53%), consolidation (20%) or a combination of both (33%). [Id., citing Das, K M, et al. Am. J. Roentgenol. (2015) 204: 736 42]. Pleural effusion was noted in 33% of cases and was associated with a poor prognosis for MERS-CoV infection. [Id., citing Das, K M, et al. Am. J. Roentgenol. (2015) 205: W267-74].

### 3.8 Clinical Outcome

More MERS cases progressed to respiratory failure and received invasive mechanical ventilation therapy than SARS patients. The occurrence of AM, [Id., citing Saad, M. et al. Intl J. Infect. Dis. (2014) 29: 301-6] and the usage of vasopressor therapy were also more frequent in MERS patients in comparison with SARS.[Id., citing Chu, K H et al. Kidney Int. (2005) 67: 698-705; Almekhlafi, G A et al. Crit. Care (2016) 20: 123]. In a retrospective analysis, vasopressor therapy was proposed to be an independent risk factor for death in the ICU. [Id., citing Almekhlafi, G A et al. Crit. Care (2016) 20: 123].

MERS demonstrated a higher case fatality rate than SARS. Differences in host factors, such as age and underlying diseases, [Id., citing Chan, K S, et al. Respirology (2003) 8-(Suppl): S36-40); Choi, W S et al. Infect. Chemother. (2016) 48: 118-26] may explain some differences. However, differential cell line susceptibility, viral replication efficiency, ability to inhibit IFN production and receptor characteristics may also be responsible for the difference in the outcome of SARS-CoV and MERS-CoV infection. [Id., citing Lau, S K et al. J. Gen. Virol. (2013) 94: 2679-90; Chan, J F, et al. Differential cell line susceptibility to the emerging novel human beta coronavirus 2c EMC/2012: implications for disease pathogenesis and clinical manifestation. J. Infect. Dis. (2013) 207: 1743-52].

Compared with SARS and MERS, other HCoVs-associated pneumonia cases usually have relatively mild symptoms and recovered quickly.[Id., citing Woo, P C et al. Hong Kong Med. J. (2009) 15 (Supp. 9): 46-47]. Fatal cases were reported mainly in frail patients, such as neonates, the elderly and immunocompromised patients.

### 3.9 Treatment

At the moment, no specific therapy for infections with SARS-CoV, MERS-CoV and other HCoVs is available. Symptomatic and supportive treatment is the mainstay of therapy for patients infected by HCoVs.

A number of agents show effectiveness in vitro and/or in animal models and may improve the outcome in patients. Currently, the most commonly prescribed antiviral regimens in the clinical settings are ribavirin, IFNs and lopinavir/ritonavir.

To date, ribavirin and ribavirin plus various types of IFN have been the most common therapeutic interventions tried in patients with SARS and MERS. [Id., citing Morganstern, B. et al. Biochem. Biophys. Res. Commun. (2005) 326: 905-8); Al-Tawfiq, I A, et al. Int. J. Infect. Dis. (2014) 20: 42-6; Omrani, A S et al., Lancet Infect. Dis. (2014) 14: 1090-5]. Ribavirin, a nucleoside analogue, has a wide spectrum of antiviral activity by inhibiting viral RNA synthesis and mRNA capping. [Id., citing von Grotthuss, M. et al. Cell (2003) 113: 701-2]. When used alone for treatment of SARS, the clinical effect was inconsistent. Although in vitro studies show that combination with IFN-β will give both these agents better antiviral activity, the clinical effect remains controversial.

IFNs are important for host defense against viruses. In in vitro experiments, IFN products were effective in inhibiting both SARS-CoV and MRES-CoV, with best antiviral activity seen with IFN-β1b. [Id., citing Morgenstern, B. et al. Biochem. Biophys. Res. Commun. (2005) 326: 905-8; Chan, J F, et al., J. Infect. (2013) 67: 606-16]. Previous studies had shown a positive impact of various IFNs on aspects of treatment of SARS and MERS patients, such as a better oxygen saturation and rapid resolution of inflammation, but no effect on more significant outcomes like hospital stay and long-term survival. [Id., citing Al-Tawfiq, I A et al. Intl. J. Infect. Dis. (2014) 20: 42-6; Omrani, A S et al. Lancet Infect. Dis. (2014) 14: 1090-95; Loutfy, M R et al. JAMA (2003) 290: 3222-28].

Lopinavir and ritonavir are protease inhibitors that may inhibit the 3C-like protease of MERS-CoV and modulate apoptosis in human cells. Addition of lopinavir/ritonavir to ribavirin was associated with improved clinical outcome compared with ribavirin alone in SARS patients. [Id., citing Chu, C M et al., Thorax (2004) 59: 252-56] Although lopinavir only showed suboptimal 50% effective cytotoxic concentration (EC50) against MERS-CoV in vitro, [Id., citing Chan, J F et al. J. Infect. (2013) 67: 606-16] lopinavir/ritonavir experimental therapy was proved to improve the outcome of MERS-CoV infection in an animal model. [Id., citing Chan, J F, et al. J. Infect. Dis. (2015) 212: 1904-13].

Mycophenolic acid (MPA) is another potential therapeutic choice. Frequently used as an immunosuppressant drug to prevent rejection in organ transplantation by inhibiting lymphocyte proliferation, MPA also prevents replication of viral RNA. In vitro studies showed that MPA had strong inhibition activity against MERS-CoV. [Id., citing Hart, B J, et al. J. Gen. Virol. (2014) 95: 571-77]. However, use in a non-human primate model showed that all MPA-treated animals developed severe and/or fatal disease with higher mean viral loads than the untreated animals. [Id., citing Chan, J F, et al. J. Infect. Dis. (2015) 212: 1904 13].

Passive immunotherapy using convalescent phase human plasma was also used in the treatment of SARS and MERS. An exploratory meta-analysis found that convalescent plasma decreased mortality in SARS-CoV patients only if administered within 14 days of illness. [Id., citing Mair-Jenkins, J. et al. J. Infect. Dis. (2015) 211: 80-90].

Corticosteroids were used extensively during the SARS outbreak, generally in combination with ribavirin. Lessons from SARS showed that corticosteroid treatment was associated with a higher subsequent plasma viral load [Id., citing Lee, N. et al., J. Clin. Virol. (2004) 31: 304-9] with increased complications.

### 3.10 COVID 19

Since the emergence of coronavirus disease 2019 (COVID-19) (formerly known as the 2019 novel coronavirus (2019-nCoV) in Wuhan, China in December 2019, which is caused by severe acute respiratory syndrome coronavirus 2 (SARS-CoV-2), more than 165,069,258 cases have been reported in, resulting in more than 3,422,907 deaths worldwide [https://covid19.who.int/, visited 5/21/2021].

Approximately 96 COVID-19 vaccines are at various stages of clinical development. Olliaro, P. et al. The Lancet (April 20, 21. DOI: https://doi.org/10.1016/52666-5247(21)00069-0). Results from the Pfizer/BioNTech vaccine trial showed that the mRNA-based vaccine reduced the risk of COVID-19 by 95% [Polak, F P et al., N. Engl. J. Med. (2020) 383: 2603-15). The trial enrolled nearly 44,000 adults, each of whom got two shots, spaced three weeks apart; half received the vaccine and half got a placebo (a shot of saltwater). Of the 170 cases of COVID-19 that developed in the study participants, 162 were in the placebo group and eight were in the vaccine group. Nine of the 10 severe COVID cases occurred in the placebo group, suggesting that the vaccine reduced risk of both mild and severe COVID. According to the NEJM article, the vaccine was similarly effective in study participants of different races and ethnicities, body weight categories, presence or absence of coexisting medical conditions, and ages (younger and older than 65. Moderna reported 92.6% efficacy for its mRNA-1273 vaccine. [[Baden, L R et al. New Engl. J. Med. (2021) 384 (5): 403-16]. The AstraZeneca-Oxford ChAdOx1 nCov-19 vaccine reported 62.1% efficacy in participants who received two standard doses, and 90.0% efficacy in participants who received a low dose followed by a standard dose, with overall vaccine efficacy across both groups of 70.4% [Voysey, M. et al. Lancet (2021) 397: 99-111]. The J&J/Janssen vaccine was 66.3% effective in clinical trials (efficacy) at preventing laboratory-confirmed COVID-19 illness in people who had no evidence of prior infection 2 weeks after receiving the vaccine. People had the most protection 2 weeks after getting vaccinated [https://www.cdc.gov/coronavirus/2019-ncov/vaccines/different-vaccines/janssen.html#:˜:text=The %20J %26J %2FJanssen %20vaccine,weeks %20after %20getting %20vaccinated].

As of 20 May 2021, a total of 1,422,282,170 doses of vaccine have been administered worldwide However, SARSCoV-2 viral variants that are more infectious, more transmissible, or that can evade SARSCoV-2 antibodies, continue to emerge.

COVID-19 can present as an asymptomatic carrier state, acute respiratory disease, and pneumonia. Adults represent the population with the highest infection rate; however, neonates, children, and elderly patients can also be infected by SARS-CoV-2. In addition, nosocomial infection of hospitalized patients and healthcare workers, and viral transmission from asymptomatic carriers are possible. The most common finding on chest imaging among patients with pneumonia was ground-glass opacity with bilateral involvement. Severe cases are more likely to be older patients with underlying comorbidities compared to mild cases. Indeed, age and disease severity may be correlated with the outcomes of COVID-19. [Id].

### 3.11 Long-Haul or Long-COVID

The term “post-acute COVID-19” has been embraced by NIH to capture prolonged health abnormalities in those who have been infected with SARS-CoV-2, which includes not just long COVID, but also the effects of potentially overlapping sequelae like post-intensive care syndrome (PICS) a condition associated with treatment in an intensive care unit. [https://www.nih.gov/about-nih/who-we-are/nih-director/statements/nih-launches-new-initiative-study-long-covid, Feb. 23, 2021]. Post-acute COVID-19 is a syndrome characterized by persistent symptoms and/or delayed or long-term complications beyond 4 weeks from the onset of symptoms. Based on recent literature, it is further divided into two categories: (1) subacute or ongoing symptomatic COVID-19, which includes symptoms and abnormalities present from 4-12 weeks beyond acute COVID-19; and (2) chronic or post COVID-19 syndrome, which includes symptoms and abnormalities persisting or present beyond 12 weeks of the onset of acute COVID-19 and not attributable to alternative diagnoses [Nalbandian, A. et al. Nature Medicine (2021) 27: 601-615, citing Greenhalgh, T. et al. Brit. Med. J. (2020) 370: m3026; Shah, W. et al. Brit. Med. J. (2021) 372: n136].

Symptoms of “Long-Haul” or “Long COVID”, which can include fatigue, shortness of breath, “brain fog”, sleep disorders, fevers, gastrointestinal symptoms, anxiety, and depression, can persist for months and can range from mild to incapacitating. In some cases, new symptoms arise well after the time of infection or evolve over time.

Similarly to post-acute viral syndromes described in survivors of other virulent coronavirus epidemics [Nalbandian, A. et al. Nature Medicine (2021) 27: 601-615, citing Ahmed, H. et al. J. Rehabil. Med. (2020) 52: jrm00063; Hui, D S et al. Thorax (2005) 60: 401-9; Lam M H et al., Arch Intern. Med. (2009) 169: 2142-7; Lee, S H et al. Psychiatry Investig. (2019) 16: 59-64; Moldofsky, H. and Patcai, J. BMH Neurol. (2011) 11: 37; Ong, K C et al. Eur. Respir. (2004) 24: 436-42; Lee, A M et al. Can. J. Psychiatry (2007) 233-40], there are increasing reports of persistent and prolonged effects after acute COVID-19. Patient advocacy groups, many members of which self-identify as long-haulers, have helped contribute to its recognition, [Id.]

Early reports suggest residual effects of SARS-CoV-2 infection, such as fatigue, dyspnea, chest pain, cognitive disturbances, arthralgia and decline in quality of life. [Id, citing Carfi, A. et al. J. Am. Med. Assoc. (2020) 324: 603-5; Tenforde, M W et al. Morb. Mortal. Wkly Rep (2020) 69: 993-8; Huang, C. et al. Lancet (2021) 397: 220-32]. The most common reported issues are breathing problems, smell and taste disturbance, deep fatigue, joint pain, “brain fog”, and heart palpitations.

The severity of illness during acute COVID-19 has been significantly associated with the presence or persistence of symptoms in the post-acute COVID-19 setting. [Id., citing Huang, C. et al. Lancet (2021) 397: 220-32; Arnold, D T et al. Thorax: doi.org/10.1136/thoraxjnl-2020-216086 (2020); Halpin, S J et al. J. Med. Virol. (2021) 93: 1013-22].

Four months after onset of infection, the mRNA of SARS-CoV-2 and viral protein have been detected in the intestines of asymptomatic infected individuals by immunofluorescence and PCR analysis of intestinal biopsies. [Gaebler, C. et al. Nature (2021) 591: 639-44].

A post-acute COVID-19 Chinese study suggested sex differences, with women more likely to experience fatigue and anxiety/depression at 6 months follow up [Huang, C. et al. Lancet (2021) 397: 220-32], similar to SARS survivors [Nalbandian, A. et al. Nature Medicine (2021) 27: 601-615, citing Lee, A M et al. Can. J. Psychiatry (2007) 233-40}.

Post-hospital discharge care of COVID-19 survivors has been recognized as a major research priority by professional organizations [Id., citing Bai, C. et al. Eur. Respir. Rev. (2020) 29: 200287], and guidance for the management of these patients is still evolving. [Id., citing Shah, W. et al. Br. Med. J. (2021) 372: n136].

It has been estimated that 10% of COVID-19 survivors—including both hospitalized and non-hospitalized individuals—have persistent problems 12 weeks after infection. BMJ (2020) doi.org/10.1136/bmj.m3026 (Aug. 11, 2020). There are no guidelines to address their diagnosis and management. [Id., Huang, Y. et al. medRxiv. Foiz.org/10.1101/2021.03.03.21252086 (Mar. 5, 2021)].

In February 2021, WHO released a case report form to harmonize data collection https://www.who.int/teams/health-care-readiness-clinical-unit/covid-19/data-platform.

A TLC study of long COVID in the UK is to enroll at least 2,000 non-hospitalized patients who have had long COVID symptoms for at least 12 weeks and a positive SARS-CoV-2 test and compare these patients to at least 500 matched controls (i.e., individuals without a positive SARS-CoV2-test or suspected CoVID-19). The study participants will use a digital platform to provide comprehensive self-reported details of their symptoms and QOL; a subset of around 300 patients will provide blood samples and biological data [https://www.birmingham.ac.uk/research/quest/21st-century-healthcare/long-covid.aspx].

Other cohort studies with non-infected control groups are ongoing elsewhere, including in the U.S. and Canada.

Data from these studies will be key to subcategorizing long COVID patients who may be suffering from overlapping syndromes into populations and to identifying treatment(s).

### 3.12 Adaptive Immune Response to HCoVs

T cell response. MERS-CoV and SARS-CoV are β-coronaviruses that can cause fatal lower respiratory tract infections and extrapulmonary manifestations. [Li, G. et al., J. Medical Virol. (2020) 92: 424-32, citing Chan J F, et al. Clin Microbiol Rev. (2015) 28(2): 465-522; Cheng V C, et al. Clin Microbiol Rev. (2007) 20(4): 660-694; Sato K, et al. J Virol. 2018; 92(4):e01905-17]. T cells, CD4+ T cells, and CD8+ T cells particularly play a significant antiviral role by balancing the fight against pathogens and the risk of developing autoimmunity or overwhelming inflammation. [Id., citing Cecere T E, et al. Viruses. (2012) 4(5): 833-846]. CD4+ T cells promote the production of virus-specific antibodies by activating T-dependent B cells. However, CD8+ T cells are cytotoxic and can kill viral infected cells. CD8+ T cells account for about 80% of total infiltrative inflammatory cells in the pulmonary interstitium in SARS-CoV-infected patients and play a vital role in clearing CoVs in infected cells and inducing immune injury. [Id., citing Maloir Q, et al. Rev Med Liege. (2018) 73(7-8): 370-375]. In addition, by comparing T-cell-deficient BALB/c mice (transduced by ad5-hdp4) with controls and B-cell-deficient mice, some researchers determined that T cells could survive in the infected lungs and destroy the infected cells. [Id., citing Zhao J, et al. Proc Natl Acad Sci USA. (2014) 111(13): 4970-4975]. It emphasizes the important role of T cells rather than B cells in the control of pathogenesis of MERS-CoV infection. A cross-reactive T cell response leads to a decrease in MERS-CoV. [Id., citing Pascal K E, et al. Proc Natl Acad Sci USA. (2015) 112(28): 8738-8743] However, CD4+ T cells are more susceptible to MERS-CoV infection. The depletion of CD8+ T cells do not affect and delay viral replication at the time of infection with SARS-CoV. [Id., citing Channappanavar R, et al. J Virol. (2014) 88(19): 11034-11044, Ng O W, et al. Vaccine (2016) 34(17): 2008-2014]. Depletion of CD4+ T cells is associated with reduced pulmonary recruitment of lymphocytes and neutralizing antibody and cytokine production, resulting in a strong immune-mediated interstitial pneumonitis and delayed clearance of SARS-CoV from lungs. [Id., citing Chen J, et al. J Virol. (2010) 84(3): 1289-1301] Additionally, T helper cells produce proinflammatory cytokines via the NF-kB signaling pathway. [Id., citing Manni M L, et al. Expert review of respiratory medicine. (2014) 8(1): 25-42] IL-17 cytokines recruit monocytes and neutrophils to the site of infection with inflammation and activate other downstream cytokine and chemokine cascades, such as IL-1, LL-6, IL-8, IL-21, TNF-β, and MCP-1. [Id., citing Bunte K, Beikler T. Int J Mol Sci. 2019; 20(14): 3394, Dutzan N, Abusleme L. Adv Exp Med Biol. (2019) 1197: 107-117].

On the other hand, MERS-CoV induces T cell apoptosis by activating the intrinsic and extrinsic apoptosis pathways. A novel BH3-like region located in the C-terminal cytosolic domain of SARS-CoV protein mediates its binding to Bcl-xL and induced T-cell apoptosis. [Id., citing Yang Y, et al. Biochem J. 2005; 392(Pt 1): 135-143]. During the later stage of infection, depletion of T cells having antiviral effects may prolong the infection and promote viral survival. [Id., citing Mubarak A, et al. J Immunol Res. 2019; 2019: 6491738 11].

The reappearance of SARS-CoV is another problem. SARS-CoV-specific T cells have been screened in SARS convalescent patients. All the detected memory T cell responses are directed at SARS-CoV structural proteins. Two CD8+ T cell responses to SARS-CoV membrane (M) and Nucleocapsid (N) protein are characterized by measuring their HLA restriction and minimal T cell epitope regions. Further, these reactions are found to last up to 11 years after infection. Absence of cross-reactivity of these CD8+ T cell responses against the MERS-CoV is also demonstrated. [Id., citing Ng, O W, et al. Vaccine (2016) 34 (17): 2008-14]. It has been reported that the T cell response to S protein and other structural proteins (including the M and N proteins) is long-lasting and persistent. [Id.]

Cytotoxic lymphocytes, such as CTLs and natural killer (NK) cells are necessary for the control of viral infection. Zheng et al showed that the total number of NK and CD8+ T cells was decreased markedly in patients with SARS-CoV-2 infection, responsible for coronavirus disease 2019 (COVID-19), and suggested that SARS-CoV-2 may break down antiviral immunity at an early stage. [Zheng, M. et al., Cellular & Molec. Immunol. (2020) doi.or/10.1038/x41423-020-0402-2] The function of NK and CD8+ cells was exhausted with the increased expression of NKG2A in COVID-19 patients. In patients convalescing after therapy, the number of NK and CD8+ T cells was restored with reduced expression of NKG2A. As an inhibitory receptor, NKG2A has been shown to induce NK cell exhaustion in chronic viral infections. [Id., citing Li, F. et al. Gastroenterology (2013) 144: 392-401]. Lower percentages of CD107a+ NK, IFN-γ+ NK, IL2+ NK and TNFα-NK cells and mean fluorescence intensity (MFI) of granzyme B+0 NK cells was found in COVID-19 patients than those in healthy controls. Consistent with these findings, COVID-19 patients also showed decreased percentages of CD107a+ CD8+, IFN-γ+ CD8+, and IL-22+ CD8+ T cells and MFI of granzyme B+ CD8+ T cells, compared with those in healthy controls. Although exhaustion of T and NK cells occurs in human chronic infection and tumorigenesis, T cell apoptosis, which is regarded as the host mechanism involved in chronic infection and cancer, also occurs in SARS-CoV infection. [Id., citing Barathan, M. et al. Cells (2018) 7: 165]. In COVID-19 patients with severe pulmonary inflammation, SARS-CoV-induced NKG2A expression therefore may be correlated with functional exhaustion of cytotoxic lymphocytes at an early stage, which may result in disease progression. Using a correlation network analysis, variables significantly related to COVID-10 disease progression, including age, chronic ailment, loss of functional diversity in CD4+ T cells and increased expression of regulatory molecules, especially TIGIT, in CD8+ T cells were identified. These factors better distinguished healthy, mild and severe patients, independent of age and chronic ailments. [Id.]

Diao et al. reported that not only are T cell counts reduced significantly in COVID-19 patients, but the surviving T cells appear functionally exhausted. [Diao, B. et al.org/10.1101/2020.02.18.20024364]. They retrospectively reviewed the counts of total T cells, CD4+, CD8+ T cell subsets, and serum cytokine concentration from inpatient data of 522 patients with laboratory-confirmed COVID-19, admitted into two hospitals in Wuhan from December 2019-January 2020, and 40 healthy controls. They found that the number of total T cells, CD4+ and CD8+ T cells were dramatically reduced in COVID-19 patients, especially among patients ≥60 years of age, and in patients requiring ICU care. Counts of total T cells, CD8+ T cells or CD4+ T cells lower than 800/μL, 300/μL, or 400/μL, respectively, were negatively correlated with patient survival. Statistical analysis further demonstrated that T cell numbers were negatively correlated to serum IL-6, IL-10, and TNF-α concentration, suggesting that these cytokines may promote the T cell decrease in COVID-19 patients. To explain this observation, they suggested that the secretion of these cytokines, which may originate from non-T cell sources, produces a cytokine storm that leads to T cell reduction. T cells from COVID-19 patients also had significantly higher levels of the T cell exhaustion marker PD-1, compared to healthy controls; increasing PD-1 and Tim-3 was seen as patients progressed from prodromal to overtly symptomatic stages, further indicative of T cell exhaustion. They suggested, based on these results, that antiviral treatments to prevent the progression to T cell exhaustion in susceptible patients may be critical to their recovery.

### 3.13 Humoral Immune Responses

B cell subsets with phenotypes characteristic of naïve, non-isotype-switched, memory cells and antibody-secreting cells accumulate in CoVs. [Li, et al., J. Med. Virol. (2020) 92: 424-432, citing Ababneh M, et al. Vet World. 2019; 12(10): 1554-1562]. The antigen stimulation of MERS-CoV infection was clarified by using the specific 9-mer peptide “CYSSLILDY”, which located at position 437 to 445 within the region of the S glycoprotein. [Id., citing Ababneh M, et al. Vet World. 2019; 12(10): 1554-1562]. The sequence has the highest B cell antigenicity plot and has the ability to form the greatest number of interactions with MHCI alleles in a computerized simulation. [Id., citing Tuhin ali M, et al. Bioinformation. 2014; 10(8): 533-538]. Reports show that humoral immunity is essential to control the persistent phase of CoV infection. More antibodies isolated from patients who have survived MERS-CoV infection have been described, including MCA1, CDC-C2, CSC-05, CDC-A2, CDC-A10, MERS-GD27, and MERS-GD33. [Id., citing Niu P, et al. J Infect Dis. 2018; 218(8): 1249-1260; Chen Z, et al. J Infect Dis. 2017; 215(12): 1807-1815; Niu P, et al. Science China Life Sciences. 2018; 61(10): 1280-1282].

The complement system plays a vital role in the host immune response to CoV infection. Primitively identified as a host-sensitive and nonspecific complement to adaptive immune pathways, the complement system provides a way for the innate immune system to detect and respond to foreign antigens. [Id., citing Baker S, et al. PLOS One. 2019; 14(6):e0217626]. Given its potential to damage the host tissues, the complement system is tightly controlled by inhibiting proteins in the serum. Virus encoded proteins help them evade the detection of the complement system, suggesting that complements are vital to the antiviral response. C3a and C5a have potent proinflammatory properties and can trigger inflammatory cell recruitment and neutrophil activation. C3a and C5a blockade acts as a treatment for acute lung injury, and anti-C5a antibody shows to protect mice from infection with MERS-CoV. [Id., citing Sun S, et al. Am J Respir Cell Mol Biol. 2013; 49(2): 221-230]. SARA-CoV infection activates the complement pathway and complement signaling contributes to disease.[Id., citing Gralinski L E, et al. mBio. 2018; 9(5):e01753-18].

### 3.14 Antibody Responses to Coronavirus Infections

The antibody response in vivo is a dynamic and complex mixture of monoclonal antibodies (mAbs), which work together to target different antigenic domains on the envelope glycoprotein of the virus. It is important to determine whether the antibodies are powerful in the adaptive immune responses to MERS-CoV infection. Research from all over the world has described more than 20 kinds of monoclonal antibodies, most of which are human or humanized antibodies. The virus uses its spike proteins as an adhesion factor to facilitate host entry through a special receptor called dipeptidyl peptidase-4 (DPP4). This receptor is considered a key factor in the signal transmission and activation of acquired and innate immune responses in infected patients. Thus, compared with the time-consuming vaccine preparation, the design of monoclonal antibodies against these proteins has a better protective effect. [Id.]

Human monoclonal antibody (m336) isolated from the phage display library interacts with the receptor-binding region of MES coronavirus spike protein and displays strong neutralization activity to MES-CoV in vitro. [Id., citing Ying T, et al. J Virol. 2014; 88(14): 7796-7805]. Human monoclonal antibody m336 shows high neutralization activity to MERS-CoV in vitro. m336 reduces the RNA titer of lung by 40 000 to 90 000 folds. [Id., citing Houser K V, et al. Infection. J Infect Dis. 2016; 213(10): 1557-1561]. After infection with MERS-CoV, monkeys were treated with high-titer hyperimmune plasma or monoclonal antibody m336. Both groups had relieved symptoms of clinical diseases, but the reduction of respiratory viral load was only found in the hyperimmune plasma group. Although both super immune plasma and m336 therapy show to mitigate the disease of the common marmoset, neither has the ability to prevent the disease completely. [Id., citing van Doremalen N, et al. Antiviral Res. 2017; 143: 30-37]. Yet, HMab m336 is found to significantly reduce the viral RNA titers and viral-associated pathological changes in rabbit lung tissue. [Id., citing Houser K V, et al. J Infect Dis. 2016; 213(10): 1557-1561]. Mice inoculated with S nanoparticles produced high-level neutralizing antibodies against homologous viruses, and these antibodies have no cross-protection with heteroviruses. [Id., citing Coleman C M, et al. Vaccine. 2014; 32(26): 3169-3174] After being stimulated by SARS-CoV, immunized ferrets produced more rapid and stronger neutralizing antibody reaction than the control animals; however, the strong inflammatory reaction is observed in liver tissue. All this suggests that the expression of SARS-CoV S protein is associated with enhanced hepatitis. [Id., citing Weingart H, et al. J Virol. 2004; 78(22): 12672-12676]. On the other hand, the time course of SARS-CoV viremia and antibody response has been studied. [Id., citing Chen W. J Med Microbiol. 2004; 53(Pt 5): 435-438]. SARS-CoV viremia is not detected in the blood samples of convalescent patients. In the peak period of viremia, 75% of the blood samples of patients diagnosed as SARS in the first 1 to 2 weeks before detection can detect virus RNA. The prolongation of IgG production may indicate the significance of IgG in both humoral immune response to acute SARS-CoV infection and clearance of the remaining virus sources during recovery.[Id.]

### 4. N-Acetylcysteine (NAC) and Viral Infections

Oxidative stress is implicated in the pathogenesis of pulmonary damage during viral infections. [Ghezzi, P., Ungheri, D., Intl. J. Immunopathol & Pharmacol. (2004) 17 (1): 99-102]. The generation of free radicals by phagocytes involved in the inflammatory process and alterations of the immune response play a key role in viral infections. [De Flora, S. et al. Eur. Respir. J. (1997) 10: 1535-41, citing Rouse, B. T., Horohov, D W. Rev. Infect. Dis. (1986) 8: 850-873; Maeda, H., Akaike, T. Proc. Soc. Exp. Biol. Med. (1991) 198: 721-27]. Inflammatory mediators, including cytokines, such as interleukins, IFNγ and TNF, have pleiotropic effects both locally and systematically. [Id., citing Hennett, T. et al., J. Immunol. (1992) 149: 932-9]. The rationale for clinical use of antioxidants in viral infections has been aimed at normalizing the altered cell redox equilibrium, and at preventing and/or treating the clinical manifestations of immunological dysfunction in such infections [Id. citing Peristeres, et al. Cell Immunol. (1992) 140: 390-9].

Glutathione (γ-glutamylcysteinylglycine, or “GSH”), a crucial intracellular tripeptide that is normally found in all animal cells and most plants and bacteria at relatively high (1-10 mM) concentrations, helps to protect cells against oxidative damage that would otherwise be caused by free radicals and reactive oxidative intermediates (ROIs) produced during cell metabolism. It is a major scavenger of reactive oxidative intermediates present in all eukaryotic forms of life and is generally required to protect cells against damage by oxidants. Glutathione reduces (and thereby detoxifies) intracellular oxidants; and is oxidized to the disulfide linked dimer (GSSG), which is actively pumped out of cells and becomes largely unavailable for reconversion to reduced glutathione. Thus, unless glutathione is resynthesized through other pathways, its utilization is associated with a reduction in the amount of glutathione available. The antioxidant effects of glutathione are also mediated less directly by its role in maintaining other antioxidants in reduced form.

Because GSH is depleted in these reactions, it must continually be replenished to maintain cell and organ viability and to support normal cellular functions. Synthesis of GSH requires cysteine, a conditionally essential amino acid that must be obtained from dietary sources or by conversion of dietary methionine via the cystathionase pathway. If the supply of cysteine is adequate, normal GSH levels are maintained. In contrast, if supplies of cysteine are inadequate to maintain GSH homeostasis in the face of increased GSH consumption, GSH depletion occurs. Acute GSH depletion causes severe—often fatal—oxidative and/or alkylation injury.

GSH depletion impacts a wide variety of cellular processes, ranging from DNA synthesis and gene expression to sugar metabolism and lactate production. The pleiotropic activity of this key intracellular molecule, which arose very early in evolution, derives from its participation in the energy economy and the synthetic and catabolic activities of virtually all cells. In higher animals, it also participates in regulating the expression or activity of extracellular molecules, including many of the cytokines and adhesion molecules implicated in inflammatory reactions and other disease processes.

N-acetylcysteine (NAC) has been used by many physicians for treatment of hepatic failure of any etiology, and is the accepted antidote for preventing hepatic injury in acetaminophen overdose. Because NAC provides sulfhydryl groups and acts both as a precursor of reduced glutathione and as a direct reactive oxygen species (ROS) scavenger, it regulates redox status in the cells. [Sadowska, A M et al., Intl J. Chron Obstruct. Pulm. Dis. (2006) 1(4): 425-34]. It provides the cysteine necessary to replenish a crucial intracellular GSH, which is depleted during detoxification of excessive amounts of acetaminophen. Since orally-administered NAC is rapidly converted by first-pass metabolism to cysteine, it results in replenishment of GSH as well as supplying cysteine for additional metabolic and protein synthetic processes. NAC is also a source of SH groups which can stimulate glutathione synthesis, enhance glutathione-S-transferase activity, and/or promote detoxification by liver and lung tissue of some direct-acting mutagens.

Mucolytics are responsible for the disruption of the mucous gel, generally by altering the degree of the cross-linking or the interactions between molecules in the gel. [Sadowska, A M et al., Intl J. Chron Obstruct. Pulm. Dis. (2006) 1(4): 425-34]. Classical mucolytics, like NAC and other thiol reducing agents, degrade the three-dimensional network that forms the mucus by reducing the disulfide bonds (S—S) to a sulfhydryl (SH) bond (—SH) that no longer participates in the cross-linking. They may act on the mucus elasticity and viscosity as well as modulate its production and secretion [Id., citing Livingstone C R, et al. J Pharm Pharmacol. (1990) 42:73-80; King M, Rubin B K. Adv Drug Deliv Rev. (2002) 54:1475-902]. NAC has been reported to reduce the viscosity of sputum in both cystic fibrosis and COPD, facilitating the removal of pulmonary secretions [Ventresca, G P et al. Drugs in bronchial mucology. New York: Raven Pr; (1989) pp. 77-102]. Moreover, by maintaining the airway clearance, it prevents bacterial stimulation of mucin production and hence mucus hypersecretion [Adler, K B et al. Am. J. Pathol. (1986) 125: 501-14]. As a mucolytic drug, it may, by means of decreasing viscosity of the sputum, clean the bronchi leading to a decrease in dyspnea and improved lung function.

### 4.1 NAC in HIV-Infected Individuals

Glutathione deficiency is common in HIV-infected individuals and is associated with impaired T cell function and impaired survival. [DeRosa, S C et al, Eur. J. Clinical Investigation (2000) 30 (10): 915-29]. Glutathione regulates T and NK cell function, for example: low GSH in T cells impairs IL-2 production, IL-2 responses and cytotoxic T cell activity [Id., citing Droge, W. et al., Immunology Today (1992) 13: 211-141 Yim, C-Y et al, J. Immunol. (1994) 152: 5796-805; Hargrove, M E, et al., Cell Immunol. (1993) 149: 433 443; Iwara, S. et al., J. Immunol (1994) 152: 5633-42; Chen, G. et al., Intl J. Immunopharmacol. (1994) 16: 755-60; Jeannin, P. et al. J. Exp. Med. (1995) 182: 1785-92]; low GSH in NK cells impairs killing activity [Id., citing Tsuyuki, S. et al. Clin. Immunol. Immunopathol. (1998) 88: 192-8; Tsuyuki, S. et al. Int. Immunol. (1998) 10: 15001-8]; and low GSH in antigen presenting cells (APCs) impairs IL-12 production and favors TH2 over TH1 responses [Id., citing Peterson, J D, et al. Proc Nat. Acad. Sci. USA (1998) 95: 3071-6]. In HIV infection, GSH levels are low in plasma and other body fluids [Id., citing Eck, H P, et al. Biol. Chem. Hoppe-Seyler (1989) 370: 101-8; Buhl, R., et al. Lancet (1989) ii: 1294-8; deQuay, B. et al. AIDS (1992) 6: 815-9; Helbling, B. et al. Eur. J. Clin. Invest. (1996) 26: 38-44; Ehret, A. et al. J. Virol (1996) 70: 6502-7; Pacht, E R et al. Chest (1997) 112: 785-8; Walmsley, S L et al. AIDS (1997) 11: 1689-97; Aukrust, P. et al. J. Infect. Dis. (1999) 179: 74-82; Jahoor, F. et al. Amer. J. Physiol Endocrinol. Met. (1999) 39: E205-E211]. Furthermore GSH levels in erythrocytes and in individual T cell subsets (detected, respectively by HPLC and with FACS as the GSH conjugate glutathione-S-bimane (GSB), decrease as HIV disease progresses. [Id., citing Jahoor, F. et al. Amer. J. Physiol Endocrinol. Met. (1999) 39: E205-E211, Roederer, M. et al. Intl Immunol. (1991) 3: 933-7; Staal, F L T, et al. AIDS Res. Human Retrov. (1992) 8: 305-14); Staal, F J T, Lancet (1992) 339: 909-12; Herzenberg, L A, et al. Proc. Nat. Acad. Sci. (1997) 94: 1967-72].

In a placebo-controlled clinical trial, NAC treatment for 8 weeks safely replenished whole blood GSH and T cell GSH in HIV-infected individuals. Entry into the trial requires that subjects had adhered to a stable antiretroviral therapy schedule for at least four months prior to entry into the trial; subjects were required to maintain that therapy schedule for the duration of the trial. NAC treatment during the trial did not result in a significant change in either CD4 T cell count (P>0.9) or viral load (P>0.1, and did not significantly affect CD8 T cell counts, CD4/CD8 ratio, hematocrit, other hematological markers or β2 microglobulin levels (P>0.1 in all cases). NAC administration was associated with increased survival; for the 73 subjects for whom survival data were obtained, P=0.0003, risk ratio=0.3 (0.1-0.6). The median NAC administration time computed for all subjects who took NAC was 24 weeks; the median NAC dose was 5.3 g day-1. [DeRosa, S C et al, Eur. J. Clinical Investigation (2000) 30 (10): 915-29].

NAC was demonstrated to counteract the in vitro stimulatory effects of TNFα or phorbol 12-myristate 13-acetate (PMA) on HIV-directed gene expression and to inhibit HIV replication in a cell line 293.27.2 that has a stably integrated HIV-LacZ fusion construct; HIV LTR directed expression of the bacterial lacZ gene, measured as β-galactosidase activity either in individual viable cells by FACS-Gal or in cell lysates by a fluorometric assay. [Roederer, et al. Proc. Nat. Acad. Sci. USA (1990) 87: 4884-88]. NAC similarly counteracted the in vivo stimulatory effects of TNFα or PMA on HIV-directed gene expression and inhibited HIV replication in a stably transfected clone of H9, a CD4+ T cell line. The synergistic stimulation by TNFα and PMA together was effectively blocked by NAC. These findings demonstrate (i) that the mechanism ultimately responsible for viral stimulation is not saturated by either TNFα or PMA acting alone; (ii) that TNF α and PMA stimulate via at least partially independent pathways; and (iii) that NAC inhibits the independent segments of each of these pathways, because the two pathways are differentially sensitive to NAC. It was suggested that NAC inhibition of stimulation of HIV by TNFα and PMA is consistent with the idea that intracellular thiol levels influence the regulation of genes whose expression is modulated by NF-κB or other transcription factors. [Id.]

NAC also blocked stimulation of HIV replication by TNFα and PMA and reduced the basal replication of the virus in a transformed CD4+ T cell line (MOLT-4) and in phytohemagglutinin-stimulated PBMCs from HIV negative subjects. TNFα stimulation increases the production of infectious virions and the HIV core protein P24 found in the supernatant of MOLT-4 cultures acutely infected with HIV. NAC blocks this stimulation at doses similar to those that block HIV-directed β-galactosidase expression in 293.27.2 cells. Because NAC also reduced HIV replication in MOLT-4 cells infected in the absence of exogenous TNFα, it was suggested that factors responsible for the basal level of viral replication also appear to be influenced by the intracellular thiol levels of the MOLT-4 cells. [Roederer, et al. Proc. Nat. Acad. Sci. USA (1990) 87: 4884-88].

NAC also complemented the antiviral activity of AZT. Since elevated serum levels of TNFα in AIDS patients may counter the therapeutic effectiveness of AZT, it was suggested that raising depleted GSH levels in AIDS patients with NAC may provide an effective adjunct to AZT therapy. PMA mimics interleukin-mediated activation of T cells, and such stimulation is thought to be responsible for evolution of HIV infection from latency to the symptomatic stages of AIDS; accordingly, the findings with PBMCs suggested that NAC may be broadly effective in maintaining latency or in preventing further progression of disease in a symptomatic individual. [Roederer, et al. Proc. Nat. Acad. Sci. USA (1990) 87: 4884-88]. It was suggested that treatment of HIV-infected individuals with NAC therefore may serve both to restore lower thiol levels to counter the TNFα-GSH spiral, and to inhibit viral replication stimulated by TNFα and other cytokines. Such treatment could be value for maintaining latency in asymptomatic patients. [Roederer, et al. Proc. Nat. Acad. Sci. USA (1990) 87: 4884-88].

### 4.2 NAC and Influenza-Infected Individuals

Severe influenza is defined by the World Health Organization as clinical or radiographic evidence of lower respiratory tract disease (e.g. dyspnea, tachypnea, radiographic pneumonia, etc.), central nervous system involvement (e.g. encephalopathy, encephalitis), severe dehydration, influenza associated with certain complications (e.g. renal failure, multiorgan failure, septic shock, rhabdomyolysis and myocarditis), or influenza that causes exacerbation of underlying chronic disease (e.g. asthma, chronic obstructive pulmonary disease, diabetes, or cardiovascular conditions such as congestive cardiac failure) or any other condition requiring hospital admission. [Hui, D. et al. Antiviral Res. (2018) 150: 202-216]. However, most studies simply define severe influenza as influenza requiring hospitalization. Many patients hospitalized with influenza are elderly subjects with co-morbid illness requiring drug treatment with some immuno-modulating properties (e.g. statins, PPAR agonist, and COX-2 inhibitors). [Id.] A severe inflammatory immune response with hypercytokinemia was reported in patients hospitalized with severe influenza, such as avian influenza A (H5N1), A (H7N9) and seasonal A (H1N1)pdm09 virus infections. [Hui, D S et al. Antiviral Res. (2018) 150: 202-16].

High levels of pro-inflammatory cytokines have been reported in patients with severe influenza A(H1N1)pdm09 virus infection [Id., citing Lee, N. et al. Antivir Ther. (2011) 16:237-247, To et al., 2010; Bradley-Stewart et al., 2013], A(H5N1) infection [Id., citing To, K K et al., J Med Virol. 2001; 63:242-246; Peiris, J S et al. Lancet. 2004; 363:617-619; de Jong, M D et al. Nat Med. 2006; 12:1203-1207 6], and A(H7N9) infection [Id., citing Zhou, Jet al., Nature. 2013; 499:500-503; Chi, Y. et al., J Infect Dis. 2013; 208:1962-1967]. These cytokines may be caused by the viral infection (primary cytokines), or the immune response (secondary cytokines) [Id., citing Guo, X J et al., Semin Immunopathol. 2017; 39:541-550 7].

NAC was shown to inhibit the production of pro-inflammatory molecules in lung epithelial cells infected with the highly pathogenic influenza A (H5N1) viruses [Id., citing Geiler, J. et al., Biochem Pharmacol. 2010; 79:413-420] and inhibit mucin synthesis and pro-inflammatory mediators in alveolar type II epithelial cells infected with influenza viruses A and B [Id., citing Mata, M et al., Biochem Pharmacol. 2011; 82:548-555].

In a murine model infected with an influenza virus strain A/PR8(H1N1) adapted in mice, NAC demonstrated synergy with oseltamivir in protecting mice from lethal influenza infection, with a survival rate of 100% for the combination therapy vs 60% for oseltamivir alone [Id., citing Garozzo, A et al., Int J Immunopathol Pharmacol. 2007; 20:349-354]. NAC also demonstrated synergy with ribavirin, with a survival rate of 92% for the combination therapy vs. 58% with ribavirin alone, which suggested that antioxidant therapy can increase survival either by improving the defenses against the virus or by protecting from the pathogenesis of lung inflammation. [Ghezzi, P., Ungheri D. Intl. J. Immnopathol. & Pharmacol. (2004) 17 (1) 99-102]. In BALB/c mice inoculated intra-nasally with A/swine/HeBei/012/2008 (H9N2) viruses with or without NAC, NAC reduced pulmonary inflammation, pulmonary edema, myeloperoxidase activity, total cells, neutrophils, macrophages, TNF-α, IL-6, IL-1β and CXCL-10 in broncho-alveolar lavage (BAL) fluid. In addition, NAC significantly inhibited the levels of TLR4 protein and TLR4 mRNA in the lungs, while pharmacological inhibitors of TLR4 (E5564) led to similar effects as those determined for NAC in A(H9N2) swine influenza virus-infected mice [Hui, D. et al. Antiviral Res. (2018) 150: 202-216, citing Zhang, R H et al., Int Immunopharmacol. 2014; 22:1-8].

Despite the scientific basis, there is only one case report that high dose NAC, administered at 100 mg/kg daily as a continuous IV infusion, appeared to be effective in improving the clinical status by reducing C-reactive protein and oxygen requirement in a 48-year-old previously healthy female, who had presented with severe pneumonia and septic shock due to A(H1N1)pdm09 influenza. [Id., citing Lai, K Y et al., Ann Intern Med. 2010; 152:687-688]. However, it was difficult to interpret the efficacy of NAC in this case report as there was concomitant treatment with higher than licensed dose of oseltamivir (150 mg twice-daily).

The effect of NAC on proliferation of human PBMCs isolated from healthy blood donors after in vitro stimulation with influenza virus antigens (Influvac 97/98) and on antibody production by B cells in vitro was studied. NAC at a concentration of 1 mM was shown to increase influenza virus specific T lymphocyte proliferation and interferon-gamma (IFNγ) production in vitro, and enhanced the effector function of two influenza specific CD8+ cytotoxic T-lymphocyte clones directed toward HLA-A*0201 and HLA-B*2705 restricted epitopes. [Boon, A C M, et al., Scand. J. Immunol. (2002) 55: 24-32].

Administration of N-acetylcysteine during the winter was reported to provide a significant attenuation of influenza and influenza-like episodes, especially in elderly high-risk individuals. [De Flora, S. et al. Eur. Respir. J. (1997) 10: 1535-41] N-acetylcysteine did not prevent A/H1N1 virus influenza infection but significantly reduced the incidence of clinically apparent disease. A total of 262 subjects of both sexes (78%> or =65 yrs, and 62% suffering from nonrespiratory chronic degenerative diseases) were enrolled in a randomized, double-blind trial involving 20 Italian Centres. They were randomized to receive either placebo or NAC effervescent tablets (600 mg) twice daily for 6 months. Patients suffering from chronic respiratory diseases were not eligible, to avoid possible confounding by an effect of NAC on respiratory symptoms. NAC treatment was well tolerated and resulted in a significant decrease in the frequency of influenza-like episodes, severity, and length of time confined to bed. Both local and systemic symptoms were sharply and significantly reduced in the NAC group. Frequency of seroconversion towards A/H1N1 Singapore 6/86 influenza virus was similar in the two groups, but only 25% of virus-infected subjects under NAC treatment developed a symptomatic form, versus 79% in the placebo group. Evaluation of cell-mediated immunity showed a progressive, significant shift from anergy (meaning an absence of the normal immune response to a particular antigen) to normergy (meaning a normal immune response) following NAC treatment.

Other clinical applications for NAC supplementation supported by varying levels of scientific evidence include prevention of chronic obstructive pulmonary disease exacerbation, prevention of contrast-induced kidney damage during imaging procedures, attenuation of illness from influenza virus when started before infection, treatment of pulmonary fibrosis, treatment of infertility in patients with clomiphene-resistant polycystic ovary syndrome. [Millea, P J. Am. Family Physician (2009) 80(3): 265-269].

### 5. Cannabinoids

Cannabinoids are terpenophenolic secondary metabolites produced by Cannabis. Fischedick et al., Phytochemistry 71:2058-73 (2010). Cannabis strains that are used produce cannabinoids are dioecious, with cannabinoids particularly accumulating on the unfertilized female inflorescence. Ritchens et al., PLoS ONE 13:e0201119 (2018). However, synthesis and accumulation of cannabinoids occurs in trichomes on the surfaces of not only inflorescences, but on the leaves as well. Happyana et al., Phytochemistry 87:51-59 (2013). Cannabinoids also are found in low amounts in plant seeds, roots, and pollen. Andre et al., Front. Plant Sci. 7:19, doi:10.3389/fpls.2016.00019 (2016). Cannabinoids have also been found in plants from the Radula and Helichrysum genera. Appendino et al., J. Nat. Prod. 71:1427-30 (2008).

More than 140 different cannabinoids have been reported, although some of these are breakdown products, and they are generally classified into 11 subclasses. Berman et al., Sci. Rep. 8:14280 (2018). The predominant compounds are Δ⁹-tetrahydrocannabinolic acid (THCA), cannabidiolic acid (CBDA), cannabinolic acid (CBNA), cannabigerolic acid (CBGA), cannabichromenic acid (CBCA), and cannabinodiolic acid (CBNDA). THCA is the major cannabinoid in the drug-type Cannabis, while CBDA predominates in fiber-type hemps.

Cannabinoid precursors originate from two distinct biosynthetic pathways: (1) the polyketide pathway, producing olivetolic acid (OLA), and (2) the plastidal 2-C-methyl-D-erythritol4-phosphate (MEP) pathway, producing geranyldiphosphate (GPP). Andre et al. (2016). OLA is formed from hexanoyl-CoA, derived from the short-chain fatty acid hexanoate, by aldol condensation with three molecules of malonyl-CoA, in a reaction catalyzed by a polyketide synthase (PKS) and an olivetolic acid cyclase (OAC). Geranylpyrophosphate:olivetolate geranyltransferase catalyzes alkylation of OLA with GPP forming CBGA, a precursor of numerous cannabinoids. Three oxidocyclases then dictate cannabinoid diversity: (1) THCA synthase (THCAS) converts CBGA to THCA, (2) CBDA synthase (CBDAS) forms CBDA, and (3) CBCA synthase (CBCAS) produces CBCA. Andre et al. (2016). The bioactive forms of the cannabinoids are produced following a light or heat induced decarboxylation reaction to generate cannabigerol (CBG), Δ⁹-tetrahydrocannabinol (THC), cannabidiol (CBD), or cannabichromene (CBC). de Meijer et al., Genetics 163:335-46 (2003). Other cannabinoids include a further oxidation product of THC, cannabinol (CBN), and cannabinoids produced from condensation of geranyl diphosphate with a variant of olivetolic acid, divarinic acid, which has a propyl side chain instead of the pentyl side chain, and produces in Δ⁹-tetrahydrocannabivarin (THCV). Ritchens et al. (2018). Inflorescence content of cannabinoids in Cannabis strains range from 3.03±0.37 (Juanita) to 21.53±2.04 (Holy Power) % flower dry wt THC, 0.00±0.00 (Platinum Scout) to 9.84±1.15 (Thunderstruck) % flower dry wt CBD, 0.05±0.00 (Bohdi Tree) to 0.62±0.06 (Thunderstruck) % flower dry wt CBC, 0.05±0.00 (Sour Willie) to 2.08±0.17 (Twisted Velvet) % flower dry wt CBG, and 0.02±0.00 (Sour Willie, Bohdi Tree, Juanita) to 1.30±0.05 (Platinum Buffalo) % flower dry wt THCV. Andre et al. (2016) (low and high content strains noted in parenthesis; for full content of strains, see Andre et al. (2016) Table 3). Leaf content of cannabinoids in Cannabis strains range from 0.26±0.03 (Love Lace) to 2.69±0.49 (Crystal Cookies) % leaf dry wt THC, 0.00±0.00 (Crystal Cookies, Twisted Velvet, Platinum Buffalo, Platinum Scout, Blue Cherry Pie, Purple Fat Pie, Holy Power, Lavender Jones 2017) to 1.24±0.28 (Thunderstruck) % leaf dry wt CBD, 0.03±0.01 (Bohdi Tree) to 0.69±0.07 (Platinum Scout) % leaf dry wt CBC, 0.00±0.00 (Platinum Scout, Blue Cherry Pie, White Widow, Sour Willie, Purple Fat Pie, Bohdi Tree, Lavender Jones 2017, Juanita, Love Lave) to 0.04±0.04 (Thunderstruck) % leaf dry wt CBG, and 0.00±0.00 (Platinum Scout, Blue Cherry Pie, Purple Fat Pie, Holy Power, Alien Blues, Thunderstruck, Lavender Jones 2017, Love Lace) to 0.01±0.02 (Crystal Cookies) % leaf dry wt THCV. Andre et al. (2016) (low and high content strains noted in parenthesis; for full content of strains, see Andre et al. (2016) Table 4).

Based on the diversity of cannabinoid content, five unique chemotypes have been described: chemotype I includes strains with primarily THCV, chemotype II includes strains with similar levels of CBD and THCV, chemotype III includes the hemp strains with virtually no THCV and primarily CBD; chemotypes IV and V are less frequent and include strains with no THCV and primarily CBG and strains with no cannabinoids. Ritchens et al. (2018). The distribution of CPD/THC ratios in most populations probably underlies a discrete inheritance of the chemotype trait. de Meijer et al. (2003). The cannabinoid profile of a Cannabis plant, and relatedly a plant's CBD/THC ratio, is primarily dependent on the plant's genetic background and that each individual plant invariably belongs to its distinct chemical group throughout its life cycle. Beutler et al., Econ. Bot. 32:387-94 (1978).

In addition to the cannabinoids, Cannabis are also rich in bioactive terpenoids.

Cannabis terpenoid composition provides information about the origin of the plant, as unique chemical abundances of specific terpenoids are predicted to be associated with chemotypes and species level taxa. Fischedick et al. (2010). Analysis of 72 Cannabis strains showed that the total terpenoid content ranged between 0.6 and 3.3%, while the total cannabinoid content ranged between 12.6±31.5%. Ritchens et al. (2018).

Numerous methods are known to extract cannabinoids and terpenoids from Cannabis. Sonication is the most common and is the method recommended by both United Nations Office on Drugs and Crime (UNDOC) and the American Herbal Pharmacopoeia (AHP). Giese et al., J. AOAC Int. 98:1503-22 (2015). Methods in the AHP monograph recommend drying and powdering the sample first and require a separate moisture determination to accurately assess the content of the initial inflorescence. It is also noted that such drying and powdering alters the terpenoid content. Giese et al. (2015); Swift et al., PLoS ONE 8:e70052 (2013). There are three notable limitations of sonication: injection of Cannabis extracts for analysis by GC typically results in decarboxylation in the injection port, and consequently it is only the decarboxylated phytocannabinoids that are measured directly by these techniques; while derivatization of the metabolite extract via silylation enables the measurement of both acid and neutral phytocannabinoids, a complete derivatization yield is difficult to obtain and thus quantification is less reliable; and it has been suggested that phytocannabinoids may thermally degrade (oxidize, isomerize) in the injector port and column. Berman et al. (2018).

Most cellular cannabinoid effects are mediated by two G protein-coupled receptors (GPCRs), CB₁ and CB₂. CB₁ receptors are present in very high levels in the brain and in lower amounts in a more widespread fashion, and they mediate most psychoactive effects of cannabinoids. CB₂ receptors are more limited in distribution, being found in certain immune cells and neurons. Mackie, J. Neuroendocrinol. 20(Supp. 1):10-14 (2008). Cannabinoids mediate both inhibitory and stimulatory effects on the immune system by modulating cytokine expression. [Raduner, S. et al., J. Biol. Chem. (2006) 281 (20): 14192-14206, citing Klein, T., et al. J. Leukocyte Biol. (2003) 74: 486-96; Croxford, J. L. and Yamamura, t. J. Neuroimmunol (2005) 166: 3-18]. Other GPCRs, ion channels, and nuclear receptors also interact with cannabinoids. Zou et al., Int. J. Mol. Sci. 19:833 (2018). N-arachidonoyl-ethanolamine and 2-arachidonoylglycerol are endogenous agonists of cannabinoid receptors. Zou et al. (2018). A putative third CB receptor, GPR55, shares only 13.5% sequence identity to CB₁ and 14.4% sequence identity to CB₂. Lauckner et al., Proc. Natl. Acad. Sci. USA 105:2699-2704 (2008). GPR55 shares some ligands with the CB₁ and CB₂, it has additional agonist ligands with novel chemotypes. Kotsikorou et al., Biochemistry 52:9456-69 (2013).

In addition to the cannabinoids, endogenous agonists for CB₁ and CB₂ (and in some cases GPR55) include arachidonoyl ethanolamide (anandamide), 2-arachidonoyl glycerol, and 2-arachidonyl glyceryl ether (noladin ether). Pertwee et al., Prostaglandins Leukot. Essent. Fatty Acids 66:101-21 (2002). There are also three classes of synthetic agonists: classical, which are similar to THC; bicyclic; and aminoalkylindole cannabinoids. Hourani et al., Brain Neurosci. Adv. 2:1-8 (2018). Many of these synthetic cannabinoids are far more potent than THC, and they also display greater efficacy. Selective, synthetic agonists for CB₁ and CB₂ include SR141716A, LY320135, SR144528, 6-iodopravadoline (AM630), Nabilone, CP55940, and R-(+)-WIN55212-2. Pertwee et al. (2002); Hourani et al. (2018). SR141716A and LY320135 are highly selective for CB1, and SR144528 and AM630 are highly selective for CB2. Pertwee et al. (2002). Ligands specific for GPR55 include a morpholinosulfonylphenylamide (ML186; CID15945391) with 305 nM potency for GPR55 and greater than 100-fold selectivity against CB₁ and CB₂; a tricyclic triazoloquinoline (ML185; CID1374043) with 658 nM potency for GPR55 and greater than 48-fold selectivity against CB₁ and CB2; a piperazine (ML184; CID2440433) with 263 nM potency for GPR55 and 57-fold selectivity against CB₁ and CB₂; 3-fluoro-4-(4-{[4′-fluoro-4-(methylsulfonyl)-2-biphenylyl]carbonyl}-1-piperazinyl)aniline; 1-{2-fluoro-4-[1-(methyloxy)ethyl]phenyl}-4-{[4′-fluoro-4-(methylsulfonyl)-2-biphenylyl]carbonyl}piperazine; and ML191 (CID23612552), ML192 (CID1434953), and ML193 (CID1261822), each with no observed agonism or antagonism against CB₁ and CB₂ up to 20 μM. Heynen-Genel et al., Screening for Selective Ligands for GPR55-Antagonists. Probe Reports from the NIH Molecular Libraries Program; Bethesda, Md. (2010); Brown et al., J. Pharmacol. Exper. Therapeut. 337:236-46 (2011); Kotsikorou et al. (2013).

Both phytocannabinoids and synthetic cannabinoids can directly impact the endocannabinoid system via a variety of pharmacological mechanisms, including agonism, antagonism, and allosteric modulation (for detailed reviews of cannabinoid pharmacology. Bonn-Miller et al., Int. Rev. Psychiatry 30:277-84 (2018).

There are currently three oral formulations of THC (dronabinol) commercially available by prescription in the United States. Dronabinol is available commercially as MARINOL® soft gelatin capsules, SYNDROS® liquid, and NAMISOL® is available as sublingual tablets, have been approved by the Food and Drug Administration (FDA) for the control of nausea and vomiting associated with chemotherapy and for appetite stimulation in AIDS patients suffering from the wasting syndrome. MARINOL® is formulated by dissolving THC in sesame oil to manufacture soft gelatin capsules suitable for oral administration. MARINOL® gelatin capsules exhibit full therapeutic potency approximately one hour following their administration. U.S. Pat. No. 8,808,734. For anorexia associated with weight loss in AIDS patients, recommended adult starting dosage of SYNDROS® is 2.1 mg orally twice daily, one hour before lunch and one hour before dinner. For nausea and vomiting associated with chemotherapy, the recommended starting dosage of SYNDROS® is 4.2 mg/m² orally administered 1 to 3 hours prior to chemotherapy and then every 2 to 4 hours after chemotherapy for a total of 4 to 6 doses per day. FDA Full Prescribing Information for SYNDROS®, Ref. ID 4103077 (2017).

Onset of therapeutic potency for Dronabinol is shorter, approximately 0.5 to 1 hour after oral administration, with a peak therapeutic effect lasting for a time period of 2-4 hours post administration. However, the amount of Dronabinol reaching the blood stream by absorption through the digestive system is only 10-20% of the administered dose. Fasting or food deprivation may further decrease the rate of absorption of Dronabinol. On the other hand, NAMISOL® has a rapid uptake through the sublingual mucosa. However, the tablet, must be kept under the tongue for the time it takes to dissolve and stimulates the flow of saliva. This make it difficult for patients to avoid swallowing the tablet when substantial amounts of saliva are produced. U.S. Pat. No. 8,808,734.

Oral formulations of synthetic cannabinoids are also available commercially. For instance, Nabilone is a synthetic cannabinoid marketed as CESAMET® in Canada, the United States, the United Kingdom, and Mexico. Nabilone is formulated as capsules suitable for oral administration. NAMISOL® is approved for use as an antiemetic and analgesic for neuropathic pain. SAVITEX® is a mouth spray containing THC and CBD. U.S. Pat. No. 8,808,734. It is approved for the treatment of spasticity due to multiple sclerosis and as adjunct analgesic in advanced cancer patients. Paudel et al., Drug Dev. Indus. Pharm. 36:1088-97 (2010). Administration of synthetic cannabinoid formulations show fewer undesirable side effects than THC. U.S. Pat. No. 8,808,734.

Cannabinoids are lipophilic substances that are known to be poorly soluble in water (less than 1 μg/mL). As an example, CBD is soluble in ethanol (36 mg/mL) and dimethylsulfoxide (60 mg/mL). Bioavailability of pharmaceutical substances taken per orally depends on the extent to which the pharmaceutically active substance is absorbed from the intestinal environment across the intestinal mucosa. Lipophilic pharmaceutical substances are generally poorly absorbed from the intestinal environment, inter alia because of their poor solubility and/or dispersibility in water. Bioavailability of a pharmaceutical substance taken per orally furthermore depends on the susceptibility of the substance to the so-called first pass effect. Substances absorbed from the intestine, before being distributed throughout the body, must pass the liver first where they may be metabolized immediately. CBD is generally assumed to be rather susceptible to first-pass liver metabolization. Oral bioavailability of CBD is low and unpredictable, and CBD is unstable. Zhornitsky et al., Pharmaceuticals 5:529-52 (2012); Poortman et al., Forensic Sci. Int. 101:1-8 (1999).

EPIDIOLEX® is the first FDA approved CBD pharmaceutical (Greenwich Biosciences Inc, Carlsbad, Calif.), with approved use in patients two years and older with Dravet syndrome or Lennox-Gastaut syndrome. Four randomized, double-blind, multicenter clinical trials evaluated the use of CBD in patients with Dravet syndrome or Lennox-Gastaut syndrome regarding the efficacy and safety in convulsive and drop seizure, respectively. All trials demonstrated a significant absolute reduction in seizure frequency. [Devinsky et al., N. Engl. J. Med. 376:2011-20 (2017); Devinsky et al., N. Eng. J. Med. 378:1888-97 (2018); Thiele et al., Lancet 1085-96 (2018)].

### 5.1 Terpenes

Cannabis contains many monoterpene and sesquiterpene compounds, together called terpenoids or terpenes, which are aromatic compounds synthesized in trichomes. In the plant, these compounds (i.e., more than 120 terpenes) synthesized alongside phytocannabinoids are important volatile constituents that are responsible for the plant's characteristic smell and also serve for different organic functions, such as insect repellent, repellent to herbivore attack, and attractive to pollinators. However, the presence of terpenoids has not been restricted to Cannabis sativa. These compounds normally occur in several other plant species, such as Mirabilis jalapa, Lithophragm glabrum, Cordia verbenacea, Eucalyptus globus, Syzygium aromaticum, Senna didymobotrya, Cymbopogon citratus, Pterodon emarginatus, Artemisia campestris, Lantana camara, Centella asiatica, Cyanthillium cinereum, Croton bonplandianus, and Citrus limon. Gonçalves et al., Molecules 25:1567 (2020).

B-caryophyllene and α-caryophyllene are the major sesquiterpenes of Cannabis. Booth et al., PLos ONE 12:e0173911 (2017). Caryophyllenes are phytocannabinoids with strong affinity to CB₂ but not CB₁. Gonsalves et al. (2020). Caryophyllenes have been reported to be repellent, antimicrobial, antibacterial, anticancer, antiproliferative, antifungal, AChE inhibitors, antioxidant, anti-inflammatory. Fidyt et al., Cancer Med. 5:3007-17 (2016); Sabulal et al., Phytochemistry 67:2469-73 (2006); Su et al., Nat. Prod. Commun. 11:845-48 (2016); Sarvmeili et al., Res. Pharm. Sci. 11:476-83 (2016); Memariani et al., Oncol. Lett. 11:1353-60 (2016); Segat et al., Neuropharmacology 125:207-19 (2017); Bento et al., Am. J. Pathol. 178:1153-66 (2011); Gertsch et al., Proc. Natl. Acad. Sci. USA 105:9099-9104 (2008); Alberti et al., J. Ethnopharmacol. 155:485-94 (2014).

Limonene ((4R)-1-methyl-4-prop-1-en-2-ylcyclohexene) is the most common natural monoterpene found in nature and is found in Cannabis sativa oilseed and in orange, lemon, and tangerine oils. Araujo-Filho et al., Neuroscience 358:158-69 (2017). Limonene does not interact with either CB1 or CB2. Santiago et al., Cannabis Cannabinoid Res. 4:165 76 (2019). Limonene has been reported to be anti-inflammatory, gastro-protective, anti-nociceptive, anti-tumor, neuroprotective, anti-hyperalgesic, anti-depressive, and anxiolytic. Araujo-Filho et al. (2017); Al-Ghezi et al., Front. Immunol. 10:1921 (2019); Sun, Altern. Med. Rev. J. Clin. Ther. 12:259-64 (2007); Shah et al., Anim. Models Exp. Med. 1:328-33 (2018); d'Allessio et al., Life Sci. 92:1151-56 (2013); de Almeida et al., Inflammation 40:511-22 (2017); do Amaral et al., Biol. Pharm. Bull. 30:1217-20 (2007); Piccinelli et al., Nutr. Neurosci. 18:217-24 (2015).

Linalool (3,7-dimethylocta-1,6-dien-3-ol) is a monoterpene compound present in several medicinal plants and fruits, including Cannabis sativa, and is used in cosmetics and flavoring ingredients. Zhang et al., Enzym. Microb. Technol. 134:109462 (2020). Linalool has been reported to be anti-inflammatory, anticancer, anxiolytic, neuroprotective, UV-protective, and pain reductive. Kim et al., Int. Immunopharmacol. 74:105706 (2019); Sabogal-Guaqueta et al., Biomed. Pharmacother. 118:109295 (2019); Harada et al., Front. Behay. Neurosci. 12:241 (2018); Xu et al., Life Sci. 174:21-27 (2017); Iwasaki et al., World J. Gastroenterol. 22:9765-74 (2016); Gunsaeelan et al., Photochem. Photobiol. Sci. Off. J. Eur. Photochem. Assoc. Eur. Soc. Photobiol. 15:851-60 (2016); Katsuyama et al., Biomed. Res. 33:175-81 (2012).

Terpineol (2-(4-methylcyclohex-3-en-1-yl)propan-2-ol) is a volatile monoterpene found in Cannabis sativa as well as cajuput, pine, and petitgrain oils. Gonsalves et al. (2020). Terpineol has been reported to be antinociceptive, antifungal, anti-inflammatory, antidiarrheal, pain reductive, memory enhancing, algeacidic, insect repellent, anti-proliferative, and anticancer. de Oliveira et al., Chem. Biol. Interact. 254:54-62 (2016); Chaudhari et al., Food Chem. 311:126010 (2020); de Oliveira et al., Basic Clin. Pharmacol. Toxicol. 111:120-25 (2012); dos Santos Negreiros et al., Biomed. Pharmacother. 110:631-40 (2019); Gouveia et al., Biomed. Pharmacother. 105:652-61 (2018); Parvardeh et al., Iran. J. Basic Med. Sci. 19:201-08 (2016); Kim et al., Biosci. Biotechnol. Biochem. 70:1821-26 (2006); Nogueira et al., Inflamm. Res. 63:769-78 (2014); Jing et al., Bot. Stud. 56:35 (2015); Chen et al., Ecotoxicol. Environ. Safi 167:435-40 (2019); Wua et al., Nat. Prod. Commun. 9:1515-18 (2014); Villa-Ruano et al., Chem. Biodivers. 15:e1800354 (2018); Hassan et al., Anticancer Res. 30:1911-19 (2010).

γ-terpinene (1-methyl-4-propan-2-ylcyclohexa-1,4-diene) is a monoterpene structurally similar to 1.8-cineol (eucalyptol) and is found in the essential oils of Cannabis sativa and other plants including the Eucalyptus genus (Myrtaceae), Cupressus cashmeriana, Lippia microphylla, Lavandula angustifolia, and Citrus myrtifolia. Gonçalves et al. (2020). γ-terpinene has been reported to be anti-inflammatory, antimicrobial, analgesic, and anticancer. Djenane et al., Food Sci. Technol. Int. 17:505-15 (2011); Ramalho et al., Planta Med. 81:1248-54 (2015); da Silva Lima et al., Eur. J. Pharmacol. 699:112-17 (2013); Guimaraes et al., Phytother. Res. PTR 27:1-15 (2013); Siveen et al., Can. J. Physiol. Pharmacol. 89:691-703 (2011); Ramalho et al., Axis. Planta Med. 82:1341-45 (2016); Baldissera et al., Exp. Parasitol. 162:43-48 (2016); Assmann et al., Biomed. Pharmacother. 103:1253-61 (2018).

α-pinene is found not only in Cannabis sativa but also in essential oils of many aromatic plants, such as Lavender angustifolia, Rosmarinus officinalis, and coniferous trees. Begum et al., Acta Sci. Polonorum. Technol. Aliment. 12:61-73 (2013). α-pinene has been reported to be antioxidant, antimicrobial, anti-tumor, hypnotic, anxiolytic, neuroprotective, cytoprotective, and antinociceptive. Zhao et al., Chemotherapy 63:1-7 (2018); Ibrahim et al., Planta Med. 85:431-38 (2019); Nissen et al., Fitoerapia 81:413-19 (2010); Yang et al., Mol. Pharmacol. 90:530-39 (2016); Satou et al., Phytother. Res. PTR 28:1284-87 (2014); Mercier et al., Int. J. Occup. Med. Environ. Health 22:331-42 (2009); Karthikeyan et al., Life Sci. 212:150-58 (2018); Karthikeyan et al., Life Sci. 217:110-18 (2019); Bouzenna et al., Biomed. Pharmacother. 93:961-6 (2017).

β-pinene, is found in many plants essential oils and can be obtained commercially by distillation or by α-pinene conversion. Iseppi et al., Molecules 24:2302 (2019). β-pinene has been reported to be antimicrobial, antioxidant, anti-immobility, and anti-adhesive. Mahajan et al., Environ. Sci. Pollut. Res. Int. 26:456-63 (2019); Guzman-Gutierrez et al., Life Sci. 128:24-29 (2015); Astani et al., Iran. J. Microbiol. 6:149-55 (2014); de Macêdo Andrade et al., Curr. Top. Med. Chem. 18:2481-90 (2018); Jia et al., Antimicrob. Agents Chemother. 46:947-57 (2002); da Silva et al., Molecules 17:6035-16 (2012).

β-elemene (1-methyl-1-vinyl-2,4-diisopropenyl-cyclohexane) is a derivative, which may arise due to oxidation or due to thermal- or UV-induced rearrangements during processing or storage. Booth et al. (2017). β-elemene has been reported to be anticancer. Deng et al., Phytother. Res. PTR 33:2248-56 (2019); Wu et al., Exp. Ther. Med. 13:3153-57 (2017); Fang et al., Oncol. Lett. 15:3957-64 (2018); Cai et al., Oncol. Lett. 16:6019-25 (2018); Liu et al., Oncol. Rep. 32:2635-47 (2014); Li et al., Anticancer Res. 33:65-76 (2013); Wei et al., Oncol. Rep. 37:3159-66 (2017); Yoshida et al., Lab. Investig. J. Tech. Methods Pathol. 93:1184-93 (2013); Liu et al., Biomed. Pharmacother. 95:1789-98 (2017); Zhang et al., Int. Immunopharmacol. 10:738-43 (2010).

β-ocimene (3,7-dimethyl-1,3,6-octatriene) is acyclic monoterpene that serves as a chemical cue to attract natural enemies of phytophagous insect in several plant species. Booth et al. (2017). β-ocimene has been reported to be antitumor, antifungal, and anticonvulsant. Bomfim et al., Basic Clin. Pharmacol. Toxicol. 118:208-13 (2016); Sayyah et al., J. Enthnopharmacol. 94:283-87 (2004).

Camphene (2,2-dimethyl-3-methylidenebicyclo(2.2.1)heptane) is a cyclic monoterpene present in Cannabis inflorescence in low titer but abundant in Thymus vulgaris oil. Gonçalves et al. (2020). Camphene has been reported to be expectorant, spasmolytic, and antimicrobial. Baser et al., Handbook of Essential Oils: Science, Technology, and Applications; CRC Press/Taylor & Francis: Boca Raton, Fla. (2010); Feng et al., Environ. Sci. Pollut. Res. Int. 26:16157-65 (2019); Benelli et al., Ecotoxicol. Environ. Saf 148:781-86 (2018); Benelli et al., Environ. Sci. Pollut. Res. Int. 25:10383-91 (2018).

Nerolidol ((6E)-3,7,11-trimethyldodeca-1,6,10-trien-3-ol; peruviol) is a noncyclic sesquiterpene alkene alcohol common to citrus peels, Piper claussenianum, Baccharis dracunculifolia, and Cannabis. Baldissera et al., Naunyn-Schmiedeberg's Arch. Pharmacol. 391:753-59 (2018). Nerolidol has been reported to be antimicrobial and anti-inflammatory. Alonso et al., Biochim. Biophys. Acta Biomembr. 1861:1049-56 (2019); Zhang et al., Phytother. Res. PTR 31:459-65 (2017); Iqubal et al., 236:116867 (2019); Iqubal et al., Eur. J. Pharmacol. 863:172666 (2019).

Euphol, a tetracyclic triterpene and a minor Cannabis component, is usually extracted in alcoholic preparations. Pellati et al., Biomed. Res. Int. 2018:1691428 (2018). Euphol has been report to be anticancer and anti-inflammatory. Betancur-Galvis et al., Mem. Inst. Oswaldo Cruz 97:541-46 (2002); Prinsloo et al., J. Ethnopharmacol. 210:133 55 (2018); Mazior et al., Fur Naturforschung. Cjournal Biosci. 66:360-66 (2011); Silva et al., Exp. Ther. Med. 16:557-66 (2018); Cruz et al., Phytomedicine: Int. J. Phytother. Phytopharm. 47:105-112 (2018); Wang et al., Mol. Med. Rep. 8:1279-85 (2013); Silva et al., Investig.

New Drugs 37:223-37 (2019).

### 5.2 Cannabis and Viral Infections/Immune Function

Studies on the effects of Cannabis use or cannabinoid treatment of viral infections are limited. One report suggests that Cannabis use is associated with decreased incidence of liver cirrhosis and lower overall treatment cost, but without a corresponding change in mortality or length of stay among Hepatitis C virus (HCV) patients. [Adejumo et al., Can. J. Gastroenterol. Hepatol. (2018) 9430953]. Preclinical studies have demonstrated that Cannabis modulates inflammatory and fibrotic processes in the liver. [Patsenker et al., Int. J. Mol. Sci. (2015)16:7057-76]. Earlier studies suggested that Cannabis use resulted in increased steatosis, fibrosis, and worsening of HCV disease, more recent studies indicate that Cannabis use does not alter HCV disease progression. [Adejumo et al., Can. J. Gastroenterol. Hepatol. (2018) 9430953]. A recent study, however, revealed that CBD induced cell death in approximately 85% of HCV infected cells in vitro, like INF-αB2 treatment. [Lowe et al., Pharmacogn. Res. (2017)9:116-18].

Substantial Cannabis use is associated with decreased frequencies of activated T cells and inflammatory antigen-presenting cell subsets, which suggests a potential immunologic benefit of cannabinoids through decreased immune activation in human immunodeficiency virus (HIV)-infected individuals. [Manuzak et al., Clin. Infect. Dis. (2018). 66:1872-82]. One way that Cannabis may exert an anti-inflammatory impact in HIV-infected individuals is by increasing the frequency of CD16⁻ monocyte subsets and decreasing the frequency of CD16⁺ monocyte subsets. [Id.]. Another study demonstrated that Cannabis use is associated with lower viremia following seroconversion. [Milloy et al., Drug Alcohol Rev. (2015) 34:135-40].

THC and synthetic THC, like dronabinol (Marinol or Syndros), exhibit potent anti-inflammatory activity and are immunosuppressive. [Tanasescu et al., Immunobiology (2010) 215:588-97; Klein et al., Immunol. Today (1998)19:373-81]. THC can suppress T-cell responses to viral infections. Reiss, Pharmaceuticals (Basel) 3:1873-86 (2010). Additionally, plasmacytoid dendritic cell secretion of IFN-α is highly sensitive to THC-mediated suppression, and plasmacytoid dendritic cells from HIV donors have increased sensitivity to THC-mediated suppression as compared to plasmacytoid dendritic cells from healthy donors. Henriquez et al., J. Acquir. Immune Defic. Syndr. 75:588-96 (2017).

CBD is highly lipophilic and subject to first-pass metabolism after oral dosing. [Nichols, J M, Kaplan, B L F. Cannabis and Cannabinoid Res. (2018) doi: 10.1.089/can.2018.0073, citing Samara, E. et al. Drug Metab. Dispos. (1988) 16: 469-72]. Data to date overwhelmingly demonstrate that CBD is immunosuppressive and anti-inflammatory. [Id.] Identification of receptors through which CBD acts in the immune system and the cell types on which those receptors are expressed that mediate the CBD effects remain unclear. [Id.] It is known that effects of CBD are mediated through activation of CB1, CB2, transient receptor potential V1 (TRPV1), known as the vanilloid receptor, adenosine A2A receptors, and PPAR-γ receptors, blockade of GPR55 receptors, and fatty acid amide hydrolase (FAAH) inhibition. [Id.] The effects of CBD on immune responses can involve innate or adaptive responses. [Id.] Targets of suppression include cytokines, such as TNF-α, IFN-γ, I1-6, IL-1β, IL-2, IL-17A, and chemokines, such as CCL-2. [Id.] The overall mechanism of CBD involves direct suppression of target cells, such as effector T cells, innate, and microglial cells, through suppression of kinase cascades and various transcription factors. However, limited data is available examining CBD's effects on various T cell subsets. While results suggest that B cells can be targets of suppression by CBD, there are only a few studies in which B cells are identified as targets of CBD. Direct suppression of target cells also includes induction of IiB, which could contribute to decreased NF-κB activity. [Id.] The involvement of regulatory cell induction by CBD is also a major part of the mechanism by which CBD controls immune responses, and CBD has been shown to induce regulatory T cells (Tregs) and myeloid-derived suppressor cells (MDSCs), the latter of which are innate, myeloid cells that possess the ability to suppress T cell responses [Id.; see Gabrilovich, D., Nagaraj, S. Nat. Rev. Immunol. (2009) 9 (3): 162-74]. CBD-induced apoptosis is likely a mechanism in many target cells. Nichols, J M, Kaplan, B L F. Cannabis and Cannabinoid Res. (2018) doi: 10.1.089/can.2018.0073]

Both elevated exhaustion levels and reduced functional diversity of T cells in peripheral blood have been suggested as potential predictors of severe progression in COVID-19 patients [Zheng, H Y et al., Cellular & Molec. Immunol (2020) doi.org/10.1038/x41423-020-0401-3]. Treatments to prevent the progression to T cell exhaustion in susceptible patients therefore may be critical to their recovery.

### 7. The Described Invention

Studies described above to explore the effectiveness of NAC alone or as an adjunct to primary therapies in the treatment of inflammatory pulmonary disease have shown that NAC is an effective complement to anti-virals in preventing further progression in patients symptomatic for HIV. Studies in animal models of influenza virus infection have also shown synergy of NAC with anti-virals.

In monocyte-NK cell co-cultures, NK cells were shown to reduce the intracellular growth of Mycobacterium tuberculosis strain 3tRv. Treatment of NK cells in vitro with the combination of IL-2, IL-12, and NAC enhanced NK cell functions to control M. tuberculosis infection, causing substantial growth inhibition of intracellular Mycobacterium tuberculosis H37Rv. [Millman, A C et al. H. Interferon & Cytokine Res. (2008) 28: 153-65].

Using a retrospective search of the scientific literature and meta-analysis of clinical trials, it was concluded that NAC taken at high doses for at least 6 months can ameliorate deterioration of lung in patients with cystic fibrosis (CF). Conrad, C. Chapter 15, in The Therapeutic Use of N-acetylcysteine (NAC) in Medicine, Frye, R E and Berk, M., Eds., Springer Nature Singapore, Pte Ltd. (2019) pp 255-276. CF is an inherited lethal disease in Caucasians, which is characterized by chronic infection and inflammation by activation of the innate immune system. [Ralhan, A., et al. J. Innate Immunol. (2016) 6 (8): 531-40]. A profound GSH depletion is believed to affect neutrophil recruitment to the lungs of CF patients and may contribute to the exuberant inflammatory response described in these patients. Conrad, C. Chapter 15, in The Therapeutic Use of N-acetylcysteine (NAC) in Medicine, Frye, R E and Berk, M., Eds., Springer Nature Singapore, Pte Ltd. (2019) pp 255 276. Evidence also points in a favorable direction for recommendation for the use of oral NAC in subjects with COPD to preserve lung function by reducing pulmonary oxidative stress and inflammation and attenuating airway and parenchymal destruction. [Id.]

A potential immunologic benefit through decreased immune activation has been suggested for cannabinoids in HIV-infected individuals. As described above, the effects of cannabinoids on the immune response can involve innate and/or adaptive responses. Cannabinoids are thought to act through activation of receptors, for example, CB1, CB2, transient receptor potential V1 (TRPV1), known as the vanilloid receptor, adenosine A2A receptors, and PPAR-γ receptors, blockade of GPR55 receptors, and fatty acid amide hydrolase (FAAH) inhibition.

The approach taken by the described invention therefore is to determine whether NAC alone, a cannabinoid or cannabimimetic alone, or the combination of NAC and a cannabinoid or cannabimimetic can improve or restore immune system health in a susceptible subject and/or a subject infected with a respiratory virus that impacts the immune system by reducing functional diversity of T cells and promoting T cell exhaustion, compared to a control.

### SUMMARY OF THE INVENTION

According to one aspect, the present disclosure provides a method for improving immune system health in a subject in need thereof comprising administering to the subject a composition comprising a therapeutic amount of an active constituent and a pharmaceutically acceptable carrier, wherein the active constituent comprises one or more of N-acetylcysteine, a botanical ingredient, or a cannabimimetic, wherein the therapeutic amount of the active constituent potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100% compared to a control.

According to one embodiment, the N-acetylcysteine directly potentiates the immune response by increasing immune diversity. According to some embodiments, when the active constituent comprises N-acetylcysteine and a botanical ingredient or cannabimimetic the therapeutic effect of the N-acetylcysteine and the botanical material or cannabinoid are complementary. According to some embodiments, a therapeutic effect of the botanical material or cannabinoid is non-psychoactive. According to some embodiments, the cannabimimetic is a terpinoid, a fatty acid derivative, a flavonoid, or is derived from derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin B1) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or Polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; or an essential oil selected from Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), or Ylang ylang (Cananga odorata(Lam.) var. genuine). According to some embodiments of the method, for each dose of N-acetylcysteine, onset of potentiation of the immune response by increasing diversity occurs within 24 hours of dosing; and for each dose of the botanical ingredient or cannabinimimetic, onset of potentiation of the immune response by increasing diversity occurs within 24 hours of dosing. According to some embodiments compared to the subject before the administering, the potentiated immune response comprises: an enhanced T cell diversity, or an enhanced B cell diversity, or an enhanced T cell diversity and an enhanced B cell diversity; or a stabilized T cell immune repertoire. According to some embodiments the potentiated immune response comprises an increased resistance to T cell exhaustion. According to some embodiments the potentiated immune response: improves clinical outcome in response to a pathogen; or reduces a burden of disease; or reduces appearance of disease; or increases health span of the subject. According to some embodiments the pathogen is a microbe selected from a bacterium, a fungus, a protozoan, a virus, or an algae. According to some embodiments the therapeutic amount of the composition comprising N-acetylcysteine further comprises a mucolytic therapeutic effect, an anti-oxidant therapeutic effect, or both. According to some embodiments the subject in need is an aged person of greater than 60 years of age. According to some embodiments the immune system of the aged person is compromised by one or more of prior illnesses, chronic illnesses, or the aging process. According to some embodiments the botanical ingredient or cannabimimetic is tetahydrocannabinol (THC) or curcumin. According to some embodiments the administering to the subject comprises alternating a composition comprising N-acetylcysteine with a composition comprising the botanical ingredient or cannabimimetic, wherein immune diversity of the immune system increases once the composition comprising the botanical ingredient or cannabimimetic is stopped. According to some embodiments the alternating is weekly. According to some embodiments the botanical ingredient or cannabimimetic comprises cannabidiol (CBD), palmitoylethanolamine (PEA), or Curcumin (CUR). According to some embodiments the alternating increases vibrancy of the immune system, stimulates a reorganizational change in the immune system, or both. According to some embodiments the subject is suffering from post-acute COVID-19 syndrome.

According to another aspect, the present disclosure provides a method for treating symptoms of a respiratory virus infection, comprising administering to a subject in need thereof a composition comprising a therapeutic amount of an active constituent and a pharmaceutically acceptable carrier, wherein the active constituent comprises one or more of N-acetylcysteine, a botanical ingredient, or a cannabimimetic, and wherein the therapeutic amount of the active constituent potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100% compared to a control.

According to one embodiment, the N-acetylcysteine directly potentiates the immune response by increasing immune diversity. According to some embodiments, when the active constituent comprises N-acetylcysteine and a botanical ingredient or cannabimimetic the therapeutic effect of the N-acetylcysteine and of the botanical material or cannabinoid are complementary. According to some embodiments, a therapeutic effect of the botanical material or cannabinoid is non-psychoactive. According to some embodiments the cannabimimetic is a terpinoid, a fatty acid derivative, a flavonoid, or is derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; or an essential oil selected from Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), or Ylang ylang (Cananga odorata(Lam.) var. genuine). According to some embodiments of the method, for each dose of N-acetylcysteine, onset of potentiation of the immune response by increasing diversity occurs within 24 hours; and for each dose of the botanical ingredient or cannabinimimetic, onset of potentiation of the immune response by increasing diversity occurs within 24 hours. According to some embodiments, compared to the subject before the administering, the potentiated immune response comprises: an enhanced T cell diversity, or an enhanced B cell diversity, or an enhanced T cell diversity and an enhanced B cell diversity; or a stabilized T cell immune repertoire. According to some embodiments the potentiated immune response comprises an increased resistance to T cell exhaustion. According to some embodiments the potentiated immune response: improves clinical outcome in response to a pathogen; or reduces a burden of disease; or reduces appearance of disease; or increases health span of the subject. According to some embodiments the pathogen is a microbe selected from a bacterium, a fungus, a protozoan, a virus, or an algae. According to some embodiments the therapeutic amount of the composition comprising N-acetylcysteine further comprises a mucolytic therapeutic effect, an anti-oxidant therapeutic effect, or both. According to some embodiments the subject in need is an aged person of greater than 60 years of age. According to some embodiments the immune system of the aged person is compromised by one or more of prior illnesses, chronic illnesses, or the aging process. According to some embodiments the therapeutic amount reduces viral load. According to some embodiments the botanical ingredient or cannabimimetic is tetramydrocannabinol (THC) or curcumin. According to some embodiments the administering to the subject comprises alternating a composition comprising N-acetylcysteine with a composition comprising the botanical ingredient or cannabimimetic, wherein immune diversity of the immune system increases once the composition comprising the botanical ingredient or cannabimimetic is stopped. According to some embodiments the alternating is weekly. According to some embodiments the botanical ingredient or cannabimimetic comprises cannabidiol (CBD), palmitoylethanolamine (PEA) or Curcumin (CUR). According to some embodiments the alternating increases vibrancy of the immune system, stimulates a reorganizational change in the immune system or both. According to some embodiments the subject is suffering from post-acute COVID-19 syndrome.

According to another aspect, the present disclosure provides a method for increasing potency or efficacy of an antiviral vaccine in increasing a subject's resistance to a viral infection, comprising administering to the subject a composition comprising a therapeutic amount of an active constituent and a pharmaceutically acceptable carrier, wherein the active constituent comprises one or more of N-acetylcysteine, a botanical ingredient, or a cannabimimetic, and wherein the therapeutic amount of the active constituent potentiates an anti-viral immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%, compared to a control.

According to one embodiment, the method according to claim 40, wherein the N-acetylcysteine directly potentiates the immune response by increasing immune diversity. According to some embodiments the antiviral vaccine is employed to help the subject's body's immune system recognize and fight infections caused by the virus in a susceptible population. According to some embodiments the virus is a polio virus, a measles virus, a mumps virus, a rubella virus, an influenza virus, a rotavirus, a human immunodeficiency virus, a SARS coronavirus, or a rabies virus. According to some embodiments when the active constituent comprises N-acetylcysteine and a botanical ingredient or cannabimimetic the therapeutic effect of the N-acetylcysteine and of the botanical material or cannabinoid are complementary. According to some embodiments, a therapeutic effect of the botanical material or cannabinoid is non-psychoactive. According to some embodiments the cannabimimetic is a terpinoid, a fatty acid derivative, a flavonoid, or is derived from turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; or an essential oil selected from Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), or Ylang ylang (Cananga odorata(Lam.) var. genuine). According to some embodiments of the method, for each dose of N-acetylcysteine, onset of potentiation of the immune response by increasing diversity occurs within 24 hours of dosing; and for each dose of the botanical ingredient or cannabimimetic, onset of potentiation of the immune response by increasing diversity occurs within 24 hours of dosing. According to some embodiments compared to the subject before the administering, the potentiated immune response comprises: an enhanced T cell diversity, or an enhanced B cell diversity, or an enhanced T cell diversity and an enhanced B cell diversity; or a stabilized T cell immune repertoire. According to some embodiments the potentiated immune response comprises an increased resistance to T cell exhaustion. According to some embodiments the potentiated immune response: improves clinical outcome in response to a pathogen; or reduces a burden of disease; or reduces appearance of disease; or increases health span of the subject. According to some embodiments the pathogen is a microbe selected from a bacterium, a fungus, a protozoan, a virus, or an algae. According to some embodiments the therapeutic amount of the composition comprising N-acetylcysteine further comprises a mucolytic therapeutic effect, an anti-oxidant therapeutic effect, or both. According to some embodiments the subject in need is an aged person of greater than 60 years of age. According to some embodiments the immune system of the aged person is compromised by one or more of prior illnesses, chronic illnesses, or the aging process. According to some embodiments the aging process comprises biological and physiological changes that result in increased susceptibility to the viral infection. According to some embodiments the botanical ingredient or cannabimimetic is tetrahydrocannabinol (THC) or curcumin. According to some embodiments the administering to the subject comprises alternating a composition comprising N-acetylcysteine with a composition comprising the botanical ingredient or cannabimimetic, wherein immune diversity of the immune system increases once the composition comprising the botanical ingredient or cannabimimetic is stopped. According to some embodiments the alternating is weekly. According to some embodiments the botanical ingredient or cannabimimetic comprises cannabidiol (CBD), palmitoylethanolamine (PEA) or Curcumin (CUR). According to some embodiments the alternating increases vibrancy of the immune system, stimulates a reorganizational change in the immune system, or both. According to some embodiments the subject is suffering from post-acute COVID-19 syndrome.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows the immune diversity map of the TCR β chain immune repertoire for subject 1 (n=9) at baseline. FIG. 1B shows the immune diversity map for TCR β chain immune repertoire for subject 1 at 24 hours after dosing with 5 mg controlled dose of cheese quick dissolve tablets (QDT) containing 4.9 mg THC, >0.1 mg THCA, >0.1 mg CBD, 0.2 mg CBG, >0.1 mg CBC, >0.1 mg THCV, >0.1 mg CBN, with pinene, limonene, beta-caryophylleme, humulene, and nerolidol. The number and size of the rounded squares indicate a single T-cell beta chain receptor clonotype and its frequency in peripheral blood. FIG. 1C is a bar graph illustrating T cell β chain diversity index 24 hours after a single dose of 5 mg Cheese for subject 1 compared to baseline. For subject 1, diversity index increased from 6.7 to 19.2 (2.9 fold increase).

FIG. 2A, 2B, 2C, 2D, 2E, 2F, and 2G are bar graphs illustrating T cell β chain diversity index after 24 hours for each of 6 subjects compared to baseline. Each subject received a controlled dose of a cannabinoid and a blood sample was tested 24 hours after dosing. FIG. 2A; shows results for subject 2, 24 hours after a single dose of 5 mg Cheese; FIG. 2B shows results for subject 3, 24 hours after a single dose of 5 mg Cheese; FIG. 2C shows results for subject 4, 24 hours after a single dose of 10 mg Cheese; FIG. 2D shows results for subject 5, 24 hours after a single dose of 5 mg Cheese; FIG. 2E shows results for subject 6, 24 hours after a single dose of Granddaddy purple tablet; FIG. 2F shows results for subject 8, 24 hours after a single dose of 5 mg Cheese; FIG. 2G shows results for subject 9, 24 hours after a single dose of 5 mg Cheese.

FIG. 3A shows the immune diversity map for the TCR β chain immune repertoire for subject 1 (n=2) at baseline; FIG. 3B shows the immune diversity map for TCR β chain immune repertoire for subject 1 at 24 hours after dosing with Jack CBD quick dissolve tablet containing CBD 7.6 mg CBD and 4.2 mg THC. FIG. 3C shows 48 hours after dosing with Jack CBD. CBD-dominant QDTs show a different repertoire response kinetics than for THC. The number and size of the rounded squares indicate a single T-cell beta chain receptor clonotype and its frequency in peripheral blood. FIG. 3D is a bar graph illustrating T cell βchain diversity index for subject 1, compared to baseline, 24 hours and 48 hours after receiving a controlled dose of Jack CBD cannabinoid.

FIG. 4 is a bar graph illustrating T cell β chain diversity index for subject 2, compared to baseline 24 hours after receiving a controlled dose of Jack CBD cannabinoid.

FIG. 5A, 5B, 5C, 5D, 5E, and 5F show immune diversity maps for the TCR βchain immune repertoire after 1 mg, 5 mg, and 10 mg controlled dose Cheese quick dissolve tablets (QDTs). Cheese QDTs contained 4.9 mg THC, >0.1 mg THCA, >0.1 mg CBD, 0.2 mg CBG, >0.1 mg CBC, >0.1 mg THCV, >0.1 mg CBN, with pinene, limonene, beta-caryophylleme, humulene, and nerolidol. FIG. 5A shows the immune diversity map for TCR β chain immune repertoire for subject E2 at baseline; FIG. 5B shows the immune diversity map for subject E2 at 24 hours for a dose of 1 mg THC. Diversity index decreased from 8.6 to 8.1 (0.9× decrease). FIG. 5C and FIG. 5D show the immune diversity map for TCR βchain immune repertoire for subject E2 at baseline (FIG. 5C) and at 24 hours (FIG. 5D) for a dose of 5 mg THC. Diversity index increased from 15.2 to 23.1 (1.5 fold increase). FIG. 5E, and FIG. 5F show the immune diversity map for TCR β chain immune repertoire for subject E2 at baseline (FIG. 5E) and at 24 hours (FIG. 5F) for a dose of 10 mg THC.

Diversity index increased from 8.7 to 17.4 (2.0 fold increase). The number and size of the rounded squares indicate a single T-cell beta chain receptor clonotype and its frequency in peripheral blood. FIG. 5G is a bar graph illustrating a dose response in T cell β chain diversity index for the subject compared to baseline 24 hours after receiving a 1 mg, a 5 mg, and a 10 mg dose of THC (Cheese QDT). FIG. 5H is a graph showing percentage change in diversity index as a function of dose.

FIG. 6A and FIG. 6B show immune diversity maps for the TCR β chain immune repertoire after a single dosage of N-acetylcysteine (NAC) (2×600 mg effervescent tablets) for subject 1 (n=2). FIG. 6A shows an immune diversity tree map at baseline for subject 1. FIG. 6B shows the effect on the immune diversity map after a single 1200 mg dose of effervescent NAC in subject 1 24 hours post dose. For subject 1, diversity index increased from 12.7 to 20.0 (1.6 fold increase). The number and size of the rounded squares indicate a single T-cell beta chain receptor clonotype and its frequency in peripheral blood. FIG. 6C is a bar graph illustrating T cell β chain diversity index compared to baseline for subject 1 24 hours after receiving a single dose of N-acetylcysteine (1200 mg).

FIG. 7A and FIG. 7B show immune diversity maps for the TCR β chain immune repertoire after a single dosage of N-acetylcsteine (NAC) (2×600 mg effervescent tablets).for subject 2 (n=2) FIG. 7A shows an immune diversity tree map at baseline for subject 2. FIG. 7B shows the effect on the immune diversity map of a single 1200 mg dose of effervescent NAC in subject 2 24 hours post dose. For subject 2, diversity index increased from 10.3 to 15.9 (1.5 fold increase). The number and size of the rounded squares indicate a single T-cell beta chain receptor clonotype and its frequency in peripheral blood. FIG. 7C is a bar graph illustrating T cell β chain diversity index compared to baseline for subject 2 after receiving a single dose of N-acetylcysteine (1200 mg).

FIG. 8A and FIG. 8B show the immune diversity maps for TCR β chain immune repertoire for a subject (n=1) at baseline (FIG. 8A) and at 72 hours (FIG. 8B) after topical application of 10 mg THC salve made with silver OG (THC dominant strain). No intoxication characteristic of THC occurred with application of salve for three days. The number and size of the rounded squares indicate a single T-cell beta chain receptor clonotype and its frequency in peripheral blood. FIG. 8C is a bar graph illustrating T cell β chain diversity index compared to baseline after applying a salve containing 10 mg THC made with Silver OG. Diversity index increased from 8.9 to 18.9 (2.0 fold increase).

FIG. 9 and FIG. 10 provide diagrams that summarize the adaptive clinical study design for both period 1 (FIG. 9) and period 2 (FIG. 10).

CBC data plots for subjects treated with NAC are shown in FIG. 11A (WBCs), FIG. 11B (RBCs), FIG. 11C (Hb), FIG. 11D (HCT), FIG. 11E (MCV), FIG. 11F (MCH), FIG. 11G (MCHC), FIG. 11H (RDW), FIG. 11I (platelets), FIG. 11J (neutrophils), FIG. 11K (lymphocytes), FIG. 11L (monocytes), FIG. 11M (eosinophils), FIG. 11N (basophils), FIG. 110 (granulocytes), FIG. 11P(NRBCs).

CBC data plots for subjects treated with Curcumin are shown in FIG. 12A (WBCs), FIG. 12B (RBCs), FIG. 12C (Hb), FIG. 12D (HCT), FIG. 12E (MCV), FIG. 12F (MCH), FIG. 12G (MCHC), FIG. 12H (RDW), FIG. 12I (platelets), FIG. 12J (neutrophils), FIG. 12K (lymphocytes), FIG. 12L (monocytes), FIG. 12M (eosinophils), FIG. 12N (basophils), FIG. 120 (granulocytes), FIG. 12P(NRBCs).

CBC data plots for subjects treated with PEA are shown in FIG. 13A (WBCs), FIG. 13B (RBCs), FIG. 13C (Hb), FIG. 13D (HCT), FIG. 13E (MCV), FIG. 13F (MCH), FIG. 13G (MCHC), FIG. 13H (RDW), FIG. 13I (platelets), FIG. 13J (neutrophils), FIG. 13K (lymphocytes), FIG. 13L (monocytes), FIG. 13M (eosinophils), FIG. 13N (basophils), FIG. 130 (granulocytes), FIG. 13P (NRBCs).

CBC data plots for subjects treated with Echinacea are shown in FIG. 14A (WBCs), FIG. 14B (RBCs), FIG. 14C (Hb), FIG. 14D (HCT), FIG. 14E (MCV), FIG. 14F (MCH), FIG. 14G (MCHC), FIG. 14H (RDW), FIG. 14I (platelets), FIG. 14J (neutrophils), FIG. 14K (lymphocytes), FIG. 14L (monocytes), FIG. 14M (eosinophils), FIG. 14N (basophils), FIG. 140 (granulocytes), FIG. 14P (NRBCs).

FIG. 15 is a schematic of the trial dosing time line and events. Blood draws were on Day 0, Day 7, Day 21 and Day 29. Day 0 to Day 7 represents the lead-in period. From Day 7 to Day 21 the subjects received daily dosing. The final dose was on day 21. From day 22 to day 29 (i.e., 7 days after dosing stopped) is defined as the washout period, there was no dosing during this period.

FIG. 16 is a schematic depicting the correlation between the trial dosing time line and events and the time line and events using bar graphs that plot percent difference value (y-axis) vs. sample states (x axis). Sample states are represented by Intervals 1 through 6.

Interval 1 is from Day 0 to Day 7. Interval 2 is from day 0 to day 21. Interval 3 is from day 0 to day 29. Interval 4 is from day 7 to day 21 (i.e., daily dosing period). Interval 5 is from day 7 to day 29. Interval 6 is from day 21 to day 29.

FIG. 17 is a schematic depicting the correlation between the trial dosing time line and the diversity index plot of diversity index versus visit. The baseline points provide an idea of natural changes in the subjects. Dosing occurred one week after the lead-in baseline. The period one week after dosing stopped on day 21 represents the reorganization process.

FIG. 18 shows a bar graph of mean Diversity Index percent difference on the Y axis. The X axis corresponds to time periods in days (d) (from left to right, visits 0 d to 7 d, 0 d to 21 d, 0d to 29 d, 7 d to 21 d, 7 d to 29 d, and 21 d to 29 d, with 0 d=visit 2, 7 d=visit 3, 21 d=visit 4 and 29 d=visit 5). At time periods 20 d to 21 d and 7 d to 21 d, the increase in diversity was up in sync during treatment with N-acetylcysteine (NAC).

FIG. 19A shows a plot of the calculated Diversity Index vs. visit number for an example participant who was treated with N-acetylcysteine (NAC). The Diversity Index is equal to 100 minus the area under the curve in a normalized frequency distribution curve plotting the percentage of total reads and the percentage of unique CDR3s, when unique CDR3s are sorted, by frequency, from largest to smallest. Visit 2 was 1 week prior to dosing, Visit 3 was immediately prior to the start of dosing, Visit 4 was after 2 weeks of daily dosing N-acetylcysteine 600 mg twice daily (1200 mg total daily dose), and Visit 5 was 1 week after dosing was stopped. FIG. 19B shows corresponding tree maps for each of visits 2, 3, 4, & 5. Each spot in the plot represents a unique CDR3 and the size of a spot denotes the relative frequency.

FIG. 20 shows a bar graph of mean Clonality percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). Clonality is calculated as (1—the Shannon Equitability Index). The Shannon Equitability Index is the ratio of the Shannon Diversity to the Shannon Entropy. The closer this number is to 1, the closer the distribution is to the most entropic distribution, i.e., all uCDR3s at frequency of 1. The lower the clonality, the closer to this distribution. A positive change in clonality represents a movement from the most entropic distribution. This tracks, trendwise, inversely with the change in unique CDR3s (uCDRs). A specific unique CDR3 may have 1 copy in a sample or may have thousands of copies. At time periods 0 d to 21 d and 7 d to 21 d, the increase in mean clonality was up in synch during treatment with N-acetylcysteine (NAC).

FIG. 21 is a bar graph showing mean ALICE significant nodes percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). The number of nodes represents the total number of unique CDR3 amino acid sequences within the networked sample. Unique sequences that do not cluster are not counted. “Significant nodes” are the number of notes with significant interactions. At time periods 0 d to 21 d and 7 d to 21 d At visits 2 and 4, the increase in mean clonality was up in synch during treatment with N-acetylcysteine (NAC).

FIG. 22 is a bar graph showing mean ALICE significant clusters percent difference on the Y axis. This is a direct measure of the number of clusters calculated within the repertoire's distribution of sequences. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). At time periods 0 d to 21 d and 7 d to 21 d, the increase in mean clusters was up in synch during treatment with N-acetylcysteine (NAC).

FIG. 23 shows a bar graph of mean Diversity Index percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). 4 At time periods 0 d to 21 d and 7 d to 21 d, the increase in diversity was up in synch during treatment with Curcumin (CUR).

FIG. 24A shows the calculated Diversity Index vs. visit number for an example participant who was treated with curcumin (CUR). The Diversity Index is equal to 100 minus the area under the curve in a normalized frequency distribution curve plotting the percentage of total reads and the percentage of unique CDR3s, when unique CDR3s are sorted, by frequency, from largest to smallest. Visit 2 was 1 week prior to dosing, Visit 3 was immediately prior to the start of dosing, Visit 4 was after 2 weeks of daily dosing curcumin 3×60 mg capsules once daily (180 mg total daily dose), and Visit 5 was 1 week after dosing was stopped. FIG. 24B shows corresponding tree maps for each of visits 2, 3, 4, & 5. Each spot in the plot represents a unique CDR3 and the size of a spot denotes the relative frequency.

FIG. 25 shows a bar graph of mean Clonality percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). (from left to right, 0 d to 7 d, 0 d to 21 d, 0 d to 29 d, 7 d to 21 d, 7 d to 29 d, 21 d to 29 d. Clonality at time points 0 d to 29 d, 7 d to 29 d, 21 d to 29 d was down on reorganization following treatment with Curcumin (CUR).

FIG. 26 is a bar graph showing mean ALICE significant nodes percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). The number of nodes represents the total number of unique CDR3 amino acid sequences within the networked sample. Unique sequences that do not cluster are not counted. “Significant nodes” are the number of notes with significant interactions. At time periods 0 d to 29 d, 7 d to 29 d, 21 d to 29 d, there was a decrease in mean nodes on reorganization during treatment with Curcumin (CUR).

FIG. 27 is a bar graph showing mean ALICE significant clusters ALICE percent difference on the Y axis. This is a direct measure of the number of clusters calculated within the repertoire's distribution of sequences. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). Note that the cluster data for Curcumin is quite different from that of NAC. At time periods 0 d-21 d and 7 d to 21 d significant clusters are up in sync during treatment while at time periods 0 d to 29 d, 7 d to 29 d, and 21 d to 29 d significant clusters are down on reorganization. Whereas NAC cluster data support a diversity increase upon treatment, the cluster data for Curcumin (CUR) supports reorganization of the repertoire post-treatment.

FIG. 28 shows a bar graph of mean Diversity Index percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). At time periods 0 d to 29 d, 7 d to 29 d, 21 d to 29 d visits 3, 5 and 6, the increase in diversity was up on reorganization post-treatment with palmitoylethanolamide (PEA).

FIG. 29A shows the calculated Diversity Index vs. visit number for an example participant who was treated with palmitoylethanolamide (PEA). The Diversity Index is equal to 100 minus the area under the curve in a normalized frequency distribution curve plotting the percentage of total reads and the percentage of unique CDR3s, when unique CDR3s are sorted, by frequency, from largest to smallest. Visit 2 was 1 week prior to dosing, Visit 3 was immediately prior to the start of dosing, Visit 4 was after 2 weeks of daily dosing PEA 3×400 mg capsules once daily (1200 mg total daily dose), and Visit 5 was 1 week after dosing was stopped. FIG. 29B shows corresponding tree maps for each of visits 2, 3, 4, & 5. Each spot in the plot represents a unique CDR3 and the size of a spot denotes the relative frequency.

FIG. 30 shows a bar graph of mean Clonality percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). Clonality at time periods 0 d to 29 d, 7 d to 29 d, 21 d to 29 d was down on reorganization following treatment with palmitoylethanolamide (PEA).

FIG. 31 is a bar graph showing mean ALICE significant clusters ALICE percent difference on the Y axis. This is a direct measure of the number of clusters calculated within the repertoire's distribution of sequences. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). (from left to right, 0 d to 7 d, 0 d to 21 d, 0 d to 29 d, 7 d to 21 d, 7 d to 29 d, 21 d to 29 d. At time periods 0 d to 21 d, 7 d to 21 d 2 and 4 clustering decreases. At time periods 0 d to 29 d, 7 d to 29 d, 21 d to 29 d, there is a dramatic increase in clustering with reorganization following treatment with palmitoylethanolamide (PEA).

FIG. 32A shows the calculated Diversity Index vs. visit number for an example participant who was treated with Echinacea pupurea (ECH). The Diversity Index is equal to 100 minus the area under the curve in a normalized frequency distribution curve plotting the percentage of total reads and the percentage of unique CDR3s, when unique CDR3s are sorted, by frequency, from largest to smallest. Visit 2 was 1 week prior to dosing, Visit 3 was immediately prior to the start of dosing, Visit 4 was after 2 weeks of daily dosing of Echinacea-one 252 mg capsule twice daily (504 mg total daily dose), and Visit 5 was 1 week after dosing was stopped. FIG. 32B shows corresponding tree maps for each of visits 2, 3, 4, & 5. Each spot in the plot represents a unique CDR3 and the size of a spot denotes the relative frequency.

### DETAILED DESCRIPTION OF THE INVENTION

### 1. Definitions

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. For example, a mature B cell can be activated by an encounter with an antigen that expresses epitopes that are recognized by its cell surface immunoglobulin Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγl, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The soluble product of an activated B lymphocyte is immunoglobulins (antibodies). The soluble product of an activated T lymphocyte is lymphokines.

The term “active” refers to the ingredient, component or constituent of the compositions of the described invention responsible for the intended therapeutic effect.

The term “adaptive immunity” as used herein refers to the protection of a host organism from a pathogen or toxin which is mediated by B cells and T cells, and is characterized by immunological memory. Adaptive immunity is highly specific to a given antigen and is highly adaptable.

The phrase “additional active ingredient” as used herein refers to an agent, other than a compound of the described composition that exerts a pharmacological, dermatological or any other beneficial activity. It is to be understood that “other beneficial activity” can be one that is only perceived as such by the subject using the inventive compositions. Such additional active agents include, but are not limited to, a carrier oil, an antifungal agent, an antibiotic, an antiviral agent, an antiprotozoal agent, an anesthetic agent, a chemotherapeutic agent, a vitamin, a hormone, or a steroid.

The terms “adjunct therapy” or “adjunctive therapy” are used interchangeably to refer to another treatment used together with a primary treatment to assist the primary treatment.

The term “administer” as used herein means to give or to apply. The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing the conventional nontoxic pharmaceutically acceptable carriers, adjuvants, and vehicles as desired, or may be locally administered. Additional administration may be performed, for example, intravenously, transmucosally, transdermally, intramuscularly, subcutaneously, intraperitoneally, intrathecally, intralymphatically, intralesionally, or epidurally. Administering can be performed, for example, once, a plurality of times, and/or over one or more extended periods either as individual unit doses or in the form of a treatment regimen comprising multiple unit doses of multiple substances.

The term “aging” as used herein refers to the process of growing or appearing older. The term “physiological aging” and its various grammatical forms as used herein is a measure of biological age in relation to changes that affect biological function and the ability to adapt to metabolic stress. Factors that play a role in determining physiological aging include, without limitation, chronological age, genetics, lifestyle, nutrition, diseases, and other conditions. Aging is associated with changes in dynamic biological, physiological, environmental, psychological, behavioral, and social processes. Some age-related changes are benign, such as graying hair. Others result in declines in function of the senses and activities of daily life and increased susceptibility to and frequency of disease, frailty, or disability. According to some embodiments, advancing age is a risk factor for a number of chronic diseases in humans.

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The term “allosteric modulation” as used herein refers to the process of modulating a receptor by the binding of allosteric modulators at a different site (i.e., regulatory site) other than of the endogenous ligand (orthosteric ligand) of the receptor and enhancing or inhibiting the effects of the endogenous ligand. It normally acts by causing a conformational change in a receptor molecule, which results in a change in the binding affinity of the ligand. Thus, an allosteric ligand “modulates” its activation by a primary “ligand” and can adjust the intensity of the receptor's activation. Many allosteric enzymes are regulated by their substrate, such a substrate is considered a “homotropic allosteric modulator.” Non-substrate regulatory molecules are called “heterotropic allosteric modulators.”

The term “allosteric regulation” is the regulation of an enzyme or other protein by binding an effector molecule at the proteins allosteric site (meaning a site other than the protein's active site). Effectors that enhance the protein's activity are referred to as “allosteric activators”, whereas those that decrease the protein's activity are called “allosteric inhibitors.” Thus, “allosteric activation” occurs when the binding of one ligand enhances the attraction between substrate molecules and other binding sites; “allosteric inhibition” occurs when the binding of one ligand decrease the affinity for substrate at other active sites.

The term “antagonist” as used herein refers to a substance that attenuates the effect of an agonist. It can be competitive or non-competitive, each of which can be reversible or irreversible. A competitive antagonist binds to the same site as the agonist but does not activate it, thus blocks the agonist's action. A non-competitive antagonist binds to an allosteric (non-agonist) site on the receptor to prevent activation of the receptor. A reversible antagonist binds non-covalently to the receptor, and therefore can be “washed out”. An irreversible antagonist binds covalently to the receptor and cannot be displaced by either competing ligands or washing.

The term “antibody” as used herein refers to a polypeptide or group of polypeptides comprised of at least one binding domain that is formed from the folding of polypeptide chains having three-dimensional binding spaces with internal surface shapes and charge distributions complementary to the features of an antigenic determinant of an antigen.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, κ and λ; individual molecules of immunoglobulin generally are only one or the other.

Human antibodies show two kinds of light chains, κ and λ; individual molecules of immunoglobulin generally are only one or the other. In normal serum, 60% of the molecules have been found to have κ determinants and 30 percent λ?. Many other species have been found to show two kinds of light chains, but their proportions vary. For example, in the mouse and rat, λ chains comprise but a few percent of the total; in the dog and cat, κ chains are very low; the horse does not appear to have any κ chain; rabbits may have 5 to 40% λ, depending on strain and b-locus allotype; and chicken light chains are more homologous to λ than κ.

In mammals, there are five classes of antibodies, IgA, IgD, IgE, IgG, and IgM, each with its own class of heavy chain—α (for IgA), δ (for IgD), ε (for IgE), γ (for IgG) and μ(for IgM). In addition, there are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites.

Each pentamer contains one copy of a J chain, which is covalently inserted between two adjacent tail regions.

All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies. Diverse libraries of immunoglobulin heavy (VH) and light (Vic and V)) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain FAT or scFv) can be made by randomly combining heavy and light chain V-genes using PCR. A combinatorial library then can be cloned for display on the surface of filamentous bacteriophage by fusion to a minor coat protein at the tip of the phage. The technique of guided selection is based on human immunoglobulin V gene shuffling with rodent immunoglobulin V genes. The method entails (i) shuffling a repertoire of human λlight chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety.

Antibody combining site. The antigen combining site, also called the “antigen binding site”, or “paratope”) is defined by the set of amino acid residues that make contact with the antigen. VH and VL combine by non-covalent association to form the FV region, which contains the antigen binding or combining site. Each domain contributes three hypervariable loops (HVLs) or CDRs, with CDR-L1, CDR-L2, and CDR-L3 formed by VL and CDR-H1, CDR-H2, and CDR-H3 by VH. In the FV, the two β-sheets and the non-hypervariable loops are referred to as Framework Regions (FRs). CDR-L1 and CDR-H1 HVLs correspond to the residues within the loops connecting β-strands B and C (Gilliland et al., 2012). For CDR-L2 and CDR-H2, the HVLs are formed by the loops connecting β-strands C′ and C″, and for CDR-L3 and CDR-H3, the HVLs are formed by the loops connecting β-strands F and G (Gilliland et al., 2012). Due to the large number of different V-regions that can comprise the Fv, both amino acid sequence and length can vary significantly for the HVLs. Taken from Therapeutic Antibody Engineering, Stohl, W R and Stohl L M, Eds., Woodhead Publishing Ltd. (2012)

The term “antibody construct” as used herein refers to a polypeptide comprising one or more the antigen-binding portions of the invention linked to a linker polypeptide or an immunoglobulin constant domain. Linker polypeptides comprise two or more amino acid residues joined by peptide bonds and are used to link one or more antigen-binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt. The term “chemokine” as used herein refers to a class of chemotactic cytokines that signal leukocytes to move in a specific direction. The terms “chemotaxis” or “chemotactic” refer to the directed motion of a motile cell or part along a chemical concentration gradient towards environmental conditions it deems attractive and/or away from surroundings it finds repellent.

The term “attenuate” as used herein refers to render less virulent, to weaken or reduce in force, intensity, effect or quantity.

“Binding fragments” of an antibody can be produced by recombinant DNA techniques, or by enzymatic or chemical cleavage of intact antibodies. Binding fragments include Fab, Fab′, F(ab′)2, Fv, and single-chain antibodies.

An antibody other than a “bispecific” or “bifunctional” antibody is understood to have each of its binding sites identical.

The term “binding specificity” as used herein involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners.

“Relative binding specificity” is a characteristic whereby in a biochemical system a molecule interacts with its targets or partners differentially, thereby impacting them distinctively depending on the identity of individual targets or partners.

The term “bioavailable” and its other grammatical forms as used herein refers to the ability of a substance to be absorbed and sued by the body.

The term “biocompatible” as used herein refers to a material that is generally non toxic to the recipient and does not possess any significant untoward effects to the subject and, further, that any metabolites or degradation products of the material are non-toxic to the subject. Typically a substance that is “biocompatible” causes no clinically relevant tissue irritation, injury, toxic reaction, or immunological reaction to living tissue.

The term “biodegradable” as used herein refers to a material that will erode to soluble species or that will degrade under physiologic conditions to smaller units or chemical species that are, themselves, non-toxic.

The term “biomarkers” (or “biosignatures”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “botanical raw material” as used herein refers to a fresh or processed (e.g. cleaned, frozen, dried, sliced, dissolved, or liquefied) part of a single species of plant or a fresh or processed alga or macroscopic fungus.

The term “botanical ingredient” refers to a component that originates from a botanical raw material.

The term “botanical product” refers to a finished, labeled product that contains vegetable matter, which may include plant materials, algae, macroscopic fungi, or combinations thereof. Depending in part on its intended use, a botanical product may be a food, drug, medical device or cosmetic.

The term “botanical extract” as used herein refers to a product prepared by separating, by chemical or physical process, medicinally active portions of a plan from the inactive or inert components. The botanical extracts prepared according to the present invention preferably are obtained by means of a solvent, optionally under pressure and/or heat.

The term “cannabimimetic” as used herein refers to an agent capable of mimicking (meaning imitating) or antagonizing (meaning neutralizing or counteracting) the biological effect(s) of a cannabinoid-containing composition as described herein. For example, certain fatty acid derivatives have been reported to interact with the endocannabinoid system. N-acetylethanolamines have been shown to inhibit fatty acid amide hydrolase (FAAH), thus leading to an increase in endocannabinoid tone. [Gertsch, J. et al., Br. J. Pharmacol. (2010) 160: 523-29]. N-linoleoylethanolamide and N-oleoylethanolamide, which are found in chocolate and other plants, and NAE palmitoylethanolamide inhibit anandamide breakdown. [Id., citing Maurelli, S., et al. FEBS Lett. (1995) 377: S82-S86; DiTomaso, E., et al. Nature (1996) 382: S677-S678]. Certain N-alkylamides from Echinacea spp. (e.g., Echinacea purpurea and Echinacea angustifolia) have been shown to interact functionally with the human CB2 receptor with low nM to μM Ki values [Id., citing Gertsch, J. et al, J. Recept. Signal. Transduct. Res. (2006) 26: S709-S730]. CB2 receptor binding N-alkylamides show similar anti-inflammatory effects as anandamide (e.g., inhibition of TNFα) at low nM concentrations (e.g., dodeca-2E, 4E, 8Z, 10Z-tetraenoic acid isobutylamide; dodeca-2E, 4E-dienoic acid isobutylamide) [Raduner, S. et al. J. Biol. Chem. (2006) 281: S14192-S15206). Certain Echinacea N-alkylamides inhibit anandamide reuptake in vitro [Gertsch, J. et al., Br. J. Pharmacol. (2010) 160: S23-29, citing Chicca, A., et al. Int. Immunopharmacol. (2009) 9: S850-S858], and target peroxisome proliferator-activated protein (PPAR)-gamma [Id., citing Spelman, K. et al. Int. Immunopharmacol. (2009) 9: S1260-S1264]. The polyacetylenic polyyne falcarinol, which is found in plants of the Apiaceae family (e.g., carrots) shows significant binding interactions with both cannabinoid receptors, but appears to selectively undergo an alkylation reaction with the CB1 receptor (I value <1 μM), leading to relatively potent inverse agonistic and pro-inflammatory effects in human skin [Id., citing Leonti, M. et al. Biochem. Pharmacol. (2010). Doi:10.1016/j.bcp.2010.02.015]. Certain flavonoids inhibit fatty acid amide hydrolase (FAAH), which is the enzyme responsible for the breakdown of the endogenous CB receptor ligand anandamide [Id., citing Thors, L. et al. Br. J. Pharmacol. (2007) 10: 5951-5960; Thors, L. et al. Br. J. Pharmacol (2008) 155: 5244-252]. Both the isoflavonoid genistein and the flavonoids kaempferol, 7-hydroxyflavone, and 3,7-dihydroxyflavone have been shown to concentration-dependently inhibit anandamide hydrolysis in rat brain homogenates, with 7-hydroxyflavone being the most potent inhibitor (IC50<1 μM. Other plant polyphenols include trans-resveratrol, curcumin, catechins, and kaemferol-type flavonoids.

Cannabimimetics also may be derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; Essential Oils, for example: Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), and Ylang ylang (Cananga odorata(Lam.) var. genuine).

There are two entirely different chemical scaffolds able to differentially target CB receptors in Cannabis: cannabinoids, and terpenes (see below). The term “cannabinoid” refers to a chemical compound that shows direct or indirect activity at a cannabinoid receptor. The term “phytocannabinoid” as used herein refers to any plant-derived product capable of either directly interacting with a cannabinoid receptor, sharing chemical similarity with cannabinoids, or both. “Direct cannabinoid receptor ligands” are compounds that show binding affinities in the lower nM range for cannabinoid receptors and exert discrete functional effects (i.e., agonism, neutral antagonism, or inverse antagonism). Indirect cannabinoid receptor ligands target either key proteins within the endocannabinoid system that regulate tissue levels of endocannabinoids or allosteric sites on the CB1 receptor. [Gertsch, J. et al., Br. J. Pharmacol. (2010) 160: 523-29].

The cannabis plant contains more than 60 different active synthetic ligands for CB1 and CB2, with Δ⁹-THC being the major psychoactive molecule among them. [Kendall, D A, Yudowski, G A, Frontiers Cellular ZNeurosci. (2017) 10: 294]. Exemplary cannabinoids include, but are not limited to, the Cannabichromenes (e.g., Cannabichromene (CBC), Cannabichromenic acid (CBCA), Cannabichromevarin (CBCV), Cannabichromevarinic acid (CBCVA)), Cannabicyclols (e.g., Cannabicyclol (CBL), Cannabicyclolic acid (CBLA), Cannabicyclovarin (CBLV)), Cannabidiols (e.g., Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA)), Cannabielsoins (e.g., Cannabielsoic acid B (CBEA-B), Cannabielsoin (CBE), Cannabielsoin acid A (CBEA-A)), Cannabigerols (e.g., Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA)), Cannabinols and cannabinodiols (e.g., Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV)), Cannabitriols (e.g., 10-Ethoxy-9-hydroxy-delta-6a-tetrahydrocannabinol, 8,9-Dihydroxy-delta-6a-tetrahydrocannabinol, Cannabitriol (CBT), Cannabitriolvarin (CBTV)), Δ⁸-tetrahydrocannabinols (e.g., Δ⁸-tetrahydrocannabinol (48-THC), Δ⁸-tetrahydrocannabinolic acid (48-THCA)), Δ⁹-tetrahydrocannabinols (e.g., Δ⁹-tetrahydrocannabinol (THC), Δ⁹-tetrahydrocannabinol-C4 (THC-C4), Δ⁹-tetrahydrocannabinolic acid A (THCA-A), Δ⁹-tetrahydrocannabinolic acid B (THCA-B), Δ⁹-tetrahydrocannabinolic acid-C4 (THCA-C4), Δ⁹-tetrahydrocannabiorcol (THC-C1), Δ⁹-tetrahydrocannabiorcolic acid (THCA-C1), Δ⁹-tetrahydrocannabivarin (THCV), Δ⁹-tetrahydrocannabivarinic acid (THCVA)), and miscellaneous cannabinoids (e.g., 10-Oxo-Δ⁶a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Δ⁹-cis-tetrahydrocannabinol (cis-THC), tryhydroxy-Δ⁹-tetrahydrocannabinol (triOH-THC), 3,4,5,6-tetrahydro-7-hydroxy-α-α-2-trimethyl-9-n-propyl-2,6-methano-2H-1-benzoxocin-5-methanol (OH-iso-HIHCV)).

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of the compositions described herein. Carriers and vehicles useful herein include any such materials know in the art which are nontoxic and do not interact with other components.

The term “CD94/NKG2A” as used herein refers to an inhibitory receptor that recognizes HLA-E and is expressed by NK cells and a subset of T cells.

The term “chemotype” as used herein refers to character of a plant or microorganism based on a metabolite distinct from that found in other members of the same species.

The term “chronological age” as used herein refers to the time passed from birth to a given date. Chronological ages are commonly grouped into a small number of crude age ranges, reflecting the major stages of development and aging categories: According to some embodiments, the age brackets for humans are: Young: from infant to young adult, i.e., Infant: 0-2; Preschool: 2-5; Child: 5-12; Adolescent: 12-19; Young adult: 19-24; adult: from 24-44; Middle aged: 44-60; and Aged: over 60 years. For the mouse, the chronological age categories by consensus are young (3 months); middle-aged (12 months) and old (24 months).

The term “clonotyping” as used herein refers to a process to identify the unique nucleotide CDR3 sequences of a TCR chain. This generally involves PCR amplification of the cDNA using V-region specific primers and either constant region (C) specific or J-region-specific primer pairs, followed by nucleotide sequences of the amplicon. [Yassai, M B et al. Immnogenetics (2009) 61 (7): 493-502]

The term “combination” as used herein refers to an assemblage of separate parts or qualities. The term “combining” as used herein refers to putting or adding together.

The term “combinatorial diversity” as used herein refers to a component of antibody and TCR diversity that is generated by recombination of variable (V), diversity (D, for immunoglobulin heavy chains, and for TCR β and γ chains) and joining (J) gene segments).

The term “compatible” as used herein means that the components of a composition are capable of being combined with each other in a manner such that there is no interaction that would substantially reduce the efficacy of the composition under ordinary use conditions.

The term “complementary” as used herein refers to combining in such a way as to enhance or emphasize the qualities of each other or another.

The term “component” as used herein refers to a constituent part, element or ingredient.

The term “composition” as used herein refers to a mixture of ingredients.

The term “complement” as used herein refers to a system of soluble pattern recognition receptors and effector molecules that detect and destroy microorganisms. In the presence of pathogens or of antibody bound to pathogens, soluble plasma proteins that in the absence of infection circulate in an inactive form becomes activated, so that particular complement proteins interact with each other to form the pathways of complement activation, which are initiated in different ways. The classical pathway is initiated when complement component C1, which comprises a recognition protein (C1q) associated with proteases (C1r and C1s) either recognizes a microbial surface directly or binds to antibodies already bound to a pathogen. The alternative pathway can be initiated by spontaneous hydrolysis and activation of complement component C3, which can then bind directly to microbial surfaces. The lectin pathway is initiated by soluble carbohydrate-binding proteins—mannose-binding lectin (MBL) and the ficolins—that bind to particular carbohydrate structures on microbial surfaces. MBL-associated serine proteases (MASPs), which associate with these recognition proteins, then trigger cleavage of complement proteins and activation of the pathway. These three pathways converge at the step whereby enzymatic activity of a C3 convertase is generated. The C3 convertase is bound covalently to the pathogen surface, where it cleaves C3 to generate large amounts of C3b, the main effector molecule of the complement system, and C3a, a small peptide that binds to specific receptors and helps induce inflammation. Cleavage of C3 is the critical step in complement activation and leads directly or indirectly to all the effector activities of the complement system. All three pathways have the final outcome of killing the pathogen, either directly or by facilitating its phagocytosis, and inducing inflammatory responses that help to fight infection.

Besides acting in innate immunity, the complement system also influences adaptive immunity. For example, opsonization of pathogens (meaning the coating of the surface of a pathogen that makes it more easily ingested by phagocytes) by complement facilitates their uptake by phagocytic APCs that express complement receptors, which enhances presentation of pathogen antigens to T cells. B cells express receptors for complement proteins that enhance their responses to complement-coated antigens. Several complement fragments also can act to influence cytokine production by APCs, thereby influencing the direction and extent of the subsequent adaptive immune response. [Janeway's Immunology, 9th Ed. 2017, Garland Science, New York, Chapter 2, 49-51].

The term “condition”, as used herein, refers to a variety of health states and is meant to include disorders or diseases caused by any underlying mechanism or disorder, injury, and the promotion of healthy tissues and organs.

The term “contact” and its various grammatical forms as used herein refers to a state or condition of touching or of immediate or local proximity. Contacting a composition to a target destination, such as, but not limited to, an organ, a tissue, or a cell, may occur by any means of administration known to the skilled artisan.

The term “controlled release” is intended to refer to any active-containing formulation in which the manner and profile of release of the active from the formulation are regulated. This refers to immediate as well as non-immediate release formulations, with non-immediate release formulations including, but not limited to, sustained release and delayed release formulations. The term “delayed release” is used herein in its conventional sense to refer to a formulation in which there is a time delay between administration of the formulation and the release of the active therefrom. “Delayed release” may or may not involve gradual release of the active over an extended period of time, and thus may or may not be “sustained release.”

The term “cytokine” as used herein refers to small soluble protein substances secreted by cells, which have a variety of effects on other cells. Cytokines mediate many important physiological functions, including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (TNF)-related molecules, including TNFα and lymphotoxin; immunoglobulin super-family members, including interleukin 1 (IL-1); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “dendritic cell” as used herein refers to discrete leukocyte population(s) of antigen presenting cells that initiate specific T-lymphocyte activation and proliferation. Their key properties include (1) the ability to take up, process, and present antigen; (2) the ability to migrate selectively through tissues; and (3) the ability to interact with, stimulate and direct T-lymphocyte responses. [Hart, DNJ, Blood 90(9): 3245-87, 3245 (1997)]. The encounter with an antigen induces the dendritic cell to mature from an antigen-capturing cell to an antigen-presenting cell that can activate T cells. [Alberts, B. et al., Molecular Biology of the Cell, 4th Ed., Garland Science, NY (2002), p. 1394].

DCs exhibit several features necessary for the generation of T-cell-mediated antitumor immunity [Dermime S, et al., British Medical Bulletin (2002) 62: 149-162; Cella M, et al., Curr. Opin. Immunol. (1997) 9: 10-16]. They efficiently capture and take up antigens in peripheral tissues and transport these antigens to the primary and secondary lymphoid organs where they express high levels of MHC class I and II molecules that present the processed peptides to T-cells for the priming of antigen-specific responses. Specifically, a DC acquires polypeptide antigens, where these antigens can be acquired from outside of the DC, or biosynthesized inside of the DC by an infecting organism. The DC processes the polypeptide, resulting in peptides of about ten amino acids in length, transfers the peptides to either MHC class I or MHC class II to form a complex, and shuttles the complex to the surface of the DC. When a DC bearing a MHC class I/peptide complex contacts a CD8+ T-cell, the result is activation and proliferation of the CD8+ T-cell. When a DC bearing a MHC class II/peptide complex contacts a CD 4+ T-cell, the outcome is activation and proliferation of the CD4+ T-cell [Munz, et al. (2010) Curr. Opin. Immunol. 22:89-93; Monaco (1995) J. Leukocyte Biol. 57:543-547; Robinson, et al (2002) Immunology 105:252-262]. Although dendritic cells presenting antigen to a T-cell can “activate” that T-cell, the activated T-cell might not be capable of mounting an effective immune response. An effective immune response by the CD8+ T-cell, for example, often requires prior stimulation of the DC by one or more of a number of interactions. These interactions include direct contact of a CD4+ T-cell to the DC (by way of contact of the CD4+ T-cell's CD40 ligand to the DCs CD40 receptor), or direct contact of a toll-like receptor (TLR) agonist to one of the dendritic cell's toll-like receptors (TLRs).

The term “derivative” as used herein means a compound that may be produced from another compound of similar structure in one or more steps that retains at least a degree of the desired function of the parent compound. Accordingly, an alternate term for “derivative” may be “functional derivative.” Derivatives can include chemical modifications, including, without limitation, akylation, acylation, carbamylation, etc. Free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives.

As used herein, the term “derived from” is meant to encompass any method for receiving, obtaining, or modifying something from a source of origin.

The terms “disease” or “disorder” as used herein refer to an impairment of health or a condition of abnormal functioning.

As used herein, “dispersed system” or “dispersion” refers to a two-phase system in which one phase is distributed as particles or droplets in the second, or continuous phase.

The term “diversity” as used herein refers to the number of classes, degree of dispersion among classes, species richness, variety, or a multiformity. For example, antibodies with an enormous diversity of antigen-binding sites are produced by B cells. Such antibody diversity is generated from the large number of V, J, D, and C genes that enable the immune system to generate an almost unlimited number of different light and heavy chains by joining these separate gene segments together before they are transcribed. The range of different TCRs expressed is likewise the result of recombination. TCRs are heterodimers that fall into two classes: TCR-αβ and TCR-γ8. The TCR α- and γ-chains constitute a variable (V), joining (J) and constant region (C). The TCR β- and δ-chains are also made up of a V, J and C region, with an additional diversity (D) region. One segment from each region is recombined, with additional nucleotide additions and/or deletions, to generate each rearranged TCR. This recombination generates high T-cell diversity and enables T cell recognition of millions of antigens.

The term “diversity measurement” refers to the number of species (clonotypes) present in a biological entity. [Aversa, I. et al. Intl J. Molec. Sci. (2020) 21: 238].

The term “dose” as used herein refers to the quantity of medicine prescribed to be taken at one time.

The term “effective amount” or “effective dose” refers to the amount necessary or sufficient to realize a desired biologic effect.

The term “efficacy (Emax)” as used herein refers to the maximum effect which can be expected from a drug (i.e. when this magnitude of effect is reached, increasing the dose will not produce a greater magnitude of effect). The term “potency” as used herein refers to the concentration (EC50) or dose (ED50) of a drug required to produce 50% of that drug's maximal effect.

The term “endogenous cannabinoids” as used herein refers to endogenous lipids that engage cannabinoid receptors. [Lu, H-C, and Mackie, H. Biol. Psychiatry (2016) 79 (7): 516-25] The first discovered and best-characterized endocannabinoids are anandamide (arachidonoyl ethanolamide) and 2-arachidonoyl glycerol (2-AG). The precursors of these endocannabinoids are present in lipid membranes. Upon demand (typically by activation of certain G protein-coupled receptors or by depolarization), endocannabinoids are liberated in one or two rapid enzymatic steps and released into the extracellular space. This contrasts with classical neurotransmitters that are synthesized ahead of time and stored in synaptic vesicles. The intrinsic efficacy of the endogenous cannabinoids varies-2-AG is a high efficacy agonist for both CB1 and CB2 receptors, however anandamide is a low efficacy agonist at CB1 receptors and a very low efficacy agonist at CB2 receptors [Id., citing Gonsiorek W, et al. Mol. Pharmacol. (2000) 57: 1045-50; Luk, T. et al., Br. J. Pharmacol. (2004) 142: 495 500] The effects of endocannabinoids are primarily mediated by CB1 and CB2 cannabinoid receptors (Id., citing Howlett, A C, et al. Pharacol. Rev. (2002) 54: 161-202), with other receptors (such as PPAR's and Transient Receptor Potential (TRP)) channels also mediating some endocannabinoid actions, particularly of the acylethanolamides. TRP channels, especially TRPV1, are activated by anandamide under certain conditions [Id, citing Zygmunt P M, et al, Nature (1999) 400: 452-57]. The relative roles of cannabinoid receptors and TRP channels in anandamide's actions appear variable. Anandamide also activates PPAR alpha and gamma, with significant effects on gene transcription [Id., citing Bouaboula M et al. Eur. J. Pharmacol. (2005) 517: 174-81; O'Sullivan S E. Br. J. Pharacol. (2007) 152: 576-82]. It is important to keep in mind that increasing anandamide by decreasing its degradation by inhibition of fatty acid aminohydrolase (FAAH) also increases levels of other N-acylamides, which can modulate PPARα [Id., citing Fu, J. et al. Nature (2003) 425: 90-93; Luchicchi, a. et al., Addict. Biol. (2010) 15: 277-88].

The term “health span” as used herein refers to the portion of life spent in good health.

The term “immunological repertoire” refers to the collection of transmembrane antigen-receptor proteins located on the surface of T and B cells. (Benichou, J. et al. Immunology (2011) 135: 183-191). The combinatorial mechanism that is responsible for encoding the receptors does so by reshuffling the genetic code, with a potential to generate more than 10¹⁸ different T cell receptors (TCRs) in humans (Id., citing Venturi, Y. et al. Nat. Rev. Immunol. (2008) 8: 231-8), and a much more diverse B-cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen. (Id.) The highly diverse junctional region of the TCR chain, also known as the complementarily-determining region 3 (CDR3) is an important determinant of antigen recognition. [Aversa, I. et al. (2010) Int. J. Mol. Sci. 21: 238; doi:10.3390/ijms21072378, citing Xu, J L and Davis, M M. Immunity (2000) 13: 37-45]. The CDR3 sequence is essentially unique for each newly formed T cell, since it is highly unlikely that two T cells will express the same CDR3 nucleotide sequence [Id., citing Turner, S J et al. Nat. Rev. Immunol. (2006) 6: 883-94]. At the same time, when a T cell is activated and undergoes a clonal expansion, all the cells of the clonal lineage are equipped with an identical CDR3, which therefore acts as a natural identifier of the clonality of the lymphocytes [Id., citing Kirsch, I. et al. Mol. Oncol. (2015) 9: 2063-700].

The terms “immune response” and “immune-mediated” are used interchangeably herein to refer to any functional expression of a subject's immune system, against either foreign or self-antigens, whether the consequences of these reactions are beneficial or harmful to the subject.

The term “immune system” as used herein refers to the body's system of defenses against disease. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens.

The term “improve” (or improving) as used herein refers to bring into a more desirable or excellent condition.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity. The term “innate immunity”, as used herein refers to the various resistance mechanisms encountered first by a pathogen, before adaptive immunity is induced, include anatomical barriers, antimicrobial peptides, the complement system, and macrophages and neutrophils carrying nonspecific pathogen-recognition receptors. Innate immunity is present in all individuals at all times, does not increase with repeated exposure to a given pathogen, and discriminates between groups of similar pathogens, rather than responding to a particular pathogen.

The terms “immunomodulatory”, “immune modulator” and “immune modulatory” are used interchangeably herein to refer to a substance, agent, or cell that is capable of augmenting or diminishing immune responses directly or indirectly, e.g., by expressing chemokines, cytokines and other mediators of immune responses.

The term “immunomodulatory cell(s)” as used herein refer(s) to cell(s) that are capable of augmenting or diminishing immune responses by expressing chemokines, cytokines and other mediators of immune responses.

The term “immunosuppressive agent” as used herein refers to an agent that decreases the body's immune responses.

The term “immunosuppression” as sued herein refers to a state of decreased immunity or a lowering of the body's immune response. The term “immunosuppressive therapy” as used herein refers to a treatment that lowers the activity of the body's immune system.

The term “inflammatory cytokines” or “inflammatory mediators” as used herein refers to the molecular mediators of the inflammatory process, which may modulate being either pro- or anti-inflammatory in their effect. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, pro-inflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12).

The term “interleukin (IL)” as used herein refers to a cytokine secreted by, and acting on, leukocytes. Interleukins regulate cell growth, differentiation, and motility, and stimulates immune responses, such as inflammation. Examples of interleukins include interleukin-1 (IL-1), interleukin 2 (IL-2), interleukin-lβ (IL-1β), interleukin-6 (IL-6), interleukin-8 (IL-8), and interleukin-12 (IL-12).

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses. The term “acute inflammation” as used herein refers to the rapid, short-lived (minutes to days), relatively uniform response to acute injury characterized by accumulations of fluid, plasma proteins, and neutrophilic leukocytes. The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “inflammatory mediators” or “inflammatory cytokines” as used herein refers to molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12).

The term “innate lymphoid cells (ILCs) as used herein refers to the innate counterparts of T lymphocytes. They lack adaptive antigen receptors generated by the recombination of genetic elements. [Vivier, E. et al. Cell (2018) 174: 1054-66, citing Spits, et al. Nat. Rev. Immunol. (2013) 13: 145-49; Eberl, G et al. Science (2015) 348: aaa6566; Artis, D. and Spits, H. Nature (2015) 517: 293-301]. ILC1s, ILC2s, and ILC3s mirror CD4+ T helper (Th)1, Th2, and Th17 cells, respectively, in terms of function, whereas natural killer (NK) cells mirror the functions of CD8+ cytotoxic T cells. ILCs and T cells play key roles in orchestrating the most appropriate immune response to the threat faced by the individual. ILC1s and Th1 cells react to intracellular pathogens, such as viruses, and to tumors; ILC2s and Th2 cells respond to large extracellular parasites and allergens; and ILC3s and Th17 cells combat extracellular microbes, such as bacteria and fungi. Myeloid and non-hematopoietic cells instruct the ILCs and T cells, which belong to the lymphoid lineage, about the type of threat they will confront. The ILCs and T cells react by providing positive and negative feedbacks and through immune regulatory and effector functions. ILCs act early in the immune response by reacting promptly to signals, or inducer cytokines, expressed by tissue-resident cells. ILCs play a key role in homeostasis, due to the rapidity with which they react and their presence in normal healthy tissues, including the intestine, lung and adipose tissues. ILCs are also involved in tissue tolerance and regeneration in response to tissue damage. In the intestine and thymus, ILC3s mediate tolerance to chemical toxins and irradiation through the activation of epithelial cells, whereas ILC2s produce amphiregulin (AREG), a member of the EGF family, which is involved in epithelial cell regulation.

ILC1s are generally non-cytotoxic or weakly cytotoxic and function as a first line of defense against infections with viruses and certain bacteria, such as T. gondii [Id., citing Klose, C S N et al. Cell (2014) 157: 340-56] or C. difficile [Id., citing Abt, M C et al. Cell Host Microbe (2015) 18: 27-37]. NK cells and ILC1s have several features in common.

Both these cell types produce IFN-γ as their principal cytokine output and require the Th1 transcription factor Tbet for this function. However, they have different developmental paths. In both humans and in mice, NK cells develop from a common innate lymphoid progenitor (CILP) via an NK cell precursor (NKP), whereas ILC1s develop from CILPs via an innate lymphoid cell precursor (ILCP) (Id., citing Constantinides, M G et al., Nature (2014) 508: 397-401, Klose, C S N et al. Cell (2014) 157: 340-56, Lim, A I et al. Cell (2017) 168: 1086 1100.e10, Renoux, V M et al. Immunity (2015) 43: 394-407, Scoville, S D et al. Immunity (2016) 44: 1140-50). NK cells and ILC1s are functionally different, as NK cells are dedicated cytotoxic cells strongly expressing perforin, whereas ILC1s have low levels of perforin expression. However, regardless of these developmental and functional differences, the phenotypic characterization of ILC1s is often problematic. ILC1s preferentially express CD49a and TRAIL in both humans and mice, but the specificity of these markers is often lost upon cell activation and is tissue-dependent.

ICL2s are defined by their capacity to produce type 2 cytokines IL-4, IL-5, and IL-13 [Id., citing Moro, K. et al Nature Immunol. (2010) 17: 76-86; Neill, D R et al. Nature (2010) 464: 1367-70 (2010); Price, A E et al. Proc. Nat. Acad. Sci. USA (2010) 107: 11489 94]; and are tissue resident [Id., citing Gasteiger, G. et al Science (2015) 350: 981-86; Moro, K. et al. Nat. Immunol. (2016) 17: 76-96]. They respond to the cytokines IL-25, TSLP, and IL-33. ICL2s are involved in the innate immune response to parasites. ICL3s are abundant at mucosal sites, and are involved in the innate immune response to extracellular bacteria and the containment of intestinal commensals [Id., citing Cella, M, et al. Nature(2009) 457: 722 25; Luci C. et al. Nat. Immunol. (2009) 10: 75-82; Cupedo, T. et al Nat. Immunol. (2009) 10: 66-74; Satoh-Takayama, N. et al. Immunity (2008) 29: 958-70; Buonocore S., et al. Nature (2010) 464: 1371-75; Sonnenberg, G F, et al., Immunity (2011) 34:122-34; Sonnenberg, GF Science (2012) 336: 1321-25; Rankin, L C et al. Nat Immunol. (2016) 17: 179-86]. The predominant homeostatic cytokine produced by ILC3s is IL-22.

There is increasing evidence to suggest that like T helper cell subsets, ILC subsets also display a certain degree of plasticity. Thus, ILC subsets can change their phenotype and functional capacities and this requires accessible polarizing signals in the tissue in which conversion occurs, together with the expression of cognate cytokine receptors and key transcription factors in the responding ILCs.

The compositions described herein may comprise isolated molecules. An “isolated molecule” is a molecule that is substantially pure and is free of other substances with which it is ordinarily found in nature or in vivo systems to an extent practical and appropriate for its intended use. In particular, the compositions are sufficiently pure and are sufficiently free from other biological constituents of host cells so as to be useful in, for example, producing pharmaceutical preparations or sequencing if the composition is a nucleic acid, peptide, or polysaccharide. Because compositions may be admixed with a pharmaceutically-acceptable carrier in a pharmaceutical preparation, the compositions may comprise only a small percentage by weight of the preparation. The composition is nonetheless substantially pure in that it has been substantially separated from the substances with which it may be associated in living systems or during synthesis. As used herein, the term “substantially pure” refers purity of at least 75%, at least 80%, at least 85%, at least 90%, at least 95% or at least 99% pure as determined by an analytical protocol. Such protocols may include, for example, but are not limited to, FACS, HPLC, gel electrophoresis, chromatography, and the like.

The terms “long COVID” or “COVID long-haulers” are used interchangeably to refer to patients who have recovered from a SARS-CoV-2 infection that experience a diverse, often debilitating, set of symptoms after their recovery.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions.

Two broad classes of lymphocytes are recognized: the B-lymphocytes (B-cells), which are precursors of antibody-secreting cells, and T-lymphocytes (T-cells),

### B-Lymphocytes

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses. (Paul, W. E., “Chapter 1: The immune system: an introduction,” Fundamental Immunology, 4th Edition, Ed. Paul, W. E., Lippincott-Raven Publishers, Philadelphia (1999)).

Cross-linkage dependent B-cell activation requires that the antigen express multiple copies of the epitope complementary to the binding site of the cell surface receptors because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes. (Paul, W. E., “Chapter 1: The immune system: an introduction,” Fundamental Immunology, 4th Edition, Ed. Paul, W. E., Lippicott-Raven Publishers, Philadelphia (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4+ T-cells. The CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell. (Paul, W. E., “Chapter 1: The immune system: an introduction,” Fundamental Immunology, 4th Edition, Ed. Paul, W. E., Lippicott-Raven Publishers, Philadelphia (1999)).

During cognate help for antibody production, the CD40 ligand is transiently expressed on activated CD4+T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in the pathogenic autoantibody production in human SLE patients. (Desai-Mehta, A. et al., “Hyperexpression of CD40 ligand by B and T cells in human lupus and its role in pathogenic autoantibody production,” J. Clin. Invest., 97(9): 2063-2073 (1996)).

### T-Lymphocytes

T-lymphocytes derive from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. (Paul, W. E., “Chapter 1: The immune system: an introduction,” Fundamental Immunology, 4th Edition, Ed. Paul, W. E., Lippincott-Raven Publishers, Philadelphia (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. Antibody and B-cell receptors evolved to bind to and to protect against microorganisms in extracellular fluids. In contrast, T cells recognize antigens on the surface of other cells and mediate their functions by interacting with, and altering, the behavior of these antigen-presenting cells (APCs). There are three main types of antigen-presenting cells in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an antigen-presenting cell (APC) that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the antigen-presenting cell (APC) for long enough to become activated. (“Chapter 24: The adaptive immune system,” Molecular Biology of the Cell, Alberts, B. et al., Garland Science, N Y, 2002).

T-cells are subdivided into two distinct classes based on the cell surface receptors they express. The majority of T cells express T cell receptors (TCR) consisting of α and β chains. A small group of T cells express receptors made of 8 and 8 chains. Among the α/β T cells are two important sublineages: those that express the coreceptor molecule CD4 (CD4+ T cells); and those that express CD8 (CD8+ T cells). These cells differ in how they recognize antigen and in their effector and regulatory functions.

CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells particularly CD8+ T cells, can develop into cytotoxic T-lymphocytes (CTLs) capable of efficiently lysing target cells that express antigens recognized by the CTLs. [Paul, W. E., “Chapter 1: The immune system: an introduction,” Fundamental Immunology, 4th Edition, Ed. Paul, W. E., Lippincott-Raven Publishers, Philadelphia (1999)].

T cell receptors (TCRs) recognize a complex consisting of a peptide derived by proteolysis of the antigen bound to a specialized groove of a class II or class I MHC protein. The CD4+ T cells recognize only peptide/class II complexes while the CD8+ T cells recognize peptide/class I complexes. [Paul, W. E., “Chapter 1: The immune system: an introduction,” Fundamental Immunology, 4th Edition, Ed. Paul, W. E., Lippincott-Raven Publishers, Philadelphia (1999)].

The TCR's ligand (i.e., the peptide/MHC protein complex) is created within antigen-presenting cells (APCs). In general, class II MHC molecules bind peptides derived from proteins that have been taken up by the APC through an endocytic process. These peptide-loaded class II molecules are then expressed on the surface of the cell, where they are available to be bound by CD4+ T cells with TCRs capable of recognizing the expressed cell surface complex. Thus, CD4+ T cells are specialized to react with antigens derived from extracellular sources. [Paul, W. E., “Chapter 1: The immune system: an introduction,” Fundamental Immunology, 4th Edition, Ed. Paul, W. E., Lippincott-Raven Publishers, Philadelphia (1999)]. Stimulation of the MHC II pathway leads to induction of a wide range of adaptive immune responses, including activation of macrophages and activation of B cells to secrete antibodies, as well as activation of cytotoxic T cells to kill targeted cells.

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally nine amino acids in length, are bound into the class I MHC molecules and are brought to the cell surface, where they can be recognized by CD8+ T cells expressing appropriate receptors. This gives the T cell system, particularly CD8+ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted. [Paul, W. E., “Chapter 1: The immune system: an introduction,” Fundamental Immunology, 4th Edition, Ed. Paul, W. E., Lippincott-Raven Publishers, Philadelphia (1999)]. Activation of the MHC I pathway leads to induction of cytotoxic CD8+ T cells only.

T cells can also be classified based on their function as helper T cells; T cells involved in inducing cellular immunity; suppressor T cells; and cytotoxic T cells.

### Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. [Paul, W. E., “Chapter 1: The immune system: an introduction,” Fundamental Immunology, 4th Edition, Ed. Paul, W. E., Lippincott-Raven Publishers, Philadelphia (1999)].

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching (of the Ig class being expressed) either depend or are enhanced by the actions of T cell-derived cytokines. [Paul, W. E., “Chapter 1: The immune system: an introduction,” Fundamental Immunology, 4th Edition, Ed. Paul, W. E., Lippincott-Raven Publishers, Philadelphia (1999)].

CD4+ T cells tend to differentiate into cells that principally secrete the cytokines IL-4, IL-5, IL-6, and IL-10 (TH2 cells) or into cells that mainly produce IL-2, IFN-□, and lymphotoxin (TH1 cells). The TH2 cells are very effective in helping B-cells develop into antibody-producing cells, whereas the TH1 cells are effective inducers of cellular immune responses, involving enhancement of microbicidal activity of monocytes and macrophages, and consequent increased efficiency in lysing microorganisms in intracellular vesicular compartments. Although the CD4+ T cells with the phenotype of TH2 cells (i.e., IL-4, IL-5, IL-6 and IL-10) are efficient helper cells, TH1 cells also have the capacity to be helpers. [Paul, W. E., “Chapter 1: The immune system: an introduction,” Fundamental Immunology, 4th Edition, Ed. Paul, W. E., Lippincott-Raven Publishers, Philadelphia (1999)].

### T Cells Involved in Induction of Cellular Immunity

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. The TH1 cells are effective in enhancing the microbicidal action because they produce IFN-γ. By contrast, two of the major cytokines produced by TH2 cells, IL-4 and IL-10, block these activities. [Paul, W. E., “Chapter 1: The immune system: an introduction,” Fundamental Immunology, 4th Edition, Ed. Paul, W. E., Lippincott-Raven Publishers, Philadelphia (1999)].

### Suppressor or Regulatory T (Treg) Cells

A controlled balance between initiation and downregulation of the immune response is important to maintain immune homeostasis. Both apoptosis and T cell anergy (a tolerance mechanism in which the T cells are intrinsically functionally inactivated following an antigen encounter [Schwartz, R. H., “T cell anergy,” Annu. Rev. Immunol., 21: 305-334 (2003)) are important mechanisms that contribute to the downregulation of the immune response. A third mechanism is provided by active suppression of activated T cells by suppressor or regulatory CD4+T (Treg) cells. [Reviewed in Kronenberg, M. et al., “Regulation of immunity by self-reactive T cells,” Nature 435: 598-604 (2005)]. CD4+ Tregs that constitutively express the IL-2 receptor alpha (IL-2Rα) chain (CD4+CD25+) are a naturally occurring T cell subset that are anergic and suppressive. [Taams, L. S. et al., “Human anergic/suppressive CD4+CD25+ T cells: a highly differentiated and apoptosis-prone population,” Eur. J. Immunol., 31: 1122-1131 (2001)]. Depletion of CD4+CD25+ Tregs results in systemic autoimmune disease in mice. Furthermore, transfer of these Tregs prevents development of autoimmune disease. Human CD4+CD25+ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4+CD25+T cells can be split into suppressive (CD25high) and nonsuppressive (CD25low) cells, according to the level of CD25 expression. A member of the forkhead family of transcription factors, FOXP3, has been shown to be expressed in murine and human CD4+CD25+ Tregs and appears to be a master gene controlling CD4+CD25+ Treg development. [Battaglia, M. et al., “Rapamycin promotes expansion of functional CD4+CD25+Foxp3+ regulator T cells of both healthy subjects and type 1 diabetic patients,” J. Immunol., 177: 8338-8347 (200)].

### Cytotoxic T Lymphocytes (CTL)

The CD8+ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by a series of enzymes produced by activated CTLs, referred to as granzymes. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

### Natural Killer (NK) Cells

Natural killer cells are prototypic members of the innate lymphoid cell (ILC) family and characterized in humans by expression of the phenotypic marker CD56 in the absence of CD3. They are usually further divided into two subsets based on their surface level expression of CD56 [Van Acker, H H, et al. Front. Immunol. (2017) 8: 892, citing Cheng, M. et al. Cell Mol. Immunol. (2013) 10(3): 230-52]. Whereas most NK cells in peripheral blood are CD56dim, CD56bright NK cells are more abundant in tissues. CD56bright NK cells respond better to soluble factors, while the CD56dim subset responds better to receptors binding ligands anchored on other cells [Id., citing Long, E O et al. Annu. Rev. Immnol. (2013) 31: 227-58]. NK cells have a central role in the cellular immune response, comprising tumor-cell surveillance as demonstrated in the setting of hematopoietic stem cell transplantation (HSCT).

NK cells play a role in protection against tumorigenesis and viral infections. [Borrego, F. et al. J. Immunol. (2002) 169 (11) 6102-6111, citing Soloski, M. J. 2001. Curr. Opin. Immunol. 13: 154; Biron, C. A., L. Brossay. 2001. Curr. Opin. Immunol. 13: 458]. They express inhibitory receptors that inhibit cytotoxicity and cytokine production upon recognition of MHC class I molecules [Id., citing Lanier, L. L. 1998. Annu. Rev. Immunol. 16: 359; Borrego, F., et al. 2002. Mol. Immunol. 38: 637]. By this mechanism, NK cells detect the integrity of cells through their loss of expression of MHC class I protein, a feature known as the missing self-hypothesis [Id., citing Ljunggren, H. G., Karre, K. Immunol. Today (1990) 11: 237]. In humans, three distinct families of genes encoding inhibitory receptors for HLA class I molecules have been defined [Id., citing Borrego, F., et al. 2002. Mol. Immunol. 38: 637]. The first family of receptors are type I transmembrane molecules belonging to the Ig superfamily and are called killer cell Ig-like receptors. The second group of receptors, also belonging to the Ig superfamily, named Ig-like transcripts, are expressed mainly on B, T, dendritic, and myeloid cells; however, some members of this group are also expressed on NK cells. The ligands for the killer cell Ig-like receptor and some of the Ig-like transcript receptors include classical (class Ia) HLA class I molecules, as well the nonclassical (class Ib) HLA-G molecule and the human CMV-encoded class I-like molecule UL18. The third family of HLA class I receptors are C-type lectin family members and are composed of heterodimers of CD94 covalently associated with a member of the NKG2 family of molecules [Id., citing Borrego, F., et al., 2002. Mol. Immunol. 38: 637; Brooks, A. G., et al. 1997. J. Exp. Med. 185: 795]. The ligand for the inhibitory members of this family is the nonclassical class I molecule HLA-E [Id., citing Braud, V. M., et al 1998. Nature 391: 795 8, Borrego, F., et al. 1998. J. Exp. Med. 187: 813, Lee, N., et al. 1998. Proc. Natl. Acad. Sci. USA 95: 5199].

NK inhibitory receptors share a common regulatory sequence of amino acids in their cytoplasmic tails, the immunoreceptor tyrosine-based inhibitory motifs (ITIMs). Human NKG2A has two ITIMs which contain tyrosine residues that are phosphorylated, presumably by a src tyrosine kinase, following interaction of CD94/NKG2A with HLA-E expressed on target cells. The phosphorylated ITIMs can recruit and activate the phosphatase Src homology 2 domain-bearing tyrosine phosphatase-1 (SHP-1), which suppresses the signal generated from cell surface-activating receptors [Id., citing Carretero, M. et al. Eur. J. Immuno (1998) 28: 1280; Kabat, J. et al. J. Immunol. (2002) 169: 1948].

NK cells express a large variety of activating receptors that recognize ligands expressed by normal cells, as well as tumor and infected cells [Id., citing Moretta, A. et al. Annu. Rev. Immunol. (2001) 19: 197]. Cross-linking of these receptors activates signaling cascades that result in cytokine production and killing of target cells. Under normal conditions, NK cells express at least one inhibitory receptor capable of interacting with autologous MHC class I molecules, which are a hallmark of normal somatic cells [Id., citing Valiante, N M, et al. Immunity (1997) 7: 739]. The maintenance of adequate levels of inhibitory receptors on the cell surface of NK cells is required to suppress the constant stimulation of NK cells provided by the ligation of activating receptors [Id., citing Zmoretta, A. et al. Nat. Immunol. (2002) 3: 6].

### NOD-Like Receptors (NLRs)

The term “Nucleotide-binding Oligomerization Domain (NOD)-like receptors” or “NLRs” refers to innate sensors that detect microbial products or cellular damage in the cytoplasm or activate signaling pathways, and are expressed in cells that are routinely exposed to bacteria, such as epithelial cells, macrophages and dendritic cells.

Some NLRs activate NFκB to initiate the same inflammatory responses as the TLRs, while others trigger a distinct pathway that induces cell death and the production of pro-inflammatory cytokines. Id. at 96.

Subfamilies of NLRs can be distinguished based on the other protein domains they contain. For example the NOD subfamily has an amino-terminal caspase recruitment domain (CARD), which is structurally related to the T1R death domain in MyD88, and can dimerize with CARD domains on other proteins to induce signaling. NOD proteins recognize fragments of bacterial cell wall peptidoglycans, although it is not known if they do so through direct binding or through accessory proteins. Id. At 96. NOD1 senses γ-glutamyl diaminopimelic acid (iE-DAP), a breakdown product of peptidoglycans of Gram negative and some Gram positive bacteria, whereas NOD2 recognizes muramyl dipeptide (MDP), which is present in the peptidoglycans of most bacteria. Id. Other members of the NOD family, including NLRX1 and NLRC5, have been identified, but their function is less well understood. Id. at 96-98.

When NOD1 or NOD2 recognizes its ligand, it recruits the CARD-containing serine-threonine kinase RIP2 (also known as RICK and RIPK2), which associates with the E3 ligases cIAP1, CIAP2, and XIAP, whose activity generates a polyubiquitin scaffold, which recruits TAK1 and IKK and results in activation of NFκB. NFκB then induces the expression of genes for inflammatory cytokines and for enzymes involved in the production of NO. Id. At 97.

Macrophages and dendritic cells express both TLRs and NOD1 and NOD2, and are activated by both pathways. In epithelial cells, NOD1 may also function as a systemic activator of innate immunity. NOD2 is strongly expressed in the Paneth cells of the gut where it regulates the expression of potent anti-microbial peptides such as the α- and β-defensins. Id. At 97.

Other members of the NOD family, including NLRX1 and NLRC5, have been identified, but their function is less well understood. Id. At 96-98

The NLRP family, another subfamily of NLR proteins, has a pyrin domain in place of the CARD domain at their amino termini. Humans have 14 NLR proteins containing pyrin domains, of which NLRP3 (also known as NAPL3 or cryopyrin) is the best characterized. NLRP3 resides in an inactive form in the cytoplasm, where its leucine rich repeat (LRR) domains are thought to bind the head-shock chaperone protein HSP90 and the co-chaperone SGT1. NRLP3 signaling is induced by reduced intracellular potassium, the generation of reactive oxygen species, or the disruption of lysosomes by particulate or crystalline matter. For example, death of nearby cells can release ATP into the extracellular space, which would active the purinergic receptor P2X7, which is a potassium channel, and allow potassium ion efflux. A model proposed for ROS-induced NLRP3 activation involves intermediate oxidation of sensor proteins collectively called thioredoxin (TRX). Normally TRX proteins are bound to thioredoxin-interacting protein (TXNIP). Oxidation of TRX by ROS causes dissociation of TXNIP from TRX. The free TXNIP may then displace HSP90 and SGT1 from NLRP3, again causing its activation. In both cases, NLRP3 activation involves aggregation of multiple monomers via their leucine-rich repeat (LRR) and NOD domains to induce signaling. Phagocytosis of particulate matter (e.g. the adjuvant alum), may lead to the rupture of lysosomes and release of the active protease cathepsin B, which can activate NLRP3. Id. At 98-99.

NLR signaling, as exemplified by NLRP3, leads to the generation of pro-inflammatory cytokines and to cell death through formation of an inflammasome, a multiprotein complex. Activation of the inflammasome proceeds in several stages: (1) Aggregation of NLRP molecules triggers autocleavage of procaspase I, which releases active caspase 1—Aggregation of LRR domains of several NLRP3 molecules, or other NLRP molecules by a specific trigger or recognition event, which induces the pyrin domains of NLRP3 to interact with pyrin domains of ASC (also called PYCARD), an adaptor protein composed of an amino terminal pyrin domain and a carboxyterminal CARD domain, which further drives the formation of a polymeric ASC filament, with the pyrin domains in the center and the CARD domains facing outward; the CARD domains then interact with CARD domains of the inactive protease pro-caspase 1, initiating its CARD-dependent polymerization into discrete caspase 1 filaments. Active caspase 1 then carries out ATP-dependent proteolytic processing of proinflammatory cytokines, particularly 1L-1β and IL-18, into their active forms, and induces a form of cell death (pyroptosis) associated with inflammation because of the release of these pro-inflammatory cytokines upon cell rupture. Id. At 99-100.

A priming step, which can result from TLR signaling, must first occur in which cells inducer and translate the mRNAs that encode the pro-forms of IL-1, IL-18 or other cytokines for inflammasome activation to produce inflammatory cytokines. For example, the TLR-3 agonist poly I:C can be used experimentally to prime cells for triggering of the inflammasome. Id. At 100.

Inflammasome activation also can involve proteins of the PYHIN family, which have an H inversion (HIN) domain in place of an LRR domain. There are four PYIN proteins in humans. Id. At 100.

A noncanonical inflammasome (caspase I-independent) pathway uses the protease caspase 11, which therefore is both a sensor and an effector molecule, to detect intracellular LPS. Id. At 101.

Besides activating effector functions and cytokine production, another outcome of the activation of innate sensing pathways is the induction of co-stimulatory molecules on tissue dendritic cells and macrophages. B7.1 (CD80) and B7.2 (CD86), for example, which are induced on macrophages and tissue dendritic cells by innate sensors such as TLRs in response to pathogenic recognition, are recognized by specific co-stimulatory receptors expressed by cells of the adaptive immune response, particularly CD4 T cells, and their activation by B7 is an important step in activating adaptive immune responses. Id. At 105.

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups-class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and βchains (designated A and B) of the MHC class II molecules HLA-DR, HLA-DP, and HLA-DQ in humans. Also in the MHC class II region are the genes for the TAP1:TAP2 peptide transporter, the PSMB (or LMP) genes that encode proteasome subunits, the genes encoding the DMα and BMβ chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes connecting the subunits of the HLA-DM molecule that catalyzes peptide binding to MHC class II molecules are related to the MHC class II genes, as are the DOA and DOB genes that encode the subunits of the regulatory HLA-DO molecule. [Janeway's Immunobiology. 9th ed., GS, Garland Science, Taylor & Francis Group, 2017. pps. 232-233].

MHC-like molecules, while not encoded by the same gene group as true MHCs, have the same folding and overall structure of MHCs, and specifically MHC class I molecules, and thus possess similar biological functions such as antigen presentation. The CD1 family of molecules is an example of a MHC-like molecule. It consists of two groups based on amino acid homology: group 1, which includes CD1a, b, and c; and group 2, which consists of CD1d. Group 1 CD1s can present antigens to a wide variety of T cells, whereas CD1d presents antigens mostly to NKT cells. (Brutkiewicz. “CD1d Ligands: The Good, the Bad, and the Ugly.” The Journal of Immunology (2006) 177 (2) 769-775). While CD1d structurally resembles MHC Class I molecules, it traffics through the endosome of the exogenous antigen presentation pathway. The binding groove of the CD1d molecules tethers the lipid tail of a glycolipid antigen, while the carbohydrate head group of the antigen projects out of the groove for recognition by the TCR of the NKT cell. [Wah, MakTak, et al. “Chapter 11: NK, γ8 T and NKT Cells.” Primer to the Immune Response. Elsevier, 2014].

CD1d presents lipid antigens, and requires the presence of particular mechanisms to induce uptake of these molecules by APCs and subsequent loading onto CD1d molecules. Lipid transfer protein such as apolipoprotein E and fatty acid amide hydrolase (FAAH) have been shown to enhance the presentation of certain antigens by CD1d. Loading efficiency can be enhanced by specific proteins, such as saposins and microsomal triglyceride transfer protein, present in the endosomal and lysosomal compartments of cells by promoting lipid antigen exchange. Similar to MHC antigens, lipid antigens can also be processed by lysosomal enzymes to yield active compounds, as demonstrated in the case of CD1d for synthetic antigens, microbial antigens, and self-antigens. [Giradi and Zajonc (2012). “Molecular basis of lipid antigen presentation by CD1d and recognition by natural killer T cells.” Immunol Rev. 250(1): 167-179].

MHC Class I-like molecules are nonclassical MHC type molecules, while including Cdld also include CD1a, CD1b, CD1c, CD1e, and MR1 are also expressed on APCs and can activate various subsets of T cells. [Kumar and Delovitch (2014) “Different subsets of natural killer T cells may vary in their roles in health and disease.” Immunology 142: 321-336]. Other non-classical histocompatibility molecules include MR1, which activate MAIT cells.

The term “memory cells” as used herein refers to T and B lymphocytes that mediate immunological memory. They are more sensitive than naïve lymphocytes to antigen and respond rapidly on reexposure to the antigen that originally induced them.

The term “microbe” as used herein defines a minute organism typically visible under a microscope, e.g., bacteria, fungi, viruses, algae, and protozoan parasites.

The term “mimetic” refers to chemicals containing chemical moieties that mimic the function of another chemical. For example, if a peptide contains two charged chemical moieties having functional activity, a mimetic places two charged chemical moieties in a spatial orientation and constrained structure so that the charged chemical function is maintained in three-dimensional space.

The terms “modify” and “modulate” as used herein mean to regulate, alter, adapt, or adjust to a certain measure or proportion.

The terms “neutral antagonist” or “silent antagonist” as used herein refer to a drug that attenuates the effects of agonists or inverse agonists, producing a functional reduction in signal transduction. A neutral antagonist affects only ligand-dependent receptor activation and displays no intrinsic activity itself.

The abbreviation “NFκB” as used herein refers to which is a proinflammatory transcription factor. It switches on multiple inflammatory genes, including cytokines, chemokines, proteases, and inhibitors of apoptosis, resulting in amplification of the inflammatory response [Barnes, PJ, (2016) Pharmacol. Rev. 68: 788-815]. The molecular pathways involved in NF-κB activation include several kinases. The classic (canonical) pathway for inflammatory stimuli and infections to activate NF-κB signaling involve the IKK (inhibitor of KB kinase) complex, which is composed of two catalytic subunits, IKK-α and IKK-β, and a regulatory subunit IKK-γ (or NFκB essential modulator [Id., citing Hayden, M S and Ghosh, S (2012) Genes Dev. 26: 203-234]. The IKK complex phosphorylates Nf-κB-bound IκBs, targeting them for degradation by the proteasome and thereby releasing NF-κB dimers that are composed of p65 and p50 subunits, which translocate to the nucleus where they bind to KB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases Re1B and processes p100 to p52 in response to certain members of the TNF family, such as lymphotoxin-β [Id., citing Sun, S C. (2012) Immunol. Rev. 246: 125-140]. This pathway switches on different gene sets and may mediate different immune functions from the canonical pathway. Dominant-negative IKK-βinhibits most of the proinflammatory functions of NF-κB, whereas inhibiting IKK-α has a role only in response to limited stimuli and in certain cells such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151: 179-89].

The term “normal healthy subject” refers to a subject having no symptoms or other evidence of a disorder.

The terms “psychoactive” and “psychotropic” as used herein refer to producing an effect (such as changes in perception or behavior) on the mind or on mental processes.

The term “pathogen” as used herein means a microbe that can cause disease.

The term “pattern recognition receptors” or “PRRs” as used herein, refers to receptors that are present at the cell surface to recognize extracellular pathogens in the endosomes where they sense intracellular invaders, and finally in the cytoplasm. They recognize conserved molecular structures of pathogens, called pathogen associated molecular patterns (PAMPs) specific to the microorganism and essential for its viability. PRRs are divided into four families: toll-like receptors (TLR); nucleotide oligomerization receptors (NLR); C-type leptin receptors (CLR), and RIG-1 like receptors (RLR).

The term “peptide” is used herein to designate a series of amino acid residues, connected one to the other typically by peptide bonds between the alpha-amino and carbonyl groups of the adjacent amino acids. The peptides are typically 9 amino acids in length, but can be as short as 8 amino acids in length, and as long as 14 amino acids in length. The series of amino acids are consider an “oligopeptide” when the amino acid length is greater than about 14 amino acids in length, typically up to about 30 to 40 residues in length. When the amino acid residue length exceeds 40 amino acid residues, the series of amino acid residues is termed “polypeptide”.

The term “peroxisome proliferator-activated receptors (PPARs)” as used herein refers to ligand-activated transcription factors of the nuclear hormone receptor superfamily comprising the following three subtypes: PPARα, PPARγ, and PPARβ/8. Activation of PPAR-α reduces triglyceride level and is involved in regulation of energy homeostasis. Activation of PPAR-γ causes insulin sensitization and enhances glucose metabolism, whereas activation of PPAR-β/8 enhances fatty acids metabolism. Thus, the PPAR family of nuclear receptors plays a major regulatory role in energy homeostasis and metabolic function.

The term “pharmaceutical composition” is used herein to refer to a composition that is employed to prevent, reduce in intensity, cure or otherwise treat a target condition or disease. The terms “formulation” and “composition” are used interchangeably herein to refer to a product of the described invention that comprises all active and inert ingredients.

The term “pharmaceutically acceptable,” is used to refer to a carrier, diluent or excipient being compatible with the other ingredients of the formulation or composition (meaning capable of being combined with each other in a manner such that there is no interaction that would substantially reduce the efficacy of the composition under ordinary use conditions) and not deleterious to the recipient thereof. The carrier must be of sufficiently high purity and of sufficiently low toxicity to render it suitable for administration to the subject being treated. The carrier further should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use.

The term “potentiate” and its other grammatical forms as used herein means to increase the power, effect, or potency, of; to enhance, to augment the activity of.

The term “prevent” as used herein refers to effectual stoppage of action or progress.

The term “priming” as used herein refers to the process whereby T cells and B cell precursors encounter the antigen for which they are specific. For example, before helper T cells and B cells can interact to produce specific antibody, the antigen-specific T cell precursors must be primed. Priming involves several steps: antigen uptake, processing, and cell surface expression bound to class II MHC molecules by an antigen presenting cell, recirculation and antigen-specific trapping of helper T cell precursors in lymphoid tissue, and T cell proliferation and differentiation. [Janeway, C A, Jr., “The priming of helper T cells, Semin. Immunol. 1(1): 13-20 (1989)]. Helper T cells express CD4, but not all CD4 T cells are helper cells. Id. The signals required for clonal expansion of helper T cells differ from those required by other CD4 T cells. The critical antigen-presenting cell for helper T cell priming appears to be a macrophage; and the critical second signal for helper T cell growth is the macrophage product interleukin 1 (IL-1). Id. If the primed T cells and/or B cells receive a second, co-stimulatory signal, they become activated T cells or B cells.

The term “protect” as used herein refers to defend, preserve, or guard from attack, invasion, loss, insult, injury or harm.

The term “protection” as used herein refers to the act of protecting or the state of being protected.

The term “purification” and its various grammatical forms as used herein refers to the process of isolating or freeing from foreign, extraneous, or objectionable elements.

The term “reduce” and its various grammatical forms as used herein refers to a diminution, a decrease, an attenuation or abatement of the degree, intensity, extent, size, amount, or occurrence of a disorder in individuals at risk of developing the disorder.

The term “restore” as used herein refers to putting back to a former or normal condition; to bring back to health, strength.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. A “suspension” is a dispersion (mixture) in which a finely-divided species is combined with another species, with the former being so finely divided and mixed that it doesn't rapidly settle out. In everyday life, the most common suspensions are those of solids in liquid. Among the acceptable vehicles and solvents that may be employed are water, Ringer's solution, and isotonic sodium chloride solution.

The term “solubilizing agents” as used herein refers to those substances that enable solutes to dissolve.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).The term “T cell anergy” as used herein refers to a tolerance mechanism in which the lymphocyte is intrinsically functionally inactivated following an antigen encounter, but remains alive for an extended period of time in a hyporesponsive state. Models of T cell anergy affecting both CD4+ and CD8+ cells fall into two broad categories. One, clonal anergy, is principally a growth arrest state, whereas the other, adaptive tolerance or in vivo anergy, represents a more generalized inhibition of proliferation and effector functions. The former arises from incomplete T cell activation, is mostly observed in previously activated T cells, is maintained by a block in the Ras/MAP kinase pathway, can be reversed by IL-2 or anti-OX40 signaling, and usually does not result in the inhibition of effector functions. The latter is most often initiated in naïve T cells in vivo by stimulation in an environment deficient in costimulation or high in coinhibition. Adaptive tolerance reverses in the absence of antigen. [Schwartz, RH. Ann. Rev. Immunol. (2003) 21: 305-34].

The term “stabilize” and its various grammatical forms as used herein refers to reducing deterioration of.

The term “stimulate” as used herein refers to activate, provoke, or spur. The term “stimulating agent” as used herein refers to a substance that exerts some force or effect.

The phrase “subject in need thereof” as used herein refers to a patient that (i) will be administered a composition of the described invention, (ii) is receiving a composition of the described invention; or (iii) has received a composition of the described invention, unless the context and usage of the phrase indicates otherwise.

The term “susceptible” as used herein refers to a member of a population at risk.

The term “susceptible population” as used herein refers to a subpopulation in the general population likely to experience a disease.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period.

The term “synergistic effect” as used herein refers to an interaction between two or more agents that causes the total effect of the agents to be greater than the sum of the individual effects of each drug

The term “symptom” as used herein refers to a sign or an indication of disorder or disease, especially when experienced by an individual as a change from normal function, sensation, or appearance.

The term “T cell exhaustion” as used herein refers to a state of T cell dysfunction that arises during many chronic infections and cancer. It is defined by poor effector function, sustained expression of inhibitory receptors and a transcriptional state distinct from that of functional effector or memory T cells. Modulating pathways overexpressed in exhaustion—for example, by targeting programmed cell death protein 1 (PD1) and cytotoxic T lymphocyte antigen 4 (CTLA4)—can reverse this dysfunctional state and reinvigorate immune responses [Wherry E J and Kurachi, M. Nature (2015) 15: 486-99, citing Wherry E J. Nat. Immunol. (2011) 131:492-499; Schietinger A, Greenberg P D. Trends Immunol. (2014) 35:51-60; Barber D L, et al. Restoring function in exhausted CD8 T cells during chronic viral infection. Nature. (2006) 439:682-687; Nguyen L T, Ohashi P S. Nat. Rev. Immunol. (2014) 15:45-56]. The level and duration of chronic antigen stimulation and infection seem to be key factors that lead to T cell exhaustion and correlate with the severity of dysfunction during chronic infection. Examples of inhibitory receptors include the inhibitory pathways mediated by PD1 in response to binding of PD1 ligand 1 (PDL1) and/or PDL2. [Id., citing Okazaki T, et al., Nature Immunol. (2013) 14:1212-1218, Odorizzi P M, Wherry E J. J. Immunol. (2012) 188:2957-2965, Araki K, et al. Cold Spring Harb. Symp. Quant. Biol. (2013) 78:239-247]. Exhausted T cells can co-express PD1 together with lymphocyte activation gene 3 protein (LAG3), 2B4 (also known as CD244), CD160, T cell immunoglobulin domain and mucin domain-containing protein 3 (TIM3; also known as HAVCR2), CTLA4 and many other inhibitory receptors [Id., citing Blackburn S D, et al. Nat. Immunol. (2009) 10:29-37]. Typically, the higher the number of inhibitory receptors co-expressed by exhausted T cells, the more severe the exhaustion. It has been suggested that inhibitory receptors such as PD1 might regulate T cell function in several ways [Id., citing Schietinger A, Greenberg P D. Trends Immunol. (2014) 35:51-60; Odorizzi P M, Wherry E J. J. Immunol. (2012) 188:2957-2965], e.g., by ectodomain competition, which refers to inhibitory receptors sequestering target receptors or ligands and/or preventing the optimal formation of microclusters and lipid rafts (for example, CTLA4); second, through modulation of intracellular mediators, which can cause local and transient intracellular attenuation of positive signals from activating receptors such as the TCR and co-stimulatory receptors [Id., citing Parry R V, et al. Molec. Cell. Biol. (2005) 25:9543-9553; Yokosuka T, et al. J. Exp. Med. (2012) 209:1201-1217; Clayton K L, et al. J. Immunol. (2014) 192:782-791]; and third, through the induction of inhibitory genes [Id., citing Quigley M, et al. Nat. Med. (2010) 16:1147-1151]. Co-stimulatory receptors also are involved in T cell exhaustion [Id., citing Odorizzi P M, Wherry E J. J. Immunol. (2012) 188:2957-2965]. For example, desensitization of co-stimulatory pathway signaling through the loss of adaptor molecules can serve as a mechanism of T cell dysfunction during chronic infection. The signaling adaptor tumor necrosis factor receptor (TNFR)-associated factor 1 (TRAF1) is downregulated in dysfunctional T cells in HIV progressors, as well as in chronic LCMV infection [Id., citing Wang C, et al. J. Exp. Med. (2012) 209:77-91]. Adoptive transfer of CD8+ T cells expressing TRAF1 enhanced control of chronic LCMV infection compared with transfer of TRAF1-deficient CD8+ T cells, which indicates a crucial role for TRAF1-dependent co-stimulatory pathways in this setting [Id., citing Wang C, et al. J. Exp. Med. (2012) 209:77 91]. It has also been possible to exploit the potential beneficial role of co-stimulation to reverse exhaustion by combining agonistic antibodies to positive co-stimulatory pathways with blockade of inhibitory pathways. 4-1BB (also known as CD137 and TNFRSF9) is a TNFR family member and positive co-stimulatory molecule that is expressed on activated T cells. Combining PD1 blockade and treatment with an agonistic antibody to 4-1BB dramatically improved exhausted T cell function and viral control [Id, citing Vezys V, et al. J. Immunol. (2011) 187:1634-1642]. Soluble molecules are a second class of signals that regulate T cell exhaustion; these include immunosuppressive cytokines such as IL-10 and transforming growth factor-β (TGFβ) and inflammatory cytokines, such as type I interferons (IFNs) and IL-6. [Id.]

The term “Tbet” as used herein refers to a Th1 cell transcription factor. Differential expression of the Th1 cell transcription factor T bet and a closely related T-box family transcription, factor particularly in CD8+ T cells, Eomesodermin (Eomes) facilitates the cooperative maintenance of the pool of antiviral CD8+ T cells during chronic viral infection. [Paley, M A et a., Science (2012) 338: 1220-125]. During chronic infections, T-bet is reduced in virus-specific CD8+ T cells; this reduction correlates with T cell dysfunction. In contrast, Eomes mRNA expression is up-regulated in exhausted CD8+ T cells during chronic infection. [Id.]

The term “TCR clonotype” as used herein refers to a unique nucleotide sequence that arises during the gene rearrangement process for that receptor. The combination of nucleotide sequences for the surface expressed receptor pair defines the T cell clonotype [Yassai, M B et al. Immnogenetics (2009) 61 (7): 493-502].

The term “TCR repertoire” as used herein refers to the whole range of different TCRs present in an organism. [Aversa, I. et al. Intl J. Molec. Sci. (2020) 21: 2378].

The term “tissue-resident memory (TRM) T cells” as used herein refers to a lymphocyte lineage that occupies tissues without recirculating. They provide a first response against infections reencountered at body surfaces, where they accelerate pathogen clearance.

The term “terpene” as used herein refers to an organic compound that is derived biosynthetically from units of isopentenyl pyrophosphate. Terpene molecules found in plants can be the primary constituents of essential oils and can produce fragrances and smells. Terpenes can be monoterpenoids, sesquiterpenoids, sesterterpenoid, sesquarterpenoids, tetraterpenoids, Triterpenoids, tetraterpenoids, Polyterpenoids, isoprenoids, and steroids. Terpenes can be: α-, β-, γ-, oxo-, isomers, or combinations thereof. Examples of terpenes include, but are not limited to, 8-dihydroionone, Acetanisole, Acetic Acid, Acetyl Cedrene, Anethole, Anisole, Benzaldehyde, Bergamotene (α-cis-Bergamotene, α-trans-Bergamotene), Bisabolol (β-Bisabolol, α-Bisabolol), Borneol, Bomyl Acetate, Butanoic/Butyric Acid, Cadinene (α-Cadinene, γ-Cadinene), Cafestol, Caffeic acid, Camphene, Camphor, Capsaicin, Carene (A-3-Carene, 8-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene, (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene, γ-Curcumene), Decanal, Dehydrovomifoliol, Diallyl Disulfide, Dihydroactinidiolide, Dimethyl Disulfide, Eicosane/Icosane, Elemene (8-Elemene), Estragole, Ethyl acetate, Ethyl Cinnamate, Ethyl maltol, Eucalyptol/1,8-Cineole, Eudesmol (α-Eudesmol, β-Eudesmol, γ-Eudesmol), Eugenol, Euphol, Farnesene, Farnesol, Fenchol (αFenchol), Fenchone, Geraniol, Geranyl acetate, Germacrenes, Germacrene B, Guaia-1(10),11-diene, Guaiacol, Guaiene (α-Guaiene), Gurjunene (α-Gurjunene), Herniarin, Hexanaldehyde, Hexanoic Acid, Humulene (α-Humulene, β-Humulene), Ionol (3-oxo-α-ionol, 13-Ionol), Ionone (α-Ionone, β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, .beta.-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (p-Myrcene, β-Myrcene), γ-Muurolene, Nepetalactone, Nerol, Nerolidol, trans-Nerolido, Neryl acetate, Nonanaldehyde, Nonanoic Acid, Ocimene, Octanel, Octanoic Acid, P-cymene, Pentyl butyrate, Phellandrene, Phenylacetaldehyde, Phenylethanethiol, Phenylacetic Acid, Phytol, Pinene (α-Pinene, β-Pinene), Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpinene, γ-Terpinene, Terpinolene, Thiophenol, Thuj one, Thymol, α-Tocopherol, Tonka Undecanone, Undecanal, Valeraldehyde/Pentanal, Verdoxan, α-Ylangene, or Umbelliferone.

Terpenes constitute the second chemical scaffold produced by Cannabis that is able to differentially target CB receptors. For example, the bicyclic sesquiterpene β-caryophyllene (trans isomer) has been shown to selectively target the CB2 receptor at nM concentrations and to act as a full agonist [Gertsch, J. et al. Br. J. Pharmacol. (2010) 160: 523-29, citing Gertsch, J. et al. Commun. Integr. Biol. (2008) 1: S26-28]. The diterpene salvinorin A from Salvia divinorum Epling & Jativa-M may interact with a putative CB recrprot/kappa-opioid receptor (KOP) heterodimer which may be formed during inflammatory conditions (Id., citing Fichna, J. et al, Neurogstroenterol. Motil. (2009) 21: S1326-Se128). Two naturally occurring quinonoid triterpenoids, pristimerin and euphol, were found to inhibit monoacylglycerol lipase (MAGL) with high potency (IC50=93 nM and 315 nM respectively) through a reversible mechanism [Id., citing King, A R et al Chem. Biol. (2009) 16: 1045-52]. Several distinct triterpenes are known to modulate immune functions [Id., citing Rios, JL J. Ethnopharmacol. (2010) 128: 1-14].

The term “terpenoid” as used herein refers to any of a large class of organic compounds, including terpenes, diterpenes, and sesquiterpenes. They have unsaturated molecules composed of linked isoprene units, generally having the formula C5H8. Terpenoids, such as limonene, myrcene, α-pinene, linalool, β-caryophyllene, caryophyllene oxide, nerolidol, and phytol, share a precursor with phytocannabinoids, and are flavor and fragrance components common in human diets that have been designated as generally recognized as safe (GRAS) by the US Food and Drug Administration and other regulatory agencies. [Da Cheng Hao, et al. (2015).Ch. 11, in Medicinal plants; Elsevier, Ltd., pp. 431 464]

The term “therapeutic agent” as used herein refers to a drug, molecule, nucleic acid, protein, metabolite, composition or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The terms “therapeutic amount”, “an “amount effective”, or “pharmaceutically effective amount” of an active agent are used interchangeably to refer to an amount that is sufficient to provide the intended benefit of treatment. Dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “effective amount” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

The term “therapeutic effect” as used herein refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation. For any therapeutic agent described herein the therapeutically effective amount may be initially determined from preliminary in vitro studies and/or animal models. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The term “therapeutic window” refers to a concentration range that provides therapeutic efficacy without unacceptable toxicity. Following administration of a dose of a drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

Instead, another dose of drug should be given to maintain concentrations within the therapeutic window. In general, the lower limit of the therapeutic range of a drug appears to be approximately equal to the drug concentration that produces about half of the greatest possible therapeutic effect, and the upper limit of the therapeutic range is such that no more than about 5% to about 10% of patients will experience a toxic effect. These figures can be highly variable, and some patients may benefit greatly from drug concentrations that exceed the therapeutic range, while others may suffer significant toxicity at much lower values. The therapeutic goal is to maintain steady-state drug levels within the therapeutic window. For most drugs, the actual concentrations associated with this desired range are not and need not be known, and it is sufficient to understand that efficacy and toxicity are generally concentration-dependent, and how drug dosage and frequency of administration affect the drug level. For a small number of drugs where there is a small (two- to three-fold) difference between concentrations resulting in efficacy and toxicity, a plasma-concentration range associated with effective therapy has been defined. A target level strategy is reasonable, wherein a desired target steady-state concentration of the drug (usually in plasma) associated with efficacy and minimal toxicity is chosen, and a dosage is computed that is expected to achieve this value. Drug concentrations subsequently are measured and dosage is adjusted if necessary to approximate the target more closely.

In most clinical situations, drugs are administered in a series of repetitive doses or as a continuous infusion to maintain a steady-state concentration of drug associated with the therapeutic window. To maintain the chosen steady-state or target concentration (“maintenance dose”), the rate of drug administration is adjusted such that the rate of input equals the rate of loss. If the clinician chooses the desired concentration of drug in plasma and knows the clearance and bioavailability for that drug in a particular patient, the appropriate dose and dosing interval can be calculated.

The terms “TH1” and “TH2” as used herein refers to subsets of effector CD4 T cells characterized by the cytokines they produce. TH1 cells are mainly involved in activating macrophages but can also help stimulate B cells to produce antibody. TH2 cells are involved in stimulating B cells to produce antibody. The term “TH17” as used herein refers to a subset of CD4 T cells characterized by production of the cytokine IL-17. They help recruit neutrophils to sites of infection.

The term “toll-like receptor (TLR)” as used herein refers to innate receptors on macrophages, dendritic cells, and some other cells, that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

The term “topical” refers to administration of a composition at, or immediately beneath, the point of application. The phrase “topically applying” describes application onto one or more surfaces(s) including epithelial surfaces.

The term “transient receptor potential (TRP) ion channels constitute a superfamily of cation permeable ion channels that integrate multiple stimuli as cellular sensors responding to broad range of stimuli.

As used herein the term “treating” includes abrogating, substantially inhibiting, slowing or reversing the progression of a condition, substantially ameliorating clinical symptoms of a condition, or substantially preventing the appearance of clinical symptoms of a condition. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “tumor necrosis factor (TNF, also referred as TNF-α)” as used herein refers to a cytokine involved in systemic inflammation; it is a member of a group of cytokines that stimulate the acute phase reaction. Studies have shown that TNF-α induces expression of IL-6 via three distinct signaling pathways inside the cell, i.e., 1) NF-κB pathway 2) MAPK pathway, and 3) death signaling pathway.

The term “unprimed cells” (also referred to as virgin, naïve, or inexperienced cells) as used herein refers to T cells and B cells that have generated an antigen receptor (TCR for T cells, BCR for B cells) of a particular specificity, but have never encountered the antigen.

The terms “variants”, “mutants”, and “derivatives” are used herein to refer to nucleotide or polypeptide sequences with substantial identity to a reference nucleotide or polypeptide sequence. The differences in the sequences may be the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

The term “vigor” as used herein refers to activity, energy, force, health, or strength.

The term “viral load” as used herein refers to a measurement of the amount of a virus in an organism, typically in the bloodstream, usually stated in virus particles per milliliter.

The term “volume/volume percentage is a measure of the concentration of a substance in a solution. It is expressed as the ratio of the volume of the solute to the total volume of the solution multiplied by 100. Volume percent (vol/vol % or v/v %) should be used whenever a solution is prepared by mixing pure liquid solutions.

The term “weight by weight percentage” or wt/wt % is used herein to refer to the ratio of weight of a solute to the total weight of the solution.

### 2. Embodiments

2.1 Therapeutic, for Immune System Health

According to one aspect, the described invention provides a method for improving immune system health in a subject in need thereof comprising administering a composition comprising a therapeutic amount of an active constituent and a pharmaceutically acceptable carrier, wherein the therapeutic amount of the active constituent potentiates an immune response, compared to a control. According to some embodiments, the control is the subject before the administering.

According to some embodiments, the active constituent comprises N-acetylcysteine. According to some embodiments, the therapeutic amount of the composition comprising N-acetylcysteine potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%.

According to some embodiments, for each dose, onset of potentiation of the immune response by increasing diversity by the N-acetylcysteine component occurs within 24 hours of dosing. According to some embodiments, the potentiated immune response comprises an enhanced T cell diversity, an enhanced B cell diversity, or both, compared to the subject before the administering. According to some embodiments, the therapeutic amount stabilizes the T cell immune repertoire. According to some embodiments, the potentiated immune response comprises an increased resistance to T cell exhaustion. According to some embodiments, the potentiated immune response improves clinical outcome in response to a pathogen. According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease. According to some embodiments, the potentiated immune response reduces appearance of disease. According to some embodiments, the potentiated immune response increases health span of the subject. According to some embodiments, the administering of the N-acetylcysteine component is orally. According to some embodiments the therapeutic amount of the composition further comprises a mucolytic therapeutic effect, an anti-oxidant therapeutic effect, or both.

According to some embodiments, the active constituent comprises a botanical ingredient, e.g., derived from Theobroma cacao, Echinaceae spp, plants of the Apiaceae family; e.g., Daucus carota L., kava. For example, the noni fruit (Morinda citrifolia) is used extensively throughout Polynesia and has been traditionally used for its health-promoting qualities. According to some embodiments, the botanical ingredient comprises a botanical extract. According to some embodiments, the cannabimimetic is a synthetic compound. According to some embodiments, the botanical ingredient comprises a cannabinoid. According to some embodiment, the botanical ingredient comprises a terpenoid (e.g., β-caryophyllene (trans isomer); the diterpene salvinorin A, pristimerin, euphol). According to some embodiments, the botanical ingredient comprises a cannibimimetic component or a derivative thereof. Exemplary cannabimimetics include, without limitation, a fatty acid derivative (e.g., an N-acetylethanolamine, such as N-linoleoylethanolamide, N-oleoylethanolamide, N-acylethanolamine (NAE) palmitoylethanolamide); N-alkylamides (e.g., dodeca-2E, 4E, 8Z, 10Z-tetraenoic acid isobutylamide; dodeca-2E, 4E-dienoic acid isobutylamide), falcarinol; or a flavonoid (.g., genistein, kaempferol, 7-hydroxyflavone, and 3,7-dihydroxyflavone; trans-resveratrol, curcumin, catechins, and kaemferol-type flavonoids).

Cannabimimetics also may be derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; Essential Oils, for example: Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), and Ylang ylang (Cananga odorata(Lam.) var. genuine).

According to some embodiments, the therapeutic amount of the composition comprising the botanical ingredient or cannabimimetic potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%. According to some embodiments, for each dose, onset of potentiation of the immune response by increasing diversity by the cannabinoid component occurs within 24 hours of dosing. According to some embodiments, the potentiated immune response comprises an enhanced T cell diversity, an enhanced B cell diversity, or both, compared to the subject before the administering. According to some embodiments, the therapeutic amount stabilizes the T cell immune repertoire. According to some embodiments, the potentiated immune response comprises an increased resistance to T cell exhaustion. According to some embodiments, the potentiated immune response improves clinical outcome in response to a pathogen. According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease. According to some embodiments, the potentiated immune response reduces appearance of disease. According to some embodiments, the potentiated immune response increases health span of the subject. According to some embodiments, the administering of the cannabinoid is topically. According to some embodiments, a therapeutic effect of the cannabinoid or cannabimimetic administered topically is non-psychoactive. According to some embodiments, the administering of the cannabinoid or cannabimimetic is orally.

According to some embodiments, the active constituent comprises N-acetylcysteine and a cannabinoid or cannabimimetic. According to some embodiments, the cannabimimetic comprises a botanical ingredient, e.g., derived from Theobroma cacao, Echinaceae spp, plants of the Apiaceae family; e.g., Daucus carota L., kava. For example, the noni fruit (Morinda citrifolia) is used extensively throughout Polynesia and has been traditionally used for its health-promoting qualities. According to some embodiments, the cannabimimetic comprises a botanical extract. According to some embodiments, the cannabimimetic is a synthetic compound. According to some embodiments, the botanical ingredient comprises a cannabinoid. According to some embodiment, the botanical ingredient comprises a terpenoid (e.g., β-caryophyllene (trans isomer); the diterpene salvinorin A, pristimerin, euphol). According to some embodiments, the botanical ingredient comprises a cannibimimetic component or a derivative thereof. Exemplary cannabimimetics include, without limitation, a fatty acid derivative (e.g., an N-acetylethanolamine, such as N-linoleoylethanolamide, N-oleoylethanolamide, N-acylethanolamine (NAE) palmitoylethanolamide); N-alkylamides (e.g., dodeca-2E, 4E, 8Z, 10Z-tetraenoic acid isobutylamide; dodeca-2E, 4E-dienoic acid isobutylamide), falcarinol; or a flavonoid (.g., genistein, kaempferol, 7-hydroxyflavone, and 3,7-dihydroxyflavone; trans-resveratrol, curcumin, catechins, and kaemferol-type flavonoids).

Cannabimimetics also may be derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; Essential Oils, for example: Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), and Ylang ylang (Cananga odorata(Lam.) var. genuine).

According to some embodiments, the amount of NAC in a single dose ranges from 200 to 2400 mg, inclusive, i.e., 200 mg, 300 mg, 400 mg, 500 mg, 600 mg, 700 mg, 800 mg, 900 mg, 1000 mg, 1100 mg, 1200 mg, 1300 mg, 1400 1500 mg, 1600 mg, 1700 mg, 1800 mg, 1900 mg, 2000 mg, 2100 mg, 2200 mg, 2300 mg, 2400 mg, and the amount of a cannabinoid or cannabinomimetic in a single dose ranges from 1 mg to 50 mg THC, inclusive, i.e., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg 50 mg, and 1 mg to 600 mg CBD, inclusive, i.e., 1 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 28 mg 0, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, with ranges to accommodate different cannabinoid ratios (THC:CBD) over varying lengths of treatment time. Number of doses per day can also vary According to some embodiments, the therapeutic amount of the composition comprising N-acetylcysteine and the cannabinoid or cannabimimetic potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%. According to some embodiments, for each dose, onset of potentiation of the immune response by increasing diversity by the composition occurs within 24 hours of dosing. According to some embodiments, the therapeutic effect of the N-acetylcysteine and of the cannabinoid are complementary. According to some embodiments, the potentiated immune response comprises an enhanced T cell diversity, an enhanced B cell diversity, or both, compared to the subject before the administering. According to some embodiments, the therapeutic amount stabilizes the T cell immune repertoire. According to some embodiments, the potentiated immune response comprises an increased resistance to T cell exhaustion. According to some embodiments, the potentiated immune response improves clinical outcome in response to a pathogen. According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease. According to some embodiments, the potentiated immune response reduces appearance of disease. According to some embodiments, the potentiated immune response increases health span of the subject. According to some embodiments, the administering of the N-acetylcysteine component is orally and the administering of the cannabinoid is topically. According to some embodiments, a therapeutic effect of the cannabinoid administered topically is non-psychoactive.

According to some embodiments, the subject in need is an aged person (defined as a person of greater than 60 years of age). According to some embodiments, the immune system of the aged person is compromised by one or more of prior illnesses, chronic illnesses, or the aging process. Exemplary preexisting chronic illnesses include, without limitation, diabetes, hypertension, chronic renal failure, chronic heart disease, pulmonary disease, and cancer. According to some embodiments, advancing age is a risk factor for the chronic diseases. According to some embodiments, the aging process comprises age-related changes in dynamic biological, physiological, environmental, psychological, behavioral, and social processes. According to some embodiments, the age-related changes result in declines in function of the senses and activities of daily life and an increased susceptibility to and frequency of disease, frailty, or disability.

2.2 Prophylaxis for Viral Infections

According to another aspect, the described invention provides a method for treating symptoms of a virus infection, wherein the virus infection is characterized by an elevated level of T cell exhaustion; reduced functional diversity of T cells in peripheral blood, and severe pulmonary inflammation, comprising administering a composition comprising a therapeutic amount of an active constituent and a pharmaceutically acceptable carrier, wherein the therapeutic amount of the active constituent potentiates an immune response, compared to a control. According to some embodiments, the control is the subject before the administering. According to some embodiments, the virus infection is an influenza virus infection. According to some embodiments, the virus infection is a human immunodeficiency virus infection. According to some embodiments, the virus infection is a human coronavirus infection. According to some embodiments, the human coronavirus infection is a COVID-19 virus infection. According to some embodiments, the viral infection is further characterized by T cell apoptosis. According to some embodiments, the therapeutic amount reduces viral load. According to some embodiments, the method for treating symptoms of a virus infection comprises monitoring viral load.

According to some embodiments, the active constituent comprises N-acetylcysteine. According to some embodiments, the therapeutic amount of the composition comprising N-acetylcysteine potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%.

According to some embodiments, the therapeutic amount stabilizes the T cell immune repertoire. According to some embodiments, for each dose, onset of potentiation of the immune response by increasing diversity by the N-acetylcysteine component occurs within 24 hours of dosing. According to some embodiments, the potentiated immune response comprises an enhanced T cell diversity, an enhanced B cell diversity, or both, compared to the subject before the administering. According to some embodiments, the potentiated immune response comprises an increased resistance to T cell exhaustion. According to some embodiments, the potentiated immune response improves clinical outcome in response to a pathogen. According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease. According to some embodiments, the potentiated immune response reduces appearance of disease. According to some embodiments, the potentiated immune response increases health span of the subject. According to some embodiments, the administering of the N-acetylcysteine component is orally. According to some embodiments the therapeutic amount of the composition further comprises a mucolytic therapeutic effect, an anti-oxidant therapeutic effect, or both.

According to some embodiments, the active constituent comprises a botanical ingredient, e.g., derived from Theobroma cacao, Echinaceae spp, plants of the Apiaceae family; e.g., Daucus carota L., kava. For example, the noni fruit (Morinda citrifolia) is used extensively throughout Polynesia and has been traditionally used for its health-promoting qualities. According to some embodiments, the botanical ingredient comprises a botanical extract. According to some embodiments, the cannabimimetic is a synthetic compound. According to some embodiments, the botanical ingredient comprises a cannabinoid. According to some embodiment, the botanical ingredient comprises a terpenoid (e.g., β-caryophyllene (trans isomer); the diterpene salvinorin A, pristimerin, euphol). According to some embodiments, the botanical ingredient comprises a cannibimimetic component or a derivative thereof. Exemplary cannabimimetics include, without limitation, a fatty acid derivative (e.g., an N-acetylethanolamine, such as N-linoleoylethanolamide, N-oleoylethanolamide, N-acylethanolamine (NAE) palmitoylethanolamide); N-alkylamides (e.g., dodeca-2E, 4E, 8Z, 10Z-tetraenoic acid isobutylamide; dodeca-2E, 4E-dienoic acid isobutylamide), falcarinol; or a flavonoid (.g., genistein, kaempferol, 7-hydroxyflavone, and 3,7-dihydroxyflavone; trans-resveratrol, curcumin, catechins, and kaemferol-type flavonoids).

Cannabimimetics also may be derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Co enzyme QlO, glutathione; Essential Oils, for example: Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), and Ylang ylang (Cananga odorata(Lam.) var. genuine).

According to some embodiments, the therapeutic amount of the composition comprising the cannabinoid or cannabimimetic potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%. According to some embodiments, the therapeutic amount stabilizes the T cell immune repertoire. According to some embodiments, for each dose, onset of potentiation of the immune response by increasing diversity occurs within 24 hours of dosing. According to some embodiments, the potentiated immune response comprises an enhanced T cell diversity, an enhanced B cell diversity, or both, compared to the subject before the administering. According to some embodiments, the potentiated immune response comprises an increased resistance to T cell exhaustion. According to some embodiments, the potentiated immune response improves clinical outcome in response to a pathogen.

According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease.

According to some embodiments, the potentiated immune response reduces appearance of disease. According to some embodiments, the potentiated immune response increases health span of the subject. According to some embodiments, the administering of the cannabinoid is topically. According to some embodiments, a therapeutic effect of the cannabinoid administered topically is non-psychoactive. According to some embodiments, the administering of the cannabinoid is orally.

According to some embodiments, the active constituent comprises N-acetylcysteine and a botanical ingredient, e.g., derived from Theobroma cacao, Echinaceae spp, plants of the Apiaceae family; e.g., Daucus carota L., kava. For example, the noni fruit (Morinda citrifolia) is used extensively throughout Polynesia and has been traditionally used for its health-promoting qualities. According to some embodiments, the botanical ingredient comprises a botanical extract. According to some embodiments, the botanical ingredient is a cannabimimetic. According to some embodiments, the cannabimimetic is a synthetic compound. According to some embodiments, the botanical ingredient comprises a cannabinoid. According to some embodiment, the botanical ingredient comprises a terpenoid (e.g., β-caryophyllene (trans isomer); the diterpene salvinorin A, pristimerin, euphol). According to some embodiments, the botanical ingredient comprises a cannibimimetic component or a derivative thereof. Exemplary cannabimimetics include, without limitation, a fatty acid derivative (e.g., an N-acetylethanolamine, such as N-linoleoylethanolamide, N-oleoylethanolamide, N-acylethanolamine (NAE) palmitoylethanolamide); N-alkylamides (e.g., dodeca-2E, 4E, 8Z, 10Z-tetraenoic acid isobutylamide; dodeca-2E, 4E-dienoic acid isobutylamide), falcarinol; or a flavonoid (.g., genistein, kaempferol, 7-hydroxyflavone, and 3,7-dihydroxyflavone; trans-resveratrol, curcumin, catechins, and kaemferol-type flavonoids).

Cannabimimetics also may be derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; Essential Oils, for example: Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), and Ylang ylang (Cananga odorata(Lam.) var. genuine).

According to some embodiments, the amount of NAC in a single dose ranges from 200 to 2400 mg, inclusive, i.e., 200 mg, 300 mg, 400 mg, 500 mg, 600 mg, 700 mg, 800 mg, 900 mg, 1000 mg, 1100 mg, 1200 mg, 1300 mg, 1400 1500 mg, 1600 mg, 1700 mg, 1800 mg, 1900 mg, 2000 mg, 2100 mg, 2200 mg, 2300 mg, 2400 mg, and the amount of a cannabinoid or cannabinomimetic in a single dose ranges from 1 mg to 50 mg THC, inclusive, i.e., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg 50 mg, and 1 mg to 600 mg CBD, inclusive, i.e., 1 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 28 mg 0, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, with ranges to accommodate different cannabinoid ratios (THC:CBD) over varying lengths of treatment time. Number of doses per day can also vary. According to some embodiments, the therapeutic amount of the composition comprising N-acetylcysteine and the cannabinoid potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%. According to some embodiments, the therapeutic amount stabilizes the T cell immune repertoire. According to some embodiments, for each dose, onset of potentiation of the immune response by increasing diversity occurs within 24 hours of dosing. According to some embodiments, the therapeutic effect of the N-acetylcysteine and of the cannabinoid are complementary. According to some embodiments, the potentiated immune response comprises an enhanced T cell diversity, an enhanced B cell diversity, or both, compared to the subject before the administering. According to some embodiments, the potentiated immune response comprises an increased resistance to T cell exhaustion. According to some embodiments, the potentiated immune response improves clinical outcome in response to a pathogen.

According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease.

According to some embodiments, the potentiated immune response reduces appearance of disease. According to some embodiments, the potentiated immune response increases health span of the subject. According to some embodiments, the administering of the N-acetylcysteine component is orally and the administering of the cannabinoid is topically. According to some embodiments, a therapeutic effect of the cannabinoid administered topically is non-psychoactive. According to some embodiments, the administering of the cannabinoid is orally.

According to some embodiments, the subject in need is an aged person (defined as a person of greater than 60 years of age). According to some embodiments, the immune system of the aged person is compromised by one or more of prior illnesses, chronic illnesses, or the aging process. Exemplary preexisting chronic illnesses include, without limitation, diabetes, hypertension, chronic renal failure, chronic heart disease, pulmonary disease, and cancer. According to some embodiments, advancing age is a risk factor for a number of chronic diseases in humans. According to some embodiments, the aging process comprises age-related changes in dynamic biological, physiological, environmental, psychological, behavioral, and social processes. According to some embodiments, the age-related biological and physiological changes result in increased susceptibility to the viral infection.

2.3 Potentiating Vaccine Efficiency

Vaccines are therapeutics used to invoke an immune response and provide immunity that are prepared from the causative agent of a disease, its products, or a synthetic substitute treated to act as an antigen without inducing the disease. They protect the recipient by inducing effector mechanisms capable of rapidly controlling replicating pathogens or of inactivating their toxic components.

While vaccination provides a cost effective measure to prevent disease and to control outbreaks of infection at herd level, vaccines currently on the market have significant shortcomings and even failures.

The first vaccine developed was one in which the wild-type disease or the wild-type version of a related disease was “killed” and delivered. While such vaccines were known to work, they carried a significant risk of severe disease or even death in the recipient.

The second type of vaccine developed was attenuated vaccines. This vaccine was based on material obtained from infected rabbit brain attenuated by drying, an uncertain process; vaccines prepared in this way frequently caused serious side effects. Attenuated vaccines are mostly now based on inactivated virus grown in tissue culture. Rabies was the first virus attenuated in a laboratory to create a human vaccine. Acquisition of the ability to grow viruses in tissue culture for an extended period led to the development of attenuated vaccines against measles, poliomyelitis, rubella, influenza, rotavirus, tuberculosis and typhoid. Because the vaccine components are alive, they can spread to non-vaccinated subjects, extending the impact of vaccination to the community at large (See generally, Greenwood B. Philosophical transactions of the Royal Society of London. Series B, Biological sciences, (2014) 369(1645): 20130433. doi:10.1098/rstb.2013.0433).

Live attenuated virus vaccines are a favored vaccination strategy, in part due to their previous success with the yellow fever virus vaccine, YF-17D, in the 1930s. [Ghaffar, K. A. et al, Vaccines (2018) 6, 77; doi: 10.3390/vaccines040077]. A single dose of YF-17D vaccine, for example, is able to induce high titers of neutralizing antibody (nAb) which confer protection on at least 95% of recipients [Id., citing Barrett A. D., Teuwen D. E. Curr. Opin. Immunol. (2009) 21: 308-313. doi: 10.1016/j.coi.2009.05.018; Bonaldo, M C et al., Hum. Vaccin. Immunother. (2014) 10: 1256-1265. doi: 10.4161/hv.28117)] This strategy has been employed with many other diseases, including polio, measles and mumps [Id., citing Plitnick L. M. Chapter 9-Global Regulatory Guidelines for Vaccines. In: Plitnick L. M., Herzyk D. J., editors. Nonclinical Development of Novel Biologics, Biosimilars, Vaccines and Specialty Biologics. Academic Press; San Diego, Calif., USA: (2013). pp. 225-241]. Moreover, the production of attenuated vaccines is cost effective and fairly simple in comparison to other vaccine strategies.

While a live attenuated vaccine has the advantage of being able to elicit immune responses with a single dose, drawbacks include its limited use in immunocompromised or pregnant patients due to the risk of adverse effects. Indeed, because these vaccines contain live virus, mutations may occur in the attenuated vaccine strain with a reversion to virulence, as seen with oral polio vaccine, which causes paralysis in about one in two million recipients. Further, they may cause significant illness in subjects with impaired immunity, as has been seen with the anti-tuberculosis vaccine Bacille Calmette Guerin (BCG) when given to immunodeficient patients, including those with human immunodeficiency virus (HIV) infection.

Next, researchers developed killed vaccines where the pathogens were killed and then used. These vaccines were usually poorly immunogenic and often caused significant side effects, so that whole-cell vaccines have largely given way to subunit vaccines, among other types of vaccines. [See generally, Greenwood B. Philosophical transactions of the Royal Society of London. Series B, Biological sciences (2014) 369(1645): 20130433. doi:10.1098/rstb.2013.0433]. Subunit vaccines comprise a fragment of a pathogen, i.e. a protein, or peptides [Ghaffar, K. A. et al. Vaccines (2018) 6, 77; doi: 10.3390/vaccines040077]. While subunit vaccines are generally a safer choice, because they tend to be less immunogenic, an adjuvant and/or multiple doses are required.

The use of mRNA vaccines is a relatively new trend that has gained popularity [Ghaffar, K. A. et al, “Fast Tracks and Roadblocks for Zika Vaccines,” (2018) Vaccines 6, 77; doi: 10.3390/vaccines040077 citing Plitnick L. M. Chapter 9-Global Regulatory Guidelines for Vaccines. In: Plitnick L. M., Herzyk D. J., editors. Nonclinical Development of Novel Biologics, Biosimilars, Vaccines and Specialty Biologics. Academic Press; San Diego, Calif., USA: (2013). pp. 225-241]. As the minimal genetic construct, mRNA contains only the elements required for expression of the specific encoded protein region. In addition, mRNA is incapable of interacting with the genome, but instead acts only as a transient carrier of information. Other advantages for its use as a vaccine platform include its safety profile [Id. citing Lundstrom, K., Future Sci. OA (2018) 4: FSO300 doi: 10.4155/fsoa-2017-0151]. However, one of the disadvantages of utilizing mRNA as an approach to vaccine design is its rapid degradation by ribonucleases.

The use of genetically engineered DNA plasmids encoding various antigens to induce both humoral and cellular responses has been explored against various infectious diseases caused by parasites [Id., citing Cherif, M S et al, Vaccine (2011) 29: 9038-9050; Cheng, P C et al., PLoS Neg. Trop Dis. (2016) 10: e00044594; doi: 10.1371/journal.pntd.0004459)] bacteria [Id., citing Li, X. et al., Clin. Vaccine Immunol. 2012; 19:723-730. doi: 10.1128/CVI.05700-11; Albrecht, M T, et al., Med. Microbiol. (2012) 65: 505-509 doi: 10.1111/j.1574-695X.2012.00974.x] and viruses [Id., citing Donnelly, J J et al., Nature Med. (1995) 1: 583-597 doi: 10.1038/nm0695-583; Porter, K R et al., Vaccine (2012) 30: 36-341 doi: 10.1016/j.vaccine.2011.10.085).]

### 2.3-1 Influenza Vaccines

The segmented genomes and error-prone RNA-dependent RNA polymerases of influenza viruses enable them to undergo antigenic shift and antigenic drift, which in turn results in evasion of the adaptive immune responses in a range of mammalian and avian species, including humans. This adaptive ability causes influenza viruses to continue to confound efforts to produce long-lasting vaccines against the disease. The protection provided by current influenza vaccines is largely dependent on the induction of neutralizing antibody against the globular head domain of the viral surface protein HA to block viral entry. Since the HA head domain is highly variable among different influenza virus strains, current seasonal vaccines are only effective against well-matching circulating virus strains. [See Zhao, C. and Xu, J., Curr. Op. Immunol. (2018) 53: 1-6].

Other vaccine efforts have been made. For example, a vaccine containing virus-like particles with tandem repeat M2e epitopes generated heterotypic immunity through the induction of antibodies, and protection correlated with IFN-γ-secreting CD8+ TRM. A Modified Vaccinia Ankara-vectored virus expressing conserved influenza nucleoprotein and matrix protein 1 elicited an IFN-γ secreting CD4+ T cell and CD8+ TRM response. Co-administration of 4-1BBL (CD137 signal) along with an influenza nucleoprotein expressing replication defective adenovirus vector via the intranasal route stimulated and boosted a lung CD8+ TRM response through the recruitment of circulating T cells. A Intranasal administration of Fc-fused IL-7 was used as a pre-treatment before influenza A infection, and demonstrated protective capacities in mice against lethal challenge. It appears that Fc-fused IL-7 recruits polyclonal circulating T cells into the lungs, which subsequently reside in the lung tissue as “TRM-like cells”. An antibody targeted vaccination strategy in which antigens are coupled to monoclonal antibodies against CD103+ or DNGR-1+ dendritic cells has also been shown to elicit a protective CD8+ TRM response. [Muruganandah, V., et al. Frontiers in Immunology (2018) 9, 1574. doi:10.3389/fimmu.2018.01574].

### 2.3-2 HIV Vaccine Development Efforts

Vaccine development efforts for HIV have been largely unsuccessful. Both antibodies and cytotoxic T lymphocytes are produced upon infection with HIV [Seabright, G. E. et al., J. Mol. Biol. (2019) 431 (12): 2223-2247]. There have been some protective immune responses to vaccines that invoke T cell-mediated immunity, polyfunctional antibody responses, antibody-dependent cellular cytotoxicity, and broadly neutralizing antibodies (bNab) [MacGregor, R. et al. AIDS (2002) 16: 2137-2143].

Despite the growing understanding of human immunodeficiency virus (HIV) disease pathogenesis and the structure of its key antigenic targets, efforts to develop effective vaccines against HIV continue to fail.

DNA vaccines that invoke T cell mediated immunity developed for HIV still lack the ability to induce long-term immune responses. For example, a DNA vaccine that encoded env and rev was shown to induce CD4+ T cell responses and poorly induce CD8+ T cell responses [MacGregor, R. et al. AIDS (2002)16: 2137-2143]. Similar results were seen in DNA vaccines that encode gag and pol genes [Tavel, J. A., et al., J. AIDS (2007) 44: 601 605].

### 2.3-3 SARS-CoV-2 (COVID-19)

All-out efforts to develop a potential SARS-CoV2 vaccine using available vaccine development methods are ongoing worldwide. Zhang, J. et al. (Vaccines (2020) 8: 153 reported that the potential fragments of S protein for use as antigens in vaccine development include the full-length S protein, the RBD domain, the S1 subunit, NTD, and FP Most of SARS-CoV-2 subunit vaccines currently under development use RBD as the antigen. Since the RBD of S protein directly interacts with the ACE2 receptor on host cells, it is thought that RBD immunization induced specific antibodies may block this recognition and thus effectively prevent the invasion of the virus. The N-terminal domain (NTD) of S proteins is also a candidate antigen for vaccine development. The S1 subunit, which contains both RBD and NTD, which is mainly involved in the S protein binding to the host receptor, is also widely used in vaccine development. The FP domain of the S2 subunit, which is involved in the membrane fusion of the virus, may also serve as a vaccine candidate antigen. M protein, which is a trans-membrane glycoprotein with a molecular weight of about 25 kDa, is involved in virus assembly, is the most abundant protein on the surface of SARS-CoV; it may be used as a candidate for developing a vaccine. So far, several institutions have successfully isolated the virus strains of SARS-CoV-2 and started whole-cell killed or live-attenuated vaccine development. Likewise, several institutions have initiated programs on the SARS-CoV-2 subunit vaccine, and almost all of them use the S protein as antigens.

According to another aspect, the described invention provides a method for increasing potency or efficacy of an antiviral vaccine in a subject, comprising administering a composition comprising a therapeutic amount of an active constituent and a pharmaceutically acceptable carrier, wherein the therapeutic amount of the active constituent potentiates an anti-viral immune response, compared to a control. According to some embodiments, the control is the subject before the administering. According to some embodiments, the therapeutic amount reduces viral load. According to some embodiments, the method for increasing potency or efficacy of an antiviral vaccine further comprises monitoring viral load.

According to some embodiments, the antiviral vaccine is employed to help the body's immune system recognize and fight infections caused by, for example, polio, measles virus, mumps virus, rubella virus, influenza virus, rotavirus, or rabies virus in a susceptible population. According to some embodiments, the antiviral vaccine is employed to help the body's immune system recognize and fight infections caused by poliovirus in a susceptible population. According to some embodiments, the antiviral vaccine is employed to help the body's immune system recognize and fight infections caused by measles virus in a susceptible population. According to some embodiments, the antiviral vaccine is employed to help the body's immune system recognize and fight infections caused by mumps virus in a susceptible population. According to some embodiments, the antiviral vaccine is employed to help the body's immune system recognize and fight infections caused by rubella in a susceptible population. According to some embodiments, the antiviral vaccine is employed to help the body's immune system recognize and fight infections caused by influenza virus in a susceptible population. According to some embodiments, the antiviral vaccine is employed to help the body's immune system recognize and fight infections caused by rotavirus in a susceptible population. According to some embodiments, the antiviral vaccine is employed to help the body's immune system recognize and fight infections caused by rabies virus in a susceptible population. According to some embodiments, the antiviral vaccine may be employed to help the body's immune system recognize and fight infections caused by human immunodeficiency virus. According to some embodiments, the antiviral vaccine may be employed to help the body's immune system recognize and fight infections caused by a coronavirus in a susceptible population. According to some embodiments, the coronavirus may be SARS, MERS, or COVID-19.

According to some embodiments, the active constituent comprises N-acetylcysteine. According to some embodiments, the therapeutic amount of the composition comprising N-acetylcysteine potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%.

According to some embodiments, the therapeutic amount of the composition stabilizes the T cell immune repertoire. According to some embodiments, for each dose of NAC, onset of potentiation of the immune response by increasing diversity occurs within 24 hours of dosing. According to some embodiments, the potentiated immune response comprises an enhanced T cell diversity, an enhanced B cell diversity, or both, compared to the subject before the administering. According to some embodiments, the potentiated immune response comprises an increased resistance to T cell exhaustion. According to some embodiments, the potentiated immune response improves clinical outcome in response to a pathogen. According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease.

According to some embodiments, the potentiated immune response reduces appearance of disease. According to some embodiments, the potentiated immune response increases health span of the subject. According to some embodiments, the administering of the N-acetylcysteine component is orally. According to some embodiments the therapeutic amount of the composition further comprises a mucolytic therapeutic effect, an anti-oxidant therapeutic effect, or both.

According to some embodiments, the active constituent comprises a botanical ingredient. According to some embodiments, the botanical ingredient comprises a cannabinoid or cannabimimetic agent component. According to some embodiments, the cannabinoid or cannabimimetic agent component comprises a biological extract. According to some embodiments, the active constituent comprises a botanical ingredient, e.g., derived from Theobroma cacao, Echinaceae spp, plants of the Apiaceae family; e.g., Daucus carota L., kava. For example, the noni fruit (Morinda citrifolia) is used extensively throughout Polynesia and has been traditionally used for its health-promoting qualities. According to some embodiments, the botanical ingredient comprises a botanical extract. According to some embodiments, the cannabimimetic is a synthetic compound. According to some embodiments, the botanical ingredient comprises a cannabinoid. According to some embodiment, the botanical ingredient comprises a terpenoid (e.g., β-caryophyllene (trans isomer); the diterpene salvinorin A, pristimerin, euphol). According to some embodiments, the botanical ingredient comprises a cannibimimetic component or a derivative thereof. Exemplary cannabimimetics include, without limitation, a fatty acid derivative (e.g., an N-acetylethanolamine, such as N-linoleoylethanolamide, N-oleoylethanolamide, N-acylethanolamine (NAE) palmitoylethanolamide); N-alkylamides (e.g., dodeca-2E, 4E, 8Z, 10Z-tetraenoic acid isobutylamide; dodeca-2E, 4E-dienoic acid isobutylamide), falcarinol; or a flavonoid (.g., genistein, kaempferol, 7-hydroxyflavone, and 3,7-dihydroxyflavone; trans-resveratrol, curcumin, catechins, and kaemferol-type flavonoids).

Cannabimimetics also may be derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; Essential Oils, for example: Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), and Ylang ylang (Cananga odorata(Lam.) var. genuine).

According to some embodiments, the therapeutic amount of the composition comprising the cannabinoid or cannabimimetic agent potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%. According to some embodiments, the therapeutic amount of the composition stabilizes the T cell immune repertoire. According to some embodiments, for each dose of the cannabinoid or the cannabimimetic, onset of potentiation of the immune response by increasing diversity occurs within 24 hours of dosing. According to some embodiments, the potentiated immune response comprises an enhanced T cell diversity, an enhanced B cell diversity, or both, compared to the subject before the administering. According to some embodiments, the potentiated immune response comprises an increased resistance to T cell exhaustion. According to some embodiments, the potentiated immune response improves clinical outcome in response to a pathogen. According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease. According to some embodiments, the potentiated immune response reduces appearance of disease. According to some embodiments, the potentiated immune response increases health span of the subject. According to some embodiments, the administering of the cannabinoid is topically. According to some embodiments, a therapeutic effect of the cannabinoid administered topically is non-psychoactive. According to some embodiments, the administering of the cannabinoid is orally.

According to some embodiments, the active constituent comprises N-acetylcysteine and a botanical ingredient, e.g., derived from Theobroma cacao, Echinaceae spp, plants of the Apiaceae family; e.g., Daucus carota L., kava. For example, the noni fruit (Morinda citrifolia) is used extensively throughout Polynesia and has been traditionally used for its health-promoting qualities. According to some embodiments, the botanical ingredient comprises a botanical extract. According to some embodiments, the cannabimimetic is a synthetic compound. According to some embodiments, the botanical ingredient comprises a cannabinoid. According to some embodiment, the botanical ingredient comprises a terpenoid (e.g., β-caryophyllene (trans isomer); the diterpene salvinorin A, pristimerin, euphol). According to some embodiments, the botanical ingredient comprises a cannibimimetic component or a derivative thereof. Exemplary cannabimimetics include, without limitation, a fatty acid derivative (e.g., an N-acetylethanolamine, such as N-linoleoylethanolamide, N-oleoylethanolamide, N-acylethanolamine (NAE) palmitoylethanolamide); N-alkylamides (e.g., dodeca-2E, 4E, 8Z, 10Z-tetraenoic acid isobutylamide; dodeca-2E, 4E-dienoic acid isobutylamide), falcarinol; or a flavonoid (.g., genistein, kaempferol, 7-hydroxyflavone, and 3,7-dihydroxyflavone; trans-resveratrol, curcumin, catechins, and kaemferol-type flavonoids).

Cannabimimetics also may be derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; Essential Oils, for example: Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), and Ylang ylang (Cananga odorata(Lam.) var. genuine).

According to some embodiments, the amount of NAC in a single dose ranges from 200 mg to 2400 mg, inclusive, i.e., 200 mg, 300 mg, 400 mg, 400 mg, 600 mg, 700 mg, 800 mg, 900 mg, 1000 mg, 1100 mg, 1200 mg, 1300 mg, 1400 1500 mg, 1600 mg, 1700 mg, 1800 mg, 1900 mg, 2000 mg, 2100 mg, 2200 mg, 2300 mg, 2400 mg, and the amount of a cannabinoid or cannabinomimetic in a single dose ranges from 1 mg to 50 mg THC, inclusive, i.e., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg 50 mg, and 1 mg to 600 mg CBD, inclusive, i.e., 1 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 28 mg 0, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, with ranges to accommodate different cannabinoid ratios (THC: CBD) over varying lengths of treatment time. Number of doses per day can also vary According to some embodiments, the therapeutic amount of the composition comprising N-acetylcysteine and the cannabinoid or cannabimimetic agent potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%. According to some embodiments, the therapeutic amount of the composition stabilizes the T cell immune repertoire. According to some embodiments, for each dose of the composition, onset of potentiation of the immune response by increasing diversity occurs within 24 hours of dosing. According to some embodiments, the therapeutic effect of the N-acetylcysteine and of the cannabinoid or cannabimimetic agent are complementary. According to some embodiments, the potentiated immune response comprises an enhanced T cell diversity, an enhanced B cell diversity, or both, compared to the subject before the administering. According to some embodiments, the potentiated immune response comprises an increased resistance to T cell exhaustion. According to some embodiments, the potentiated immune response improves clinical outcome in response to a pathogen. According to some embodiments, the pathogen is a microbe, e.g., bacteria, fungi, protozoa, viruses, or algae. According to some embodiments, the virus is a coronavirus, an influenza virus, or a human immunodeficiency virus. According to some embodiments, the potentiated immune response comprises an innate response, an adaptive response, or both. According to some embodiments, the potentiated immune response reduces a burden of disease. According to some embodiments, the potentiated immune response reduces appearance of disease. According to some embodiments, the potentiated immune response increases health span of the subject. According to some embodiments, the administering of the N-acetylcysteine component is orally and the administering of the cannabinoid or cannabimimetic agent is topically. According to some embodiments, a therapeutic effect of the cannabinoid or cannabimimetic agent administered topically is non-psychoactive. According to some embodiments, the administering of the cannabinoid or cannabimimetic agent is orally.

According to some embodiments, the subject in need is an aged person (defined as a person of greater than 60 years of age). According to some embodiments, the immune system of the aged person is compromised by one or more of prior illnesses, chronic illnesses, or the aging process. Exemplary preexisting chronic illnesses include, without limitation, diabetes, hypertension, chronic renal failure, chronic heart disease, pulmonary disease, and cancer. According to some embodiments, advancing age is a risk factor for a number of chronic diseases in humans. According to some embodiments, the aging process comprises age-related changes in dynamic biological, physiological, environmental, psychological, behavioral, and social processes. According to some embodiments, the age-related biological and physiological changes result in increased susceptibility to the viral infection.

2.4. Viral Assays

According to some embodiments, assaying for a virus may be carried out by several procedures including, but not limited to, immunoblot (Western blot), immunoprecipitation, and combined reverse transcription-polymerase chain reaction (RT-PCR) assay. According to some embodiments, these assays may be used as diagnostic procedures, to measure the reduction of the synthesis of a viral genome, viral mRNAs or their products that may present in homogenized tissue samples, nasopharyngeal washes, or secretions, obtained from infected animals or human subjects.

For example, during the life cycle of the influenza A virus (including H5N1), the viral genome RNA (vRNA) serves as a template for complementary RNA (cRNA) production, which also serves as the template for messenger RNA (mRNA) production. The influenza A genome, consisting of 8 separate RNA segments containing at least 10 open reading frames (ORFs), serves as template for both viral genome replication and subgenomic or gene-directed mRNA synthesis. For such RNA viruses, the synthesis of viral genomic RNA can be detected in RT-PCR assay methods using primers complementary to the sequences across the junctions between the ORFs. The result of this RT-PCR analysis will reflect the synthesis of the whole genomic RNA.

According to some embodiments, the methods further include assays wherein decrease or inhibition of viral replication in tissue culture is measured by quantitative real-time PCR (RTQ-PCR), Median Tissue Culture Infectious Dose (TCID50, meaning the concentration at which 50% of the cells are infected when a test tube or well plate upon which cells have been cultured is inoculated with a diluted solution of viral fluid), viral plaque assay, immunofluorescence assay, and immunohistochemistry, and the like, as known to a worker of skill in the field of virology. According to some embodiments, the methods further include assaying for the presence of virus, employing the TCID50 method to monitor the inhibition of viral replication in tissue culture, wherein the viral titer is measured by any kind of cell pathogenic endpoints, including by way of non-limiting example, cell fusion, cytopathic effect (CPE), and cell adsorption. According to some embodiments, the methods further include assays wherein viral replication in tested animals is measured by RTQ-PCR, pathology, immunohistochemistry, and re-isolation of virus.

2.5 Formulations

### 2.5-1 NAC Synthesis

NAC can be found in small quantities in plasma (˜2.8 μM), where it is present in reduced form, oxidized forms such as NAC-NAC (diacetyl cysteine), or bound to plasma proteins. Whilier et al., Redox Rep. 14:115-24 (2009). Because of low availability, NAC is usually synthesized instead of isolated from natural sources.

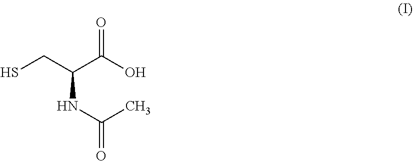

NAC (formula I) is obtained conventionally by monoacetylation of L-cysteine hydrochloride of formula (II):

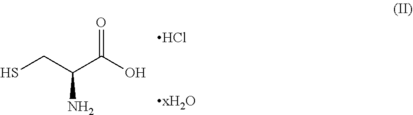

in a suitable aqueous-organic solvent. The nature of this solvent makes the yield of the reaction vary between 60 and 95%. Compound (II) can be obtained by reduction of L-cystine of formula (III):

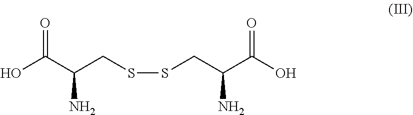 [SMILES: N[C@H](CSSC[C@H](N)C(=O)O)C(=O)O]

either using conventional reducing agents or using electrochemical reduction.

In this process, separation of (I) from the solution coming from acetylation of (II) implies handling solutions with very high salt contents in acetate that may influence the quality of the product, if its application is centered within the pharmaceutical field. On the other hand, in order to obtain (I) from (II) it is necessary that the latter product (II) is separated from product (III) used as a starting material in the synthesis thereof since traces of (III) could harm the following step of the process.

Another possibility of obtaining NAC (I) is by reduction of bis-acetyl-L-cystine of formula (IV):

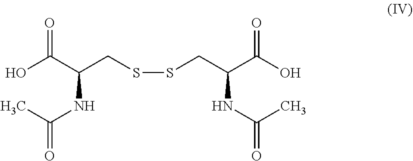 [SMILES: CC(=O)N[C@@H](CSSC[C@@H](NC(C)=O)C(=O)O)C(=O)O]

by means of using conventional reducing agents such as zinc. In turn, compound (IV) would be obtained previously by acetylation of (III).

Another possibility of obtaining NAC (I) is by the method disclosed in EP0905282. That method comprises a first phase of acetylation of the cystine of formula (III):

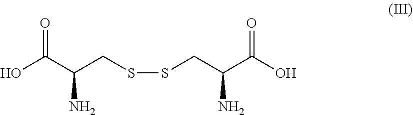 [SMILES: N[C@H](CSSC[C@H](N)C(=O)O)C(=O)O]

in an aqueous solution and with acetic anhydride. For example, (III) is dissolved in an aqueous solution of an alkali metal or alkaline earth metal hydroxide, preferably sodium hydroxide, with a pH higher than 7 and at a temperature of about 0° C.

After this acetylation step has ended, a reaction solution will be obtained containing the bis-acetyl-L-cystine (IV) produced, alkali metal or alkaline earth metal acetate (in some embodiments, sodium acetate), and water.

This solution is subjected to electrochemical treatment of desalination and reduction which can be carried out sequentially or simultaneously.

In a first alternative, the solution containing (IV) is first subjected to a desalination process by means of the use of conventional reverse or cascade electrodialysis, in order to obtain a solution that will have lost most of its salt content (alkaline or alkaline earth acetate). This solution is then subjected to an electrochemical reduction process. After said process has ended, a solution (I) with a low salt content, which can be treated to isolate the desired product (I), with a quality capable of meeting the requirements of U.S. Pharmacopoeia, is obtained.

In a second alternative, the solution containing (IV) is subjected to a simultaneous electrochemical desalination and reduction process with a suitable electrochemical reactor. A solution containing (I) with a low salt content which, as before, can be treated optimally to separate the desired product with the required quality, is obtained with it in a single electrochemical step.

NAC is also available commercially from numerous sources, including, but not limited to, Tractus (PubChem SID: 204364707, Purchasable Chemical: RTC-066653), Aurum Pharmatech LLC (PubChem SID: 184818743, Purchasable Chemical: M-5026), Pi Chemicals (PubChem SID: 322089934, Purchasable Chemical: PI-33258), Alfa Aesar (PubChem SID: 376198448, Purchasable Chemical: A15409), Yuhao Chemical (PubChem SID: 318239344, Purchasable Chemical: JZ1685), LabNetwork, a WuXi AppTec Company (PubChem SID: 346683127, Purchasable Chemical: LN00173956), Tokyo Chemical Industry (PubChem SID: 87562414, Purchasable Chemical: A0905), Sigma-Aldrich (PubChem SID: 329747210, Purchasable Chemical: 01-0870SAJ), abcr GmbH (PubChem SID: 316393992, Purchasable Chemical: AB113284), BLD Pharm (PubChem SID: 375441795, Purchasable Chemical: BD34319), LGC Standards (PubChem SID: 340515159, Purchasable Chemical: LGCFOR0024.00), VWR, Part of Avantor (PubChem SID: 384251663, Purchasable Chemical: 200001-434), MedChemexpress MCE (PubChem SID: 210281679, Purchasable Chemical: HY-B0215), Watanabe Chemical Ind. (PubChem SID: 386054937, Purchasable Chemical: K01943), Adooq BioScience (PubChem SID: 383470899, Purchasable Chemical: A10032), AA BLOCKS (PubChem SID: 374169152, Purchasable Chemical: AA003598), Founder Pharma (PubChem SID: 250196856, Purchasable Chemical: FD12357), AbaChemScene (PubChem SID: 210279356, Purchasable Chemical: CS-2160), BroadPharm (PubChem SID: 143503442, Purchasable Chemical: BP-12854), Acorn PharmaTech Product List (PubChem SID: 329992555, Purchasable Chemical: ACN-033112), Selleckchem (PubChem SID: 347912801, Purchasable Chemical: S1623), Hangzhou APIChem Technology (PubChem SID: 92714687, Purchasable Chemical: AC-16071), 3B Scientific (Wuhan) Corp (PubChem SID: 375135026, Purchasable Chemical: 3B1-01551), VladaChem (PubChem SID: 329731563, Purchasable Chemical: VL140024), Syntree (PubChem SID: 249828587, Purchasable Chemical: ST2408139), ChemTik (PubChem SID: 162630092, Purchasable Chemical: CTK2F2887), Acros Organics (PubChem SID: 376170630, Purchasable Chemical: AC160280010), Acadechem (PubChem SID: 321934656, Purchasable Chemical: ACDS-064582), Assembly Blocks Pvt. Ltd. (PubChem SID: 223380796, Purchasable Chemical: AB0013800), AK Scientific, Inc. (PubChem SID: 162178308, Purchasable Chemical: I630), eNovation Chemicals (PubChem SID: 319477565, Purchasable Chemical: D518730), ZINC (PubChem SID: 257031629, Purchasable Chemical: ZINC3589203), Sinfoo Biotech (PubChem SID: 404827453, Purchasable Chemical: S048925), Biosynth (PubChem SID: 49746643, Purchasable Chemical: A-1100), Life Chemicals (PubChem SID: 347815827, Purchasable Chemical: F1905-7178), Key Organics/BIONET (PubChem SID: 318123958, Purchasable Chemical: GS-3121), Chemenu Inc. (PubChem SID: 354292841, Purchasable Chemical: CM119375), AHH Chemical Co. (PubChem SID: 252400969, Purchasable Chemical: MB-01383), ApexBio Technology (PubChem SID: 163632623, Purchasable Chemical: A7131), labseeker (PubChem SID: 251888194, Purchasable Chemical: SC-09436), Chem-Space.com Database (PubChem SID: 318614581, Purchasable Chemical: CSC000753254), Glentham Life Sciences Ltd. (PubChem SID: 310265502, Purchasable Chemical: GM8803), ChemShuttle (PubChem SID: 322059189, Purchasable Chemical: 141416), CAPOT (PubChem SID: 152243565, Purchasable Chemical: 12963), Angene Chemical (PubChem SID: 222618519, Purchasable Chemical: AGN—PC-OODGOF), BerrChem (PubChem SID: 174478612, Purchasable Chemical: BR-46577), Norris Pharm (PubChem SID: 383227309, Purchasable Chemical: NSTH-D25131), ABBLIS Chemicals (PubChem SID: 117688592, Purchasable Chemical: AB1002786), TimTec (PubChem SID: 143857285, Purchasable Chemical: ST50824849), MuseChem (PubChem SID: 355038834, Purchasable Chemical: A000591), Hairui Chemical (PubChem SID: 375654657, Purchasable Chemical: HR137549), Lan Pharmatech (PubChem SID: 404887236, Purchasable Chemical: LQT-B38621), Finetech Industry Limited (PubChem SID: 164813631, Purchasable Chemical: FT-0629832), King Scientific (PubChem SID: 346399888, Purchasable Chemical: KS-0000026W), Aurora Fine Chemicals LLC (PubChem SID: 290066114, Purchasable Chemical: K02.100.638), EMD Millipore (PubChem SID: 163688703, Purchasable Chemical: 106425), OXCHEM CORPORATION (PubChem SID: 255395553, Purchasable Chemical: AX8034319), Debye Scientific Co. (PubChem SID: 202538550, Purchasable Chemical: DB-038288), Achemo Scientific Limited (PubChem SID: 254806107, Purchasable Chemical: AC-22398), AbovChem LLC (PubChem SID: 319554859, Purchasable Chemical: HY-B0215), Alichem (PubChem SID: 378043938, Purchasable Chemical: 471002829), Pure chemistry (PubChem SID: 253664191, Purchasable Chemical: 814796), OChem (PubChem SID: 341830217, Purchasable Chemical: 39), Enamine (PubChem SID: 131390322, Purchasable Chemical: EN300-72028), AKos Consulting & Solutions (PubChem SID: 151980428, Purchasable Chemical: AKOS015841009), MolPort (PubChem SID: 87628644, Purchasable Chemical: MolPort-000-150-826), Achemtek (PubChem SID: 381363284, Purchasable Chemical: 0101-000335), CSNpharm (PubChem SID: 384652085, Purchasable Chemical: CSN16436), Parchem (PubChem SID: 316603683, Purchasable Chemical: 337), Activate Scientific (PubChem SID: 340547010, Purchasable Chemical: AS32848), Oakwood Products (PubChem SID: 117648319, Purchasable Chemical: 003631), Anward (PubChem SID: 160801416, Purchasable Chemical: ANW-33915), Fisher Chemical (PubChem SID: 349997534, Purchasable Chemical: 0104925), AbMole Bioscience (PubChem SID: 348397075, Purchasable Chemical: M5385), Apexmol (PubChem SID: 136995853, Purchasable Chemical: AM20100502), BioCrick (PubChem SID: 382163183, Purchasable Chemical: BCC3716).

Since NAC contains sulfur, it may impart an unpleasant smell and/or taste if it is not encapsulated or if it is simply dissolved or suspended in water. Thus, compositions comprising NAC may comprise a combination of amino acid masking agents and NAC. The amino acid masking agents may contribute to the pleasant taste and smell of the NAC composition.

### 2.5-2 NAC Formulations

Solutions or suspensions of NAC used for parenteral, intra-dermal, subcutaneous, or topical application can include the following components: a sterile diluent such as water for injection, saline solution, fixed oils, polyethylene glycols, glycerine, propylene glycol, or other synthetic solvents; anti-bacterial agents such as benzyl alcohol or methyl parabens; anti oxidants such as ascorbic acid, BHA, BHT, or sodium bisulfite; chelating agents such as ethylenediaminetetraacetic acid; buffers such as acetates, citrates, or phosphates; and agents for the adjustment of tonicity such as sodium chloride or dextrose. U.S. Pat. No. 5,637,616.

NAC or salts thereof can also be applied in the form of a topical composition. The composition can be formulated in a variety of ways, for example, in a solid form such as a powder; a liquid form such as a solution or a suspension in an aqueous or oily medium; or a semi-liquid formulations such as a cream, jelly, paste, ointment, or salve. For example, NAC can be applied in the form of a solution, gel, ointment, cream, lotion, or foam, in a 1-100% by weight aqueous solution. U.S. Pat. No. 5,637,616. Vehicle or carrier for topical administration can be modified to enhance cutaneous absorption, enhance the reservoir effect, or minimize potential irritancy or neuropharmacological effects of the composition. Examples of penetration enhancers include N-methylpyrrolidine and SEPAs (1,3-dioxolanes). Arndt et al., The Pharmacology of Topical Therapy, Dermatology in General Medicine; Fitzpatrick et al., eds., 3d ed., McGraw Hill, Inc., New York, pp. 2532-2540 (1987). According to some embodiments, NAC can be administered as a solution by inhalation. The term “inhalation” as used herein refers to the act of drawing in a medicated vapor with the breath. According to some embodiments, delivery by inhalation is with an inhalation delivery device. The term “inhalation delivery device” as used herein refers to a machine/apparatus or component that produces small droplets or an aerosol from a liquid or dry powder aerosol formulation and is used for administration through the mouth in order to achieve pulmonary administration of a drug, e.g., in solution, powder, and the like. Examples of inhalation delivery device include, but are not limited to, a nebulizer, a metered-dose inhaler, and a dry powder inhaler (DPI).

NAC can be aerosolized in a variety of forms, including, without limitation, dry powder inhalants, metered dose inhalants, or liquid/liquid suspensions. The respirable particles may be liquid or solid. The particles may optionally contain other therapeutic ingredients such as amiloride, benzamil or phenamil, with the selected compound included in an amount effective to inhibit the reabsorption of water from airway mucous secretions, as described in U.S. Pat. No. 4,501,729, incorporated by reference in its entirety herein.

The particulate pharmaceutical composition may optionally be combined with a carrier to aid in dispersion or transport. A suitable carrier such as a sugar (i.e., lactose, sucrose, trehalose, mannitol) may be blended with the active compound or compounds in any suitable ratio (e.g., a 1 to 1 ratio by weight).

Particles comprising NAC according to the described invention should include particles of respirable size, that is, particles of a size sufficiently small to pass through the mouth or nose and larynx upon inhalation and into the bronchi and alveoli of the lungs. In general, particles ranging from about 1 to 10 microns, inclusive, in size (e.g., less than about 5 microns in size) are respirable. Particles of non-respirable size which are included in the aerosol tend to deposit in the throat and be swallowed, and the quantity of non-respirable particles in the aerosol may be minimized. For nasal administration (insufflation), a particle size in the range of 10-500 μM, inclusive, ensures retention in the nasal cavity.

Liquid pharmaceutical compositions for producing an aerosol may be prepared by combining the active compound with a suitable vehicle, such as sterile pyrogen free water. The hypertonic saline solutions used to carry out the present invention are sterile, pyrogen-free solutions, comprising from one to fifteen percent (by weight) of the physiologically acceptable salt, e.g., from three to seven percent by weight of the physiologically acceptable salt.

Aerosols of liquid particles comprising NAC may be produced by any suitable means, such as with a pressure-driven jet nebulizer or an ultrasonic nebulizer. See, e.g., U.S. Pat. No. 4,501,729, incorporated by reference in its entirety herein. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation.

Exemplary formulations for use in nebulizers consist of the active ingredient in a liquid carrier, the active ingredient comprising up to 40% w/w of the formulation, for example, less than 20% w/w. The carrier is typically water (and most preferably sterile, pyrogen-free water) or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate therapeutic aerosol generator. Aerosol generators for administering solid particulate therapeutics to a subject produce particles which are respirable and generate a volume of aerosol containing a predetermined metered dose of a therapeutic at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 to 200 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidant and suitable flavoring agents.

Numerous over-the-counter NAC formulations are available, e.g., Acetadote (injection, solution; 200 mg/ml; intravenous; Cumberland Pharmaceuticals Inc.); acetylcysteine (injection; 200 mg/ml; intravenous; Paddock Laboratories, LLC); acetylcysteine injection, acetylcysteine solution USP (solution; intravenous, oral, respiratory; Teligent Ou); acetylcysteine solution (solution; intravenous, oral, respiratory; Sandoz Canada Inc.); Cetylev (tablet, effervescent; 2.5 g/1, 500 mg/1; oral; Arbor Pharmaceuticals); Mucomyst (solution; 100 mg/ml, 200 mg/ml; intravenous, oral, respiratory; Bristol Myers Squibb); and Parvolex (liquid; intravenous, oral, respiratory; Mylan Pharmaceuticals). Generic NAC formulations are also available from, e.g., Fresenius Kabi USA, LLC (solution, injection; 100 mg/ml, 200 mg/ml; intravenous, oral, respiratory), Hospira, Inc. (solution; 100 mg/ml; oral, respiratory), American Regent, Inc. (inhalant; 100 mg/ml, 200 mg/ml; respiratory), Cadila Healthcare, Ltd. (injection, solution; 200 mg/ml; intravenous), Zydus Pharmaceuticals (USA) Inc. (injection, solution; 200 mg/ml; intravenous), Akorn (injection; 200 mg/ml; intravenous), and Cardinal Health (inhalant; 200 mg/ml; oral, respiratory). McEvoy, G. K. (ed.), American Hospital Formulary Service-Drug Information 2005. Bethesda, Md.; American Society of Health-System Pharmacists, Inc. 2005 (Plus Supplements).

Specific formulations are discussed in, e.g., Lee et al., Gastroenterology 137:856 64 (2011), and Gray et al., Am. J. Psychiatry 169:805-12 (2012). Lee et al. (2011) discloses use of 5% dextrose with NAC (Acetadote), with an initial loading dose of 150 mg/kg/hr of NAC over one hour, followed by 12.5 mg/kg/hour for 4 hours, then continuous infusions of 6.25 mg/kg NAC for the remaining 67 hours. Gray et al. in an eight-week double-blind randomized placebo-controlled trial, treatment-seeking cannabis-dependent adolescents (age 15-21, N=116) received 1,200 mg NAC. Similarly, Schmaal et al., Neuropsychopharmacology 37:2143-52 (2012), disclose use of 2,400 mg NAC based on previous studies showing beneficial effects of 1,200 and 2,400 mg/day NAC on treatment retention and drug use in cocaine and nicotine dependence.

### 2.5-3 Cannabis Strains and Extraction

Cannabis plant material can originate from Cannabis indica, Cannabis sativa, or Cannabis ruderalis or hybrids thereof. Known Cannabis strains include AC/DC, Afghan Goo, Atomic Northern Lights, Blackberry Kush, Blueberry, Blueberry Kush, Blueberry Muffin Top, Blueberry OG, Blue Diesel, Blue Dream, Buddha Passion, Cannatonic, Chocolate Kush, Fire OG, Jilly Bean, Gran Daddy Purple, Grape Blackberry Kush, Harle OG, Harle Tsu, Harlequin, Hope Springs, Infinite Euphoria, Long Valley Royal Kush, Medihaze, Pineapple Jack, Prize Kush, Sour Diesel, Sour Kush, Tahoe OG, Afgoo, Afghan Kush, Agent Orange, AK-47, Amnesia Haze, Atomic Jam, Atomic Northern Lights, Avidekel, BC Grapefruit, Belladonna, Berry White, Blackberry British Columbia, Blackberry Kush, Black Romulan, Black Queen, Blueberry Kush, Blueberry OG, Blue Dream, Blue Cheese, Blueberry Cheese, Blue Diesel, Blue Dream, Blue Jay Way, Blue Velvet, Boost, Bubba Kush, Bubble Gum, Buddha Passion, BW Cookies, Cadillac Purple, Canna Sue, CannaTsu, Casey Jones, Charlotte's Web, Cheese, Cheeze, Cherry AK, Cherry Cola, Cherry Pie, Chemdawg, Chem Scout, Chocolate Kush, Chocolope, Chiesel, Cinderella 99, Cotton Candy Kush, Critical Jack, Death Star, Diesel Cookies, Downtown Diesel, Double Diesel, Dream Kush, Durban Cookies, Durban Poison, Dutch Treat, Dr. Tod, Elektra, Exodus, Fern Dog, Fire OG, Frankenstein OG, G13, God's Gift, Gran Daddy Purps, Granddaddy Purple, Granny Durkel, Grape Ape, Grape Puff, Grapefruit Rom, Grapekush, Grape Blackberry Kush, Girl Scout Cookies, Green Crack, Green Goddess, Headband, Heady Kush, Harlequin, Hash Plant, Hindica, Hindu Kush, Hopesprings, Huckleberry, Hubba Bubba, Infinite Euphoria, Island Sweet Skunk, Jack Herer, Jamaican Lion, Jamaican Skunk, Jelly Bean, Jilly Bean, Kushage, LA Confidential, Larry OG, Lavender, Lemon Haze, Lemon Kush, Lemon Skunk, Liberty Haze, Lion Fire, Manawell, Mango, Mango Haze, Maplewreck, Master Kush, Maui Waui, Misty, Mr. Nice, Northern Lights, NYC Diesel, OG Afgani, OG Kush, Ol'Betsy, Orange Crush, Orange Kush, Phenom Phen, Pineapple Express, Pineapple Haze, Pineapple Jack, Pineapple Kush, Pineapple Thai, Platinum Cookies, Platinum Kush, Pomegranate Kush, Purps, Purple Diesel, Purple Goo, Purple Hash Plant, Purple Haze, Purple Jasmine, Purple Kush, Purple Nice, Purple Platinum, Purple Trainwreck, Purple Urkle, R4, Rain, Red Raspberry Kush, Romulan, Royal Cookies, Sage Diesel, Sensi Star, Sierra, Sierra Purple, Silver Diesel, Silver Dragon, Silver Haze, Skywalker, Skywalker OG, Snow Cap, Sour Boogie, Sour Diesel, Sour Kush, Sour OG, Sour Tsunami, Stinky Purple, Strawberry Cough, Sunset Sherbert, Super Lemon Haze, Super Silver Haze, Sunra, Sweetooth SFV, Tahoe OG Kush, Thin Mints, Tangerine Dream, Tora Bora, Trainwreck, Ultraviolet, Unicorn, Vanilla Kush, West Point Snow, White Erkle, White Rhino, White Russian, White Widow, and Wizard's Potion.

Cannabis plant material can include cannabis flowers, buds, trichomes, leaves, stems, portions therein or combinations thereof. Freezing the Cannabis plant material can preserve terpenes or other volatile molecules when preparing the Cannabis oil extract. In addition, freezing the Cannabis plant material and/or solvent can decrease the quantity of chlorophyll in the Cannabis oils (an unwanted byproduct of the process).

Published U.S. Patent Application No. 20160346339 discloses an overview of cannabinoid extraction. In general, the plant material and/or extraction solvent are held at a particular temperature for a period of time sufficient to ensure that the materials reach the temperature. One of skill in the art will appreciate that the length of cooling or freezing time will depend in part on factors such as the targeted freezing/cooling temperature and the quantity of materials used in the method, as well as the particular extraction solvent and cannabis strain. Accordingly, Cannabis plant material and/or extraction solvents are typically held for periods of time ranging from several minutes to several hours in length. For example, Cannabis plant material and/or extraction solvents can be held at a reduced temperature for anywhere from about 10 minutes to about 72 hours prior to extraction.

Typically, the materials used in the methods of the present invention are cooled to temperatures below ambient temperature (i.e., below about 25° C.) prior to and/or during the extraction step. For example, the cannabis plant material and/or the extraction solvent can be held at a temperature ranging from about −80° C. to about 20° C. The solvent can be a predominantly polar solvent, e.g., the solvent can be an alcohol such as ethanol. The solvent can also be a polar solvent derived from organic sources.

Many different organic solvents can be used for cannabinoid extraction. Examples of organic solvents that can be used include, but are not limited to, acetonitrile, methanol, isopropanol, 1-butanol, 2-butanol, dichloromethane, ethyl acetate, isopropyl acetate, isopropyl ether, methyl tert-butyl ether, diethyl ether, acetone, butane, hexane, heptane, and combinations thereof.

Extraction can also include eluting cannabinoids from Cannabis plant material with solvent to produce an eluate. Elution can include eluting cannabinoids from frozen Cannabis plant material frozen with frozen solvent representing an eluent. The Cannabis plant material can also be referred to as a marc. The eluate can also be referred to as a menstruum. Solvent (eluent) can be poured over the Cannabis plant material placed in the strainer and collecting the eluate or menstruum from this pouring step in the collection receptacle.

Eluate or menstruum collected from the pouring step can be poured over the same Cannabis plant material again to elute more of the cannabinoids from the Cannabis plant material. At this point, the eluate or menstruum produced by the repeated pours can be filtered to yield a first eluate. The eluate or menstruum produced by the repeated pours can be filtered by pouring through a mesh filter.

Leftover Cannabis plant material from the pouring steps can be used to produce a second eluate. Fresh portions of solvent can be poured over Cannabis plant material in a strainer to produce the second eluate. Alternatively, the Cannabis plant material can be removed from the strainer and placed into an open container.

Extraction solvent can be soaked with the Cannabis plant material before straining or the extraction solvent can be kept separate before straining. In instances where Cannabis plant material is soaked/macerated with extraction solvent, incubation time can range from less than about 1 minute to more than about 10 hours.

After soaking the Cannabis plant material in solvent, the entire contents of the open container can be poured through a strainer and then filtered to yield the second eluate. The second eluate can be collected in a glass or other container (e.g., a container made of high- or low-density polyethylene) having a lid or other closing mechanism. The second eluate can be subjected to solarization prior to further filtration as discussed below.

Solarization is a process that includes exposing the Cannabis extract to a light source to degrade any chlorophyll that has collected with the cannabinoids. The solarization process can be carried out for any amount of time suitable for degrading, or otherwise reducing, the chlorophyll in the extract. Typically, the incubation time will range from fewer than about 5 minutes to more than about 12 hours. The solarization time can depend on factors including, but not limited to, the strength of the light source used. The solarization time can also depend on the desired finished product.

Solarization involves exposing eluate to direct sunlight in order to solarize the eluate. Solarization can be accomplished using any source of light suitable for degrading chlorophyll. The light source can be, for example, the sun. Another source of light used can be non-natural light sources. Non-natural light sources can include those that emit a full light spectrum to mimic natural light, or those that only provide specific wavelengths. Non-natural light sources can also include those that vary spectral outputs and temperatures as time passes, or those that keep a constant spectral output and temperature. The solarization step can be conducted at any temperature suitable for degrading, or otherwise reducing, the chlorophyll in the extract. Typically, solarization will be conducted at a temperature ranging from about 80° C. to about 30° C.

Solarization allows oil producers to elute more of the cannabinoids from the same batch of the Cannabis plant material through the process described above. More specifically, the solarization step allows oil producers to make the Cannabis oil extract from the second eluate without leaving undesirable amounts of chlorophyll into the final product.

Generally, after the solarization step, the eluate is cooled to temperatures below ambient temperature (i.e., below about 25° C.). For example, the eluate can be held at a temperature ranging from about −80° C. to about 20° C. The length of cooling time will depend in part on factors such as the targeted freezing/cooling temperature and the quantity of materials used in the methods. Accordingly, the eluate is typically held for periods of time ranging from several minutes to several hours in length.

The second eluate can be stored for about 24 to about 48 hours at a temperature between about 0. ° C. and about −20° C. After this freezing step, the second eluate can undergo further filtration as discussed below.

Eluate, including the first eluate, the second eluate, or a combination thereof, can further be filtered with a filter to produce a filtrate.

The solvent from the filtrate can further be evaporated to produce a distillate. The filtrate can be distilled or evaporated for any length of time, depending on the desired concentration of distillate. For example, the filtrate can be distilled or evaporated for anytime ranging from about 30 minutes to about 10 hours or more.

After evaporating the solvent from the filtrate, the distillate can be optionally heated above room temperature under controlled conditions for an additional period of time. The distillate, after evaporation and optional heating, is transferred to an appropriate heating flask.

After distillation and optional heating, the distillate can be optionally filtered through a solid-phase filter medium. Examples of suitable solid-phase filter media include, but are not limited to, silica gel, activated charcoal, activated carbon, diatomaceous earth (Celite), and ion-exchange resins. The distillate can be homogenized or otherwise combined with a suitable solvent prior to the optional filtration step. The homogenized distillate can then be added to a portion of silica gel that has been conditioned (pre-run) in a suitable filter apparatus with the same solvent as added to the distillate. Once the homogenized distillate is fully absorbed on the silica, additional solvent can be added on top of the settled silica. During the silica gel filtration step, the homogenized distillate and added solvent can be pulled through the filter apparatus using a light vacuum or pushed through the filter apparatus using positive pressure applied from above. Alternatively, the homogenized distillate can proceed through the apparatus via gravity filtration. The filtrate can be collected in an appropriate flask prior to removal of solvent via evaporation, as described above.

The distillate can further be dehydrated or purged to further remove any further traces of solvent. In doing so, the dehydration produces an extract. Dehydration can be achieved using any known means in the art including the use of a food dehydrator, evaporator, or vacuum pump. In general, purging/dehydration is conducted under conditions sufficient to remove residual solvent from the Cannabis oil extract, i.e., any solvent (e.g., ethanol) used during the extraction process that remains in the extract after the elution, solarization, filtration, and evaporation steps. The removal of residual solvent can be monitored, for example, by conducting the purge/dehydration step until the weight of the extract stops decreasing (indicating that all volatile solvent has been removed).

During the purge/dehydration step, the distillate may be optionally heated to increase the efficiency of the solvent purge. The temperature used for purging/dehydration can be any temperature at or above ambient conditions. For example, heating during the purge/dehydration step can range from about 20 20° C. to about 200° C. or more.

The time of dehydration required to remove the remaining solvent will depend on the pressure and temperature of the purge/dehydration step as well as the solvent that is being removed. Typically, the time of the purge step will range from anywhere between about one 1 and about 5 days.

After obtaining the extract, the composition of the extract can be determined by a variety of the methods. For example, a portion of the extract can be analyzed by methods including, but not limited to, liquid chromatography/mass spectrometry (LC-MS), gas chromatography/mass spectrometry (GC-MS), and proton nuclear magnetic resonance spectroscopy (¹H-NMR). In addition, the composition of the extract can be organoleptically tested to ensure consistency in taste, smell, texture, coloration, or a combination thereof.

The two most common methods for cannabinoid analysis are GC-flame ionization detector (FID) and HPLC-UV, with HPLC being best for cannabinoid analysis of the native composition of a Cannabis plant. Giese et al. (2015). GC is useful for analysis of small volatile organics such as terpenoids. Additionally, the large linear range of the FID makes it possible to cover the wide range of terpene concentrations (approximately 0.01 to 1.5%) with a single injection. Giese et al. (2015). When analyzing cannabinoid acids, the cannabinoid acids must be derivatized (e.g., by silylation or methylation). Total cannabinoid content, i.e., the number of neutral cannabinoids plus the neutral cannabinoids formed by decarboxylation of the acidic cannabinoids, can be determined with GC analysis without derivatization. Brenneisen, Chemistry and analysis of phytocannabinoids and other Cannabis constituents, in Forensic Science and Medicine: Marijuana and the Cannabinoids, Chp. 2 (E1 Sohly ed., Humana Press, Inc., Totowa, N.J., 2007).

Thin layer chromatography (TLC) is also used to determine cannabinoid content. Raharjo et al., Phytochem. Analysis 15:79-94 (2004). Despite the high sensitivity and selectivity of TLC, it is uncommon in cannabinoid quantification because of an extra step as compared to GC or HPLC. TLC is widely used, however, for the detection of cannabinoid metabolites. Raharjo et al. (2004).A more recent analysis method is high throughput homogenization (HTH) of typical inflorescences as a single sample preparation method for the analysis of both terpenoids and cannabinoids. Giese et al. (2015). High throughput homogenizers have been used for homogenizing microorganisms, plant tissues, and animal tissues and have been successful in relieving bottlenecks in several high throughput screening strategies. The Giese et al. (2015) single sample extraction procedure reduces handling and solvent usage and provides an extract that can be analyzed for both terpenes and cannabinoids by GC-FID and HPLC-DAD, respectively.

### 2.5-4 Cannabinoid Formulations

Spray drying and/or freeze drying cannabinoids in a polysaccharide, most notably inulin, can be used to effectively deliver cannabinoids for pharmaceutical uses. For example, spray drying can be performed using a Buchi 190 mini spray dryer. After spray drying, the formed powder is collected and flushed with nitrogen for about 15 minutes. Published U.S. Patent Application No. 2003/0229027.

Freeze drying can performed using a Christ model Alpha 2-4 lyophilizer (Salm en Kipp, Breukelen, The Netherlands). In a typical experiment, 20 mL glass vials are charged with 2-5 mL solution. The solutions are then frozen in liquid nitrogen and subsequently lyophilized at shelf temperature of −30° C., a condenser temperature of −53° C., and a pressure of 0.220 mbar for 1-3 days. Subsequently, the shelf temperature is gradually raised to 20° C. and pressure is gradually decreased to 0.05 mBar during 6 hours. Freeze dried samples can be stored in a vacuum desiccator for at least one day. Published U.S. Patent Application No. 2003/0229027.

Extracted cannabinoids may be combined with one or more carrier oils, such as medium chain triglyceride (MCT) oil, long chain triglyceride (LCT) oil, vegetable oil, canola oil, olive oil, sunflower oil, coconut oil (including fractionated coconut oil), hemp oil, palm oils, and/or other oils suitable for consumption. The addition of carrier oils improves solubility of the cannabinoid compounds and/or facilitates homogeneous dispersion of the cannabinoid compound(s) into hydrophilic components or water-soluble matrices formed by water and water soluble agent. Further, carrier oil(s) may be useful to increase the stability of the oil-in-water emulsion, e.g., including for higher levels of cannabinoids. Coconut oil is noted for a high saturated, MCT content. Hemp oil comprises about 80% essential fatty acids and is obtained from hemp seeds, which come from a variety of the Cannabis sativa plant that does not contain a high amount of THC. U.S. Pat. No. 8,809,261.

In particular regards to CBD, this cannabinoid shows limited oral bioavailability due to its lipophilicity and extensive first-pass metabolism. CBD is also known for its high intra- and inter-subject absorption variability in humans. To overcome these limitations, a self-emulsifying drug delivery system based on VESISORB® formulation technology can be used to improve CBD oral bioavailability. Knaub et al., Molecules 24:2967 (2019).

2.6 Compositions

The formulations of the described invention may be presented conveniently in unit dosage form and may be prepared by any of the methods well known in the art of pharmacy. All methods include the step of bringing into association a therapeutic agent(s), or a pharmaceutically acceptable salt or solvate thereof (“active compound”) with the carrier which constitutes one or more accessory agents. In general, the formulations are prepared by uniformly and ultimately bringing into association the active agent with liquid carriers or finely divided solid carriers or both and then, if necessary, shaping the product into the desired formulation.

According to some embodiments, the carrier is a controlled release carrier. The term “controlled release” is intended to refer to any drug-containing formulation in which the manner and profile of drug release from the formulation are controlled. This includes immediate as well as non-immediate release formulations, with non-immediate release formulations including, but not limited to, sustained release and delayed release formulations. According to some embodiments, the controlled release of the pharmaceutical composition is mediated by changes in temperature. According to some other embodiments, the controlled release of the pharmaceutical composition is mediated by changes in pH.

The term “polymer” refers to a large molecule, or macromolecule, composed of many repeated subunits. The term “monomer” refers to a molecule that may bind chemically to other molecules to form a polymer. The term “copolymer” as used herein refers to a polymer derived from more than one species of monomer. The term “release” and its various grammatical forms, refers to dissolution of an active constituent and diffusion of the dissolved or solubilized species by a combination of the following processes: (1) hydration of a matrix, (2) diffusion of a solution into the matrix; (3) dissolution of the active; and (4) diffusion of the dissolved active out of the matrix. The polymer is selected based on the period of time over which release is desired. According to some embodiments, the polymer forms a matrix (hereinafter the polymer matrix) with the therapeutic agent so as to obtain a desired release pattern of the active ingredient. According to some embodiments, the therapeutic agent is impregnated in or the polymer matrix. According to some embodiments, the polymer matrix encapsulates the therapeutic agent. According to some embodiments, the polymer matrix is homogeneous and contains a single polymer. According to some embodiments, the polymer matrix contains a first polymer and a second polymer. According to some embodiments, more than two polymers can be present in a blend, for example, 3, 4, 5, or more polymers can be present. According to some embodiments, the polymer matrix comprises cross-linked or intertwined polymer chains.

Exemplary biocompatible biodegradable polymers include, without limitation, a poly(lactide); a poly(glycolide); a poly(lactide-co-glycolide); a poly(lactic acid); a poly(glycolic acid); a poly(lactic acid-co-glycolic acid); a poly(caprolactone); a poly(orthoester); a polyanhydride; a poly(phosphazene); a polyhydroxyalkanoate; a poly(hydroxybutyrate); a poly(hydroxybutyrate) synthetically derived; a poly(hydroxybutyrate) biologically derived; a polyester synthetically derived; a polyester biologically derived; a poly(lactide-co-caprolactone); a poly(lactide-co-glycolide-co-caprolactone); a polycarbonate; a tyrosine polycarbonate; a polyamide (including synthetic and natural polyamides, polypeptides, poly(amino acids) and the like); a polyesteramide; a polyester; a poly(dioxanone); a poly(alkylene alkylate); a polyether (such as polyethylene glycol, PEG, and polyethylene oxide, PEO); polyvinyl pyrrolidone or PVP; a polyurethane; a polyetherester; a polyacetal; a polycyanoacrylate; a poly(oxyethylene)/poly(oxypropylene) copolymer; a polyacetal, a polyketal; a polyphosphate; a (phosphorous-containing) polymer; a polyphosphoester; a polyhydroxyvalerate; a polyalkylene oxalate; a polyalkylene succinate; and a poly(maleic acid).

Exemplary biopolymers or modified biopolymers include chitin, chitosan, modified chitosan, among other biocompatible polysaccharides; or biocompatible copolymers (including block copolymers or random copolymers) herein; or combinations or mixtures or admixtures of any polymers herein.

Exemplary copolymers include block copolymers containing blocks of hydrophilic or water-soluble polymers (such as polyethylene glycol, PEG, or polyvinyl pyrrolidone, PVP) with blocks of other biocompatible or biodegradable polymers (for example, poly(lactide) or poly(lactide-co-glycolide or polycaprolcatone or combinations thereof).

Exemplary long-acting formulations prepared from copolymers include those comprised of the monomers of lactide (including L-lactide, D-lactide, and combinations thereof) or hydroxybutyrates or caprolactone or combinations thereof; long-acting formulations prepared from copolymers that are comprised of the monomers of DL-lactide, glycolide, hydroxybutyrate, and caprolactone and long-acting formulations prepared from copolymers comprised of the monomers of DL-lactide or glycolide or caprolactone or hydroxybutyrates or combinations thereof. Additionally, long-acting formulations may be prepared from admixtures containing the aforementioned copolymers (comprised of DL-lactide or glycolide or caprolactone or hydroxybutyrates or combinations therein) along with other biodegradable polymers including poly(DL-lactide-co-glycolide) or poly(DL-lactide) or PHA's, among others. Long-acting formulations also may be prepared from block copolymers comprising blocks of either hydrophobic or hydrophilic biocompatible polymers or biopolymers or biodegradable polymers such as polyethers (including polyethylene glycol, PEG; polyethylene oxide, PEO; polypropylene oxide, PPO and block copolymers comprised of combinations thereof) or polyvinyl pyrrolidone (PVP), polysaccharides, conjugated polysaccharides, modified polysaccharides, such as fatty acid conjugated polysaccharides, polylactides, polyesters, among others.

According to some embodiments, the polymer forms a matrix (hereinafter the polymer matrix) with the therapeutic agent so as to obtain a desired release pattern of the active ingredient. According to some embodiments, the therapeutic agent is impregnated in or the polymer matrix. According to some embodiments, the polymer matrix encapsulates the therapeutic agent. According to some embodiments, the polymer matrix is homogeneous and contains a single polymer. According to some embodiments, the polymer matrix contains a first polymer and a second polymer. According to some embodiments, more than two polymers can be present in a blend, for example, 3, 4, 5, or more polymers can be present. According to some embodiments, the polymer matrix comprises cross-linked or intertwined polymer chains.

Exemplary naturally-occurring biopolymers include, but are not limited to, protein polymers, collagen, polysaccharides, and photopolymerizable compounds. Exemplary protein polymers synthesized from self-assembling protein polymers include, for example, silk fibroin, elastin, collagen, and combinations thereof. Exemplary naturally-occurring polysaccharides include, but are not limited to, chitin and its derivatives, hyaluronic acid, dextran and cellulosics (which generally are not biodegradable without modification), and sucrose acetate isobutyrate (SAIB).

According to some embodiments, the carrier is a delayed release carrier.

According to some embodiment, the delayed release carrier comprises a biodegradable polymer. According to another embodiment, the biodegradable polymer is a synthetic polymer. According to another embodiment, the biodegradable polymer is a naturally occurring polymer.

According to some embodiments, the carrier is a sustained release carrier.

According to another embodiment, the sustained-release carrier comprises a biodegradable polymer. According to another embodiment, the biodegradable polymer is a synthetic polymer. According to another embodiment, the biodegradable polymer is a naturally occurring polymer.

According to some embodiments, the carrier is a short-term release carrier. The term “short-term” release, as used herein, means that an implant is constructed and arranged to deliver therapeutic levels of the active ingredient for about 1, 2, 3, 4, 5, 6, 7, 8, 9, 10, 11, 12, 13, 14, 15, 16, 17, 18, 19, 20, 21, 22, or 23 hours. According to some other embodiments, the short term release carrier delivers therapeutic levels of the active ingredient for about 1, 2, 3, or 4 days

According to some embodiments, the carrier is a long-term release carrier.

According to another embodiment, the long-term-release carrier comprises a biodegradable polymer. According to another embodiment, the biodegradable polymer is a synthetic polymer.

According to some embodiments, the carrier comprises particles. The term “particles” as used herein refers to refers to an extremely small constituent (e.g., nanoparticles, microparticles, or in some instances larger) in or on which is contained the composition as described herein.

The compositions also may contain appropriate adjuvants, including, without limitation, preservative agents, wetting agents, emulsifying agents, and dispersing agents.

Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like.

In most clinical situations, drugs are administered in a series of repetitive doses or as a continuous infusion to maintain a. steady-slate concentration of drug associated with the therapeutic window. To maintain the chosen steady-state or target concentration (“maintenance dose”), the rate-of administration is adjusted such that the rate of input equals the rate of loss. If the clinician chooses the desired concentration of an active in plasma and knows the clearance and bioavailability for that drug in a particular patient, the appropriate dose and dosing interval can be calculated.

The “loading dose” is one or a series of doses that may be given at the onset of therapy with the aim of achieving the target concentration rapidly. A loading dose may be desirable if the time required to attain steady state by the administration of drug at a constant rate is long relative to the temporal demands of the condition being treated.

The precise dose of-the active to be employed in a formulation of the described invention will depend on the route of administration and the seriousness of the disease or disorder, and should be decided according to the judgment of the practitioner and each patient's circumstances. See, for example, Goodman and Gilman's The Pharmacological Basis of Therapeutics (2001); The Physician's Desk Reference, Medical Economics Company, Inc. Oradell, N J, 1995; and to Drug Facts and Comparisons, Facts and Comparisons, Inc., St. Louis. 1993.

According to some embodiments, the therapeutic amount of NAC comprises at least 200 mg but less than 20,000 mg of N-acetylcysteine, inclusive, i.e., 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, 11000 mg, 11100 mg, 11200 mg, 11300 mg, 11400 mg, 11500 mg, 11600 mg, 11700 mg, 11800 mg, 11900 mg, 12000 mg, 12100 mg, 12200 mg, 12300 mg, 12400 mg, 12500 mg, 12600 mg, 12700 mg, 12800 mg, 12900 mg, 13000 mg, 13100 mg, 13200 mg, 13300 mg, 13400 mg, 13500 mg, 13600 mg, 13700 mg, 13800 mg, 13900 mg, 14000 mg, 14100 mg, 14200 mg, 14300 mg, 14400 mg, 14500 mg, 14600 mg, 14700 mg, 14800 mg, 14900 mg, 15000 mg, 15100 mg, 15200 mg, 15300 mg, 15400 mg, 15500 mg, 15600 mg, 15700 mg, 15800 mg, 15900 mg, 16000 mg, 16100 mg, 16200 mg, 16300 mg, 16400 mg, 16500 mg, 16600 mg, 16700 mg, 16800 mg, 16900 mg, 17000 mg, 17100 mg, 17200 mg, 17300 mg, 17400 mg, 17500 mg, 17600 mg, 17700 mg, 17800 mg, 17900 mg, 18000 mg, 18100 mg, 18200 mg, 18300 mg, 18400 mg, 18500 mg, 18600 mg, 18700 mg, 18800 mg, 18900 mg, 19000 mg, 19100 mg, 19200 mg, 19300 mg, 10400 mg, 19500 mg, 19600 mg, 19700 mg, 19800 mg, 19900 mg, or 20000 mg.

According to some embodiments, the therapeutic amount of NAC ranges from about 900 mg to about 2,700 mg, inclusive, of N-acetylcysteine, i.e., 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, or 2700 mg,

Patients on therapy known to deplete cysteine/glutathione or produce oxidative stress [see, e.g., Ghezzi, P. et al., in Frye, R. E., M. Berk (Eds), in The Therapeutic Use of N-acetylcysteine (NAC) in Medicine, doi.org/10.1007/978-10-5311-5_20], the contents of which are incorporated herein by reference] may benefit from higher amounts of NAC.

According to some embodiments, the therapeutic amount of a cannabinoid or a cannabilnimetic ranges from [1 mg 50 mg for THC, inclusive, 1 mg-600 mg CBD, inclusive or a ratio of 20:1 (CBD:THC), or for cannabimimetics, an equivalent that provides the same or similar therapeutic effect.

According to some embodiments, the time frame for a therapeutic, prophylactic or potentiating effect compared to a control is within at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 clays, at least 16 clays, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days or at least 30 days of administration.

### 2.6-1 Oral Compositions

According to some embodiments, a composition comprising an active constituent may be in a form suitable for oral use, for example, as tablets, troches, lozenges, aqueous or oily suspensions, dispersible powders or granules, emulsions, hard or soft capsules or syrups or elixirs. As used herein, the terms “oral” or “orally” refer to the introduction into the body by mouth whereby absorption occurs in one or more of the following areas of the body: the mouth, stomach, small intestine, lungs (also specifically referred to as inhalation), and the small blood vessels under the tongue (also specifically referred to as sublingually). Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient(s) in admixture with non-toxic pharmaceutically-acceptable excipients which are suitable for the manufacture of tablets. These excipients may be. for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate.-stearic acid or talc. The tablets may be uncoated or they may be coated by known techniques, for example, to delay disintegration and absorption in the gastrointestinal tract and thereby provide a sustained action over a longer period, to protect the composition from oxidation or photodegradation; or for controlled release. For example, a time delay material such as glyceryl monostearate or glyceryl “distearate may be employed.

Compositions of the described invention also may be formulated for oral use as hard gelatin capsules, where the active ingredient(s) is (are) mixed with an inert solid diluent, for example, calcium carbonate., calcium phosphate or kaolin, or soft gelatin capsules wherein the active ingredient(s) is (are) mixed with water or an oil medium, for example, peanut oil, liquid paraffin, or olive oil.

The compositions of the described invention may be formulated as aqueous suspensions wherein the active ingredient(s) is (are) in admixture with excipients suitable for the manufacture of aqueous suspensions. Such excipients arc suspending agents, for example, sodium carboxymethylcellulose, methylcellulose, hydroxy-propylmethylcellulose, sodium alginate, polyvinylpyrrolidone, gum tragacanth. and gum acacia; dispersing or wetting agents may be a naturally-occurring phosphatide such as lecithin., or condensation products of an alkylene oxide with fatty acids, for example, polyoxyethylene stearate, or condensation products of ethylene oxide with long chain aliphatic alcohols, for example, heptadecaethylene octanol, or condensation products of ethylene oxide with partial esters derived from fatty acids and a hexitol such as polyoxyethylene sorbitol monooleate. or condensation products of ethylene oxide with partial esters derived from fatty acids and hexitol anhydrides, for example polyethylene sorbitan monooleate. The aqueous suspensions also may contain one or more coloring agents, one or more flavoring agents, and one or more sweetening agents, such as sucrose or saccharin.

Compositions of the described invention may be formulated as oily suspensions by suspending the active ingredient in a vegetable oil, for example arachis oil. olive oil. sesame oil or coconut oil, or in a mineral oil. such as liquid paraffin. The oily suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Compositions of the described invention may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, for example, sweetening, flavoring and coloring agents also may be present.

The compositions of the invention also may be in the form of an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that arc basically non-aqueous, for example, anionic and cationic surfactants of the non-aqueous immiscible system glycerin and olive oil. Thus, the compositions of the invention may be in the form of an oil-in-water emulsion. The oily phase may be a vegetable oil, for example, olive oil or arachis oil, or a mineral oil. for example a liquid paraffin, or a mixture thereof. Suitable emulsifying agents may be naturally-occurring gums, for example, gum acacia or gum tragacanth, naturally-occurring phosphatides, for example soy bean., lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

The compositions of the invention also may be formulated as syrups and elixirs.

Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

For buccal administration, the compositions of the described invention may take the form of tablets or lozenges formulated in a conventional manner.

There are three general methods of tablet preparation: the wet-granulation method: the dry-granulation method; and direct compression. The method of preparation and the added ingredients arc selected to give the tablet formulation the desirable physical characteristics allowing the rapid compression of tablets. After compression, the tablets must have a number of additional attributes such as appearance, hardness, disintegration ability, appropriate dissolution characteristics, and uniformity, which also are influenced both by the method of preparation and by the added materials present in the formulation.

According to some embodiments, the tablet is a compressed tablet (CT).

Compressed tablets are solid dosage forms formed with pressure and contain no special coating. Generally, they are made from powdered, crystalline, or granular materials, alone or in combination with binders, disintegrants, controlled-release polymers, lubricants, diluents and colorants.

According to some embodiments, the tablet is a sugar-coated tablet. These are compressed tablets containing a sugar coating. Such coatings may be colored and are beneficial in covering up drug substances possessing objectionable tastes or odors and in protecting materials sensitive to oxidation.

According to some embodiments, the tablet is a film-coated tablet. These compressed tablets are covered with a thin layer or film of a water-soluble material. Numerous polymeric substances with film-forming properties known in the art may be used.

According to some embodiments, the tablet is an enteric-coated tablet. These compressed tablets are coated with substances that resist solution in gastric fluid but disintegrate in the intestine.

According to some embodiments, the tablet is a multiple compressed tablet. These tablets are made by more than one compression cycle. Layered tablets are prepared by compressing additional tablet granulation on a previously compressed granulation. The operation may be repeated to produce multilayered tablets of two or three layers. Press-coated tablets (dry-coated) are prepared by feeding previously compressed tablets into a special tableting machine and compressing another granulation layer around the preformed tablets.

According to some embodiments, the tablet is a controlled-release tablet.

Compressed tablets can be formulated to release the drug slowly over a prolonged period of time. Hence, these dosage forms have been referred to as prolonged-release or sustained-release dosage forms.

According to another embodiment, the tablet is a tablet for solution. These compressed tablets may be used to prepare solutions or to impart given characteristics to solutions.

According to some such embodiments, the tablet is an effervescent tablet. In addition to the drug, these tablets contain sodium bicarbonate and an organic acid such as tartaric acid or citric acid. In the presence of water, these additives react, liberating carbon dioxide that acts as a disintegrator and produce effervescence.

According to another embodiment, the tablet is a buccal and or sublingual tablet. Generally, these arc small, flat, oval tablets intended for buccal administration that by inserting into the buccal pouch may dissolve or erode slowly.

According to another embodiment, the tablet is a molded tablet or tablet triturate.

According to some embodiments, the tablet comprises a compressed core comprising at least one component of the described formulation; and a membrane forming composition. Formulations utilizing membrane forming compositions are known to those of skill in the art (see. for example. Remington's Pharmaceutical Sciences, 20th Ed., 2000). Such membrane forming compositions may include, for example, a polymer, such as, but not limited to, cellulose ester, cellulose ether, and cellulose ester-ether polymers, an amphiphilic triblock copolymer surfactant, such as ethylene oxide-propylene oxide-ethylene oxide, and a solvent, such as acetone, which forms a membrane over the core. The compressed core may contain a bi-layer core including a drug layer and a push layer.

According to some embodiments, the unit dose for oral administration is individually wrapped to avoid oxidation.

### 2.6-2 Topical Compositions

According to some embodiments, the active constituent of the described invention can be administered topically. the composition may be applied by pouring, dropping, or spraying, if a liquid; rubbing on, if an ointment, lotion, cream, salve, gel, or the like; dusting, if a powder; spraying, if a liquid or aerosol composition; or by any other appropriate means.

Substances are applied to the skin to elicit one or more of four general effects: an effect on the skin surface, an effect within the stratum corneum; an effect requiring penetration into the epidermis and dermis; or a systemic effect resulting from delivery of sufficient amounts of a given substance through the epidermis and the dermis to the vasculature to produce therapeutic systemic concentrations. One example of an effect on the skin surface is formation of a film. Film formation may be protective (e.g., sunscreen) and/or occlusive (e.g., to provide a moisturizing effect by diminishing loss of moisture from the skin surface). One example of an effect within the stratum corneum is skin moisturization; which may involve the hydration of dry outer cells by surface films or the intercalation of water in the lipid-rich intercellular laminae; the stratum corneum also may serve as a reservoir phase or depot wherein topically applied substances accumulate due to partitioning into, or binding with, skin components.

It generally is recognized that short-term penetration occurs through the hair follicles and the sebaceous apparatus of the skin, while long term penetration occurs across cells. Penetration of a substance into the viable epidermis and dermis may be difficult to achieve, but once it has occurred, the continued diffusion of the substance into the dermis is likely to result in its transfer into the microcirculation of the dermis and then into the general circulation. It is possible, however, to formulate delivery systems that provide substantial localized delivery.

Percutaneous absorption is the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug include the establishment of a concentration gradient, which provides a driving force for drug movement across the skin, the release of drug from the vehicle into the skin-partition coefficient and drug diffusion across the layers of the skin-diffusion coefficient. The relationship of these factors to one another is summarized by the following equation:

J=Cᵥₑₕ×Kₘ·D/x [Formula V]

where J=rate of absorption; Cᵥₑₕ=concentration of drug in vehicle; Kₘ=partition coefficient; and D=diffusion coefficient.

The many factors that affect the rate of percutaneous absorption of a substance include, without limitation, the following: (i) Concentration. The more concentrated the substance, the greater the absorption rate, (ii) Size of skin surface area. The wider the contact area of the skin to which the substance is applied, the greater the absorption rate, (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body, (iv) Hydration. Hydration (meaning increasing the water content of the skin) causes the stratum corneum to swell which increases permeability, (v) Skin temperature. Increased skin temperature increases permeability. (vi) Composition. The composition of the compound and of the vehicle also determines the absorbency of a substance.

Most substances applied topically are incorporated into bases or vehicles. The vehicle chosen for a topical application will greatly influence absorption, and may itself have a beneficial effect on the skin. Factors that determine the choice of vehicle and the transfer rate across the skin are the substance's partition coefficient, molecular weight and water solubility. The protein portion of the stratum corneum is most permeable to water soluble substances and the lipid portion of the stratum corneum is most permeable to lipid soluble substances. It follows that substances having both lipid and aqueous solubility may traverse the stratum corneum more readily. (See Dermal Exposure Assessment: Principles and Applications, EPA/600/8-91/01 1 b, January 1992, Interim Report—Exposure Assessment Group, Office of Health and Environmental Assessment, U.S. Environmental Protection Agency, Washington, D.C. 20460).

Formulations for topical application can take the compositional form of a liquid, a semisolid dosage form (e.g., a paste, a salve, a cream, a lotion, a powder, an ointment or a gel) or a patch.

According to some embodiments, the composition of the described invention is characterized by controlled release of locally sustained levels of a minimum effective concentration (MEC) of an active constituent.

The intensity of effect of a drug (y-axis) can be plotted as a function of the dose of drug administered (X-axis). Goodman & Gilman's The Pharmacological Basis of Therapeutics, Ed. Joel G. Hardman, Lee E. Limbird, Eds., 10th Ed., McGraw Hill, New York (2001), p. 25, 50). These plots are referred to as dose-effect curves. Such a curve can be resolved into simpler curves for each of its components. These concentration-effect relationships can be viewed as having four characteristic variables: potency, slope, maximal efficacy, and individual variation.

The location of the dose-effect curve along the concentration axis is an expression of the potency of a drug. Id. If the drug is to be administered by transdermal absorption, a highly potent drug is required, since the capacity of the skin to absorb drugs is limited.

The slope of the dose-effect curve reflects the mechanism of action of a drug. The steepness of the curve dictates the range of doses useful for achieving a clinical effect.

Maximal or clinical efficacy refers to the maximal effect that can be produced by a drug. Maximal efficacy is determined principally by the properties of the drug and its receptor-effector system and is reflected in the plateau of the curve. In clinical use, a drug's dosage may be limited by undesired effects.

Biological variability. An effect of varying intensity may occur in different individuals at a specified concentration or a drug. It follows that a range of concentrations may be required to produce an effect of specified intensity in all subjects.

Lastly, different individuals may vary in the magnitude of their response to the same concentration of a drug when the appropriate correction has been made for differences in potency, maximal efficacy and slope.

The duration of a drug's action is determined by the time period over which concentrations exceed the MEC. Following administration of a dose of drug, its effects usually show a characteristic temporal pattern. A plot of drug effect vs. time illustrates the temporal characteristics of drug effect and its relationship to the therapeutic window. A lag period is present before the drug concentration exceeds the minimum effective concentration (MEC) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. The therapeutic window reflects a concentration range that provides efficacy without unacceptable toxicity. Accordingly another dose of drug should be given to maintain concentrations within the therapeutic window.

According to some embodiments, the concentration of the active constituent is at least 0.1%, 0.2%, 0.3%, 0.4%, 0.5%, 0.6%, 0.7%, 0.8% n, 0.9%, 1%, 2%, 3%, 4%, 5%, 6%, 7%, 8%, 9%, 11%; 19%; 20%, 30%, 40%, 50%, or at least 10%; >12%; 13%; 14%; 18%; >60% w/w of the topical composition. According to some embodiments, the concentration of the active agent is from about 1% to about 10%, inclusive, w/w of the composition, i.e., at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 16%, at least 17%, at least 18% at least 19%, or at least 20% w/w of the topical composition.

According to some embodiments, the content of the active agent retained on skin and its permeation/flux into the skin can be measured as a function of time. According to some embodiments, flux is determined using one of many available artificial membranes (e.g., Cuprophan) attached to a Franz diffusion cell. According to some embodiments, permeation and retention are determined using human cadaver skin attached to a Franz diffusion cell. According to some embodiments, the retained concentration is correlated to the minimum effective concentration.

According to some embodiments, the composition can be applied directly to skin. According to some embodiments, the surface area of the skin ranges from small to large. According to some embodiments, the surface area of the skin ranges from 0.5 cm² to 10 cm²′ inclusive. According to some embodiments, the surface area is about 0.5 cm², 0.6 cm², 0.7 cm², 0.8 cm², 0.9 cm², 1.0 cm², 1.1 cm², 1.2 cm², 1.3 cm², 1.4 cm², 1.5 cm², 1.6 cm², 1.7 cm², 1.8 cm², 1.9 cm², 2.0 cm², 2.1 cm², 2.2 cm², 2.3 cm², 2.4 cm², 2.5 cm², 2.6 cm², 2.7 c cm², 2.8 cm², 2.9 cm², 3.0 cm², 3.1 cm², 3.2 cm², 3.3 cm², 3.4 cm², 3.5 cm², 3.6 cm², 3.7 cm², 3.8 cm², 3.9 cm², 4.0 cm², 4.1 cm², 4.2 cm², 4.3 cm², 4.4 cm², 4.5 cm², 4.6 cm², 4.7 cm², 4.8 c cm², 4.9 cm², 5.0 cm², 5.1 cm², 5.2 cm², 5.3 cm², 5.4 cm², 5.5 cm², 5.6 cm², 5.7 cm², 5.8 cm², 5.9 cm², 6.0 cm², 6.1 cm², 6.2 cm², 6.3 cm², 6.4 cm², 6.5 cm², 6.6 cm², 6.7 cm², 6.8 cm², 6.9 cm², 7.0 cm²′ 7.0 cm², 7.1 cm², 7.2 cm², 7.3 cm², 7.4 cm², 7.5 cm², 7.6 cm², 7.7 cm², 7.8 cm², 7.9 cm², 8.0 cm². 8.1 cm², 8.2 cm², 8.3 cm², 8.4 cm², 8.5 cm², 8.6 cm², 8.7 cm², 8.8 cm², 8.9 cm², 9.0 cm² 9.1 cm², 9.2 cm², 9.3 cm², 9.4 cm², 9.5 cm², 9.6 cm², 9.7 cm², 9.8 cm², 9.9 cm², or 10.0 cm².

### 2.6-3 Additional Active Ingredients

According to some embodiments, the compositions of the described invention further comprise additional active ingredients. Such additional active agents cam include, but are not limited to, a carrier oil, an antifungal agent, an antibiotic, an antiviral agent, an antiprotozoal agent, an anesthetic agent, a chemotherapeutic agent, a vitamin, a hormone, and a steroid.

Exemplary carrier oils are described in WO2020044118, which is incorporated herein by reference. According to some embodiments, the carrier oil is a lipid carrier comprising one or more lipids selected from the group consisting of: camelina oil; a marine phospholipid; a krill oil; a fish oil; chia seed oil; flaxseed oil; an oil comprising an omeg-3 to omega-6 ratio of about 1.0 or higher, about 1.5 or higher, about 2.0 or higher, or about 2 2 or higher; a hydrolyzed oil; a vegetable oil; hemp oil; an oil containing medium-chain fatty acids (MCFAs); and an oil containing long-chain fatty acids (LCFAs).

According to some embodiments, the lipid carrier comprises camelina oil. Camelina oil is an edible oil derived from the seeds of Camelina sativa. It has been found to contain high amounts of omega-3 fatty acids and vitamin E, making it suitable for use as a nutritional supplement and as a general purpose oil. Due to its high vitamin E content, Camelina oil may be used as an antioxidant and as an anti-inflammatory. Camelina oil may also be prepared as an extract of a cultivated Camelina sativa plant crop.

According to some embodiments, the carrier oil may be derived from a plant (plant-based). Examples of plant-based carrier oils include without limitation almond oil, apricot kernel oil, avocado oil, borage seed oil, castor oil, chia seed oil, cranberry seed oil, corn oil, coconut oil, hazelnut oil, hemp oil, evening primrose oil, grapeseed oil, jojoba oil, linseed oil, macadamia nut oil, mustard seed oil, sesame oil, olive oil, cotton oil, peanut oil, pecan oil, pomegranate seed oil, poppy seed oil, rosehip oil, soybean oil, sunflower oil, and watermelon seed oil.

According to some embodiments, the plant-based carrier oil may be an essential oil or volatile oil, such as bay oil, bergamot oil, balm oil, cedarwood oil, cherry oil, cinnamon oil, clove oil, peppermint oil, and walnut oil.

According to some embodiments, the carrier oil may be derived from an animal (animal-based). Examples of animal-based carrier oils include without limitation poultry oil, emu oil, oleo-oil, tallow oil, fish oil, fish liver oil, and cod liver oil.

According to some embodiments, the carrier oil may comprise one or more long chain fatty acids. Exemplary long chain fatty acids may have a straight or branched chain, for example, comprising 13 or more carbon atoms. Without being limited by theory, since long chain fatty acids are absorbed by the lymphatic system, formulations comprising one or more long chain fatty acids may therefore improve absorption when combined with the described compositions. According to some embodiments, the carrier oil may comprise one or more medium chain fatty acids. Exemplary medium chain fatty acids may have a straight or branched chain, for example, comprising 6-12 carbon atoms. According to some embodiments the carrier oil may comprise one or more long chain fatty acids and one or more medium chain fatty acids in combination.

According to some embodiments, the carrier oil may comprise MCT oil. MCT Oil contains caprylic acid (C8:0) and capric acid (C10:0). MCT Oil may be derived from coconut/palm kernel oil. Alternatively, MCT Oil is widely available from commercial sources (e.g., Now Foods).

According to some embodiments, the carrier oil may comprise a marine oil. Marine oils comprise oils derived from ocean organisms including vertebrates (e.g. fish) and non-vertebrates (e.g. invertebrates, bacteria and sea-plants). An exemplary carrier oil for use in the described invention is oil extracted from krill. For example, Neptune Krill Oil (NKO) has unique fatty acid profile, which includes EPA and DHA, plus phospholipids. In addition, NKO contains astaxanthin, a free radical scavenger and immune-supporting carotenoid. Krill oil, including NKO, is widely available from commercial sources (e.g., Now Foods).

The term “anti-fungal agent” as used herein means any of a group of chemical substances having the capacity to inhibit the growth of or to destroy fungi. Anti-fungal agents include, but are not limited to, Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, Pyrrolnitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconazole, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “anti-infective agent” as used herein means any of a group of chemical substances having the capacity to inhibit the growth of, or to destroy microorganisms, used chiefly in the treatment of infectious diseases. Examples of anti-infective agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Remdecivir, Sorivudine, Stavudine, Trifluridine, Valacyclovir, Vidarabine, Zalcitabine, Acemannan, Acetylleucine, Amantadine, Amidinomycin, Delavirdine, Foscamet, Indinavir, Interferons (e.g., IFN-alpha), Kethoxal, Lysozyme, Methisazone, Moroxydine, Nevirapine, Podophyllotoxin, Remdecivir; Ribavirin, Rimantadine, Ritonavir2, Saquinavir, Stailimycin, Statolon, Tromantadine, Zidovudine (AZT) and Xenazoic Acid.

The term “anti-protozoal agent” as used herein means any of a group of chemical substances having the capacity to inhibit the growth of or to destroy protozoans used chiefly in the treatment of protozoal diseases. Examples of antiprotozoal agents, without limitation, include pyrimethamine (Daraprim®) sulfadiazine, and Leucovorin.

The term “anesthetic agent” as used herein refers to an agent that produces a reduction or loss of sensation. Non-limiting examples of anesthetic agents that are suitable for use in the context of the described invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

The term “chemotherapeutic agent” as used herein refers to chemicals useful in the treatment or control of a disease. Non-limiting examples of chemotherapeutic agents usable in context of the described invention include temozolomide, busulfan, ifosamide, melphalan, carmustine, lomustine, mesna, 5-fluorouracil, capecitabine, gemcitabine, floxuridine, decitabine, mercaptopurine, pemetrexed disodium, methotrexate, vincristine, vinblastine, vinorelbine tartrate, paclitaxel, docetaxel, ixabepilone, daunorubicin, epirubicin, doxorubicin, idarubicin, amrubicin, pirarubicin, mitoxantrone, etoposide, etoposide phosphate, teniposide, mitomycin C, actinomycin D, colchicine, topotecan, irinotecan, gemcitabine cyclosporin, verapamil, valspodor, probenecid, MK571, GF120918, LY335979, biricodar, terfenadine, quinidine, pervilleine A and XR9576.

The term “vitamin” as used herein, refers to any of various organic substances essential in minute quantities to the nutrition of most animals act especially as coenzymes and precursors of coenzymes in the regulation of metabolic processes. Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

The term “hormone” as used herein refers to natural substances produced by organs of the body that travel by blood to trigger activity in other locations or their synthetic analogs. Suitable hormones for use in the context of the present invention include, but are not limited to, any hormone produced by neurosecretory cells, including gonadotropin releasing hormone (GnRH), corticotropin releasing hormone (CRH), thyrotropin releasing hormone (TRH), prolactin inhibiting hormone (dopamine) and orexin (hypocretin), as well as recombinant hormones, meaning hormones produced by a process using DNA engineered to contain sequences that normally would not occur together and introducing that DNA into the cells of a host.

The term “steroid” or “steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

2.7 Kits

The compositions described herein can be packaged in suitable containers labeled, for example, for improving immune system health, for treating symptoms of a virus infection (prophylaxis) or for potentiation of an immune response.

Accordingly, packaged products (e.g., sterile containers) can contain a composition comprising NAC, a composition comprising a cannabimimetic, or both at concentrated or ready-to-use concentrations and can be packaged for storage, shipment, or sale. A product can include a container (e.g., a vial, jar, bottle, bag, or the like) containing the one or more compositions of the invention. In addition, a kit further may include, for example, packaging materials, instructions for use, syringes, delivery devices, buffers or other control reagents for treating or monitoring the condition for which prophylaxis or treatment is required.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Cannabis Extraction Methods

La'au Pono, Inc. cultivates medical cannabis under a license from the State of Hawaii.

Cannabis plants were harvested at peak maturity. Harvested plants were extracted with ice water to separate and collect the trichome heads from the cannabis plant. The trichome heads were separated from broken plant material and other contamination by filtration. After rinsing, the trichomes were collected and dried by lyophilization. The dry, lyophilized trichomes were loaded into micron filter bags; a rosin press then was used to extract the cannabis oil containing the acid forms of the cannabinoids present. For non-acid forms of the cannabinoids, the extract is decarboxylated with heat. Once decarboxylation is complete, a sample is prepared and a cannabinoid concentration analysis performed. Powder-based products are manufactured by dissolving the cannabis extract comprising all active compounds in ethanol loaded with mannitol. The cannabis base powder is then dried.

Chemotypes Data

Extracted flower strains Animal, Cheese, CD-1, and Jack Herer were subjected to chemical potency analysis. The results are shown in the tables below.

### Example 1.1 Sample Name: Animal; Sample Type: Flower

Cannabinoid standard potency analysis using ultra-high performance liquid chromatography is shown in Table 1. PGP-26,T2,M Table 1.

TABLE 1

| Analyte | | | | Mg/g | | |
|---|---|---|---|---|---|---|
| * | % | Mg/g | % (Dry) | (Dry) | LOD mg/g | LOQ mg/g |
| CBC | ND | ND | ND | ND | 0.0691 | 0.138 |
| CBD | ND | ND | ND | ND | 0.0691 | 0.0725 |
| CBDA | 0.0360 | 0.360 | 0.0412 | 0.412 | 0.0691 | 0.124 |
| CBDV | ND | ND | ND | ND | 0.0691 | 0.149 |
| CBDVA | ND | ND | ND | ND | 0.0691 | 0.169 |
| CBG | 0.0404 | 0.404 | 0.0463 | 0.463 | 0.0691 | 0.0794 |
| CBGA | 0.397 | 3.97 | 0.455 | 4.55 | 0.0691 | 0.0691 |
| CBN | ND | ND | ND | ND | 0.0691 | 0.0691 |
| THC | 0.282 | 2.82 | 0.323 | 3.23 | 0.0691 | 0.0691 |
| Delta-8- | ND | ND | ND | ND | 0.0691 | 0.104 |
| THC | | | | | | |
| THCA | 15.4 | 154 | 17.6 | 176 | 0.0691 | 0.159 |
| THCV | ND | ND | ND | ND | 0.0691 | 0.104 |
| THCVA | 0.460 | 0.460 | 0.0528 | 0.52 | 0.0691 | 0.145 |
| Total | 16.2 | 16.2 | 18.5 | 18.5 | | |

*ND: Not Detected; NT: Not tested: LOQ: Limit of Quantitation; LOD: Limit of Detection

| TOTAL THC | TOTAL CBD |
|---|---|
| 13.7% | 0.0315% |
| 137 mg/g | 0.315 mg/g |
| 15.8% (Dry) | 0.0362% (Dry) |
| 158 mg/g (Dry) | 0.362 mg/g (Dry) |

Total THC = [THCA × 0.877] + [THC]
Total CBD = [CBDA × 0.877] + [CBD]

### Example 1.2—Sample Name: Cheese; Product Type: Flower

Cannabinoid standard potency analysis using ultrahigh performance liquid chromatography is shown in Table 2.

TABLE 2

| Analyte | Weight % | Mg/g |
|---|---|---|
| Delta 9-THC | 0.2 | 2.2 |
| THCA | 26.7 | 267.3 |
| CBD | <LOQ | <LOQ |
| CBDA | <LOQ | <LOQ |
| CBG | <LOQ | <LOQ |
| CBN | <LOQ | <LOQ |
| THCV | <LOQ | <LOQ |
| CBDV | <LOQ | <LOQ |
| CBDVA | <LOQ | <LOQ |
| CBGA | 0.8 | 7.9 |
| CBC | <LOQ | <LOQ |
| CBL | <LOQ | <LOQ |
| Theoretical delta 9-THC* | 23.7 | 236.6 |
| Theoretical CBD* | <LOQ | <LOQ |

Theoretical Δ9-THC and CBD calculations account for decarboxylation of THCA to THC and CBDA to CBD, respectively.
Theoretical delta-THC = (0.877 × THCA) + THC
Theoretical CBD = (0.877 × CBDA) + CBD

### Example 1.3 CD-1; Sample Type: Flower

Cannabinoid standard potency analysis using ultrahigh performance liquid chromatography is shown in Table 3.

TABLE 3

| Cannabinoid | % | Mg/g | LOD mg/g | LOQ mg/g |
|---|---|---|---|---|
| CBD | 0.071 | 0.71 | 0.073 | 0.077 |
| CBDA | 4.9 | 49 | 0.073 | 0.132 |
| CBG | 0.024 | 0.24 | 0.073 | 0.084 |
| CBN | ND | ND | 0.073 | 0.073 |
| THC | ND | ND | 0.073 | 0.073 |
| THCA | 0.20 | 2.0 | 0.073 | 0.169 |
| Total Measured | 5.2 | 52 | | |

### Example 1.4: Jack Herer CBD; Sample Type: Flower

Cannabinoid standard potency analysis using ultrahigh performance liquid chromatography [need to know column composition and mobile phase] is shown in Table 4.

TABLE 4

| | | | % | Mg/g | LOD | LOQ |
|---|---|---|---|---|---|---|
| Analyte | % | Mg/g | (Dry) | (Dry) | mg/g | mg/g |
| CBC | ND | ND | ND | ND | 0.072 | 0.145 |
| CBD | ND | ND | ND | ND | 0.072 | 0.076 |
| CBDA | 9.9 | 99 | 11.3 | 113 | 0.072 | 0.130 |
| CBGDV | ND | ND | ND | ND | 0.072 | 0.155 |
| CBDVA | ND | ND | ND | ND | 0.072 | 0.177 |
| CBG | 0.034 | 0.34 | 0.039 | 0.39 | 0.072 | 0.083 |
| CBN | ND | ND | ND | ND | 0.072 | 0.072 |
| THC | 0.25 | 2.5 | 0.29 | 2.9 | 0.072 | 0.072 |
| Delta 8- | ND | ND | ND | ND | 0.072 | 0.108 |
| THC | | | | | | |
| THCA | 5.5 | 55 | 6.3 | 63 | 0.072 | 0.166 |
| THCV | ND | ND | ND | ND | 0.072 | 0.108 |
| THCVA | 0.055 | 0.55 | 0.063 | 0.63 | 0.072 | 0.108 |
| Total | 15.7 | 157 | 18 | 180 | | |

| TOTAL THC | TOTAL CBD |
|---|---|
| 5.0% | 8.7% |
| 50 mg/g | 87 mg/g |
| 5.8% (Dry) | 9.9% (Dry) |
| 58 mg/g (Dry) | 99 mg/g (Dry) |

Total THC = [THCA × 0.877] + [THC]
Total CBD = [CBDA × 0.877] + [CBD]

Terpinoids were determined by standard terpene analysis utilizing liquid chromatography-mass spectrometry. The results are shown in Table 5.

TABLE 5

| Analyte | % | Mg/g | LOD mg/g | LOQ mg/g |
|---|---|---|---|---|
| Caryophyllene | ND | ND | 0.116 | 0.181 |
| oxide | | | | |
| B-caryophyllene | 0.48 | 4.8 | 0.116 | 0.177 |
| cinronellol | ND | ND | 0.116 | 0.199 |
| α-Humulene | 0.41 | 4.1 | 0.116 | 0.170 |
| Linalool | ND | ND | 0.116 | 0.173 |
| B-Myrcene | 0.26 | 2.6 | 0.116 | 0.163 |
| Total | 1.15 | 11.5 | | |

### Example 2. Immune Repertoire Testing of NAC and Cannabinoids

### 2.1 Background

Antigen receptors with diverse binding activities are the hallmark of T and B cells of the adaptive immune system. These are generated by genomic rearrangement of variable (V), diversity (D) and joining (J) gene segments separated by highly variable junction regions. [Boyd, S C et al. Sci. Trans. Med. (2009) 1(12): 12ra23, citing Schatz, DG. Semin. Immunol. (2004) 16: 245-56]. The complex repertoire of immune receptors generated by T and B calls enables recognition of diverse threats to the host organism. [Boyd, S C et al. Sci. Trans. Med. (2009) 1(12): 12ra23]. Expanded clones of B cells with useful antigen specificities persist over time to enable rapid responses to antigens previously detected by the immune system.

T cell receptors (TCRs) are dimeric (αβ or γ8) highly variable T lymphocyte membrane proteins that recognize antigenic peptides presented on heterologous cells by the major histocompatibility complex (MHC) [Freeman, J D, et al. Genome Res. (2009) 19: 1817-24, citing Davis, M M and Bjorkman, P J Nature (1988) 334: 395-402; Bassing, C H et al Cell (2002) 109 (Suppl): S45-SSS]. Recognition specificity for diverse peptide-MHC (pMHC) complexes is provided by the three complementarity determining regions (CDRs) of the TCR. CDR1 and CDR2) are coded for by germline sequences, while CDR3, the highly polymorphic principal recognition site, is created when TCR genomic loci undergo somatic recombination between gene segments during development of T lymphocytes in the thymus [Id., citing Gellert, M. Annu. Rev. Genet. (1992) 26: 425-446; Gellert, M. Ann. Rev. Biochem. (2002) 71: 101-32; Gellert, 1992, 2002; Jung, D. and Alt, F W. Cell (2004) 116: 299-311]. The CDR3 of each of the β and 8 two receptor chains defines the clonal specificity. For αβ T cells, the CDR3 is in most contact with the peptide bound to the MHC. [Id., citing Rudolph, M G et al. Annu. Rev. Immunol. (2006) 24: 419-66]. For the α locus and the γlocus, recombination occurs between variable (V) and joining (J) segments. For the 8 locus and the β locus, there is recombination between V and J segments, but also the inclusion of one of two short diversity (D) segments. At CDR3 recombination junctions, further complexity is generated through the deletion of germline-encoded bases and the addition of random nontemplated bases. The resulting hypervariable sequences of the CDR3 make possible the recognition of diverse peptide-MHC (pMHC) complexes. During T cell maturation, all T cells expressing rearranged receptors capable of binding pMHC with high enough affinity to be biologically relevant are retained (positive selection), but only T cells with rearranged receptors that do not interact strongly with self-pMHC complexes ultimately exit the thymus (negative selection). [Freeman, J D, et al. Genome Res. (2009) 19: 1817-24]. The V(D)J recombination is not entirely random, and the prevalence of specific gene segments and combinations of gene segments shows marked variation in the repertoire. Contributes to this bias are introduced even before thymic selection, through variation in the efficiency of recombination of different gene segments. [Id., citing Manfras, B J et al. Hum. Immunol. (1999) 60: 1090-1100; Krangel, M S. Nat. Immunol. (2003) 4: 624-30].

Accordingly, there are two subsets of T cells based on the exact pair of receptor chains expressed. These are either the alpha (α) and beta (β) chain pair, or the gamma (γ) and delta (8) chain pair, identifying the αβ or γ8 T cell subsets, respectively. The expression of the β and 8 chain is limited to one chain in each of their respective subsets by allelic exclusion [Yassi, M. B. et al. Immunogenetics (2009) 61: 493-502., citing Bluthman et al 1988; Uematsu et al 1988]. These two chains are also characterized by the use of an additional DNA segment, referred to as the diversity (D) region during the rearrangement process. The D region is flanked by N nucleotides, which constitutes the NDN region of the CDR3 in these two chains. [Id.]

The initial phase of the adaptive immune response involves B and T cell clonal selection on the basis of the structural complementarity of antigen-specific receptors to pathogen-derived epitopes. [Yassi, M. B. et al. Immunogenetics (2009) 61: 493-502, citing Davis, M M and Chien, Y H. In Paul, W E ed. Fundamental Immunology, 5^{th} Ed. (2003) Lippincott Williams & Wilkins, Philadelphia, pp. 227-58; Kolar, G R and Capra, JD In Paul, WE ed. Fundamental Immunology, 5th Ed. (2003) Lippincott Williams & Wilkins, Philadelphia, pp. 47-68]. After pathogen clearance, a proportion of these cells will be retained as memory. Memory provides more rapid and effective immune protection against recurring pathogen present in the environment. The collection of cells that respond to a particular pathogen is referred to as the repertoire. The repertoire recognizing a molecule would be the sum of the repertoires responding against all the component epitopes of the molecule. Likewise, the repertoire against an organism would be the sum of all the repertoires against all the molecules from the pathogen. [Id.]

Wang et al. estimated 0.47×10⁶ TCR-α unique nucleotide sequences and 0.35×10⁶ TCR-β sequences. [Benichou, J. et al., Immunology (2011) 135: 183-91, citing Wang, C., et al. Proc. Natl Acad. Sci. USA (2010) 107: 1518-23]. Robins et al. suggested that CD8+ T cells express <0.1% of the combinatorial landscape of the β chain (5×10¹¹). [Id., citing Robins, H S., et al. Blood (2009) 114: 4099-4107]. These are only lower limits to the actual size of the repertoire, and any individual expresses only a small fraction of the potential diversity. [Id.].

Antibody paratropes are found at the hypervariable region of a light and heavy chain heterodimer. Each chain contributes three loops to a spatial cluster of complementarity determining regions (CDRs). CDR1 1 and 2 are encoded in germline V-segment loci: S1 Vh and 70 Vic/2, loci, each with unique amino acid encodings exist in a typical human haplotype [Glanville, J. et al., Proc. Natl Acad. Sci USA 106 (48): 20216-21, citing Huber, C. et al. Eur. J. Immunol. (1993) 23: 2868-75; Kawasaki, K. et al. Genome Res. (1995) 5: 125-35; Matsuda, F. et al. J. Exp. Med. (1998) 188: 2151-62]. Diversity in each chain is determined by combinatorial VH-(DH)-JH (for the heavy) or Vκ/λ-Jκ/λ (for the light) rearrangements, P and N-addition, junctional flexibility, and somatic hypermutation of variable domain nucleotides, with a concentration on CDR encoding regions [Id., citing Tonegawa, S. Nature (1983) 302: 575-81; Wu, TT, Kabat, EA. J. Exp. Med. 132: 211-50]. The combinatorial association of such stochastically generated light and heavy chains has the potential to generate many orders of magnitude more diversity than can be uniquely displayed on the 10¹¹ B cells in a single individual's lymphocyte population [Id., citing Perelson, A S, Oster, G F. J. Theor. Biol. 81: 645-70; Trepel, F. Klin. Wochenschrift (1974) 52: 511-15]. With each antibody variable fragment (Fv) encoded by at least 650 base pairs, the presented repertoire is potentially 4 orders of magnitude larger than the entire human diploid genome (6.4×10⁹ bp).

The combination of all of these sources of diversity generates a vast repertoire of T cell and B cell specificities. The average human immune system can recognize 10¹²-10¹⁵ antigens, meaning that the immune arsenal already stores the means to recognize virtually any foreign molecule. Large numbers of different T-cell and B-cell receptors or clonotypes enable the immune system to protect against many different types of pathogens. As needed, populations of one or more receptor combinations can be expanded to eliminate a new pathogen. With age however, this surveillance system becomes spent and less flexible to mount an immune defense. Any underlying conditions further jeopardize immune preparedness.

A T cell clonotype is a unique nucleotide sequence that arises during the gene rearrangement process for that receptor. The combination of nucleotide sequences for the surface expressed receptor pair would define the T cell clonotype. Clonotyping, the process used to identify the unique nucleotide CDR sequences of a TCR chain, involves PCR amplification of the cDNA using V region-specific primers and either constant region (C) or J region-specific primer pairs, followed by nucleotide sequencing of the amplicon. The diversity index therefore is an expression of the CDR3 clonotype of the T cell β chain out of all the repertoires. It is noted that referring to only one T-cell receptor chain ignores that the actual clonotype of a T-cell consists of the combination of both alpha and beta receptor chains. A further complication is when homodimers form receptors, e.g., alpha:alpha and beta:beta. These considerations would actually increase the potential diversity.

### 2.2 Methods

Subjects: Tested individuals were generally healthy thirty-something males and females, mostly regular cannabis users. These individuals had good immune diversity baselines.

### 2.2-1 Criteria for Individual Testing

Subjects abstained from cannabis use for prior three days.

Baseline and sample blood was taken using the finger-prick method at designated times.

Blood sample collection: The sample collection method used for blood spots was as follows. The iRepertoire® home test kit collects a sample of blood on filter paper that can be stored at room temperature. The subject pricks their finger and then bleeds onto the sample collecting cart, filling four circles approximately 1-2 cm in diameter completely. The sample card is then stored in a plastic bag with a desiccant, and shipped back to iRepertoire's laboratory for processing once the protocol is complete. Alternatively, a blood sample is collected in a PAXgene vacutainer tube. RNA was prepared and shipped to iRepertoire on dry ice [how prepared].

Samples were sent on a weekly basis to iRepertoire for analyses, where they were batch analyzed.

### 2.2-2 Dosing

Cannabis strains tested: Cheese, Granddaddy Purple, Jack CBD; and CBD-1 30 mg CBD capsules.

Oral dosage forms include, without limitation, quick dissolve tablets, effervescent tablets, and capsules. Quick dissolve tablets (QDTs) are solid unit dosage forms, which disintegrate or dissolve rapidly in the mouth without chewing and water well-known in the art. Actives belonging to Biopharmaceutical Classification System Class II, i.e., drugs with poor solubility and high permeability have been formulated as QDTs in a dose of 125 and 250 mg. [See Parkash, V. et al, J. Adv. Pharm. Technol. Res. (2011) 2(4): 223-235, citing Amidon, G L et al. Pharm Res. (1995) 12: 413-20; Lindenberg, M. et al., Eur. J. Pharm Biopharm. (2004) 58: 265-78] Tizanidine HCl [Id., citing Zade, P S et al. Inter. J. Pharm. Tech Res. (2009) 1: 34-42], Oxybutynin HCl, [Id., citing Ishikawa, T. et al. Chem. Pharm. Bull (1999) 47: 1451-54], Rofecoxib [Id., citing Omaima, SA, AAPS Pharm. Sci. Tech. (2006) 7: E1-E9], Ibuprofen [Id., citing Simone, S., Peter, CS Eur. J. Pharm. Sci. (2002) 15: 295-305], Promethazine Theoclate [Id., citing Sharma, S., Gupta, GD. Asian J. Pharm. (2008) 2: 70 72], prednisone [Id., citing Dario, L. et al. AAPS Pharm. Sci. Tech (2007) 8: 221-8], Indomethacin [Id., citing Singh J. et al. AAPS Pharm. Sci. Tech. (2008) 9: 60-6], Glyburide [Id., citing Marziaaa, C. et al. Drug Deliv. (2007) 14: 247-55] Fentanyl citrate [Id., citing Sussanne, B. et al. Eur. J. Pharm. Sci. (2003) 20: 327-34] Griseofulvin [Id., citing Iman, SA, Mona, HA. Eur. J. Pharm. Sci (2007) 32: 58-68] Hydrochlorothiazide [Id., citing Sam C., Jean, PR. Int. J. Pharm. (1997) 152: 215-25] Crystallized Paracetamol [Id., citing Sbdelbary, G et al. Intl J. Pharm. (2004) 278: 423-33] and Nimesulide [Id., citing Raguia, A S et al. Eur. J. Pharm. Biopharm. (2009) 73: 73: 162-71] are just a few examples of drugs that has been formulated as fast-dissolving drug delivery system.

Formulation processes, including freeze drying, compression molding, direct compression, granulation methods (wet granulation, dry granulation, melt granulation), and spray-drying methods are described in Parkash, V. et al, J. Adv. Pharm. Technol. Res. (2011) 2(4): 223-235, which is incorporated herein by reference in its entirety.

Flavoring for taste masking purposes can be obtained from natural or synthetic sources. Conventional natural products include fruit juices, aromatic oils such as peppermint and lemon oils, herbs, spices, and distilled fractions of these. They are available as concentrated extracts, alcoholic or aqueous solutions, syrups, or spirit.

Topical dosage forms include ointments, lotions, creams, gels, powders or salves. The term “ointment” as used herein refers to a semisolid oil-based preparation. The term “lotion” as used herein refers to a thick, smooth liquid preparation to be applied to the skin The term “cream” as used herein refers to a viscous liquid or semisolid emulsion of either the oil-in-water or water-in-oil type. The term “gel” refers to a semisolid jelly. The term “powder refers to fine, dry particles produced by the grinding, crushing, or disintegration of a solid substance. The term “salve” as used herein refers to an oily adhesive substance.

Blood was sampled at different 24 hour increments up to 168 hours (7 days). Blood was sampled at the same time each day to control for circadian rhythms.

iRepertoire process: Samples are extracted to obtain nucleic acids (DNA or RNA). Following extraction, the amount and quality of DNA or RNA is determined as an A260/280 value. “Pure” DNA generally reports an A260/A280 reading of 1.8; whereas RNA is closer to 2.0. A sequencing library is prepared from the RNA or DNA sample by (1) amplification to yield a pool of appropriately sized target sequences; and (2) the addition of sequencing adapters that later will interact with the next generation sequencing (NGS) platform. If RNA is the starting template, the RNA is first converted to cDNA by reverse transcription.

PCR amplification yields a collection of specifically sized DNA fragments (a library) that are compatible with the sequencing system to be used. The adapter ligation step bookends the amplified DNA or cDNA fragments, called amplicons, with specific oligonucleotide sequences that will interact with the surface of a sequencing flow cells. If multiple samples are to be sequenced in a single sequencing run, a unique identifier (or barcode) is additionally ligated to the amplicon. The resulting completed libraries can be pooled into a single sequencing run that is then “demultiplexed” during data analysis.

In step 3, parallel sequencing is performed using an NGS platform. The library is loaded onto the sequencer which then “reads” the nucleotides one by one. In step 4, after sequencing is complete, first, the reads are filtered for quality, amplicon size, and agreement between paired ends. The reads then are assembled and aligned to a reference genome. Finally reads (assembled or raw) are compared to a reference sequence or to reads from another sample to identify variants. If reads are aligned with a reference genome, variant annotation can be used to associate variants with known genes or regulatory sequences.

iRepertoire®'s multiplex PCR amplification technologies were specially designed to amplify the B cell receptor (BCR) and T cell receptor (TCR) chains of the immune system. It offers two different multiplex PCR approaches, both of which amplify all of the V(D)Js in a sample, including the highly variable CDR2 region.

The first multiplex PCR approach, Arm-PCR, provides an inclusive, semi-quantitative overview of the immune repertoire with respect to particular chains.

Amplicon rescued multiplex PCR (arm-PCR, U.S. Pat. No. 7,999,092), amplifies the chain of the requester's choosing from human or mouse samples to include all of the diversity present in a sample. It uses a high concentration of hundreds of nested inside and outside gene-specific primers in the initial combined reverse transcription (RT) plus PCR round 1. Both the reverse outside and inside primers can contribute to first strand synthesis during RT, which is important if there are any secondary RNA structures that make the inner primer binding site inaccessible during RT. The outside primers help to improve the sensitivity of the reaction by increasing target template abundance for the inside primer to bind. Because the nested primer mix goes through many binding and extension cycles, arm-PCR is a great technological solution for rare clonotype discovery. When RT-PCR1 is complete, target amplicons are rescued, and PCR round 2 is performed using fresh enzymes. For PCR2, communal primers that recognize the shared tag sequence introduce during the first round of amplification are used for further amplification. In the first round of arm-PCR, inside and outside TCR/BCR-specific primers amplify the targets of interest, increasing sensitivity and appending communal primary binding sites. In the second round, communal primers exponentially amplify the target amplicons generated in the first round.

The second multiplex PCR approach, Dam-PCR, provides a more quantitative look at the frequency of particular clonotypes of interest for any or all BCR and TCR chains.

Dimer avoided multiplex PCR (dam-PCR) allows the researcher to select any combination of TCR and BCR chains for simultaneous amplification in one reaction. This is made possible by single-cycle binding and extension steps an stringent clean up steps in-between, which omit harmful dimer formation. First, only the 3′ primer is added, and one binding and extension step is performed. Unincorporated 3′ primers are washed away, and then the 5′ primers are added. After another single cycle binding and extension protocol, the 5′ primers are washed away. Primers that bind to the communal primer sites introduced in the first two steps are added for multi-cycle, exponential amplification. Dam-PCR also allows for the inclusion of unique molecular indices (UMIs) so that each strand of RNA can be tagged for direct quantification and both PCR and/or sequencing error removal, which increases confidence in the sequenced targets, and the quantification allows for investigation of interchain ratios in the immune adaptome.

iRepertoire® multiplex primers were used to sequence millions of DNA strands simultaneously to create a set of unique CDR3 strands for TCR β chains in each sample in order to measure the diversity of T cells comprising αβ TCRs in the sample. The measured immune system diversity represents the capacity of the individual for an adaptive immune system response.

### 2.2-3 Results

There is a circadian rhythm to the indices as well as between individual measurements over time. Circadian rhythm was controlled by taking measurements at the same time each day.

### 2.2-3 (a) Controlled Dose, 5 mg, 10 mg

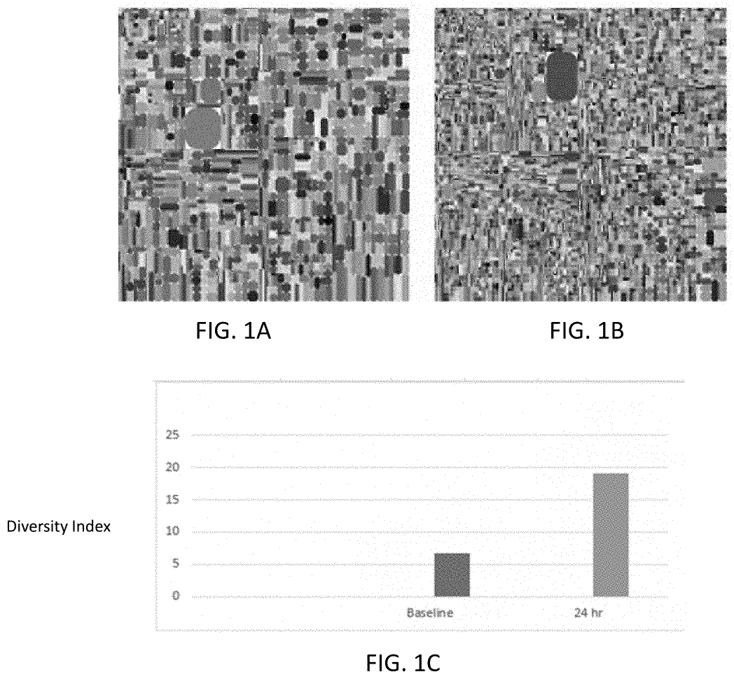

FIG. 1A shows the immune diversity map of the TCR β chain immune repertoire for subject 1 (n=9) at baseline. FIG. 1B shows the immune diversity map for TCR β chain immune repertoire for subject 1 at 24 hours after dosing with 5 mg controlled dose of cheese QDT containing 4.9 mg THC, >0.1 mg THCA, >0.1 mg CBD, 0.2 mg CBG, >0.1 mg CBC, >0.1 mg THCV, >0.1 mg CBN, with pinene, limonene, beta-caryophylleme, humulene, and nerolidol. The number and size of the rounded squares indicate a single T-cell beta chain receptor clonotype and its frequency in peripheral blood. FIG. 1C is a bar graph illustrating T cell β chain diversity index 24 hours after a single dose of 5 mg Cheese for subject 1 compared to baseline. For subject 1, diversity index increased from 6.7 to 19.2 (2.9 fold increase).

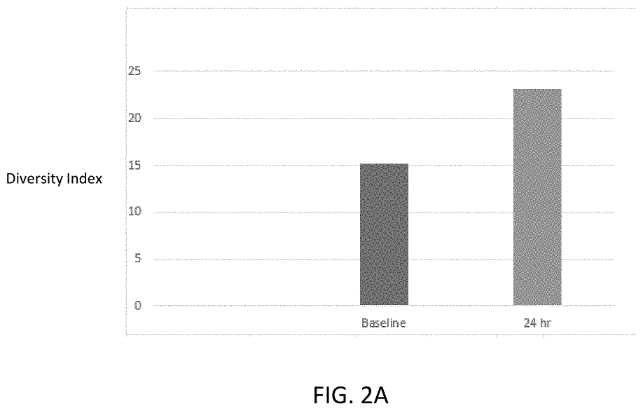

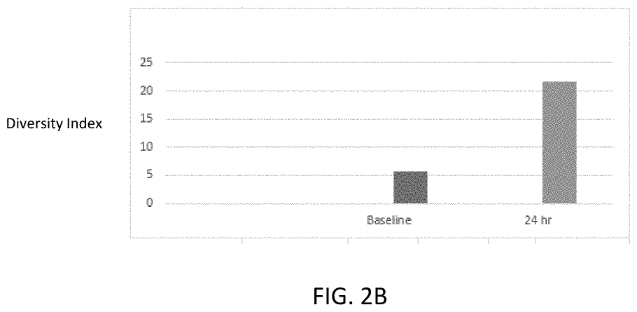

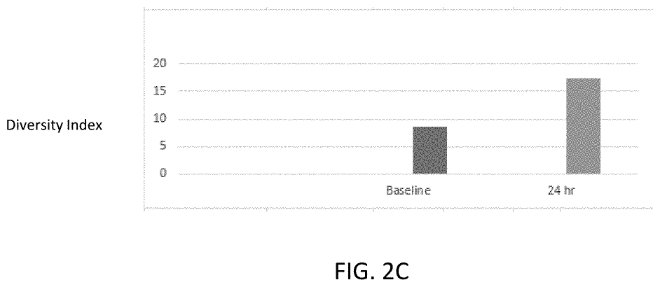

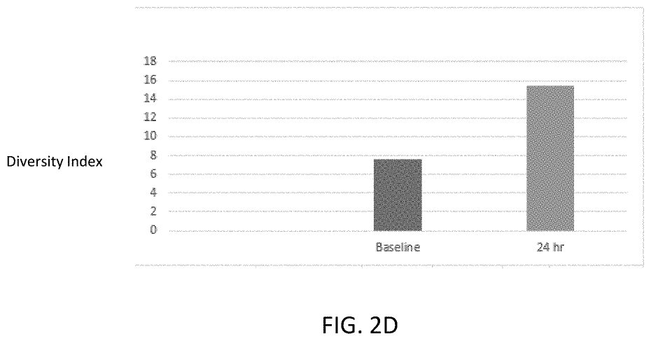

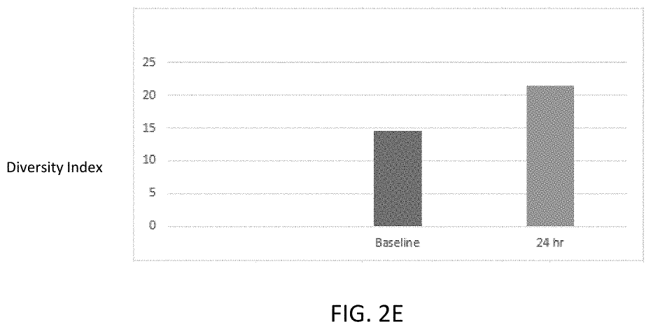

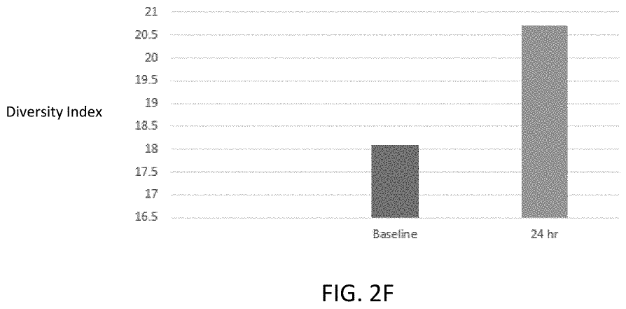

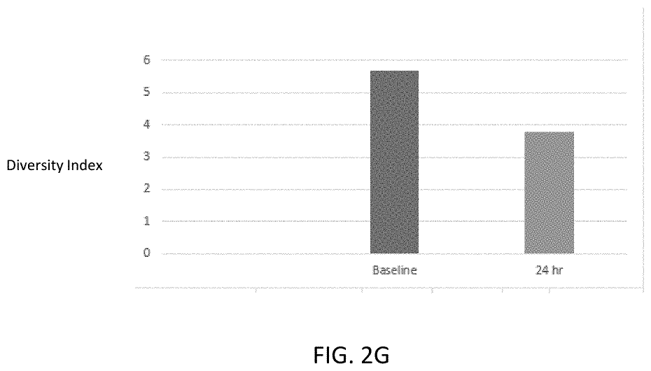

The immune diversity maps for subjects 27, 8 and 9 are not shown. FIG. 2A, 2B, 2C, 2D, 2E, 2F, and 2G are bar graphs illustrating T cell β chain diversity index after 24 hours for each of 7 subjects compared to baseline. Each subject received a controlled dose of a cannabinoid and a blood sample was tested 24 hours after dosing. FIG. 2A; shows results for subject 2, 24 hours after a single dose of 5 mg Cheese. For subject 2, the diversity index after dosing with 5 mg controlled dose of cheese QDT increased from 15.2 to 23.1 (1.5 fold increase). FIG. 2B shows results for subject 3, 24 hours after a single dose of 5 mg Cheese For subject 3, the diversity index after dosing with 5 mg controlled dose of cheese QDT increased from 5.7 to 21.7 (3.8 fold increase). FIG. 2C shows results for subject 4, 24 hours after a single dose of 10 mg Cheese For subject 4, the diversity index after dosing with 10 mg controlled dose of chesse increased from 8.7 to 17.4 (2.0 fold increase). FIG. 2D shows results for subject 5, 24 hours after a single dose of 5 mg Cheese For subject 5, the diversity index after dosing with 10 mg cheese increased from 7.6 to 15.4 (2.0 fold increase). FIG. 2E shows results for subject 6, 24 hours after a single dose of Granddaddy purple tablet For subject 6, the diversity index after dosing with Granddaddy Purple Effervescent Tablet containing 4.7 mg THC, 0.8 mg CBC, 0.8 mg CBN with some limonene, myrcene, and pinene increased from 14.6 to 21.4 (1.5 fold increase). FIG. 2F shows results for subject 8, 24 hours after a single dose of 5 mg Cheese For subject 8, the diversity index after dosing with 5 mg cheese increased from 18.1 to 20.7 (no significant effect). FIG. 2G shows results for subject 9, 24 hours after a single dose of 5 mg Cheese For subject 9, the diversity index after dosing with 5 mg cheese decreased from 5.7 to 3.98 (0.7 fold decrease).

### 2.2-3 (b) Dose Response, Controlled Dose, Jack CBD QDT

The moving baseline reflects individual variability over time. Deviations reflect and monitor the wellness of the individual over time. Measures were in early evening (lower point in circadian rhythm); time relative to menstrual cycles were noted.

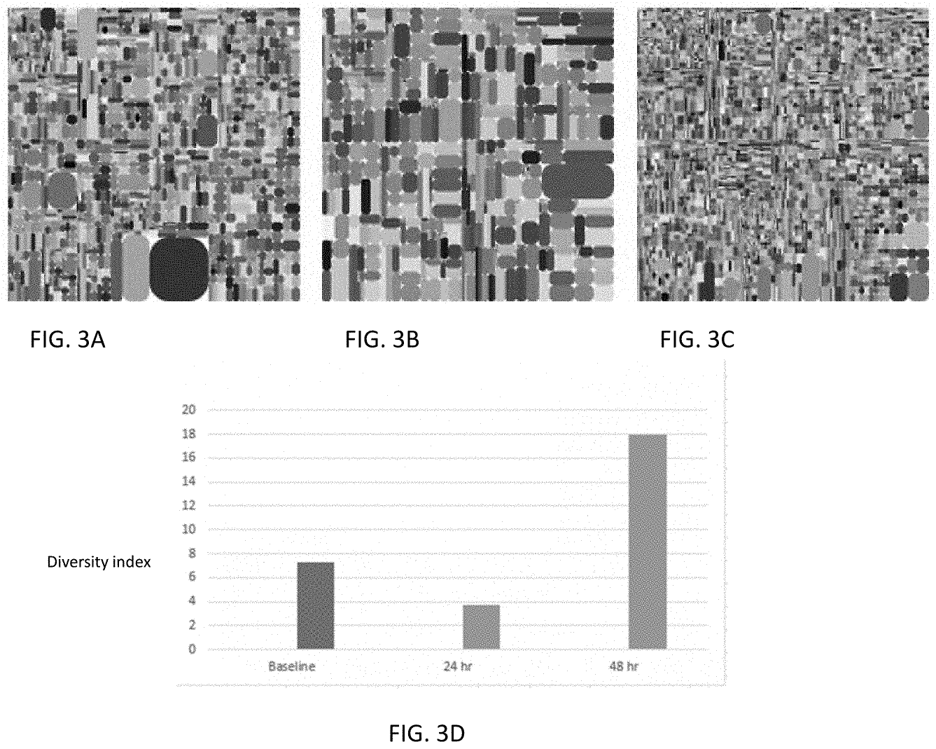

FIG. 3A shows the immune diversity map for the TCR β chain immune repertoire for subject 1 (n=2) at baseline; FIG. 3B shows the immune diversity map for TCR β chain immune repertoire for subject 1 at 24 hours after dosing with Jack CBD QDT containing CBD 7.6 mg CBD and 4.2 mg THC. FIG. 3C shows 48 hours after dosing with Jack CBD. CBD-dominant QDTs show a different repertoire response kinetics than for THC. The number and size of the rounded squares indicate a single T-cell beta chain receptor clonotype and its frequency in peripheral blood. FIG. 3D is a bar graph illustrating T cell β chain diversity index for subject 1, compared to baseline, 24 hours and 48 hours after receiving a controlled dose of Jack CBD cannabinoid. For subject 1, the diversity index after dosing with Jack CBD QDT changed from 7.3 to 3.7 at 24 hr (0.5 fold decrease) and then 48 hours after dosing increased to 18 (2.5 fold increase).

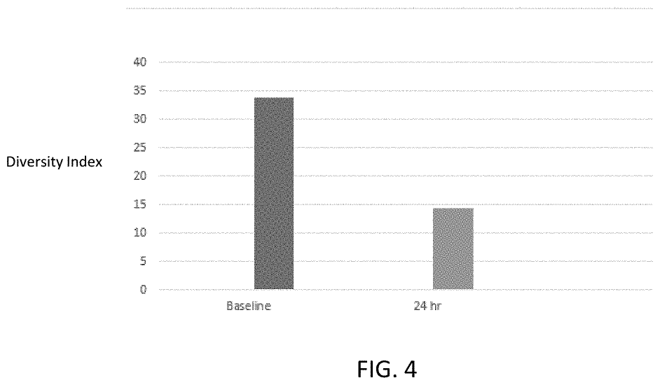

An immune diversity map is not shown for subject 2. FIG. 4 is a bar graph illustrating T cell β chain diversity index for subject 2, compared to baseline 24 hours after receiving a controlled dose of Jack CBD cannabinoid. For subject 2, the diversity index after dosing with Jack CBD QDT decreased from 33.9 to 14.4 (0.4 to 0.8 fold decrease.

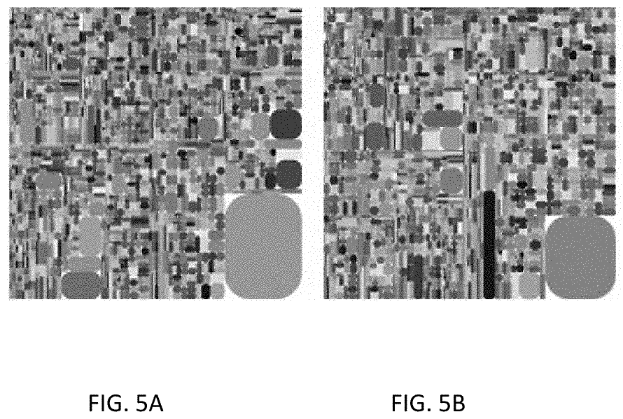

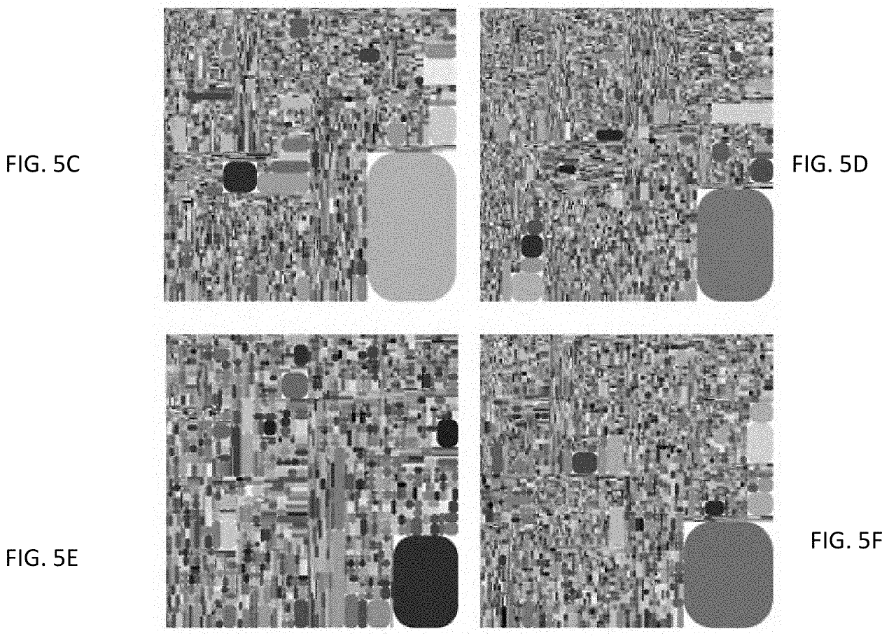

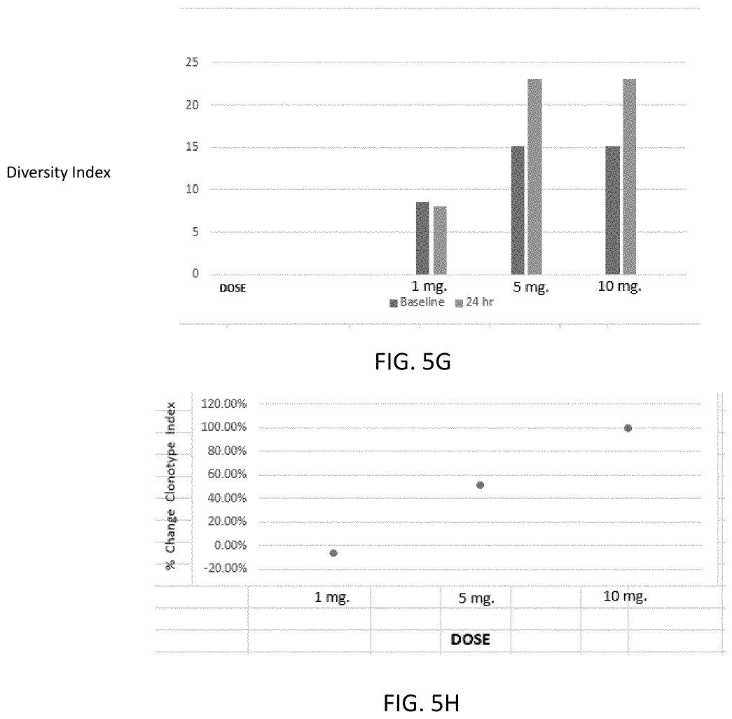

FIG. 5A, 5B, 5C, 5D, 5E, 5F, 5G and 5H show results of dose response experiments with 1 mg, 5 mg, and 10 mg controlled dose Cheese QDTs. Cheese QDTs contained 4.9 mg THC, >0.1 mg THCA, >0.1 mg CBD, 0.2 mg CBG, >0.1 mg CBC, >0.1 mg THCV, >0.1 mg CBN, with pinene, limonene, beta-caryophylleme, humulene, and nerolidol.

FIG. 5A, 5B, 5C, 5D, 5E, and 5F show immune diversity maps for the TCR βchain immune repertoire after 1 mg, 5 mg, and 10 mg controlled dose Cheese QDTs. Each dose is tested at different times. These are longitudinal studies in humans, so each dose is compared with a baseline when the kinetic measurements are started. FIG. 5A shows the immune diversity map for the TCR β chain immune repertoire for subject E2 at baseline;

FIG. 53B shows the immune diversity map for subject E2 at 24 hours after a dose of 1 mg THC. The diversity index decreased from 8.6 to 8.1 (0.9 fold decrease). FIG. 5C and FIG. 5D show the immune diversity map for the TCR β chain immune repertoire for subject E2 at baseline (FIG. 5C) and at 24 hours (FIG. 5D) for a dose of 5 mg THC. The diversity index increased from 15.2 to 23.1 (1.5 fold increase). FIG. 5E, and FIG. 5F show the immune diversity map for the TCR β chain immune repertoire for subject E2 at baseline (FIG. 5E) and at 24 hours (FIG. 5F) for a dose of 10 mg THC. The diversity index increased from 8.7 to 17.4 (2.0 fold increase). The number and size of the rounded squares indicate a single T-cell beta chain receptor clonotype and its frequency in peripheral blood. FIG. 5G is a bar graph illustrating a dose response in T cell β chain diversity index for the subject compared to baseline 24 hours after receiving a 1 mg, a 5 mg, and a 10 mg dose of THC (Cheese QDT). FIG. 5H is a graph showing percentage change in diversity index as a function of dose.

### 2.2-3(c)N-Acetylcysteine, Single Dose

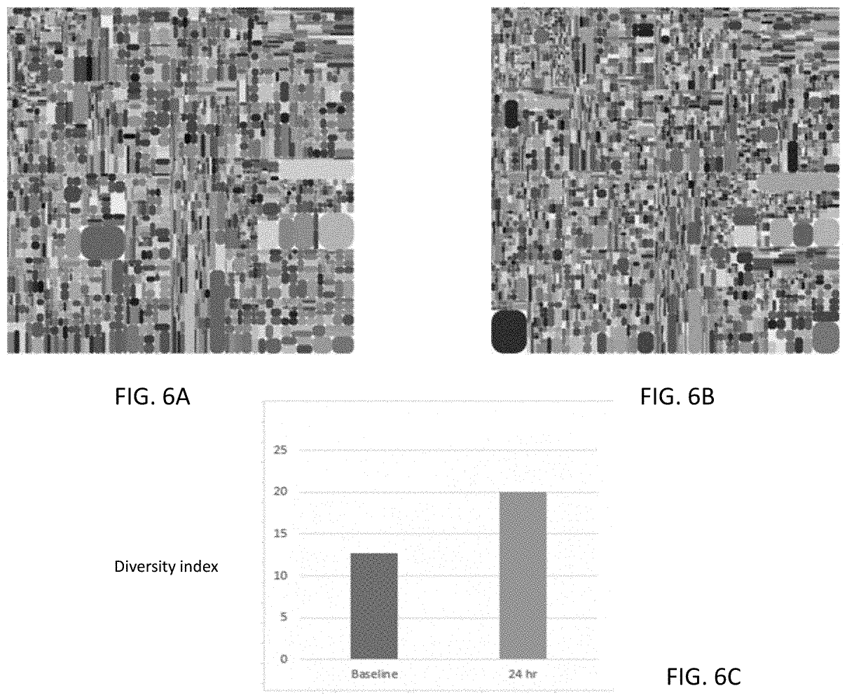

A single dosage of NAC (2×600 mg) effervescent tablets show an increase in immune repertoire similar to cannabis except repertoire returns to baseline in two to three days. FIG. 6A and FIG. 6B show immune diversity maps for the TCR β chain immune repertoire after a single dosage of N-acetylcsteine (NAC) (2×600 mg effervescent tablets) for subject 1 (n=2). NAC causes an increase in immune repertoire similar to cannabis, except the immune repertoire with cannabis returns to baseline in two to three days. After a single dose of NAC, immune diversity returns to baseline after 24 hours. FIG. 6A shows an immune diversity tree map at baseline for subject 1. FIG. 6B shows the effect on the immune diversity map after a single 1200 mg dose of effervescent NAC in subject 1 24 hours post dose. For subject 1, diversity index increased from 12.7 to 20.0 (1.6 fold increase). The number and size of the rounded squares indicate a single T-cell beta chain receptor clonotype and its frequency in peripheral blood. FIG. 6C is a bar graph illustrating T cell β chain diversity index compared to baseline for subject 1) 24 hours after receiving a single dose of N-acetylcysteine (1200 mg).

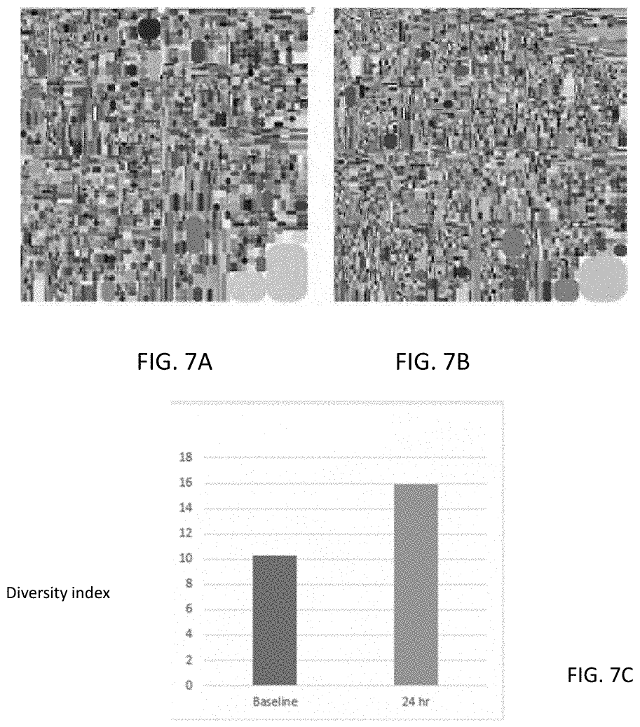

FIG. 7A and FIG. 7B show immune diversity maps for the TCR β chain immune repertoire after a single dosage of N-acetylcysteine (NAC) (2×600 mg effervescent tablets).for subject 2 (n=2) FIG. 7A shows an immune diversity tree map at baseline for subject 2. FIG. 7B shows the effect on the immune diversity map of a single 1200 mg dose of effervescent NAC in subject 2 24 hours post dose. For subject 2, diversity index increased from 10.3 to 15.9 (1.5 fold increase). The number and size of the rounded squares indicate a single T-cell beta chain receptor clonotype and its frequency in peripheral blood. FIG. 7C is a bar graph illustrating T cell β chain diversity index compared to baseline for subject 2 after receiving a single dose of N-acetylcysteine (1200 mg).

### 2.2-3(d) Topical Application

A salve comprising a decarboxylated cannabis extract in rosin format is prepared. The cannabis extract is slowly incorporated into a cosmetic base comprising an emollient, a humectant, and a preservative, heated to 80° C., and stirred until the rosin has been completely incorporated into the oil. The term “emollient” as used herein refers to fats or oils in a two-phase system (meaning one liquid is dispersed in the form of small droplets throughout another liquid). Emollients soften the skin by forming an occlusive oil film on the stratum corneum, preventing drying from evaporation in the deeper layers of skin. Thus, emollients are employed as protectives and as agents for softening the skin, rendering it more pliable. Emollients also serve as vehicles for delivery of hydrophobic compounds. Common emollients used in the manufacture of cosmetics include, but are not limited to, butters, such as Aloe Butter, Almond Butter, Avocado Butter, Cocoa Butter, Coffee Butter, Hemp Seed Butter, Kokum Butter, Mango Butter, Mowrah Butter, Olive Butter, Sal Butter, Shea Butter, glycerin, and oils, such as Almond Oil, Aloe Vera Oil, Apricot Kernel Oil, Avocado Oil, Babassu Oil, Black Cumin Seed Oil, Borage Seed Oil, Brazil Nut Oil, Camellia Oil, Castor Oil, Coconut Oil, Emu Oil, Evening Primrose Seed Oil, Flaxseed Oil, Grape Seed Oil, Hazelnut Oil, Hemp Seed Oil, Jojoba Oil, Kukui Nut Oil, Macadamia Nut Oil, Meadowfoam Seed Oil, Mineral Oil, Neem Seed Oil, Olive Oil, Palm Oil, Palm Kernel Oil, Peach Kernel Oil, Peanut Oil, Plum Kernel Oil, Pomegranate Seed Oil, Poppy Seed Oil, Pumpkin Seed Oil, Rice Bran Oil, Rosehip Seed Oil, Safflower Oil, Sea Buckthorn Oil, Sesame Seed Oil, Shea Nut Oil, Soybean Oil, Sunflower Oil, Tamanu Oil, Turkey Red Oil, Walnut Oil, Wheatgerm Oil. Exemplary natural humectants include glycerine, honey, aloe vera, hyaluronic acid, beeswax. Exemplary natural preservatives include, for example, tincture of benzoin made from friar's balsam, benzoin essential oil, rosemary oil extracts, phytocide aspen bark, Leucidal® liquid SF, and beeswax.

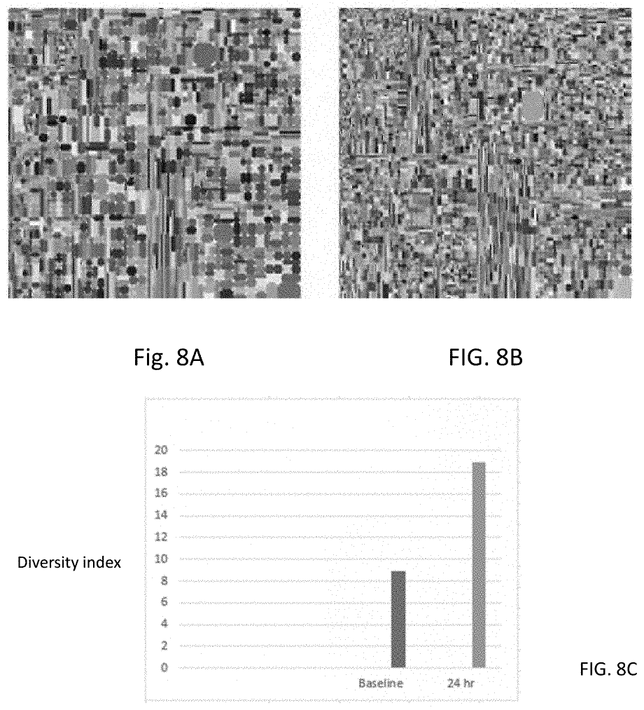

FIG. 8A and FIG. 8B show the immune diversity maps for TCR β chain immune repertoire for a subject (n=1) at baseline (FIG. 8A) and at 72 hours (FIG. 8B) after topical application of 10 mg THC salve made with silver OG (THC dominant strain). Diversity index increased from 8.9 to 18.9 (2.0 fold increase). No intoxication characteristic of THC occurred with application of salve for three days. The number and size of the rounded squares indicate a single T-cell beta chain receptor clonotype and its frequency in peripheral blood. FIG. 8C is a bar graph illustrating T cell β chain diversity index compared to baseline for the subject of FIG. 6 after applying a salve containing 10 mg THC made with Silver OG. Diversity index increased from 8.9 to 18.9 (2.0 fold increase).

Table 6 summarizes the diversity index data presented in FIG. 1-8.

| | | | Diversity index | | | |
|---|---|---|---|---|---|---|
| | | Sub- | | | 48 | Fold |
| Figure | Cannabinoid | ject | Baseline | Dosed | hr | change |
| FIG. | Cheese, 5 mg | 1 | 6.7 | 19.2 | | 2.9 |
| 1A/B/C | | | | | | |
| FIG. 2A | Cheese, 5 mg | 2 | 15.2 | 23.1 | | 1.5 |
| FIG. 2B | Cheese, 5 mg | 3 | 5.7 | 21.7 | | 3.8 |
| FIG. 2C | Cheese, 10 mg | 4 | 8.7 | 17.4 | | 2.0 |
| FIG. 2D | Cheese, 10 mg | 5 | 7.6 | 15.4 | | 2.0 |
| FIG. 2E | Granddaddy | 6 | 14.6 | 21.4 | | 1.5 |
| | Purple tablet | | | | | |
| — | — | 7 | — | — | | — |
| FIG. 2F | Cheese, 5 mg | 8 | 18.1 | 20.7 | | 0 |
| FIG. 2G | Cheese, 5 mg | 9 | 5.7 | 3.8 | | (−0.7) |
| FIG. | Jack CBD | 1 | 7.3 | 3.7 | 18 | 2.5 |
| 3A/B/C/D | | | | 24 hr (B) | | |
| | | | | (−0.5) | | |
| FIG. 4 | Jack CBD | 2 | 33.9 | 14.4 | | (−.4-−.8) |
| FIG. 5A/B | 1 mg THC | 1 | 8.6 | 8.1 | | (−0.6) |
| | (Cheese QDT) | | | | | |
| | 24 h | | | | | |
| FIG. 5C/D | 5 mg THC | 1 | 15.2 | 23.1 | | 1.5 |
| | (Cheese QDT) | | | | | |
| | 24 h | | | | | |
| FIG. 5E/F | 10 mg | 1 | 8.7 | 17.4 | | 2.0 |
| | THC (Cheese | | | | | |
| | QDT) 24 hr | | | | | |
| FIG. 6A/B | NAC 1200 | 1 | 12.7 | 20.0 | | 1.6 |
| FIG. 7 A/B | NAC1200 | 2 | 10.3 | 15.9 | | 1.5 |
| FIG. 8A/B | THC salve, | 1 | 8.9 | 18.9 | | 2.0 |
| | 10 mg made | | | | | |
| | with silver OG | | | | | |

### 2.2-4 Conclusions

With THC, there is a dose response for increasing immune diversity. In a cohort of nearly equal numbers of males and females, THC rapidly increases the total number of clonotypes in most individuals within 24-48 hr of applied dose. This effect on immune diversity is most observable when individuals have a relatively lower diversity index, controlling for circadian rhythm. CBD modulates the increase in diversity index response. NAC also increases the number of clonotypes. The immune repertoire diversity increase lasts longer with cannabinoids than with NAC. Oral cannabis and oral NAC taken together are complementary, and increase immune diversity. Topical (salve) delivery of high THC also increases diversity index, but without intoxication/spsychoactive effects.

### Example 3. Phase IV, Open Label, Adaptive 4-Arm Multidose, Single Center Exploratory Trial to Assess the Immune Diversity Response to Oral Administration of Four Natural Health Products in Healthy Adults, 60-70 Years of Age

### 3.1 Synopsis

Investigational Products: Palmitoylethanolamide, Echinacea purpurea, Curcumin, N-Acetyl-L-cysteine

Number of Participants: 32 healthy adults, aged 60-70 years

Study Design: Phase IV, open-label, randomized, adaptive, 4-arm, multi-dose study

Objective: Exploration of the effect of Natural Health Products (Palmitoylethanolamide, Echinacea purpurea, Curcumin, N-Acetyl-L-cysteine) on immune response-related/antibody diversity parameters.

Under Health Canada's Natural Health Products Regulations, which came into effect on Jan. 1, 2004, natural health products (NHPs) are defined as: Probiotics, Herbal remedies, Vitamins and minerals, Homeopathic medicines, Traditional medicines such as traditional Chinese medicines, and Other products like amino acids and essential fatty acids. NHPs must be safe to use as over-the-counter products and do not need a prescription to be sold. The Licensed Natural Health Products Database contains information about natural health products that have been issued a product license by Health Canada. Products with a license have been assessed by Health Canada and found to be safe, effective and of high quality under their recommended conditions of use.

### 3.2 Endpoints

Primary Endpoint: Laboratory measurements of the effect of the Natural Health Products (Palmitoylethanolamide [PEA], Echinacea purpurea, Curcumin, N-Acetyl-L-cysteine), orally administered daily for 14 days, on immune repertoire changes.

Secondary Endpoint: Safety assessment of Palmitoylethanolamide, Echinacea purpurea, Curcumin, N-Acetyl-L-cysteine, orally administered daily for 14 days, through clinical chemistry, emergence of adverse events and PI's assessment.

Population: Healthy males and females between the age of 60 to 70 years

### 3.3 Inclusion Criteria

Participants must meet all of the following criteria to be considered eligible for admission to the study.

1. Healthy male or female between the age of 60-70 years; “healthy” defined as participants with:

i. no active medical conditions, and/or;

ii. chronic prescription use which, in the opinion of the PI, is stable and does not negatively impact the participant's safety or study outcomes, or;

iii. Stable medical conditions, whether being treated with medications or not, which do not negatively impact participant safety or study outcomes in the PI's opinion.

2. Written informed consent obtained and signed by participant, demonstrating willingness and ability for participant to comply with all requirements and scheduled assessments of the study;

3. BMI between 18.5 and 29.9 kg/m², with a stable weight over the last 3 months as assessed by PI at screening;

4. Agrees not to use any over the counter, prescription, health supplement or dietary supplement from screening until the end of the study period unless approved for use by the PI for a stable medical condition;

5. Agrees to refrain from receiving vaccinations throughout the study period;

6. Agrees to maintain current dietary habits and level of physical activity for the trial duration;

7. Agrees to limit consumption of coffee/tea/other caffeinated beverages to no more than 2 cups per day for the duration of the trial [1], [2];

8. Does not regularly consume more than 2 standard* alcoholic beverages a day as assessed through verbal confirmation at screening, and agrees not to consume >2 standard* alcoholic beverages per day for the trial duration; A standard alcoholic beverage is defined as 12 ounces of beer, 5 ounces of wine, or 1.5 ounces of liquor

9. In good health as determined by lack of clinically significant abnormalities in health assessments (medical history, physical examination, clinical chemistry) performed at screening, and as assessed by the Principal Investigator.

### 3.4 Exclusion Criteria

1. Known sensitivity or allergy to any of the study products, or ingredients;

2. Self-reported smokers;

3. Females of childbearing potential;

4. Receipt or use of an investigational product in another research study within 30 days prior to Baseline (Visit 2) or currently participating in another research study;

5. Receipt of a vaccination of any type within 60 days prior to enrollment;

6. Difficulty with venipuncture and/or poor venous access;

7. Any major surgery within 1 year of enrollment; as assessed by PI;

8. Anticipated major surgeries or major life events (planned major travel or change in lifestyle conditions) intended to occur during the trial period;

9. Blood transfusion within 8 weeks of enrollment;

10. Donation of blood or plasma to a blood bank or clinical study within 8 weeks of enrollment and during the study except venipuncture as part of trial related activities;

11. Any clinically significant abnormalities in vital signs, as assessed by PI;

12. Hypertension or on anti-hypertensive medication, as assessed by PI;

13. History of, or current clinically significant cerebrovascular, metabolic, pulmonary, neurological, hematological, autoimmune, endocrine disorders, including individuals with Type I or Type II diabetes, or other clinically significant medical condition that, in the opinion of the Principal Investigator, may preclude safe study participation, or interfere with study objectives;

14. History of HIV/AIDS, viral or immune hepatitis, or current immune disorder or condition, as assessed by PI;

15. History of alcohol or substance abuse in the 12 months prior to screening, as assessed by PI;

16. History of any cannabis product use, including CBD, in the 3 months prior to screening, as assessed by PI;

17. Use of antibiotics within 14 days of enrollment;

17. Prescription medications for cardiovascular and autoimmune conditions will be excluded; any other prescription medication will be assessed by the PI on a case-by-case basis;

19. Any other medical, social or other condition that, in the opinion of the Principal Investigator, would preclude provision of informed consent, make participation in the study unsafe, complicate interpretation of study outcome data, or otherwise interfere with achieving the study objectives.

TABLE 6
Schedule of Assessments

| | In advance of | | | | |
|---|---|---|---|---|---|
| | study start | | | | |
| | (e.g., day 37 | Day- | Day | Day | Day |
| Treatment Day | to day-10) | 7 | 1 | 15 | 22 |
| Visit Number | 1 | 2 | 3 | 4 | 5 |
| Informed Consent | X | | | | |
| History and demographics | X | | | | |
| Physical exam | X | | | | |
| Vital signs | X | | | | |
| Clinical laboratory tests | X | | | | |
| Dispense study product | | | X | | |
| Blood draws (CBC, plasma, and | | X | X | X | X |
| iRepertoire ™ | | | | | |
| Provide Diary | | X | X | X | |
| Study product compliance and/or | | | X | X | X |
| diary review | | | | | |
| Visit Number | 1 | 2 | 3 | 4 | 5 |
| Adverse event (AE) assessment | | X | X | X | X |
| Concomitant medication check | X | X | X | X | X |
| Study termination assessments | | | | | X |

### 3.5 Background

Accumulating evidence suggests that over a lifetime the human immune system becomes less efficient [3] [4]. For example, the immune system can become depleted over time. As such, the immune surveillance system becomes less diverse, less flexible, and more vulnerable towards many diseases with age [7]. Research has been done to determine age-related changes in antibody and receptor repertoire, and the immune cell frequencies [5], [6].

Sponsor seeks to understand the human immune system and how different compounds have the potential to modulate, support and enhance the natural surveillance system. It therefore is planning to conduct an adaptive pilot clinical study to explore the effects of four Natural Health Products on the parameters of immune diversity; all products to be used in this study are licensed by Health Canada, and will be used under their authorized conditions of use.

The endpoints of the pilot clinical study are laboratory measurements and are not intended to provide evidence of treatment, prevention or cure of a disease or condition. This pilot study is not intended to be powered to support a Natural Health Product claim, and applying adaptive design allows modifications [9].

The objective is to explore the effect of Natural Health Products on immune response-related/antibody diversity parameters and the study design does not include any formal hypothesis testing. This pilot clinical study has an adaptive design: the second part of the study will be implemented only if the results of the first part support this methodological approach to the evaluation of immune modulation. An Exploratory Data Analysis (EDA) approach will be used to analyze data sets and to summarize their main characteristics [10].

As such, the statistical analysis plan for this pilot study is simply to provide descriptive statistics for all laboratory measurements as a tool for the Sponsor to conduct product development and for hypothesis generation for future studies.

### 3.6 Study Objective

Objective:

Exploration of the effect of Natural Health Products (Palmitoylethanolamide [PEA], Echinacea purpurea, Curcumin, N-Acetyl-L-cysteine) on immune response-related/antibody diversity parameters.

Endpoints:

Primary Endpoint: Laboratory measurements of the effect of the Natural Health Products (Palmitoylethanolamide, Echinacea purpurea, Curcumin, N-Acetyl-L-cysteine), orally administered daily for 14 days, on immune repertoire changes.

Secondary Endpoint: Safety assessment of Palmitoylethanolamide, Echinacea purpurea, Curcumin, N-Acetyl-L-cysteine, orally administered daily for 14 days, through clinical chemistry emergence of adverse events and PI's assessment.

### 3.7 Selection of Study Population

Inclusion Criteria

Participants must meet all of the following criteria to be considered eligible for admission to the study.

1. Healthy male or female between the ages of 60-70 years; “healthy” defined as participants with:

i. no active medical conditions, and/or;

ii. chronic prescription use which, in the opinion of the PI, is stable and does not negatively impact the participant's safety or study outcomes, or;

iii. Stable medical conditions, whether being treated with medications or not, which do not negatively impact participant safety or study outcomes in the PI's opinion.

2. Written informed consent obtained and signed by participant, demonstrating willingness and ability for participant to comply with all requirements and scheduled assessments of the study;

3. BMI between 18.5 and 29.9 kg/m², with a stable weight over the last 3 months as assessed by PI at screening;

4. Agrees not to use any over the counter, prescription, health supplement or dietary supplement from screening until the end of the study period unless approved for use by the PI for a stable medical condition;

5. Agrees to refrain from receiving vaccinations throughout the study period;

6. Agrees to maintain current dietary habits and level of physical activity for the trial duration;

7. Agrees to limit consumption of coffee/tea/other caffeinated beverages to no more than 2 cups per day for the duration of the trial;

8. Does not regularly consume more than 2 standard* alcoholic beverages a day as assessed through verbal confirmation at screening, and agrees not to consume >2 standard* alcoholic beverages per day for the trial duration;

9. In good health as determined by lack of clinically significant abnormalities in health assessments (medical history, physical examination, clinical chemistry) performed at screening, and as assessed by the Principal Investigator.

* A standard alcoholic beverage is defined as 12 ounces of beer, 5 ounces of wine, or 1.5 ounces of liquor

Exclusion Criteria

Participants meeting any of the following criteria will not be eligible for admission to the study:

1. Known sensitivity or allergy to any of the study products, or ingredients;

2. Self-reported smokers;

3. Females of childbearing potential;

4. Receipt or use of an investigational product in another research study within 30 days prior to Baseline (Visit 2) or currently participating in another research study;

5. Receipt of a vaccination of any type within 60 days prior to enrollment;

6. Difficulty with venipuncture and/or poor venous access;

7. Any major surgery within 1 year of enrollment; as assessed by PI;

8. Anticipated major surgeries or major life events (planned major travel or change in lifestyle conditions) intended to occur during the trial period;

9. Blood transfusion within 8 weeks of enrollment;

10. Donation of blood or plasma to a blood bank or clinical study within 8 weeks of enrollment and during the study except venipuncture as part of trial related activities;

11. Any clinically significant abnormalities in vital signs, as assessed by PI;

12. Hypertension or on anti-hypertensive medication, as assessed by PI;

13. History of, or current clinically significant cerebrovascular, metabolic, pulmonary, neurological, hematological, autoimmune, endocrine disorders, including individuals with Type I or Type II diabetes, or other clinically significant medical condition that, in the opinion of the Principal Investigator, may preclude safe study participation, or interfere with study objectives;

14. History of HIV/AIDS, viral or immune hepatitis, or current immune disorder or condition, as assessed by PI;

15. History of alcohol or substance abuse in the 12 months prior to screening, as assessed by PI;

16. History of any cannabis product use, including CBD, in the 3 months prior to screening, as assessed by PI;

17. Use of antibiotics within 14 days of enrollment;

18. Prescription medications for cardiovascular and autoimmune conditions will be excluded; any other prescription medication will be assessed by the PI on a case-by-case basis;

19. Any other medical, social or other condition that, in the opinion of the Principal Investigator, would preclude provision of informed consent, make participation in the study unsafe, complicate interpretation of study outcome data, or otherwise interfere with achieving the study objectives.

Any deviation from these inclusion and exclusion criteria must be approved by the Investigator on a case-by-case basis prior to enrollment of the participant. All eligibility waivers must be approved by the Sponsor. The protocol deviation waiver must be documented by the investigator and/or the Sponsor.

### 3.8 Concomitant Medications

Any medication taken by the participant throughout the study period will be regarded as concomitant treatment. During the study, participants shall be instructed not to take medications unless absolutely necessary. If the use of any concomitant treatment becomes necessary during the study, the treatment will be recorded in the CRF, including the name of the drug or treatment, indication for use, dosage, route, date, and time of treatment.

### 3.9 Early Withdrawal

Removal by Principal Investigator: The removal of a study participant by the Principal Investigator will be based on the Clinical and Protocol Violation conditions outlined below. If possible, any evaluations or assessments planned for the end of the study will be completed at an Early Termination (ET) Visit. Criteria for participant removal at the PI's discretion are:

### 3.10 Clinical

A participant may be withdrawn from the study if, in the opinion of the PI, it is not in the best interest of the participant to continue. Participants who experience an adverse event (AE) or severe adverse event (SAE) will be assessed by the PI and determine if removal from the study is necessary.

### 3.11 Protocol Violation

Protocol violations will be assessed by the PI on a case-by-case basis with removal from the study if necessary.

Participants can be withdrawn from the study by the PI at any time at the discretion of the Sponsor.

### 3.12 Investigational Product (IP)

Manufacturing and Storage

The test articles will be manufactured in accordance with the Natural Health Product Regulations. They will be stored in a secure area with limited access at the research site.

### Labelling

All investigational products will have labels according to applicable Regulations. IPs are licensed Natural Health Products and will be used within their approved indication as per Health Canada Regulations.

### Directions

Directions for the administration of each IP will be indicated on the participant daily diary and explained by the delegated site staff. Participants will be instructed on dosing requirements and asked to follow the dosing instructions for their respective IP, indicating any deviations on their daily study diary. Summarized below are the directions for each IP:

TABLE 7

| Investigational Product | | |
|---|---|---|
| (Name and NPN) | Branded Name | Dosing Instructions |
| Palmitoylethanolamide- | Natural Factors- | Participants will be instructed |
| 80097472 | PEA400 | to take 3 capsules (400 mg |
| | | each) once daily with water |
| Echinacea Purpurea- | Natural Factors- | Participants will be instructed |
| | Echinamide ® | to take 1 capsule (250 mg |
| | | each) twice daily with water |
| Curcumin-80043455 | Natural Factors- | Participants will be instructed |
| | Theracurmin ® | to take 3 capsules (60 mg |
| | | each) once daily with water |
| N-Acetyl-L-cysteine- | Sisu-NAC | Participants will be instructed |
| 80004844 | | to take 1 capsules (600 mg |
| | | each) twice daily with water |

### 3.13 Randomization

Randomization will be applied to this study to fill all study arms simultaneously without bias.

### 3.14 Study Design

In this study immune repertoire changes will be measured by genetic sequencing of segments of mRNA in the participant's blood that correspond to the coding regions for the Complementarity Determining Region—3 (CDR-3) of T- and B-cell surface receptors. CDR-3 receptor segments are the highly variable recognition elements of the T- and B-cell receptors responsible for recognition of foreign cells and other entities. This recognition step is an early step in the adaptive immune response. Longitudinal changes in immune diversity and dynamics, as indicated by changes in the CDR-3 sequence information will be measured over a number of timeframes. Baseline dynamics and diversity will be measured in comparing the results from Visits 2 and 3. The impact of the IPs will be evident from comparing results between Visits 3 and 4. Finally, the impact of stopping the IP dosing will be evident in comparing the results from Visit 5 to those of 4, 3, and 2. The techniques being used to analyze the sequence information are exploratory. A general discussion of evaluation techniques can be found in: “The Changing Landscape of Naive T Cell Receptor Repertoire With Human Aging.” [11], [12] and “Overview of methodologies for T-cell receptor repertoire analysis.” [12].

This adaptive research study focuses on exploring the effect of eight licensed Natural Health Products (NHPs) on the immune system using eight arms and multiple products, across 14 days of intervention in two periods. The adaptive, two-period study design enables continuation of the study based on interim data analysis of the first period including four investigational products (Palmitoylethanolamide, Echinacea purpurea, Curcumin, N-Acetyl-L-cysteine). Data from the first study period will be assessed by the Sponsor and will determine if conducting the second period of the study (including four new sets of IPs) is necessary; this makes the clinical trial more flexible, efficient and fast.

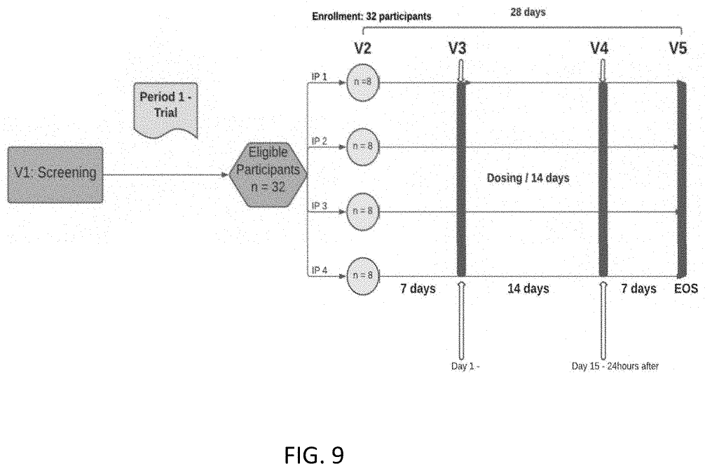

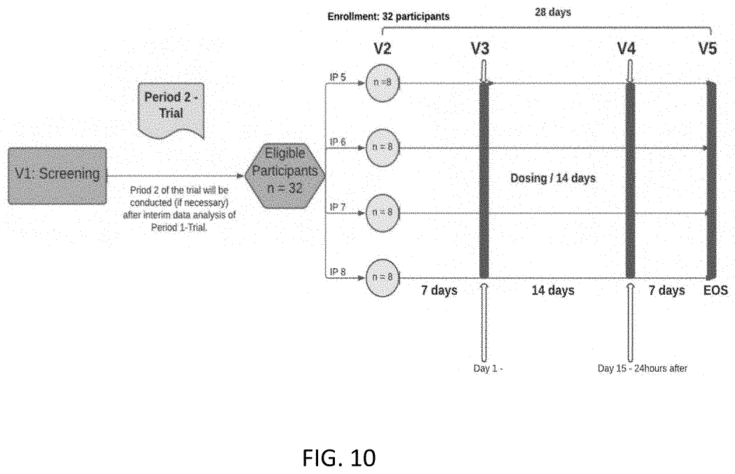

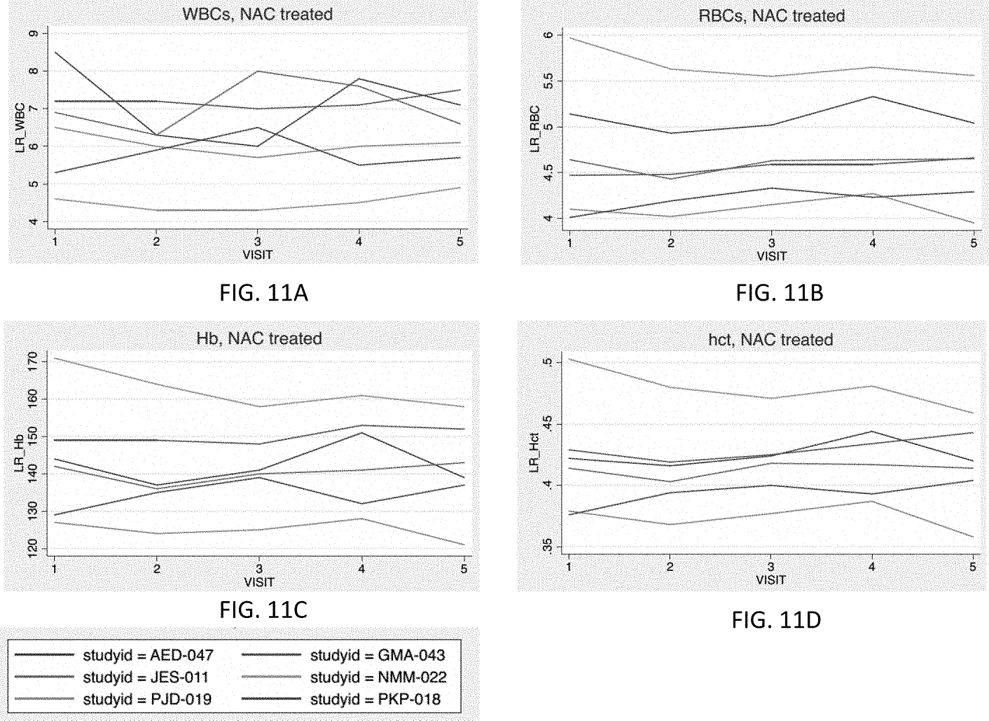

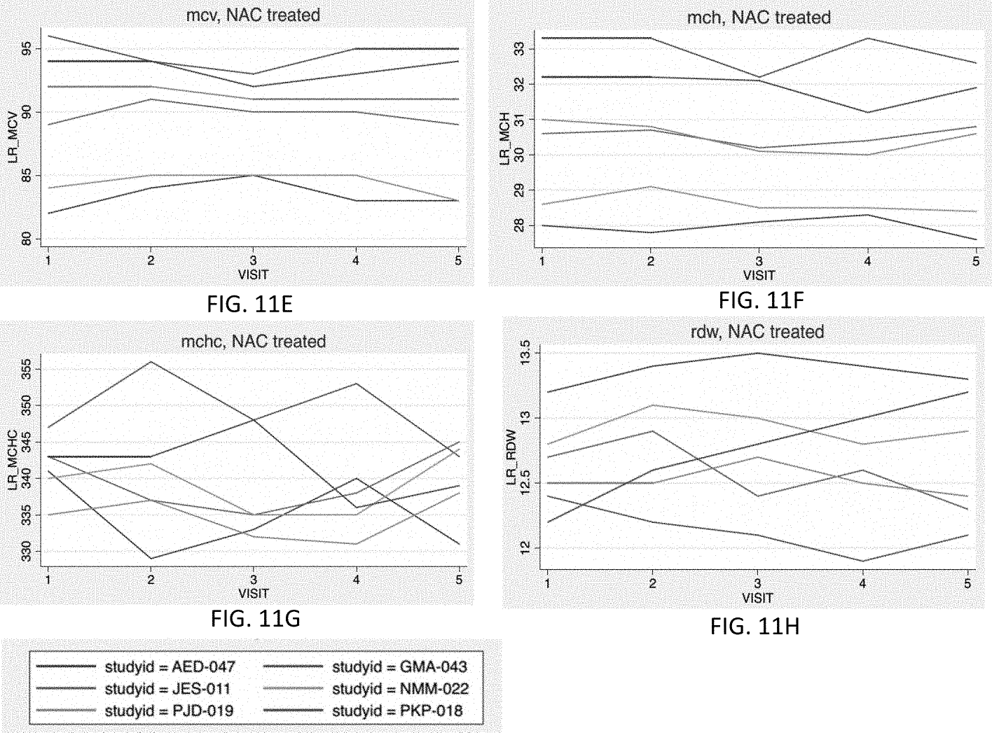

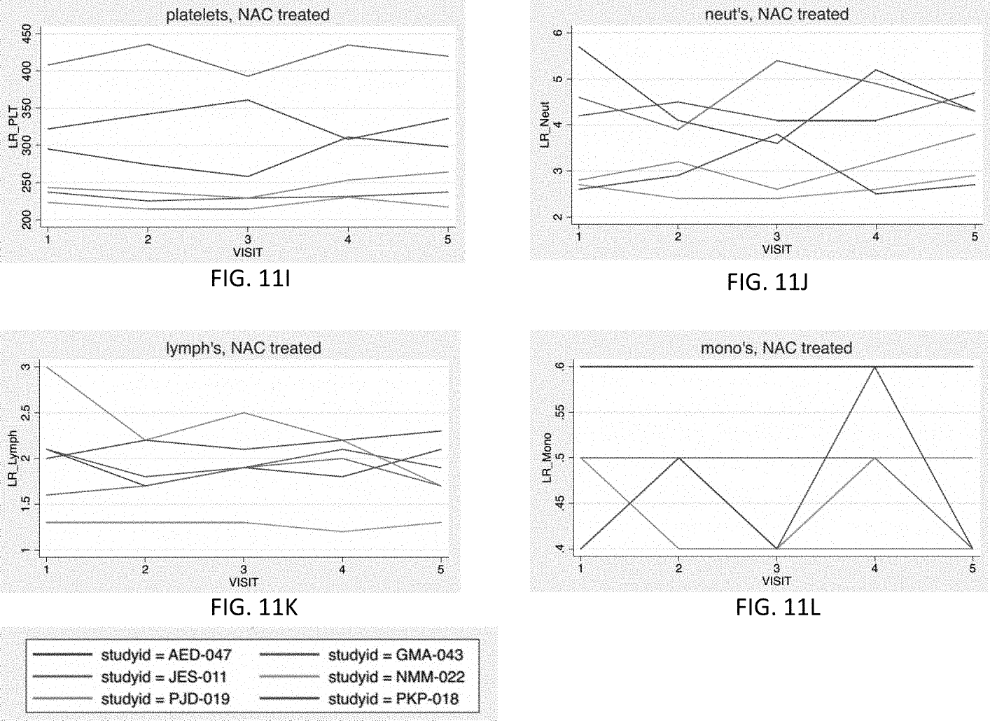

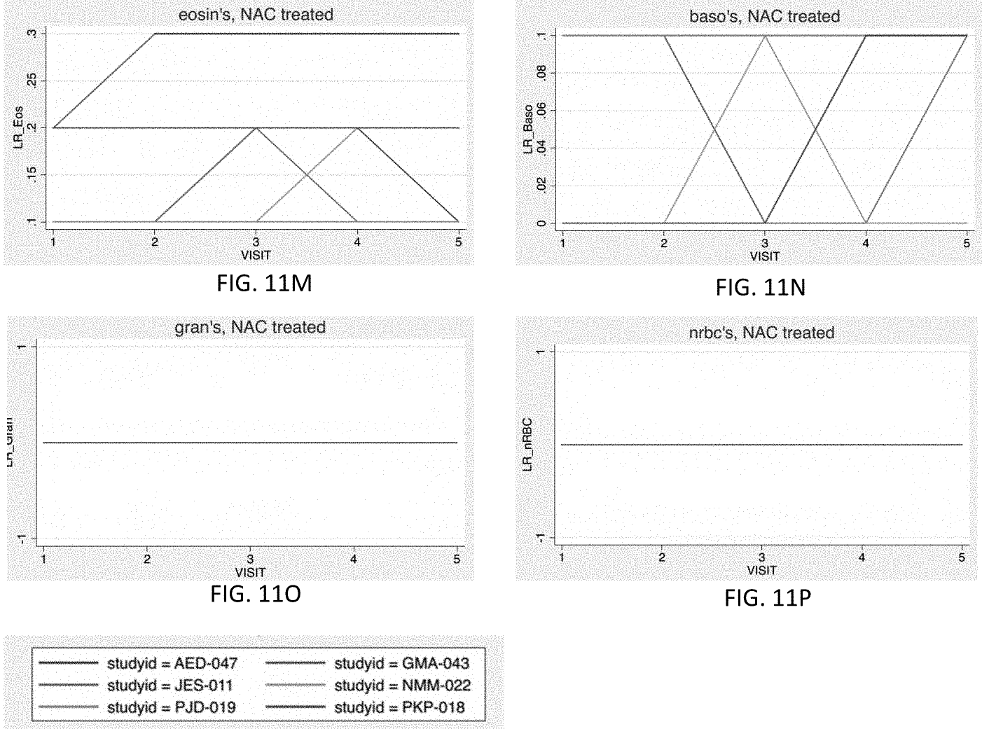

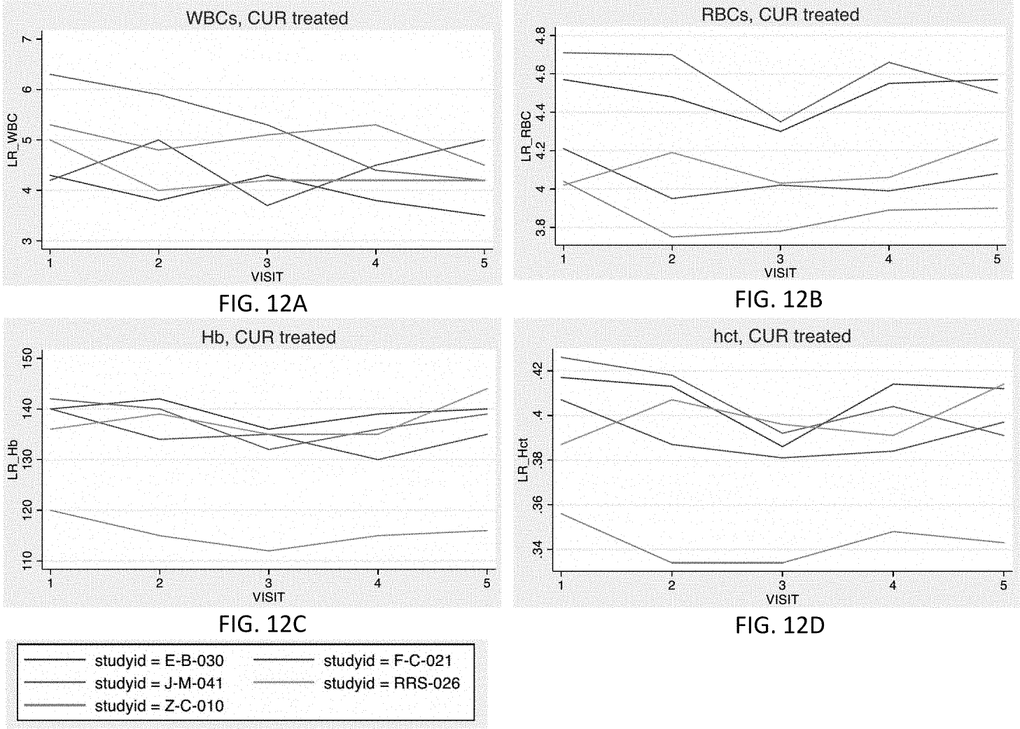

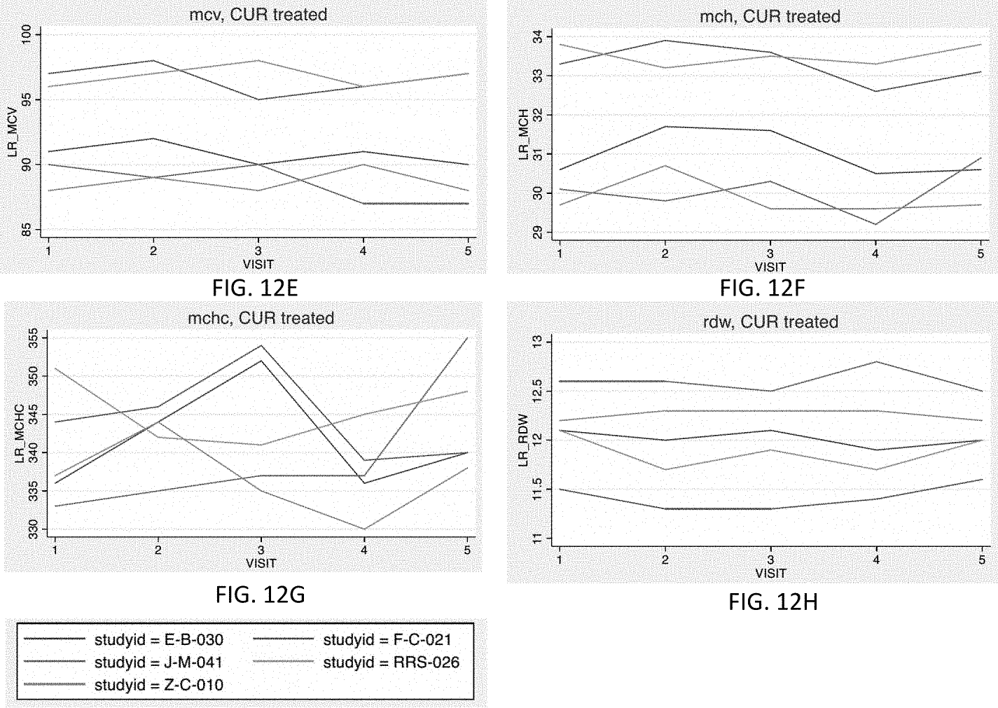

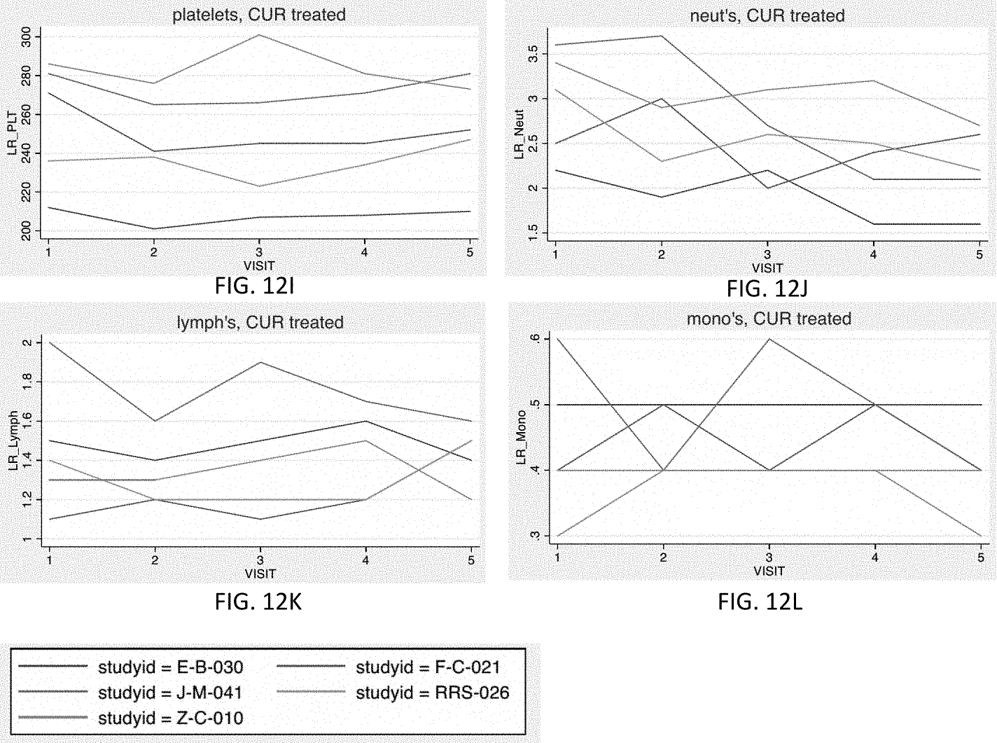

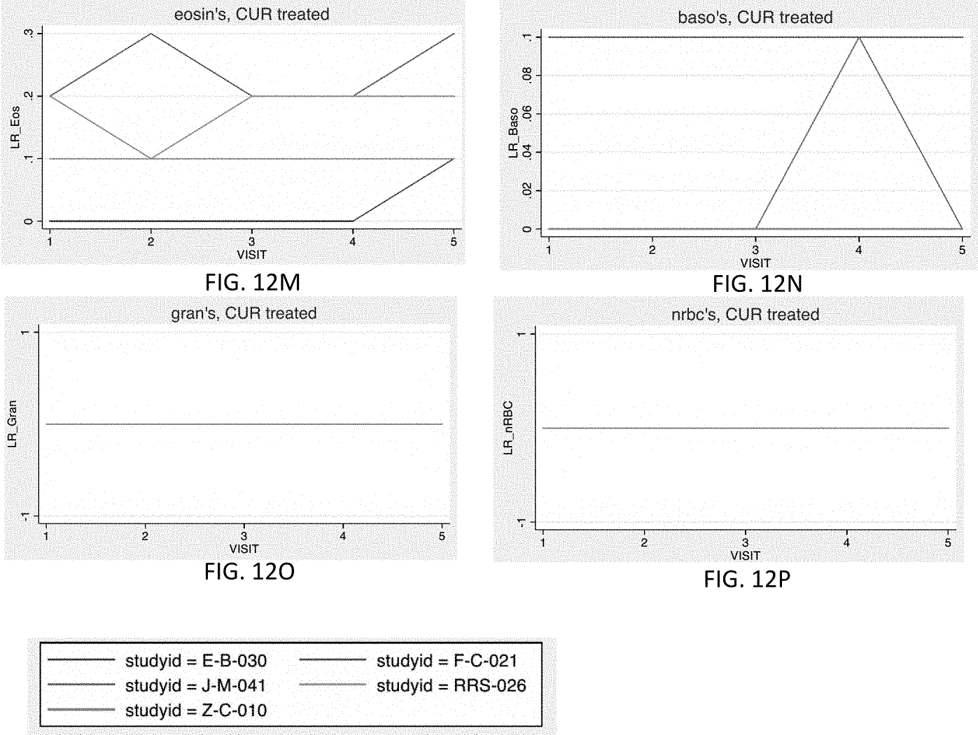

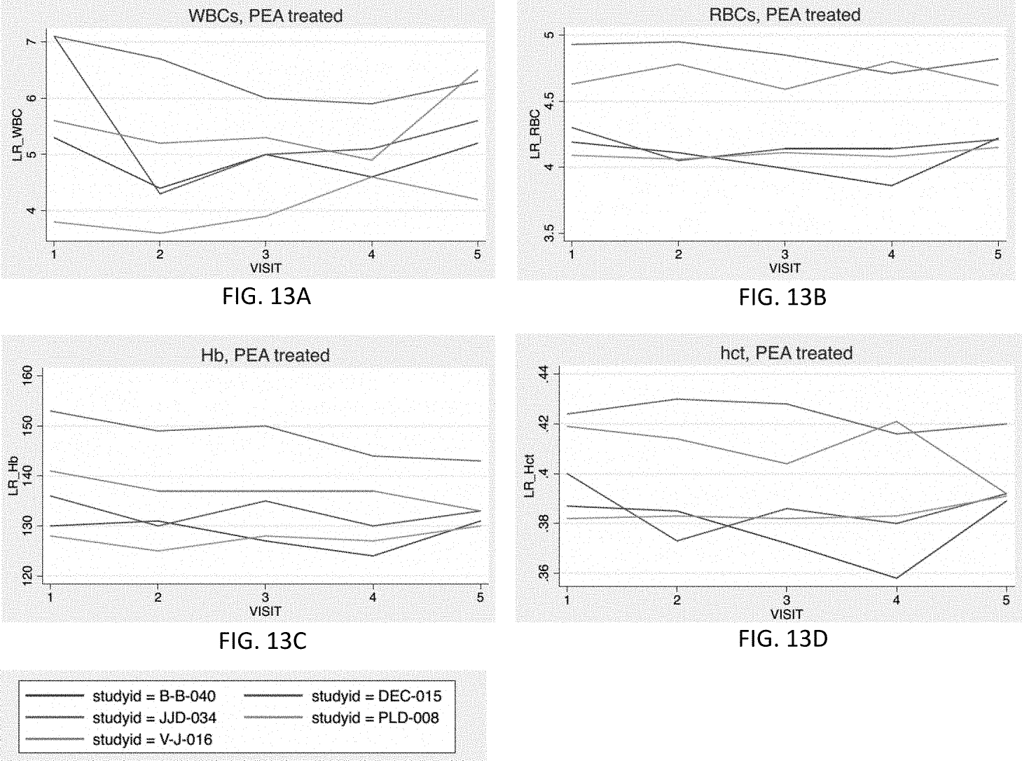

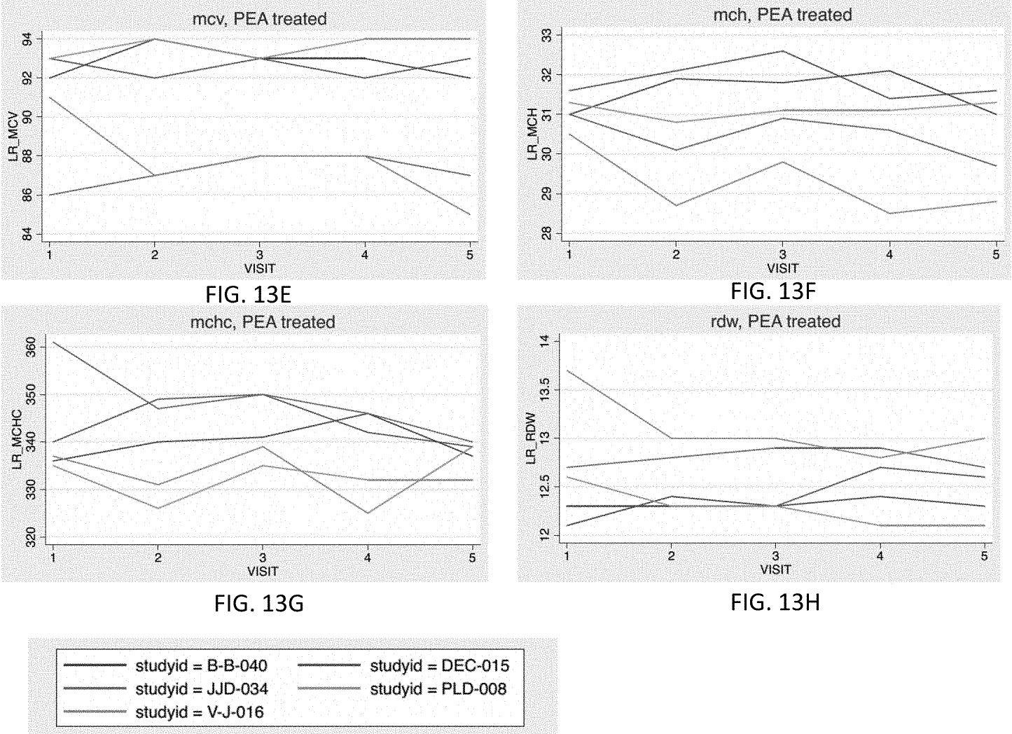

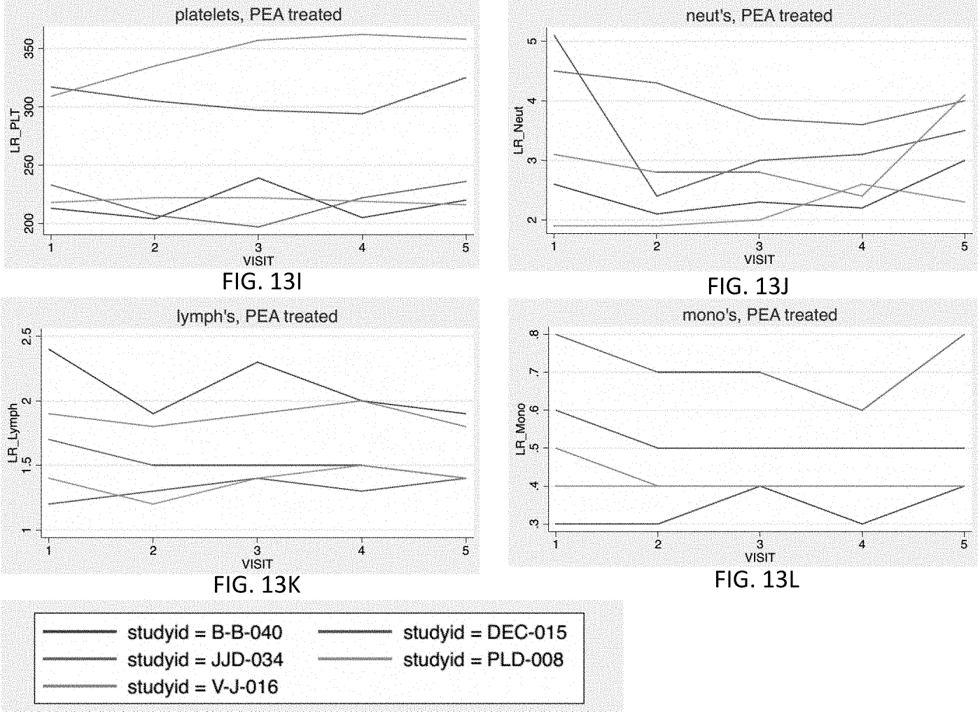

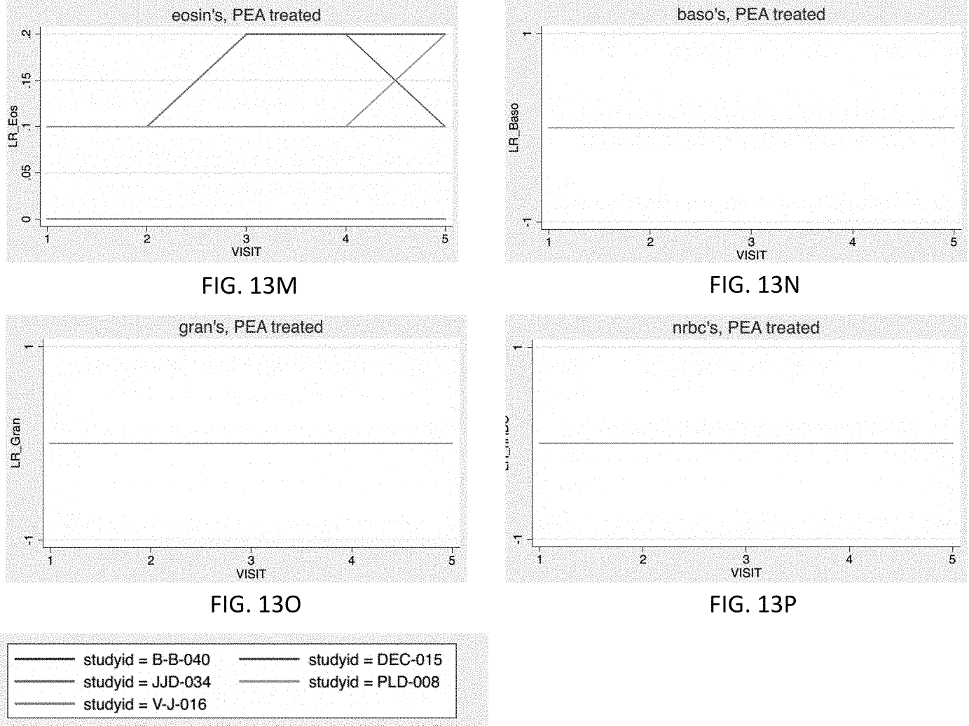

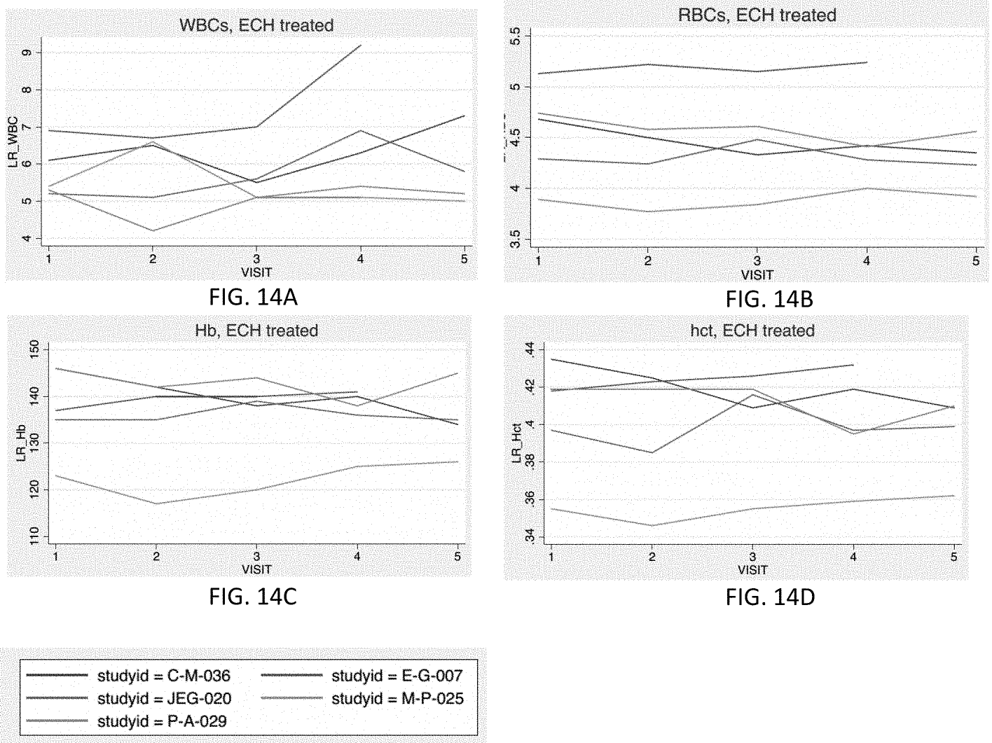

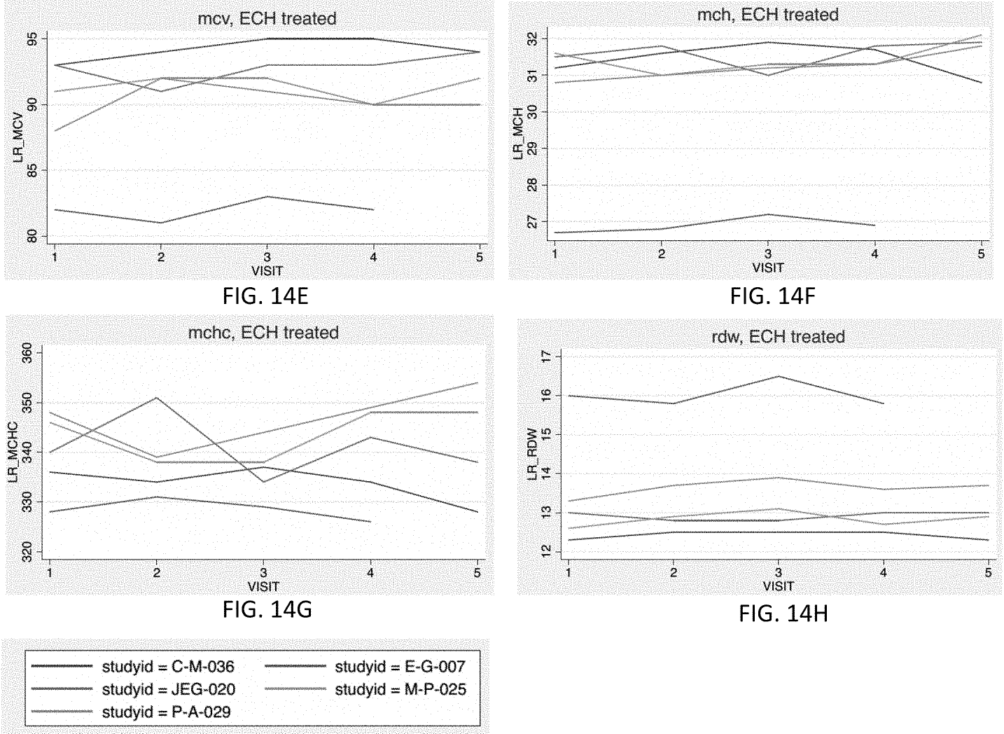

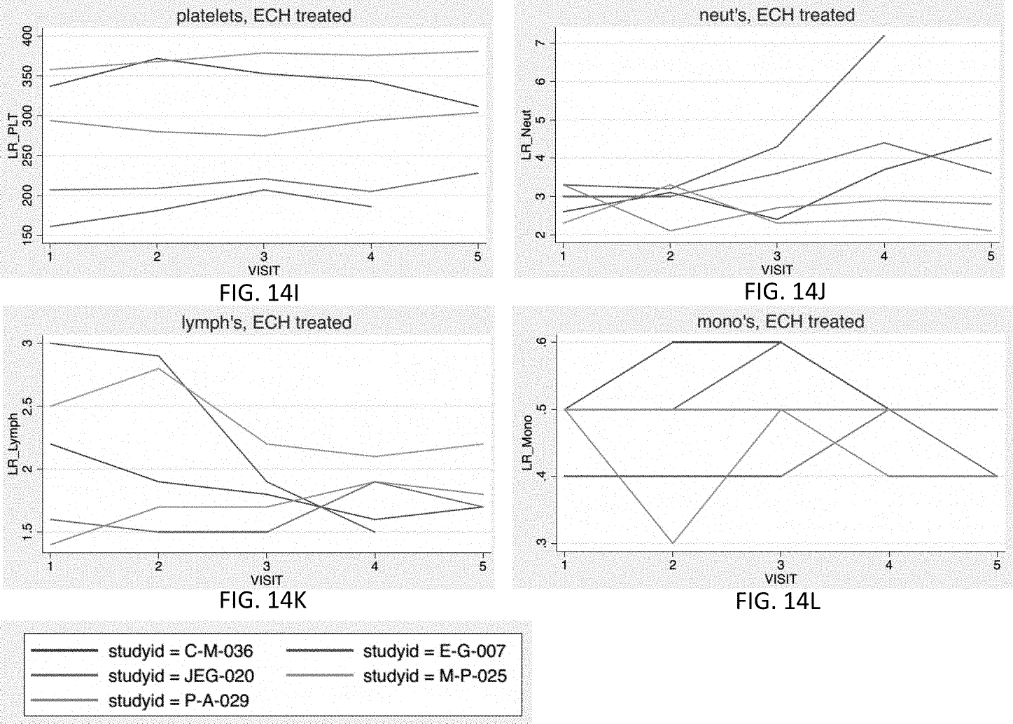

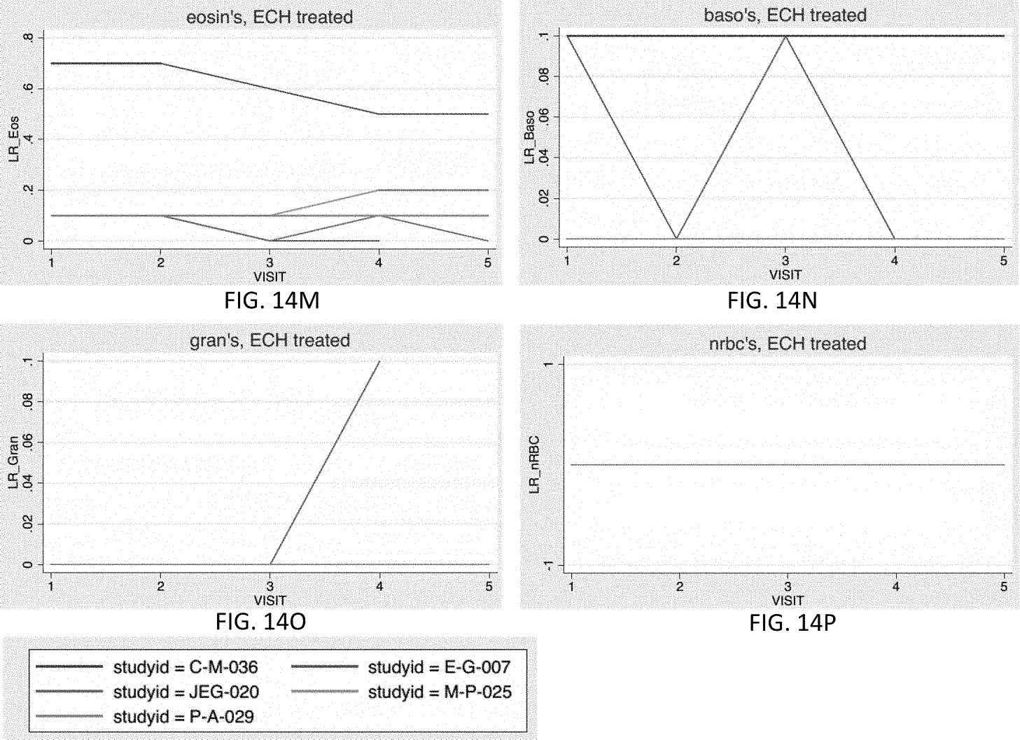

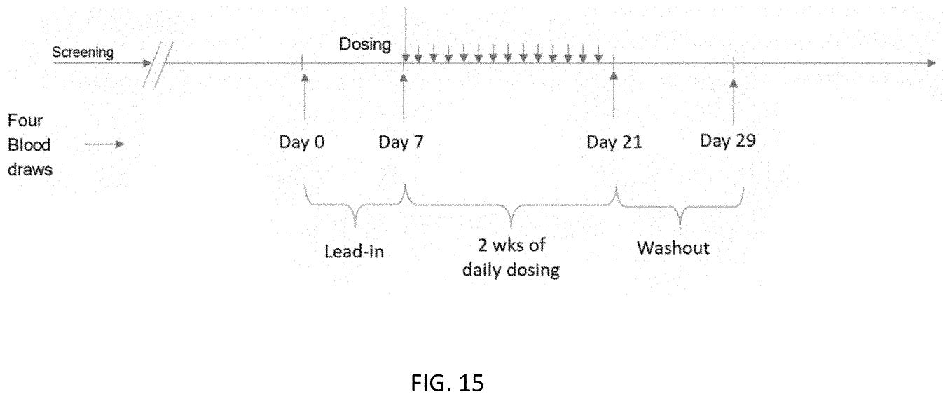

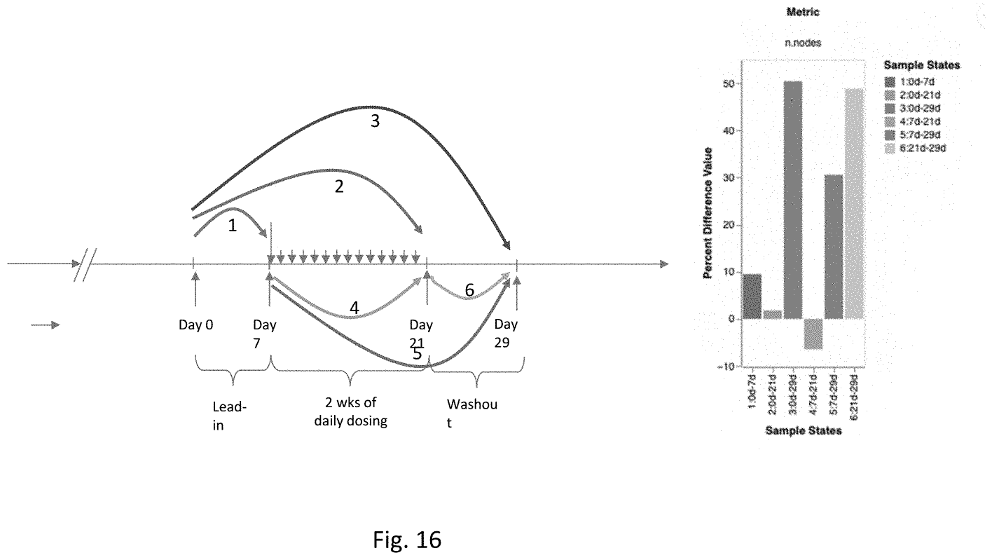

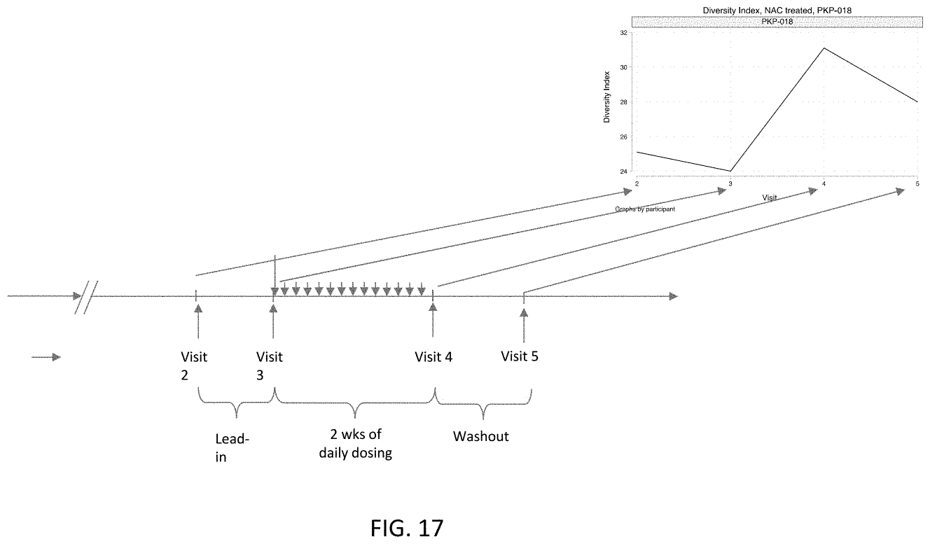

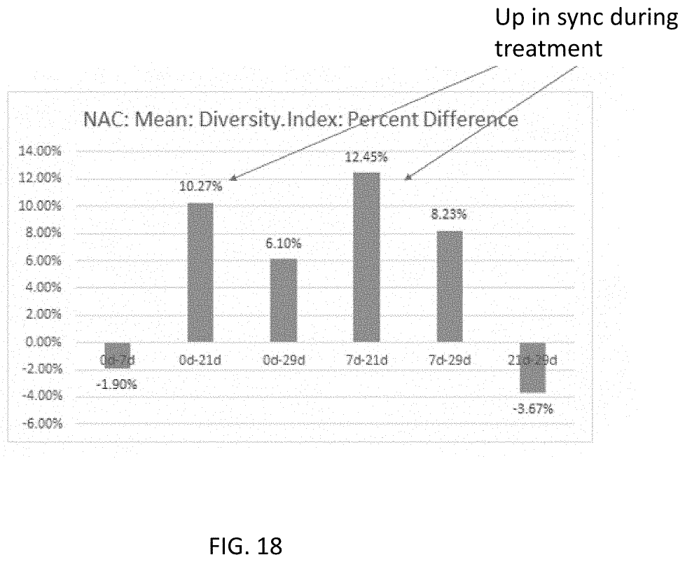

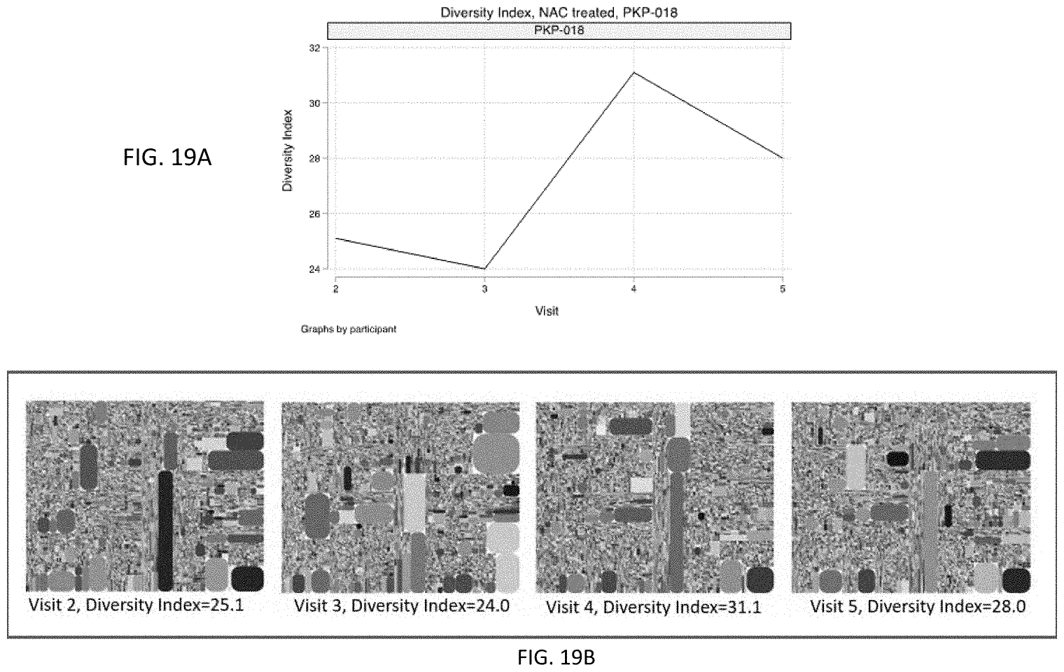

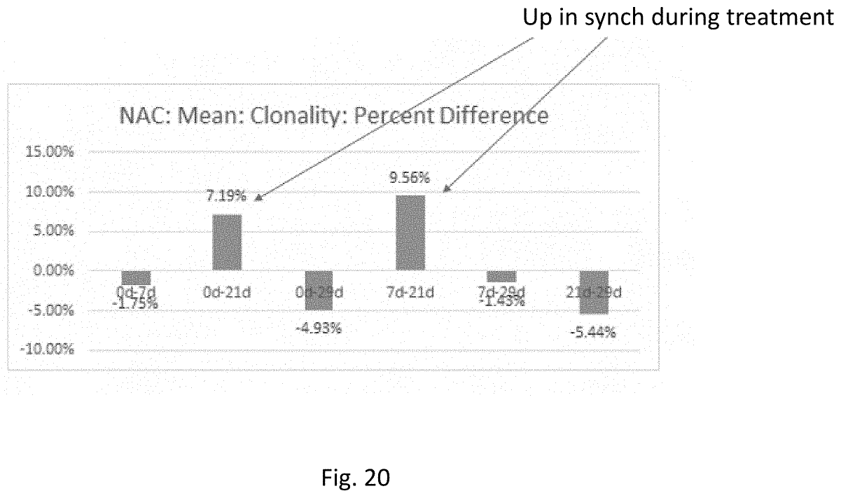

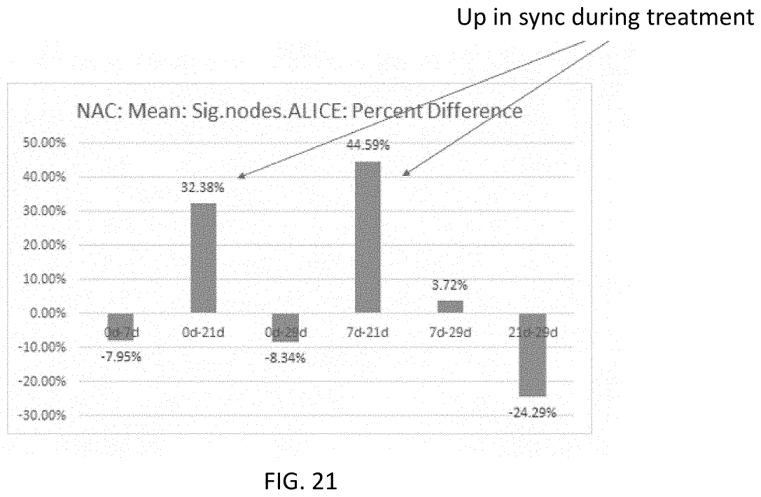

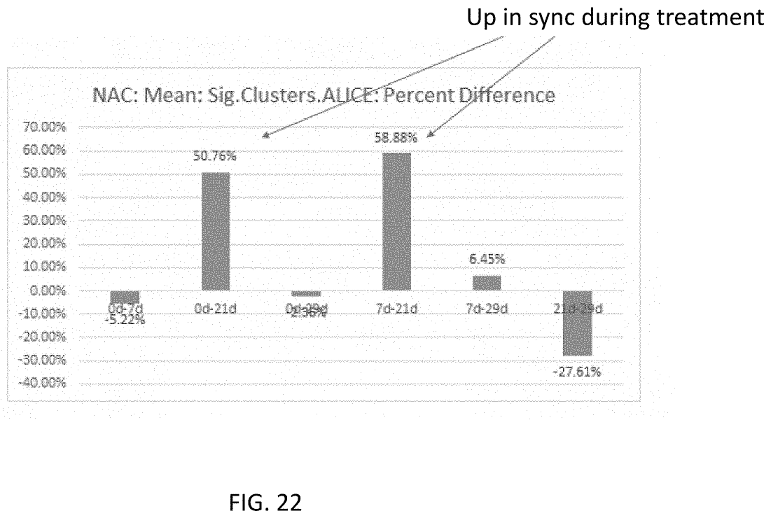

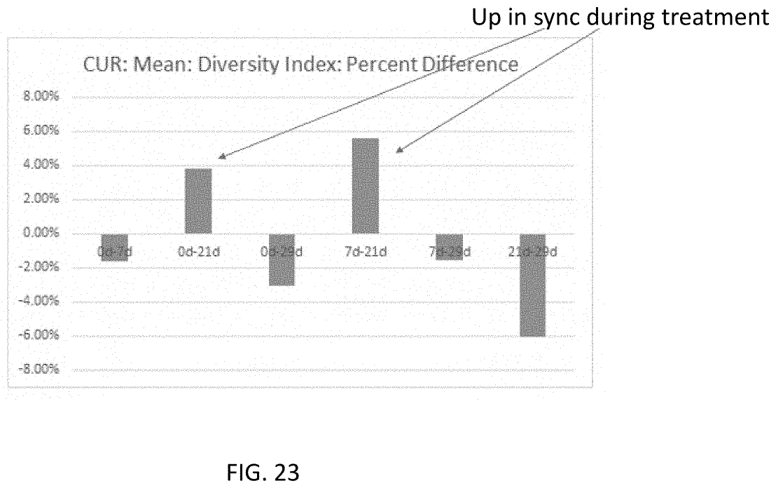

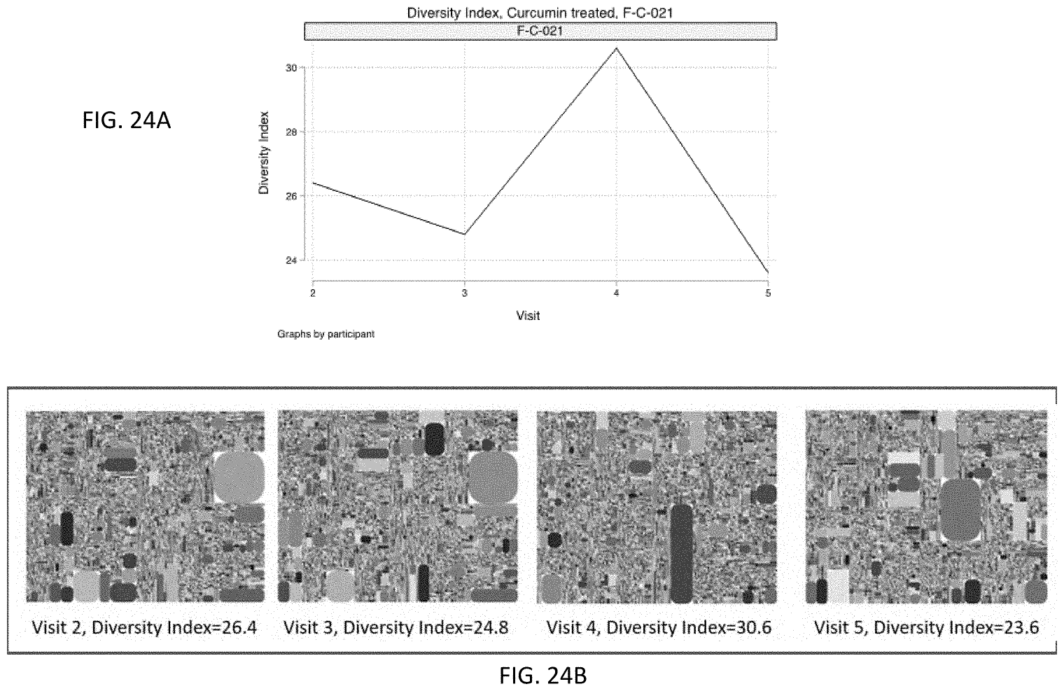

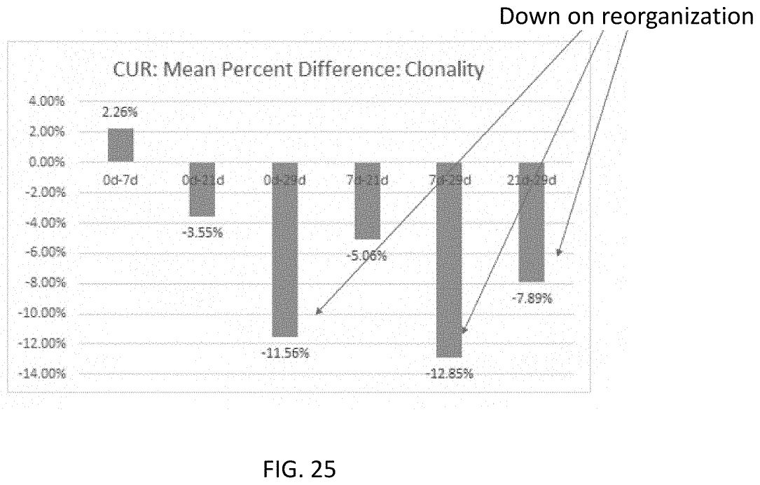

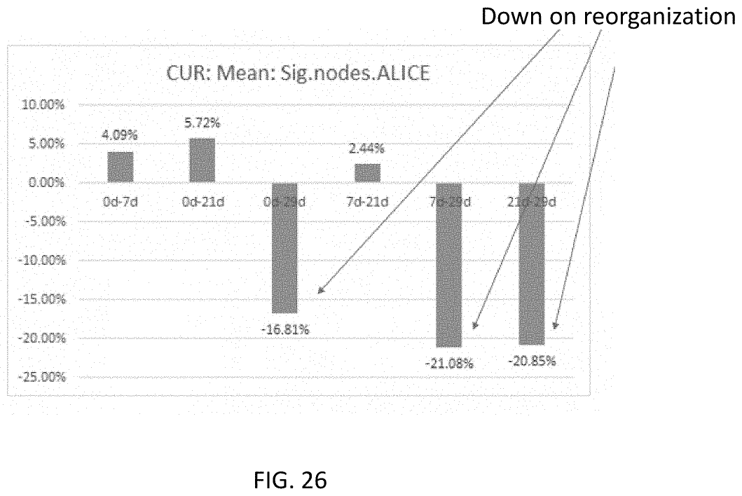

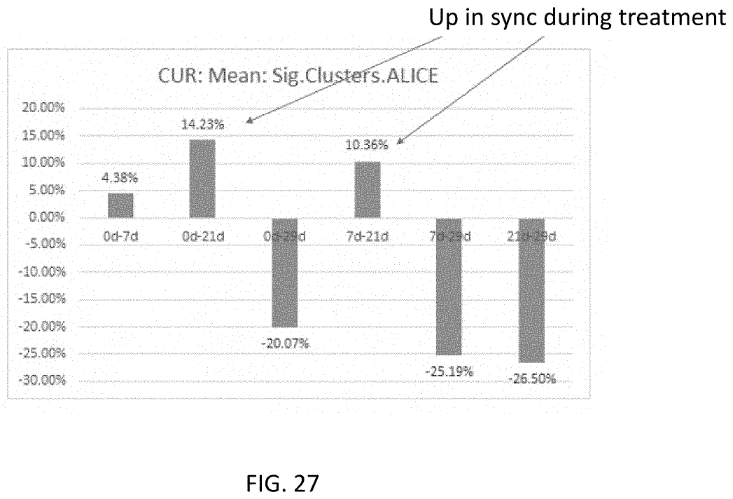

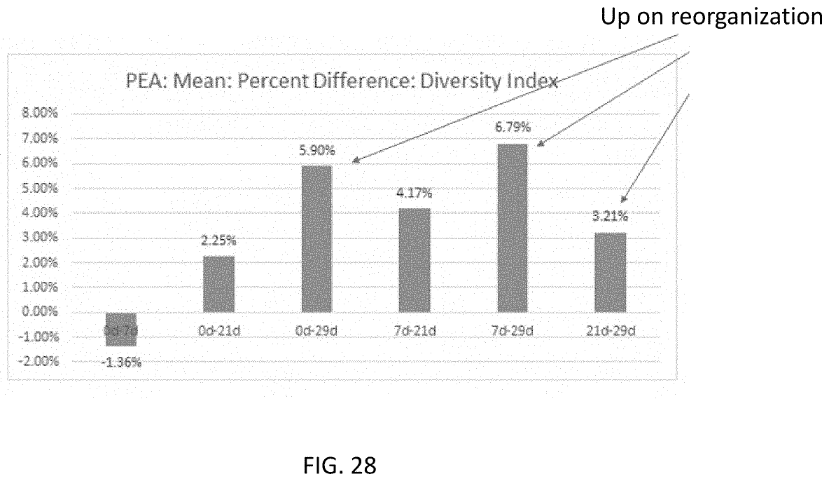

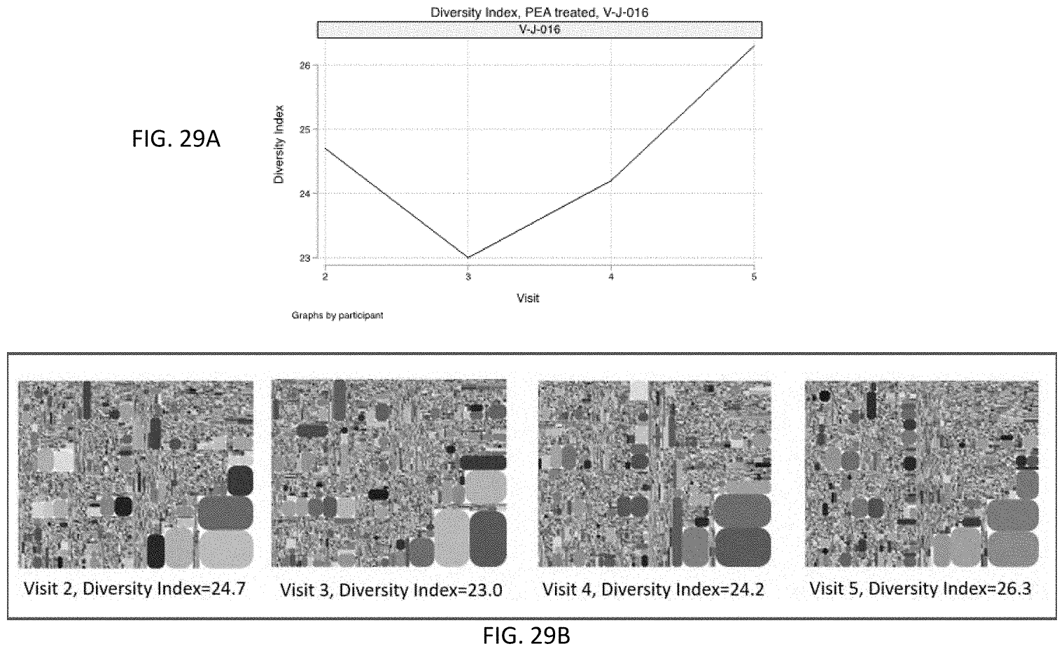

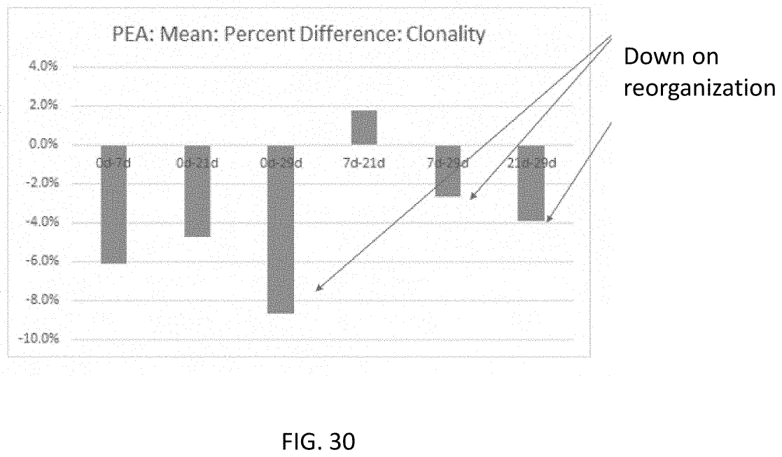

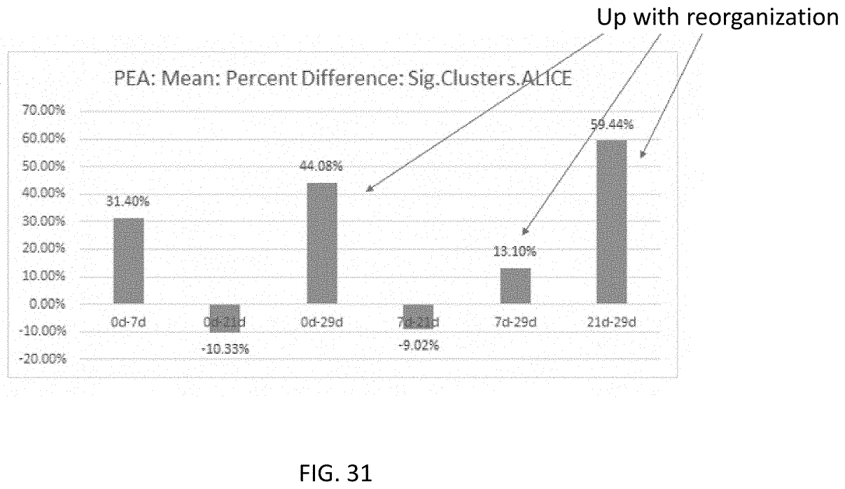

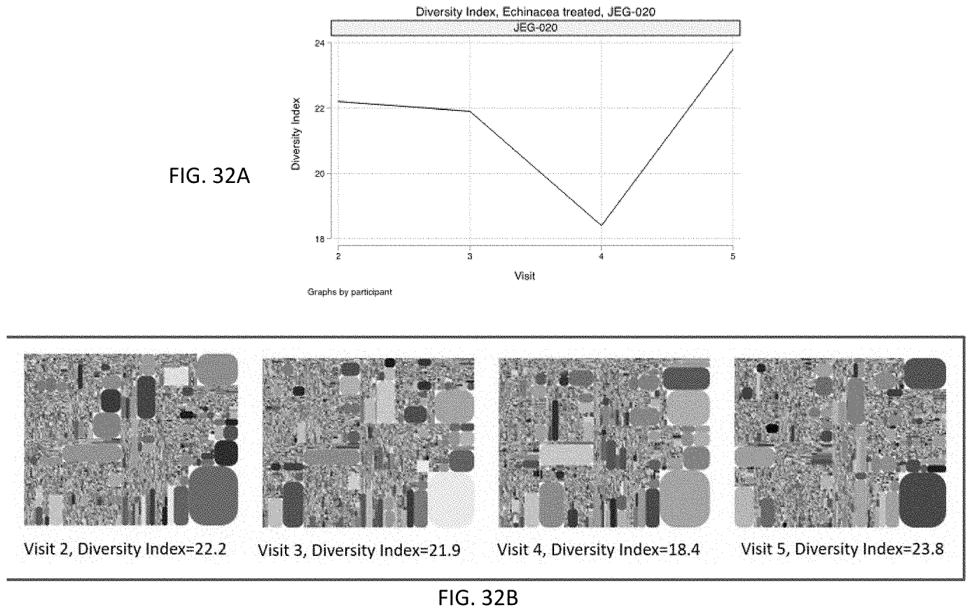

This protocol explains details of the first period of the trial (four arms and four IPs as shown in FIG. 9) and will reference the adaptations that may take place later (second period), depending on the results of the data collected during the first period. A protocol modification will be submitted to the IRB, if the Sponsor will intend to continue the second period of the research study with the additional four IPs with four arms as shown in FIG. 10.

FIG. 9 and FIG. 10 provide diagrams that summarize the adaptive study design for both periods.

### 3.15 Study Visits

### 3.15(a) Visit 1 (V1)—Screening

Participants will not be required to fast prior to this visit. During this visit, the delegated study staff will present orally and in writing the objectives, the process, and the modalities of participation in the research. The study staff will answer any questions the participant may have.

Before participation in the study, and after a sufficient period of reflection, the written and informed consent of the participant will be sought. The Informed Consent Form (ICF) will be provided to the participant. Once signed by the participant and attested to by the PI or delegate, a copy of the signed original ICF will be provided to the participant.

After informed consent has been obtained, the PI and/or delegated research site staff will collect demographic information, complete medical history, and conduct a clinical examination, including a physical exam, measurements of body temperature, blood pressure, heart rate, respiratory rate, and anthropometric measurements (height, weight, calculated BMI), in order to verify the eligibility criteria (excluding clinical chemistry parameters).

If the participant meets the inclusion criteria and none of the exclusion criteria (excluding clinical chemistry), the V1 screening clinical chemistry assessments will be conducted to further assess eligibility.

At the end of the visit, if the participant is eligible for inclusion in the study (excluding the results of the pending clinical chemistry assessments), the V2 inclusion visit will be scheduled as per the protocol schedule of assessments, and as per the PI's discretion where washouts or repeated eligibility assessments are required.

Blood samples to be collected will be analyzed for the following tests: CBC [Includes: White Blood Cell Count (WBC), Red Blood Cell Count (RBC), Hemoglobin (HB), Hematocrit (HCT), MCV, MCH, MCHC, Platelet Count]

Glucose

Sodium

Potassium

Creatinine

Alanine Amino Transferase (ALT)

Alkaline Phosphatase

Bilirubin Total

Urea

Serum protein

Albumin

Gamma Glutamyl Transpeptidase (GGT)

Calcium

Phosphorus

Aspartate Amino Transferase (AST)

The results will be reviewed by the PI to determine eligibility for enrollment in the trial. The participant will be notified of their eligibility, or ineligibility pending the PI's review of these results.

### 3.15(b) Prior to Visit 2

Prior to Visit 2 and depending on the results of blood parameters, the eligibility of the participant will be determined: to be eligible, all blood parameters must be within the acceptable reference value ranges as assessed by the PI.

If the participant is ineligible, the next visit will be cancelled; the investigating team will inform the participant of the ineligibility status.

If the participant is eligible, the participant will be notified and reminded to proceed to Visit 2 at the research site.

### 3.15(c) Visit 2—Baseline—Day −7

Participants must be at least 2 hours fasted (no food or drink except water) prior to this visit. At Visit 2, the Investigator or delegated research site staff will review medical history and conduct a clinical examination, including a measurement of: body temperature, blood pressure, heart rate, respiratory rate, and weight. Participants must meet all of the inclusion criteria and none of the exclusion criteria to be enrolled into the study.

Blood samples to be collected will be analyzed for the following tests:

CBC [Includes: White Blood Cell Count (WBC), Red Blood Cell Count (RBC), Hemoglobin (HB), Hematocrit (HCT), MCV, MCH, MCHC, Platelet Count]

iRepertoire mRNA lymphocyte CDR-3 sequencing

Plasma for potential future analysis

The results will be assessed by the PI. The clinical chemistry parameters must be within the reference value ranges and/or deemed by the PI as not clinically significant. Otherwise, the participant will be contacted by the PI or delegate to further assess and determine an appropriate course of action.

At V2, the participant will be given a paper daily diary in order to record the use of any concomitant medications, and any adverse events that may have occurred. The diary will be reviewed at the research site at each visit as indicated on the Schedule of Assessments.

Immune diversity measurements have a dependence on the time of day of measurement. For longitudinal consistency all subsequent visits will be scheduled to be as close as possible to the time of Visit 2 with a target time window of +/−1 hour.

The participant will proceed to Visit 3 at the research site.

### 3.15(d) Visit 3 (V3)—Dosing—Day 1

Participants must be at least 2 hours fasted (no food or drink except water) prior to this visit. The participant will be randomly assigned to one of the four study arms and the investigational product will be dispensed for a 14-day course of dosing. The first intake of the IP should take place at the clinic during V3. The study product must be returned in its original packaging at Visit 4 to monitor and calculate IP-compliance.

Vitals, complete blood counts, clinical examination (measurement of body temperature, blood pressure, heart rate, respiratory rate, and weight) will be measured at this visit. Delegated research site staff will review the daily diary. Participants will be taking the first dose of the study product in the clinic after their blood collection and will be instructed on at-home dosing. Participants will receive a second daily study diary and be instructed on its accurate completion to capture IP administration, adverse events and concomitant medication use.

Blood samples to be collected will be analyzed for the following tests:

CBC [Includes: White Blood Cell Count (WBC), Red Blood Cell Count (RBC), Hemoglobin (HB), Hematocrit (HCT), MCV (mean corpuscular volume), MCH (mean corpuscular hemoglobin), MCHC (mean corpuscular hemoglobin concentration), Platelet Count]

iRepertoire mRNA lymphocyte CDR-3 sequencing

Plasma for potential future analysis

The results of CBC will be assessed by the PI. The clinical chemistry parameters must be within the reference value ranges and/or deemed by the PI as not clinically significant. Otherwise, the participant will be contacted by the PI or delegated site staff to further assess and determine an appropriate course of action.

The participant will proceed to Visit 4 at the research site.

### 3.15(e) Visit 4 (V4)—Follow up—Day 15+1*

Participants must be at least 2 hours fasted (no food or drink except water) prior to this visit. This visit will take place the day after the last dose administration. The study product must be returned in its original packaging (used and unused) at this visit to monitor compliance.

Vitals, complete blood counts, clinical examination (measurement of body temperature, blood pressure, heart rate, respiratory rate, and weight) will be assessed at this visit. Delegated research site staff will review the daily diary.

Blood samples to be collected will be analyzed for the following tests:

CBC [Includes: White Blood Cell Count (WBC), Red Blood Cell Count (RBC), Hemoglobin (HB), Hematocrit (HCT), MCV, MCH, MCHC, Platelet Count]

iRepertoire mRNA lymphocyte CDR-3 sequencing

Plasma for potential future analysis

The results of CBC will be assessed by the PI. The clinical chemistry parameters must be within the reference value ranges and/or deemed by the PI as not clinically significant. Otherwise, the participant will be contacted by the PI or delegated site staff to further assess and determine an appropriate course of action.

The participant will proceed to Visit 5 at the research site.

*Delegated site staff will aim to schedule participants on the target date. Under extenuating circumstances one additional day is provided to accommodate scheduling.

### 3.15(f) Visit 5 (V5)—End of Study—Day 22+1*

Participants must be at least 2 hours fasted (no food or drink except water) prior to this visit. Delegated research site staff will review the daily diary.

Safety, vitals, complete blood counts, clinical examination (measurement of body temperature, blood pressure, heart rate, respiratory rate, and weight) will be assessed at this visit.

Blood samples to be collected will be analyzed for the following tests:

CBC [Includes: White Blood Cell Count (WBC), Red Blood Cell Count (RBC), Hemoglobin (HB), Hematocrit (HCT), MCV, MCH, MCHC, Platelet Count]

Glucose

Sodium

Potassium

Creatinine

Alanine Amino Transferase (ALT)

Alkaline Phosphatase

Bilirubin Total

Urea

Serum protein

Albumin

Gamma Glutamyl Transpeptidase (GGT)

Calcium

Phosphorus

Aspartate Amino Transferase (AST)

iRepertoire mRNA lymphocyte CDR-3 sequencing

Plasma for potential future analysis

The results of the clinical chemistry will be assessed by the PI. The clinical chemistry parameters must be within the reference value ranges and/or deemed by the PI as not clinically significant. Otherwise, the participant will be contacted by the PI or delegated site staff to further assess and determine an appropriate course of action.

*Delegated site staff will aim to schedule participants on the target date. Under extenuating circumstances one additional day is provided to accommodate scheduling.

### 3.16 Clinical Assessments and Procedures

Height and Weight

Height and weight measurements will be performed as per the site's SOPs.

Participants will be weighed on a calibrated scale at specified visits.

Blood Pressure and Heart Rate

Blood pressure will be measured at each visit. The blood pressure measurement will be done according to the site's applicable SOP. Appropriately calibrated equipment will be used at every visit to measure blood pressure and heart rate. As required, a manual blood pressure cuff may be used by the PI or the delegated study staff.

Blood Sample Collection

Blood will be drawn by delegated site personnel into PAXgene tubes (for iRepertoire testing) or EDTA vacutainers (for CBC) from participants as per schedule of protocol assessments and in accordance with SOPs. PAXgene samples will be stored at −20° C. following collection and processing, until they are shipped to the Sponsor's analytical site for analysis. Clinical chemistry samples for safety and eligibility parameters will be collected and stored, refrigerated, or as otherwise indicated in the Laboratory Manual, until pick-up by local lab (daily).

All back up samples collected for potential analysis of iRepertoire mRNA lymphocyte CDR-3 sequencing will be stored as per Laboratory Manual specifications for each test, at the research site until requested by the Sponsor for analysis, or until the research site is instructed to destroy the samples by the Sponsor.

Plasma samples will be collected for potential future exploratory analysis by the Sponsor after study completion. These samples will be stored at −80° C. following collection and processing, until they are shipped to the Sponsor's analytical site for analysis.

### 3.17 Compliance

The participant will receive counselling and reminders pertaining to IP compliance and protocol requirements from delegated research site personnel.

Each participant will be provided with a daily study diary enabling them to record the daily intake of the IP, any concomitant medications, and record the occurrence of any adverse events. The participant will be required to return the IP to the research site where the number of capsules actually consumed will be assessed and IP compliance will be calculated using the following formula:

(%) as [(doses taken/doses intended)×100]

Based on these elements, the level of compliance will be assessed and documented.

Calculated IP compliance outside of 70-120% will be treated as a protocol deviation.

### 3.18 Laboratory Analysis

All laboratory samples (including back-up and future analysis samples) will be collected, processed and stored until shipment or destruction, by delegated personnel.

### 3.19 Back-up iRepertoire Samples

Back-up samples will be retained at the site for iRepertoire samples collected at Visits 2-5. Any other tests conducted will not require a back-up sample. iRepertoire mRNA lymphocyte CDR-3 sequencing analysis will be performed by an external analytical site (Sponsor's analytical site).

### 3.20 Clinical Chemistry

Clinical chemistry parameters will be analyzed by a local analytical laboratory. For each continuous laboratory parameter, results will be assessed by the Principal Investigator. All out-of-range and clinically significant laboratory results will be identified on the lab report by the analytical laboratory and/or PI, documented as adverse events as applicable.

### 3.21 Future Analysis

Any potential plasma analysis (retained from an EDTA sample collected at V2-V5) will be performed by an external analytical site (Sponsor's analytical site). An EDTA tube will be processed and plasma retained for potential future analysis at the Sponsor's request.

### 3.22 Termination of the Trial

The trial may be terminated at any time by the Sponsor, PI or applicable regulatory authority (IRB). If the trial is terminated prematurely, the PI, participants, and the IRB must all be notified of the termination promptly. Upon termination of the trial whether premature, or due to completion, site-closeout activities will be initiated including regulatory closeout from IRB.

### 3.23 Safety Measurements

Adverse Events (AEs)

The term “Adverse Event” as used herein refers to An unexpected problem that happens during the study period with an investigational product. Adverse event (AE) may be mild, moderate, or severe, and may be caused by something other than the drug or therapy being given. Adverse events must be followed up to resolution or when the condition is deemed stable by the PI.

### 3.24 Collecting, Recording, and Reporting of AEs

### 3.24(a) Collection

The PI or delegated study staff must record all adverse events in an AE form with information about:
Details of Adverse event
Date of onset (time can be recorded, if applicable)
Intensity (mild, moderate, severe)
Causal relationship to trial involvement (probable, possible, unlikely, not related)
Other actions taken
Date and time of outcome
Outcome

### 3.24(b)Reporting

The following timelines apply to the reporting of AEs/SAEs as applicable:
(1) Within 24 hours of the PI becoming aware of the event if it results in death of a participant.
(2) Within 2 weeks (10 business days) after becoming aware of the event if:

It is an AE which is related to the conduct of the study;

It is an AE that is expected (listed in the ICF as a potential side effect) but is occurring more frequently than expected;

It is an unexpected AE/SAE that is related to the conduct of the study but is not life-threatening.

(3) Annually (together with the Study Status Report) if:

It is an expected AE (listed in the ICF as a potential side effect);

It is an unexpected AE that is unlikely to be related to the conduct of the study and is not life-threatening.

### 3.24(c) Serious Adverse Events

The term “serious adverse event (SAE)” or “reaction” as used herein is any untoward medical occurrence that at any dose:

Results in death;

Is life-threatening;

Requires inpatient hospitalization;

Results in persistent or significant disability/incapacity or;

Is a medical event that may jeopardize the patient/participant and may require medical or surgical intervention.

During the course of the clinical trial, the Sponsor/CRO shall notify the Minister of Health of any serious adverse reaction and any serious unexpected adverse reaction to the natural health product that has occurred as follows:

(a) if it is neither fatal nor life threatening, within 15 days after the day on which the Sponsor/CRO becomes aware of the information; and

(b) if it is fatal or life threatening, within 7 days after the day on which the Sponsor/CRO becomes aware of the information.

The Sponsor/CRO shall, within 8 days after the day on which the Minister is notified, provide the Minister with a complete report in respect of that information that includes an assessment of the importance and implication of any findings made.

### 3.24(d)Laboratory Test Abnormalities

Occurrences of adverse events (serious and non-serious) will be assessed by the PI and/or delegate. For this purpose, a blood sample will be collected along with a clinical examination, an interview of the participant for changes in health or concomitant medications, and a review of the participant's daily diary will be carried out to study the hemodynamic parameters and assess the possible occurrence of adverse events, respectively.

At the discretion of the PI, participants may be removed from the study for safety and ethical concerns or any other reason which negatively affects the trial outcomes.

### 3.24(e) Statistical Evaluation

Determination of Sample Size

32 participants will be participating in the first period of this study. The sample size was chosen based on the minimum number of participants for a prospective pilot study and was not calculated based on a power analysis. This sample size will provide sufficient information to study the effects of each IP and to assess immune repertoire changes. 32 participants will be participating in the second period of this study, if necessary, based on an adaptive study design.

### 3.24(f) Study Population

All participants who complete the study without any major protocol violations which would render the data unreliable, will be included in the exploratory data analysis.

### 3.25 Analysis Plan

### 3.25(a) Statistical Methodology—Exploratory Data Analysis (EDA)

An EDA approach will be used to analyze data sets and descriptive statistics will be performed to conduct product development and for hypothesis generation for future studies. Data collection and cleaning will be performed to formulate assumptions to explore the effect of the mentioned NHP at approved doses on laboratory measurements of immune diversity through a set of graphical and quantitative techniques for finding patterns in data and determine optimal factor settings. The quality of data for further processing and cleaning will be checked before EDA is performed. EDA will be performed through the following steps that may include, but not be limited to:

Preview data

Check total number of entries and column types

Check any missing values

Check duplicate entries

Plot distribution of numeric data (univariate and pairwise joint distribution), if necessary

Plot count distribution of categorical data, if necessary

Analyze time series of numeric data, if necessary

### 3.25(b)Protocol Deviations

Protocol Deviation (PD) Forms must be filed for any unintended deviation from this protocol. PD's which place participants at increased risk of harm, or affect data integrity may be considered Protocol Violations (PVs) and must be reported to the IRB no later than 2 weeks (10 business days) from the time of identification. PDs will be filed in the applicable participant chart, and in the Trial Master File upon study closeout. PDs will be filed and signed by delegated study staff and reviewed and signed by the PI. The PI will determine if the PD is reportable to the IRB based on an assessment of the participant's safety and/or the integrity of the study data.

### 3.25(c) Protocol Amendments

Any changes to the protocol must be tracked and documented in the form of an amendment after it has been reviewed by the IRB. The reasons for change must be documented in writing and provided to the IRB and included in the Trial Master File. These changes will be subject to IRB approval prior to implementation. All amendments will be documented in the study report.

### 3.25(d)Data Collection and Storage

The PI and or delegated site staff agree to maintain accurate Case Report Forms (CRFs) and source documentation. Source documents are the originals of any documents which will be used by the Investigator that allow verification of the existence of the participant and substantiate the integrity of the data collected during the trial.

Either paper or electronic CRFs will be provided for each participant. CRFs will be completed only by persons delegated by the PI. Corrections will be made so as not to obliterate original data and will be identified and dated by the person who made the correction. The PI will allow designated representatives and regulatory bodies to have direct access to the source documents to verify the data reported in the CRFs.

### 3.25(e) Ethical Considerations

IRB Approval

All necessary forms, advertisements, and subject-facing study documents will be compiled into a submission to an Institutional Review Board (IRB) for approval prior to the conduct of the study. No conduct of the trial will commence until written approval has been obtained from the IRB. The requirements of the IRB must be adhered to and the IRB must be notified of any study document changes, protocol amendments, and protocol deviations/violations. Study termination must be reported to the IRB, and renewal of study approval must be obtained annually (or as per IRB's stipulations).

Informed Consent Form

Informed consent will be obtained from participants by delegated study staff. The staff will explain the study and review each page of the consent document. Participants will then be asked to review the consent document, initial each page, and sign in the appropriate section(s). The Informed Consent Form (ICF) will contain pertinent study details, a statement indicating the participant is free to withdraw from the study at any point and for any reason, contact information of the IRB (to report ethical concerns), local and applicable regulations surrounding disclosure of personal and health information of the participants, and a section explaining the potential risk(s) of participating in the study.

Risks and Procedures to Minimize Risk

Potential risks are disclosed to study participants in the ICF prior to their participation in the study. The potential risks associated with the study include venipuncture and associated risks. The risks of venipuncture (at the site) include:

Pain

Bruising

Infection

All venipunctures will be performed by certified phlebotomists and all applicable procedures will be carried out to minimize risk of infection.

### 3.26 Quality Assurance and Quality Control

Auditing

An independent, third-party clinical trial management system (CTMS) software vendor may be used by the site for laboratory report data and participant-associated document storage. All activities and actions performed on the vendor platform will be tracked and will produce an electronic audit-trail in accordance to regulatory standards. Paper documents will be used in the event that the CTMS system is not used due to any unforeseen or incidental circumstance causing disruption to data collection. All paper documentation will be subject to ICH-GCP E6(R2) and applicable guidelines.

All site-specific documentation generation and management, including documents shared with the Sponsor prior to the start of the study, follow internal SOPs for version control and approval for implementation. All procedural SOPs, reports and source documents that will be shared with the Sponsor during and after the study, including those that require changes during the course of the study, will be reviewed and approved by the appropriate site staff, in accordance with Site approval processes and standard procedures.

All material used in this clinical trial will be subject to quality control. This study will be conducted in accordance with ICH-GCP E6(R2) and all applicable Regulations and local laws. Quality assurance audits may be performed by the Sponsor or any health authority during the course of the study or after its completion.

The Investigator agrees to comply with the Sponsor and regulatory requirements for auditing the study. This includes access to the source documents for source data verification and confirmation.

Monitoring

Prior to the start of the study, the Sponsor representatives, site personnel and any third-party vendor representatives will hold at least one meeting to go over the details of the study design and plans for study execution. During the study, the Sponsor may arrange the visit of appointed site monitors to ensure that the execution of the study plan by the site meets the Sponsor's expectations and follows the study protocol objective. The site's delegated staff will monitor the conduct of the study and documentation throughout the course of the trial.

### 3.27 Data Management

Source Data and Source Documents

As defined by the International Conference on Harmonization (ICH), source data are defined as all information in original records and certified copies of original records of clinical findings, observations, or other activities in a clinical study necessary for the reconstruction and evaluation of the study.

All source data and source documents will be stored and archived according to local regulatory requirements. For this study, source data and documents include, but are not limited to:

Signed and dated Informed Consent Form (ICF);

Name, gender, date of birth and other personal information/demographic information;

Participant ID;

Date and time of each visit;

All clinical measurements and laboratory results;

Status of participant throughout the study;

Paper-based daily diary (to record any concomitant medications, pre-emergent adverse events, changes in health, dosing time, and adverse events);

Reason for discontinuation/withdrawal, if applicable.

Electronic Case Report Form (e-CRF)

An e-CRF system, provided by an independent third-party vendor, may be used to capture laboratory results and other participant data. Prior to deployment for the study, the e-CRF system will be validated and specified to address source documentation, in accordance with Sponsor and regulatory requirements. Paper CRFs will be used in the event that the eCRFs cannot be used (see section 16.1).

Data Storage and Access

Data will be entered into the system, checked for discrepancies and queried for any issues in accordance to site-approved SOPs. The database housing the e-CRF input will be hosted by the e-CRF vendor. Official corrections and/or modifications during the trial will be automatically tracked by an audit trail detailing the date, time and personnel involved/approving the modification of the e-CRF. All vendor data access and entry can only be performed by authorized users, using a unique user login and password. Login activity and data entry will be tracked in an automated audit trail. The Sponsor and dicentra will permit trial-related monitoring, audits, IRB review, and regulatory inspections, providing direct access to source data/documents. Paper CRFs will be monitored for completion, queried through internal review, identified items resolved and documented where applicable, and any required data from source document will be entered into the final database prior to locking.

Data Quality Assurance

Data cleaning will be performed to check for completeness and consistency of data using both programmed and manual edit checks. Discrepancies in data will be resolved through querying, delegation and resolution in accordance to site SOPs. All actions changing original data collection will be recorded using an electronic audit trail or done manually for paper CRFs according to ICH-GCP E6(R2).

Confidentiality of Participant Data

The PI will ensure that the confidentiality of the participant's data will be preserved to the extent permitted by law. In any documents collected after enrollment into study, the participants will only be identified by their participant ID; the ID consists of the participant's initials and an assigned number related to the sequence of received informed consent for the study. Documents that house participant information, such as signed Informed Consent Forms and personal/demographic forms, will be maintained and stored by delegated site staff under strict access.

### REFERENCES

[1] J. C. Gorski et al., “The effect of echinacea (Echinacea purpurea root) on cytochrome P450 activity in vivo,” Clin. Pharmacol. Ther., vol. 75, no. 1, pp. 89-100, January 2004, doi: 10.1016/j.clpt.2003.09.013.
[2] J. A. Carrillo and J. Benitez, “Clinically significant pharmacokinetic interactions between dietary caffeine and medications,” Clinical Pharmacokinetics, vol. 39, no. 2. Adis International Ltd, pp. 127-153, 2000, doi: 10.2165/00003088-200039020-00004.
[3] C. Lopez-Otin, M. A. Blasco, L. Partridge, M. Serrano, and G. Kroemer, “The hallmarks of aging,” Cell, vol. 153, no. 6. Cell Press, p. 1194, Jun. 6, 2013, doi: 10.1016/j.cell.2013.05.039.
[4] A. G. S. C. Jansen, E. A. M. Sanders, A. W. Hoes, A. M. Van Loon, and E. Hak, “Influenza- and respiratory syncytial virus-associated mortality and hospitalisations,” Eur. Respir. I, vol. 30, no. 6, pp. 1158-1166, December 2007, doi: 10.1183/09031936.00034407.
[5] T. Guichelaar et al., “Diversity of aging of the immune system classified in the cotton rat (Sigmodon hispidus) model of human infectious diseases,” Dev. Comp. Immunol., vol. 82, pp. 39-48, May 2018, doi: 10.1016/j.dci.2017.12.026.
[6] T. Fuchs et al., “The neutrophil recombinatorial TCR-like immune receptor is expressed across the entire human life span but repertoire diversity declines in old age,” Biochem. Biophys. Res. Commun., vol. 419, no. 2, pp. 309-315, March 2012, doi: 10.1016/j.bbrc.2012.02.017.
[7] O. V. Britanova et al., “Age-Related Decrease in TCR Repertoire Diversity Measured with Deep and Normalized Sequence Profiling,” J. Immunol., vol. 192, no. 6, pp. 2689-2698, March 2014, doi: 10.4049/jimmuno1.1302064.
[8] ImmunoFlex, “About ImmunoFlex—ImmunoFlex.” https://www.immunoflex.com/about-immunoflex.
[9] P. Pallmann et al., “Adaptive designs in clinical trials: Why use them, and how to run and report them,” BMC Medicine, vol. 16, no. 1. BioMed Central Ltd., p. 29, Feb. 28, 2018, doi: 10.1186/s12916-018-1017-7.
[10] V. Cox, Exploratory Data Analysis What Data Do I Have? Apress, Berkeley, Calif., 2017.
[11] A. M. Egorov, M. M. Ulyashova, and M. Y. Rubtsova, “Bacterial enzymes and antibiotic resistance,” Acta Naturae, vol. 10, no. 4. Acta Naturae, pp. 33-48, 2018, doi: 10.32607/20758251-2018-10-4-33-48.
[12] E. Rosati, C. M. Dowds, E. Liaskou, E. K. K. Henriksen, T. H. Karlsen, and A. Franke, “Overview of methodologies for T-cell receptor repertoire analysis,” BMC Biotechnol., vol. 17, no. 1, p. 61, July 2017, doi: 10.1186/s12896-017-0379-9.

### 3.28 Analysis Measures

Population diversity of the TCR repertoire can be quantitatively expressed by two separate factors: richness (i.e., the number of unique elements in a population) and evenness (i.e., the frequency distribution of those elements). The simplest diversity measure is species richness, which reflects the total number of species (for example, V-J rearrangements, CDR3 amino acid or nucleotide sequences; however, species richness does not take into account the relative frequency of each clonotype, thus resulting in an inadequate description of the repertoire diversity; in fact, two populations can have the same number of clonotypes (species richness), but each of them can be present with a different frequency in the TCR repertoire. [Aversa, I. et al. Intl J. Mol. Sci. (2020) 21: 2378].

Diversity measurements. The term “diversity Index” as used herein can be understood as follows. If the sample has more than ten thousand unique CDR3s, only the top 10 thousand unique CDR3s (and their respective read counts) are used. This is the case for all of our samples. A curve is created by graphing the percentage of total reads on the y-axis (scale 0 to 100) and the fraction of uCDR3s on the x-axis (scale 0 to 1) from highest frequency uCDR3 to the 10,000 uCDR3. The line y=100x represents the highest entropy distribution—would be normalized to y=x on if both axes were scaled from 0 to 1. The area between the line y=100 and the curve is the Diversity Index. The more the curve approaches the line y=100x, the more diversity the distribution with the maximum value of the Diversity index equal to 50.

Some diversity indices have been introduced that take into account both richness and evenness. These indices are all related to the Renyi entropy, a family of diversity measures initially developed for information theory, which quantifies the uncertainty in predicting the sequence identity of a random sequence from a dataset [Id., citing Mora, T W and Walczak, A. arXiv (2016) arXiv: 604.00487v00481]. When Renyi entropy function is applied to clonotype frequencies, it gives rise to a number of parameterized indices, each capturing different part of the distribution of clonotype frequencies data, i.e., some of them rely very strongly on correctly capturing the tail of rare clonotypes, while other measures systematically down-weight or undercount rarer clones [Id., citing Mora, T W and Walczak, A. arXiv (2016) arXiv: 604.00487v00481]. Therefore, these indices provide complementary information on the size-frequency distribution of clonotypes in the population, and they can be selectively used depending on the biological demand being addressed.

For example, the Shannon diversity index (Shannon's entropy) is defined as:

H=−Σᵢ₌₁^{N}pi ln pi [Formula VI]

where pi is the proportion of sequence i relative to the total N sequences. It accounts for both richness of the sample (i.e., the number of unique TCR/CDR3 sequences) and relative abundance (evenness) [Id., citing Robins, H S et al. Blood (2009) 114: 4099-4107; Carlson, C S et al. Nat. commun. (2013) 4: 2680]. A large Shannon diversity index reflects a more diverse distribution of the CDR3 sequences.

The comparison between different samples using the Shannon diversity index assumes that the distributions of the clonal frequencies of the samples are similar to each other.

The Pielou's evenness index represents a normalization of the Shannon diversity index by division of log 2 of the number of unique productive sequences:

J=H/log(S) [Formula VII]

where H is the Shannon index and S is the number of unique TCR/CDR3 sequences. The Pielou's evenness index thus allows for comparisons between samples differing in the total number of reads: high evenness (on a scale of 0 to 1) implies an almost uniform distribution, whereas low evenness is indicative of population skewing due to the biased expansion of individual T cell clonotypes.

The Inverse Simpson index is the effective number of types that is obtained when the weighted arithmetic mean is used to quantify average proportional abundance of types in the dataset of interest: High values indicate even distribution of TCR clones, and low values indicate enrichment of T cell clones.

Repertoire diversity can be also assessed using clonality scores, the simplest one being the clonal proportion, that is the fractional (percentage) composition of an individual clonotype relative to the total number of clonotypes. Most commonly, clonality scores derived from Shannon's entropy clonality can be calculated from entropy of the clonotypes frequency distribution (Shannon's entropy), and then normalized by the log of richness. The inverted metric (1-normalized entropy) result in a clonality value that ranges from 0 (the most diverse repertoire, or polyclonal repertoire, that is every T-cell in a sample contains a unique TCR), to 1 (monoclonal distribution) [Id., citing Robins, H S et al. Blood (2009) 114: 4099-4107; Carlson, C S et al. Nat. Commun. (2013) 4: 2680].

The meaning of clonality statistics is just the inverse of the diversity statistics, such that higher clonality typically means lower diversity.

Gini coefficient, commonly used as a measure of income inequality in economics, also can be used to assess the inequality of clonotype distribution within a repertoire [Id., citing Thomas, P G et al. Proc. Natl Acad. Sci. USA (2013) 110: 1839-44].

Repertoire overlap, achieved by computation of specific statistics on clonotypes shared between given repertoires, also called “public” clonotypes” is the most common approach to measure repertoire similarity. The repertoire overlap can be used to measure the change between sequential experiments. A therapeutic effect on the TCR repertoire would result in a lower degree of repertoires overlap before and after therapy, as compared to the higher overlap expected for repoertoires not impacted by the therapy. The Morisita's distance to clone count distributions is often used to quantity the overlap between two populations; precisely, Morisita's distance is an inverse measure of overlap so that two population with the greatest overlap will have a minimal Morisita's distance, while two very different repertoires will show the maximum Morisita's distance [Greiff, V. et al. Trends Immunol. (2015) 36: 738-49]. This index has been frequently used to quantify the repertoire change between sequential experiments, e.g., the repertoire of a patient before and after treatment [Id., citing Cha, E. et al. Sci. Transl. Med. (2014) 6: 238ra270].

(ALICE) Antigen-specific Lymphocyte Identification by Clustering of Expanded sequences predicts TCRs involved in the immune response from single repertoire snapshots of single individuals, using sequence similarity. [Pogorelyy, M V et al. PLOS Biology/doi.org/10.1371/journal.pbio.3000314]. TCRs recognizing the same epitopes often have similar sequences. ALICE predicts TCRs and clusters involved in immune responses. However, highly similar TCRs may also arise regardless of their binding properties, by virtue of their high generation probability by V(D)J recombination with clusters of similar TCRs found even in naive repertoires. To correct for those naïve clusters, ALICE evaluates the number of similar sequences relative to the baseline expectation from V(D)J recombination statistics, allowing it to identify clusters of TCRs responding to the same antigen. When represented graphically, vertices are TCR clonotypes observed in the repertoire, and edges connect sequences differing by at most 1 CDR3 amino acid. Essentially, ALICE calculates clusters that are based on antigen/epitope recognition. The clusters are therefore associated with historical influences on the repertoire. For each TCR amino acid sequence in the data, ALICE uses a stochastic TCR recombination model [Pogorelyy, M V et al. PLOS Biology/doi.org/10.1371/journal.pbio.3000314, citing Murugan, A. et al. Proc. Natl Acad. Sci. USA (2012) 109 (40): 16161-6; Marcu, Q. et al. Nat. Commun. (2018) 9 (1): 561] to estimate the fraction of the repertoire composed of TCR variants, called neighbors', differing by at most 1 amino acid in their Complementarity Determining Region 3 (CDR3). This makes it possible to predict theoretically the number of neighboring clonotypes (nucleotide sequences) for each TCR under the null hypothesis of no antigen-driven TCR selection and identify TCRs with a significantly higher number of neighbors in the data than the null expectation. Such significant results are referred to as ALICE signatures or hits. [Pogorelyy, M V et al. PLOS Biology/doi.org/10.1371/journal.pbio.3000314].

There are 4 metrics from ALICE of potential use in our analysis.

Sig nodes: the number of nodes (amino acid sequences) in the network with significant hits. These can be thought of as the parent of a cluster. [Id.]

Sig. Int: the number of significant convergence group interactions (vectors between nodes in a network calculated only from significant hits). This can be thought of as a measure of relatedness between clusters. [Id.]

Sig.Shannons: Shannon's Diversity Index calculated only for the sequences that are significantly enriched in a dataset. Can be from ALICE. This is a straight up diversity calculation for the population of sequences within clusters. [Id.]

Sig.Clusters: is a direct measure of the number of clusters calculated within the repertoire's distribution of sequences. [Id.]

Trial Results

In the described trial, 21 people aged 60-70, 5 or 6 per group received one of 4 supplements. The demographics of the trial participants is shown in table 8.

TABLE 8
Demographics

| | | Curcumin | Echinacea | NAC | PEA |
|---|---|---|---|---|---|
| | | N = 5 | N = 5 | N = 6 | N = 5 |
| Gender | Female | 4 (80%) | 4 (80%) | 4 (67%) | 3 (60%) |
| | Male | 1 (20%) | 1 (20%) | 2 (33%) | 2 (40%) |
| Race | Black | 0 (0%) | 1 (20%) | 0 (0%) | 0 (0%) |
| | Un- | 0 (0%) | 0 (0%) | 1 (17%) | 0 (0%) |
| | known | | | | |
| | White | 5 (100%) | 4 (80%) | 5 (83%) | 5 (100%) |
| Age | | 83.0 (3.4) | 64.2 (3.9) | 66.0 (3.0) | 61.2 (1.1) |
| Weight, | | 66.0 (5.1) | 69.1 (8.8) | 69.8 (4.4) | 70.3 (14.0) |
| kg | | | | | |
| Height, | | 167.0 (7.9) | 166.3 (10.3) | 165.9 (5.3) | 170.0 (5.7) |
| cm | | | | | |
| BMI | | 23.8 (3.1) | 24.9 (1.0) | 35.4 (1.6) | 24.2 (3.6) |

A complete blood count (CBC) test measures several components and features of the blood, including: red blood cells (RBCs), which carry oxygen; white blood cells (WBCs), which fight infection; hemoglobin (Hb), the oxygen-carrying protein in RBCs; hematocrit (hct), the proportion of red blood cells to the fluid component, or plasma, in the blood; and platelets, which help with blood clotting. It is used to monitor overall health; to diagnose a medical condition, to monitor a medical condition, and to monitor a medical treatment to determine the effect of that treatment on blood counts. The following table shows normal complete blood count results for adults:

TABLE 9
Complete Blood Count Normal Results, Adults
(https://www.mayoclinic.org/tests-procedures/complete-
blood-count/about/pac-20384919, visited May 24, 2021).

| Measure | Males | Females |
|---|---|---|
| RBC count | 4.35-5.65 trillion cells/L* | 3.92-5.13 trillion cells/L |
| | (4.35-5.65 million | (3.92-5.13 trillion cells/mcL) |
| | cells/mcL**) | |
| Hemoglobin | 13.2-16.6 grams/dL*** | 11.6-15 grams/dL |
| | (132-166 grams/L) | (116-150 grams/L) |
| Hematocrit | 38.3-48.6 percent | 35.5-44.9 percent |
| WBC count | 3.4-9.6 billion cells/L | 3.4-9.6 billion cells/L |
| | (3,400 to 9,600 cells/mcL) | (3,400 to 9,600 cells/mcL) |
| Platelet count | 135-317 billion/L | 157-371 billion/L |
| | (135,000 to 317,000/mcL) | (157,000 to 371,000/mcL) |

*L = liter;
**mcL = microliter;
***dL = deciliter

Generally, results slightly outside the normal range may not be a cause for concern and may or may not require follow-up if a subject is otherwise healthy and has no signs or symptoms of illness,

CBC data plots for subjects treated with NAC are shown in FIG. 11A (WBCs), FIG. 11B (RBCs), FIG. 11C (Hb), FIG. 11D (HCT), FIG. 11E (MCV), FIG. 11F (MCH), FIG. 11G (MCHC), FIG. 11H (RDW), FIG. 11I (platelets), FIG. 11J (neutrophils), FIG. 11K (lymphocytes), FIG. 11L (monocytes), FIG. 11M (eosinophils), FIG. 11N (basophils), FIG. 110 (granulocytes), FIG. 11P(NRBCs).

CBC data plots for subjects treated with Curcumin are shown in FIG. 12A (WBCs), FIG. 12B (RBCs), FIG. 12C (Hb), FIG. 12D (HCT), FIG. 12E (MCV), FIG. 12F (MCH), FIG. 12G (MCHC), FIG. 12H (RDW), FIG. 12I (platelets), FIG. 12J (neutrophils), FIG. 12K (lymphocytes), FIG. 12L (monocytes), FIG. 12M (eosinophils), FIG. 12N (basophils), FIG. 120 (granulocytes), FIG. 12P(NRBCs).

CBC data plots for subjects treated with PEA are shown in FIG. 13A (WBCs), FIG. 13B (RBCs), FIG. 13C (Hb), FIG. 13D (HCT), FIG. 13E (MCV), FIG. 13F (MCH), FIG. 13G (MCHC), FIG. 13H (RDW), FIG. 13I (platelets), FIG. 13J (neutrophils), FIG. 13K (lymphocytes), FIG. 13L (monocytes), FIG. 13M (eosinophils), FIG. 13N (basophils), FIG. 130 (granulocytes), FIG. 13P(NRBCs).

CBC data plots for subjects treated with Echinacea are shown in FIG. 14A (WBCs), FIG. 14B (RBCs), FIG. 14C (Hb), FIG. 14D (HCT), FIG. 14E (MCV), FIG. 14F (MCH), FIG. 14G (MCHC), FIG. 14H (RDW), FIG. 14I (platelets), FIG. 14J (neutrophils), FIG. 14K (lymphocytes), FIG. 14L (monocytes), FIG. 14M (eosinophils), FIG. 14N (basophils), FIG. 140 (granulocytes), FIG. 124(NRBCs).

The Y axis of each graph is in variable units. The x axis represents visit number. Visit 1 is for screening. Visit 2 is 1 week before dosing starts. Visit 3 is the day dosing. Visit 4 is the day after 2 weeks of dosing. Visit 5 is 1 week after dosing stops. Variable codes are shown in Table 10.

TABLE 10
CBC variables

| Variable Code | Variable Definition | Variable Units |
|---|---|---|
| STUDY ID | Unique Study Subject Identifier | |
| WBC | White Blood Cell Count | 10*9/L |
| RBC | Red Blood Cell Count | 10*12/L |
| Hb | Hemoglobin | g/L |
| HCT | Hematocrit | L/L |
| MCV | Mean Corpuscular Volume | fL |
| MCH | Mean Cell Hemoglobin | Pg |
| MCHC | Mean Corpuscular | g/L |
| | hemoglobin concentration | |
| RDW | Red Cell Distribution Width | % |
| PLT | Platelet Count | 10*9/L |
| Neut | Neutrophils | 10*9/L |
| Lymph | Lymphocytes | 10*9/L |
| Mono | Monocytes | 10*9/L |
| Eos | Eosinophils | 10*9/L |
| Baso | Basophils | 10*9/L |
| Gran | Immature Granulocytes | 10*9/L |
| nRBC | Nucleated RBCs | /100 WBC |

There were no treatment-specific trends, or trends of any type in the CBC data.

FIG. 15 is a schematic of the trial dosing time line and events. Blood draws were on Day 0, Day 7, Day 21 and Day 29. Day 0 to Day 7 represents the lead-in period. From Day 7 to Day 21 the subjects received daily dosing. The final dose was on day 21. From day 22 to day 29 (i.e., 7 days after dosing stopped) is defined as the washout period, there was no dosing during this period.

The collected data examined the frequency distributions of CDR3s in order to identify correlations between treatment and immune diversity. D50 is a measure of the diversity in the first half of the repertoire when looking at the frequency distributions. For example, an imaginary sample in which all unique CDR3s exist at the same frequency would have a maximum diversity index of 50. The closer the diversity index is to 50, the more diverse the repertoire. The second half of the distribution contains largely naïve populations with a mostly randomized somatic recombination process.

The trial did not include experiments to determine causality.

FIG. 16 is a schematic depicting the correlation between the trial dosing time line and events and the time line and events using bar graphs that plot percent difference value (y-axis) vs. sample states (x axis). Sample states are represented by Intervals 1 through 6. Interval 1 is from Day 0 to Day 7. Interval 2 is from day 0 to day 21. Interval 3 is from day 0 to day 29. Interval 4 is from day 7 to day 21 (i.e., daily dosing period). Interval 5 is from day 7 to day 29. Interval 6 is from day 21 to day 29.

FIG. 17 is a schematic depicting the correlation between the trial dosing time line and the diversity index plot of diversity index versus visit. The baseline points provide an idea of natural changes in the subjects. Dosing occurred one week after the lead-in baseline. The period one week after dosing stopped on day 21 represents the reorganization process.

The data address whether the subject's immune system reorganizes once the subject stopped taking the supplement, and if so, how. A summary of the results is shown in Tables 11 and 12 below.

TABLE 11
Trial results summary.

| Test compound | During Treatment | Post-Treatment |
|---|---|---|
| | Trend | |
| NAC | diversity increase | No effect |
| CUR | diversity increase | Reorganization |
| PEA | no effect | Reorganization |
| ECH | no effect | No effect |

TABLE 12
Trial results by measure

| Trial | Trial | Effect During | Post-Treatment |
|---|---|---|---|
| compound | measure | Treatment | Effect |
| NAC | Diversity | Up in sync | NS |
| | index (DI) | w/ treatment | |
| | Clonality¹ | Up in sync | NS |
| | | w/ treatment | |
| | Alice nodes² | Up in sync | NS |
| | | w/ treatment | |
| | Alice clusters | Up in sync | NS |
| | | w/ treatment | |
| CUR | Diversity | Up in sync | NS |
| | Index (DI) | w/ treatment | |
| | Clonality | NS | Down w/ |
| | | | reorganization |
| | Alice nodes | NS | Down w/ |
| | | | reorganization |
| | Alice clusters | Up in sync | Down w/ |
| | | w/ treatment | reorganization |
| PEA | Diversity | NS | Up with |
| | Index (DI) | | reorganization |
| | Clonality | NS | Down with |
| | | | reorganization |
| | Alice nodes | NS | NS |
| | Alice clusters | NS | Up with |
| | | | reorganization |
| ECH | Diversity | NS | NS |
| | Index (DI) | | |
| | Clonality¹ | NS | NS |
| | Alice nodes² | NS | NS |
| | Alice clusters | NS | NS |

NS: not significant

N-acetylcysteine (NAC) Results

FIG. 18 shows a bar graph of mean Diversity Index/percent difference on the Y axis. The X axis corresponds to visit number-time periods in days (d) (from left to right, visits 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). At time periods 20 d to 21 d and 47 d to 21 d, the increase in diversity was up in sync during treatment with N-acetylcysteine (NAC).

FIG. 19 shows a plot of the calculated Diversity Index vs. visit number for an example participant who was treated with N-acetylcysteine (NAC). The Diversity Index is equal to 100 minus the area under the curve in a normalized frequency distribution curve plotting the percentage of total reads and the percentage of unique CDR3s, when unique CDR3s are sorted, by frequency, from largest to smallest. Visit 2 was 1 week prior to dosing, Visit 3 was immediately prior to the start of dosing, Visit 4 was after 2 weeks of daily dosing N-acetylcysteine 600 mg twice daily (1200 mg total daily dose), and Visit 5 was 1 week after dosing was stopped. FIG. 19B shows corresponding tree maps for each of visits 2, 3, 4, & 5. Each spot in the plot represents a unique CDR3 and the size of a spot denotes the relative frequency.

Diversity index. 4 out of 4 participants receiving NAC showed the same trend of Diversity Index increasing in sync with the supplement protocol (points 2, 4). On day 21 (the day blood was taken after 2 weeks of supplement) Diversity Index was up significantly. NAc therefore increased diversity of the first 50% of a person's repertoire. Diversity Index on Day 29 (after stop taking the supplement) goes down. In summary, the immune repertoire became more diverse because treatment flattened out the first half of the repertoire. Some of the high occurring clones were lost but the immune system gained diversity of the distribution. These are CDRs being replicated in higher frequency than naïve, i.e., NAC is boosting the robustness of the past history presentation. When the supplement is stopped and the last sample taken, the data has more to do with reorganization of the immune system.

Clonality.

FIG. 20 shows a bar graph of mean Clonality percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). Clonality is calculated as (1—the Shannon Equitability Index). The Shannon Equitability Index is the ratio of the Shannon Diversity to the Shannon Entropy. The closer this number is to 1, the closer the distribution is to the most entropic distribution, i.e., all uCDR3s at frequency of 1. The lower the clonality, the closer to this distribution. A positive change in clonality represents a movement from the most entropic distribution. This tracks, trendwise, inversely with the change in unique CDR3s (uCDR3s). A specific unique CDR3 may have 1 copy in a sample or may have thousands of copies. At time periods 0 d to 21 d and 7 d to 21 d, the increase in mean clonality was up in synch during treatment with N-acetylcysteine (NAC).

ALICE Results.

FIG. 21 is a bar graph showing mean ALICE significant nodes percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). The number of nodes represents the total number of unique CDR3 amino acid sequences within the networked sample. Unique sequences that do not cluster are not counted. “Significant nodes” are the number of notes with significant interactions. At time periods 0 d to 21 d and 7 d to 21 d, the increase in mean nodes was up in synch during treatment with N-acetylcysteine (NAC).

FIG. 22 is a bar graph showing mean ALICE significant clusters ALICE percent difference on the Y axis. This is a direct measure of the number of clusters calculated within the repertoire's distribution of sequences. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). At time periods 0 d to 21 d and 7 d to 21 d. the increase in mean clusters was up in synch during treatment with N-acetylcysteine (NAC).

The results show a flattening and an increase in similarity in the first 50% of the distribution, which shows the footprint of the subject's immune memory component, which indicates that the memory aspect is being more heavily employed in the first 50% of the distribution. Therefore, as the flattened out along distribution of the first 50%, are getting a more even distribution of chemically similar CDR3s. The redistribution means some of the higher frequency CDR3s decrease, and some increase. This means that the subject's immune system is deploying in a more even way more historical information associated with the experiential immune history, germ line memory or both. Without being limited by theory, the data show that NAC treatment boosts the immune system, i.e., oxidative stress is being decreased and the immune cells are working more efficiently, thus increasing their functional efficiency.

Curcumin (CUR) Results

Diversity Index.

FIG. 23 shows a bar graph of mean Diversity Index/percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). At time periods 0 d to 21 d and 7 d to 21 d, the increase in diversity was up in synch during treatment with Curcumin (CUR).

FIG. 24A shows the calculated Diversity Index vs. visit number for an example participant who was treated with curcumin (CUR). The DI is equal to 100 minus the area under the curve in a normalized frequency distribution curve plotting the percentage of total reads and the percentage of unique CDR3s, when unique CDR3s are sorted, by frequency, from largest to smallest. Visit 2 was 1 week prior to dosing, Visit 3 was immediately prior to the start of dosing, Visit 4 was after 2 weeks of daily dosing curcumin 3×60 mg capsules once daily (180 mg total daily dose), and Visit 5 was 1 week after dosing was stopped. FIG. 24B shows corresponding tree maps for each of visits 2, 3, 4, & 5. Each spot in the plot represents a unique CDR3 and the size of a spot denotes the relative frequency.

Changes in Diversity Index in subjects receiving a Curcumin supplement were seen in the first 50% of the distribution. Like NAC, the greatest change in Diversity Index was seen at Visit 4.

Clonality. Clonality is calculated as 1—the Shannon Equitability Index. The Shannon Equitability Index is the ratio of the Shannon Diversity to the Shannon Entropy. The closer this number is to 1, the closer the distribution is to the most entropic distribution—all uCDR3s at frequency of 1. The lower the clonality, the closer to this distribution). A positive change in clonality represents a movement from the most entropic distribution.

FIG. 25 shows a bar graph of mean Clonality percent difference on the Y axis.

The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). Clonality at time points 0 d to 29 d, 7 d to 29 d, 21 d to 29 d was down on reorganization following treatment with Curcumin (CUR).

ALICE Results

FIG. 26 is a bar graph showing mean ALICE significant nodes percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). The number of nodes represents the total number of unique CDR3 amino acid sequences within the networked sample. Unique sequences that do not cluster are not counted. “Significant nodes” are the number of notes with significant interactions. At time periods 0 d to 29 d, 7 d to 29 d, 21 d to 29 d, there was a decrease in mean nodes on reorganization during treatment with Curcumin (CUR).

FIG. 27 is a bar graph showing mean ALICE significant clusters ALICE percent difference on the Y axis. This is a direct measure of the number of clusters calculated within the repertoire's distribution of sequences. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). Note that the cluster data for Curcumin is quite different from that of NAC. At time periods 0 d-21 d and 7 d to 21 d significant clusters are up in sync during treatment while at time periods 0 d to 29 d, 7 d to 29 d, and 21 d to 29 d significant clusters are down on reorganization. Whereas NAC and CUR cluster data support a diversity increase upon treatment, the cluster data for Curcumin (CUR) also supports reorganization of the repertoire post-treatment.

PEA Results

Diversity index.

FIG. 28 shows a bar graph of mean Diversity Index percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). At time periods 0 d to 29 d, 7 d to 29 d, 21 d to 29 d, the increase in diversity was up on reorganization post-treatment with palmitoylethanolamide (PEA).

FIG. 29A shows the calculated Diversity Index vs. visit number for an example participant who was treated with palmitoylethanolamide (PEA). The Diversity Index is equal to 100 minus the area under the curve in a normalized frequency distribution curve plotting the percentage of total reads and the percentage of unique CDR3s, when unique CDR3s are sorted, by frequency, from largest to smallest. Visit 2 was 1 week prior to dosing, Visit 3 was immediately prior to the start of dosing, Visit 4 was after 2 weeks of daily dosing PEA 3×400 mg capsules once daily (1200 mg total daily dose), and Visit 5 was 1 week after dosing was stopped. FIG. 29B shows corresponding tree maps for each of visits 2, 3, 4, & 5. Each spot in the plot represents a unique CDR3 and the size of a spot denotes the relative frequency.

The diversity index plot shows a huge increase in Diversity Index at visit 5, i.e., when the supplement is stopped. Accordingly, this data indicates that a big change in diversity occurs when PEA is stopped. Without being limited by theory, this is interpreted as showing reorganization of the immune system after taking this supplement.

Clonality:

Clonality is calculated as 1—the Shannon Equitability Index. The Shannon Equitability Index is the ratio of the Shannon Diversity to the Shannon Entropy. The closer this number is to 1, the closer the distribution is to the most entropic distribution—all uCDR3s at frequency of 1. The lower the clonality, the closer to this distribution). A positive change in clonality represents a movement from the most entropic distribution.

FIG. 30 shows a bar graph of mean Clonality percent difference on the Y axis. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). Clonality at time periods 0 d to 29 d, 7 d to 29 d, 21 d to 29 d was down on reorganization following treatment with palmitoylethanolamide (PEA).

Clustering.

FIG. 31 is a bar graph showing mean ALICE significant clusters ALICE percent difference on the Y axis. This is a direct measure of the number of clusters calculated within the repertoire's distribution of sequences. The X axis corresponds to time periods in days (d) (from left to right, 0 d to 7 d, 0 d to 21 d, 0 d to 29 d, 7 d to 21 d, 7 d to 29 d, and 21 d to 29 d, with 0 d=visit 2, 7 d=visit 3, 21 d=visit 4 and 29 d=visit 5). At time periods 0 d to 21 d, 7 d to 21 d clustering decreases. At time periods 0 d to 29 d, 7 d to 29 d, 21 d to 29 d there is a dramatic increase in clustering with reorganization following treatment with palmitoylethanolamide (PEA).

Echinacea Results

Diversity index.

FIG. 32A shows the calculated Diversity Index vs. visit number for an example participant who was treated with Echinacea pupurea (ECH). The Diversity Index is equal to 100 minus the area under the curve in a normalized frequency distribution curve plotting the percentage of total reads and the percentage of unique CDR3s, when unique CDR3s are sorted, by frequency, from largest to smallest. Visit 2 was 1 week prior to dosing, Visit 3 was immediately prior to the start of dosing, Visit 4 was after 2 weeks of daily dosing of echinacea-one 252 mg capsule twice daily (504 mg total daily dose), and Visit 5 was 1 week after dosing was stopped. FIG. 32B shows corresponding tree maps for each of visits 2, 3, 4, & 5. Each spot in the plot represents a unique CDR3 and the size of a spot denotes the relative frequency.

The data show that diversity of the CDR3s decreases with treatment with Echinacea. However, once Echinacea is stopped at visit 4, a huge boost in the diversity index was observed. This increase corresponds to a reorganizational change in memory CDRs. Detailed analysis of diversity metrics showed no significant trends across participants.

In summary, the trial data show two general phenomena: a direct correlation of the supplement to increased immune system diversity, exemplified by NAC and Curcumin; and an off-frequency correlation of the supplement to an increased immune system diversity, in which immune system diversity increases once the supplement is stopped, exemplified by PEA and Curcumin. Without being limited by theory, the off-frequency correlation stimulates a reorganizational change in the immune system and may exercise the immune system so that it stays vibrant.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for improving immune system health in a subject in need thereof comprising administering to the subject a composition comprising a therapeutic amount of an active constituent and a pharmaceutically acceptable carrier, wherein the active constituent comprises one or more of N-acetylcysteine, a botanical ingredient, or a cannabimimetic, wherein the therapeutic amount of the active constituent potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100% compared to a control.

2. The method according to claim 1, wherein the N-acetylcysteine directly potentiates the immune response by increasing immune diversity.

3. The method according to claim 1, wherein when the active constituent comprises N-acetylcysteine and a botanical ingredient or cannabimimetic the therapeutic effect of the N-acetylcysteine and of the botanical material or cannabinoid are complementary.

4. The method according to claim 1, wherein a therapeutic effect of the botanical material or cannabinoid is non-psychoactive.

5. The method according to claim 1, wherein the cannabimimetic is a terpinoid, a fatty acid derivative, a flavonoid, or is derived from derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin B1) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or Polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; or an essential oil selected from Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), or Ylang ylang (Cananga odorata(Lam.) var. genuine).

6. The method according to claim 1, wherein
(a) for each dose of N-acetylcysteine, onset of potentiation of the immune response by increasing diversity occurs within 24 hours of dosing; and
(b) for each dose of the botanical ingredient or cannabinimimetic, onset of potentiation of the immune response by increasing diversity occurs within 24 hours of dosing.

7. The method according to claim 1, wherein compared to the subject before the administering, the potentiated immune response comprises:
(a) an enhanced T cell diversity, or
(b) an enhanced B cell diversity, or
(c) an enhanced T cell diversity and an enhanced B cell diversity; or
(d) a stabilized T cell immune repertoire.

8. The method according to claim 1, wherein the potentiated immune response comprises an increased resistance to T cell exhaustion.

9. The method according to claim 1, wherein the potentiated immune response
(a) improves clinical outcome in response to a pathogen; or
(b) reduces a burden of disease; or
(c) reduces appearance of disease; or
(d) increases health span of the subject.

10. The method according to claim 9, wherein the pathogen is a microbe selected from a bacterium, a fungus, a protozoan, a virus, or an algae.

11. The method according to claim 1, wherein the therapeutic amount of the composition comprising N-acetylcysteine further comprises a mucolytic therapeutic effect, an anti oxidant therapeutic effect, or both.

12. The method according to claim 1, wherein the subject in need is an aged person of greater than 60 years of age.

13. The method according to claim 12, wherein the immune system of the aged person is compromised by one or more of prior illnesses, chronic illnesses, or the aging process.

14. The method according to claim 1, wherein the botanical ingredient or cannabimimetic is tetahydrocannabinol (THC) or curcumin.

15. The method according to claim 1, wherein the administering to the subject comprises alternating a composition comprising N-acetylcysteine with a composition comprising the botanical ingredient or cannabimimetic, wherein immune diversity of the immune system increases once the composition comprising the botanical ingredient or cannabimimetic is stopped.

16. The method according to claim 15, wherein the alternating is weekly.

17. The method according to claim 15, wherein the botanical ingredient or cannabimimetic comprises cannabidiol (CBD), palmitoylethanolamine (PEA), or Curcumin (CUR).

18. The method according to claim 15, wherein the alternating increases vibrancy of the immune system, stimulates a reorganizational change in the immune system, or both.

19. The method according to claim 15, wherein the subject is suffering from post-acute COVID-19 syndrome.

20. A method for treating symptoms of a respiratory virus infection, comprising administering to a subject in need thereof a composition comprising a therapeutic amount of an active constituent and a pharmaceutically acceptable carrier, wherein the active constituent comprises one or more of N-acetylcysteine, a botanical ingredient, or a cannabimimetic, and wherein the therapeutic amount of the active constituent potentiates an immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100% compared to a control.

21. The method according to claim 20, wherein the N-acetylcysteine directly potentiates the immune response by increasing immune diversity.

22. The method according to claim 20, wherein when the active constituent comprises N-acetylcysteine and a botanical ingredient or cannabimimetic the therapeutic effect of the N-acetylcysteine and of the botanical material or cannabinoid are complementary.

23. The method according to claim 20, wherein a therapeutic effect of the botanical material or cannabinoid is non-psychoactive.

24. The method according to claim 20, wherein the cannabimimetic is a terpinoid, a fatty acid derivative, a flavonoid, or is derived from, for example, turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin B1) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; or an essential oil selected from Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), or Ylang ylang (Cananga odorata(Lam.) var. genuine).

25. The method according to claim 20, wherein
a. for each dose of N-acetylcysteine, onset of potentiation of the immune response by increasing diversity occurs within 24 hours; and
b. for each dose of the botanical ingredient or cannabinimimetic, onset of potentiation of the immune response by increasing diversity occurs within 24 hours.

26. The method according to claim 20, wherein compared to the subject before the administering, the potentiated immune response comprises:
a. an enhanced T cell diversity, or
b. an enhanced B cell diversity, or
c. an enhanced T cell diversity and an enhanced B cell diversity; or
d. a stabilized T cell immune repertoire.

27. The method according to claim 20, wherein the potentiated immune response comprises an increased resistance to T cell exhaustion.

28. The method according to claim 20, wherein the potentiated immune response
a. improves clinical outcome in response to a pathogen; or
b. reduces a burden of disease; or
c. reduces appearance of disease; or
d. increases health span of the subject.

29. The method according to claim 28, wherein the pathogen is a microbe selected from a bacterium, a fungus, a protozoan, a virus, or an algae.

30. The method according to claim 20, wherein the therapeutic amount of the composition comprising N-acetylcysteine further comprises a mucolytic therapeutic effect, an anti-oxidant therapeutic effect, or both.

31. The method according to claim 20, wherein the subject in need is an aged person of greater than 60 years of age.

32. The method according to claim 31, wherein the immune system of the aged person is compromised by one or more of prior illnesses, chronic illnesses, or the aging process.

33. The method according to claim 20, wherein the therapeutic amount reduces viral load.

34. The method according to claim 20, wherein the botanical ingredient or cannabimimetic is tetramydrocannabinol (THC) or curcumin (CUR).

35. The method according to claim 20, wherein the administering to the subject comprises alternating a composition comprising N-acetylcysteine with a composition comprising the botanical ingredient or cannabimimetic, wherein immune diversity of the immune system increases once the composition comprising the botanical ingredient or cannabimimetic is stopped.

36. The method according to claim 35, wherein the alternating is weekly.

37. The method according to claim 35, wherein the botanical ingredient or cannabimimetic comprises cannabidiol (CBD), palmitoylethanolamine (PEA) or Curcumin (CUR).

38. The method according to claim 35, wherein the alternating increases vibrancy of the immune system, stimulates a reorganizational change in the immune system or both.

39. The method according to claim 35, wherein the subject is suffering from post-acute COVID-19 syndrome.

40. A method for increasing potency or efficacy of an antiviral vaccine in increasing a subject's resistance to a viral infection, comprising administering to the subject a composition comprising a therapeutic amount of an active constituent and a pharmaceutically acceptable carrier, wherein the active constituent comprises one or more of N-acetylcysteine, a botanical ingredient, or a cannabimimetic, and wherein the therapeutic amount of the active constituent potentiates an anti-viral immune response by increasing diversity of the immune repertoire comprising T cells and B cells of the subject by at least 10%, 15%, 20%, 25%, 30%, 35%, 40%, 45%, 50%, 55%, 60%, 65%, 70%, 75%, 80%, 85%, 90%, 95%, 100%, compared to a control.

41. The method according to claim 40, wherein the N-acetylcysteine directly potentiates the immune response by increasing immune diversity.

42. The method according to claim 40, wherein the antiviral vaccine is employed to help the subject's body's immune system recognize and fight infections caused by the virus in a susceptible population.

43. The method according to claim 40, wherein the virus is for example, a polio virus, a measles virus, a mumps virus, a rubella virus, an influenza virus, a rotavirus, a human immunodeficiency virus, a SARS coronavirus, or a rabies virus.

44. The method according to claim 40, wherein when the active constituent comprises N-acetylcysteine and a botanical ingredient or cannabimimetic the therapeutic effect of the N-acetylcysteine and of the botanical material or cannabinoid are complementary.

45. The method according to claim 40, wherein a therapeutic effect of the botanical material or cannabinoid is non-psychoactive.

46. The method according to claim 40, wherein the cannabimimetic is a terpinoid, a fatty acid derivative, a flavonoid, or is derived from turmeric, cawa, ginseng, frankincense, Astaxanthin, Panax quinquefolius (American ginseng), palmitoylethanolamine (PEA), zinc, DL-Phenylalanine (DLPA), Boswellic Acid (AKBA), Gamma aminobutyric acid (GABA), Acetyl-L-carni tine (ALC), Alpha lipoic acid (ALA), 5-hydroxytryptophan (5-HTP), Echinicaea, Lavender, and Melatonin. Further alternatives include Ashwagandha (root), St. John's Wort Extract (aerial), Valerian (root), Rhodiola Rosea Extract (root), Lemon Balm Extract (leaf), L-Theanine, Passion Flower (herb), cyracos, gotu kola, chamomile, skullcap, roseroot, ginkgo, Iranian borage, milk thistle, bitter orange, sage, L-lysine, L-arginine, Hops, Green Tea, calcium-magnesium, Vitamin A (beta carotene), Magnolia officinalis, Vitamin D3, Pyridoxal-5-phosphate (P5P), St John's wort, Cayenne, pepper, wasabi, evening primrose, Arnica Oil, Ephedra, White Willow, Ginger, Cinnamon, Peppermint Oil, Thiamin (Vitamin Bl) (as thiamin mononitrate), Riboflavin (Vitamin B2), Niacin (Vitamin B3) (as nicotinamide), Vitamin B6 (pyridoxine HCl), Vitamin B12 (cyanocobalamin), California Poppy, Mullein Verbascum thapsus (L.), Kava Piper methysticum (G. Forst.), Linden Tilia cordata (Mill.), Catnip Nepeta cataria (L.), Magnesium, D-Ribose, Rhodiola Rosea, caffeine, Branched-Chain Amino Acids Wheatgrass Shot, Cordyceps, Schisandra Berry, Siberian Ginseng (Eleuthero root), Yerba Mate Tea, Spirulina, Maca Root, Reishi Mushroom, Probiotics, Astragalus, He Shou Wu (Fallopia multiflora or polygonum multiflorum), Cola acuminata (Kola nut), Vitamin C, Centella asiatica (Gotu kola), L-tryosine, Glycine, Pinine, Alpha-pinene, SAMe, DHEA, Coenzyme QlO, glutathione; or an essential oil selected from Anise (Pimpinella anisum(L.)), Basil (Ocimum basilicum(L.)), Bay (Laurus nobilis(L.)), Bergamot (Citrus aurantium var. bergamia (Risso)), Chamomile (German) (Matricaria recutita(L.)), Chamomile (Roman) (Chamaemelum nobile (L.) All.), Coriander (Coriandrum sativum (L.)), Lavender (Lavandula angustifolia (Mill.)), Neroli (Citrus aurantium (L.) var. amara), Rose (Rosa damascena (Mill.)), Sandalwood (Santalum album(L.)), Thyme (Thymus vulgaris (L.)), Vetiver (Vetiveria zizanioides(Nash),) Yarrow (Achillea millefolium(L.)), or Ylang ylang (Cananga odorata(Lam.) var. genuine).

47. The method according to claim 40, wherein
a. for each dose of N-acetylcysteine, onset of potentiation of the immune response by increasing diversity occurs within 24 hours of dosing; and
b. for each dose of the botanical ingredient or cannabimimetic, onset of potentiation of the immune response by increasing diversity occurs within 24 hours of dosing.

48. The method according to claim 40, wherein compared to the subject before the administering, the potentiated immune response comprises:
a. an enhanced T cell diversity, or
b. an enhanced B cell diversity, or
c. an enhanced T cell diversity and an enhanced B cell diversity; or
d. a stabilized T cell immune repertoire.

49. The method according to claim 40, wherein the potentiated immune response comprises an increased resistance to T cell exhaustion.

50. The method according to claim 40, wherein the potentiated immune response
a. improves clinical outcome in response to a pathogen; or
b. reduces a burden of disease; or
c. reduces appearance of disease; or
d. increases health span of the subject.

51. The method according to claim 50, wherein the pathogen is a microbe selected from a bacterium, a fungus, a protozoan, a virus, or an algae.

52. The method according to claim 40, wherein the therapeutic amount of the composition comprising N-acetylcysteine further comprises a mucolytic therapeutic effect, an anti-oxidant therapeutic effect, or both.

53. The method according to claim 40, wherein the subject in need is an aged person of greater than 60 years of age.

54. The method according to claim 53, wherein the immune system of the aged person is compromised by one or more of prior illnesses, chronic illnesses, or the aging process.

55. The method according to claim 54, wherein the aging process comprises biological and physiological changes that result in increased susceptibility to the viral infection.

56. The method according to claim 40, wherein the botanical ingredient or cannabimimetic is tetrahydrocannabinol (THC) or curcumin (CUR).

57. The method according to claim 40, wherein the administering to the subject comprises alternating a composition comprising N-acetylcysteine with a composition comprising the botanical ingredient or cannabimimetic, wherein immune diversity of the immune system increases once the composition comprising the botanical ingredient or cannabimimetic is stopped.

58. The method according to claim 57, wherein the alternating is weekly.

59. The method according to claim 57, wherein the botanical ingredient or cannabimimetic comprises cannabidiol (CBD), palmitoylethanolamine (PEA) or Curcumin (CUR).

60. The method according to claim 57, wherein the alternating increases vibrancy of the immune system, stimulates a reorganizational change in the immune system, or both.

61. The method according to claim 57, wherein the subject is suffering from post-acute COVID-19 syndrome.
